(19) United States
(12) Patent Application Publication
(10) Pub. No.: US 2016/0002248 A1
(43) Pub. Date: Jan. 7, 2016

(54) GLS1 INHIBITORS FOR TREATING DISEASE

(71) Applicant: Board of Regents, The University of Texas System, Austin, TX (US)

(72) Inventors: Maria Emilia Di Francesco, Houston, TX (US); Timothy Heffernan, Sugar Land, TX (US); Michael J. Soth, Sugar Land, TX (US); Kang Le, Sugar Land, TX (US); Christopher Lawrence Carroll, Houston, TX (US); Timothy McAfoos, Houston, TX (US); Jason P. Burke, Houston, TX (US); Jay Theroff, Manvel, TX (US); Zhijun Kang, Sugar Land, TX (US); Philip Jones, Houston, TX (US)

(73) Assignee: BOARD OF REGENTS, THE UNIVERSITY OF TEXAS SYSTEM, Austin, TX (US)

(21) Appl. No.: 14/791,206

(22) Filed: Jul. 2, 2015

Related U.S. Application Data
(60) Provisional application No. 62/020,524, filed on Jul. 3, 2014.

(51) Int. Cl. C07D 487/04; A61K 45/06; A61N 5/10; A61K 31/437; A61K 31/506; A61N 7/00; A61K 31/5025; C07D 471/04

(52) U.S. Cl. CPC C07D 487/04; A61K 31/5025; A61K 45/06; C07D 471/04; A61K 31/437; A61K 31/506; A61N 7/00; A61N 5/10

(57) ABSTRACT

Disclosed herein are compounds and compositions useful in the treatment of GLS1 mediated diseases, such as cancer, having the structure of Formula I:

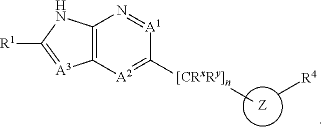

Methods of inhibition GLS1 activity in a human or animal subject are also provided.

This application claims the benefit of priority of U.S. provisional Application No. 62/020,524, filed Jul. 3, 2014, the disclosure of which is hereby incorporated by reference as if written herein in its entirety.

The present disclosure relates to new heterocyclic compounds and compositions, and their application as pharmaceuticals for the treatment of disease. Methods of inhibition of GLS1 activity in a human or animal subject are also provided for the treatment of diseases such as cancer.

Metabolic deregulation is a hallmark of cancer as tumors exhibit an increased demand for nutrients and macromolecules to fuel their rapid proliferation. Glutamine (Gln), the most abundant amino acid in circulation, plays an essential role in providing cancer cells with biosynthetic intermediates required to support proliferation and survival. Specifically, glutaminolysis, or the enzymatic conversion of glutamine to glutamate, provides proliferating cancer cells with a source of nitrogen for amino acid and nucleotide synthesis, and a carbon skeleton to fuel ATP and NADPH synthesis through the TCA cycle. In addition to supporting cell growth, glutamine metabolism plays a critical role in maintaining cellular redox homeostasis as glutamate can be converted into glutathione, the major intracellular antioxidant.

Glutaminolysis is regulated by mitochondrial glutaminase (GLS), the rate limiting enzyme that catalyzes the conversion of Gln to glutamate and ammonia. Mammalian cells contain 2 genes that encode glutaminase: the kidney-type (GLS1) and liver-type (GLS2) enzymes. Each has been detected in multiple tissue types, with GLS1 being widely distributed throughout the body. GLS1 is a phosphate-activated enzyme that exists in humans as two major splice variants, a long form (referred to as KGA) and a short form (GAC), which differ only in their C-terminal sequences. Both forms of GLS1 are thought to bind to the inner membrane of the mitochondrion in mammalian cells, although at least one report suggests that glutaminase may exist in the intramembrane space, dissociated from the membrane. GLS is frequently overexpressed in human tumors and has been shown to be positively regulated by oncogenes such as Myc. Consistent with the observed dependence of cancer cell lines on glutamine metabolism, pharmacological inhibition of GLS offers the potential to target Gln addicted tumors.

Thus, there is a need for glutaminase inhibitors that are specific and capable of being formulated for in vivo use.

### SUMMARY

Accordingly, the inventors herein disclose new compositions and methods for inhibiting glutaminase activity.

Provided is a compound of structural Formula I

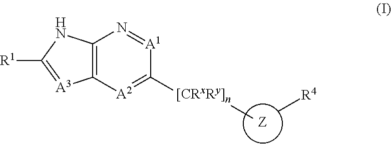

or a salt thereof, wherein: n is chosen from 3, 4, and 5; each R^{x} and R^{y} is independently chosen from alkyl, cyano, H, and halo, wherein two R^{x} groups together with the atoms to which they are attached optionally form a cycloalkyl ring; A¹ and A² are independently chosen from N and CH; A³ is chosen from N and CR²; R¹ is chosen from alkenyl, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, C(O)N(R³)₂, and C(O)C(R³)₃, wherein R¹ may be optionally substituted with between 0 and 3 R^{z} groups; R² is chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, C(O)N(R³)₂, C(O)C(R³)₃, C(O)OH, C(O)OC(R³)₃, wherein R¹ and R² together with the atoms to which they are attached optionally form an form an aryl, cycloalkyl, heteroaryl, or heterocycloalkyl ring, which may be optionally substituted with between 0 and 3 R^{z} groups; each R³ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein each R³ may be optionally substituted with between 0 and 3 R^{z} groups, wherein two R³ groups together with the atoms to which they are attached optionally form an aryl, cycloalkyl, heteroaryl, or heterocycloalkyl ring, which may be optionally substituted with between 0 and 3 R^{z} groups; R⁴ is chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, N(R³)₂, NR³C(O)C(R³)₃, NR³C(O)OC(R³)₃, NR³C(O)N(R³)₂, NR³S(O)C(R³)₃, NR³S(O)₂C(R³)₃, C(O)N(R³)₂, S(O)N(R³)₂, S(O)₂N(R³)₂, C(O)C(R³)₃, SC(R³)₃, S(O)C(R³)₃, and S(O)₂C(R³)₃, wherein R⁴ may be optionally substituted with between 0 and 3 R^{z} groups; each R^{z} group is independently chosen from alkenyl, alkoxy, alkyl, aryl, arylalkyl, cyano, cycloalkyl, cycloalkylalkyl, halo, haloalkyl, heteroaryl, heteroarylalkyl, heterocycloalkyl, heterocycloalkylalkyl, hydroxyl, oxo, N(R⁶)₂, NR⁶C(O)C(R⁶)₃, NR⁶C(O)OC(R⁶)₃, NR⁶C(O)N(R⁶)₂, NR⁶S(O)C(R⁶)₃, NR⁶S(O)₂C(R⁶)₃, C(O)N(R⁶)₂, S(O)N(R⁶)₂, S(O)₂N(R⁶)₂, C(O)C(R⁶)₃, SC(R⁶)₃, S(O)C(R⁶)₃, and S(O)₂C(R⁶)₃; each R⁶ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein two R⁶ groups together with the atoms to which they are attached optionally form an aryl, cycloalkyl, heteroaryl, or heterocycloalkyl ring, which may be optionally substituted with between 0 and 3 R^{x} groups; and Z is heteroaryl, which may be optionally substituted.

Provided is a composition comprising a compound of Formula I and a pharmaceutically acceptable carrier, adjuvant, or vehicle.

Provided is a method of inhibiting GLS1 activity in a biological sample comprising contacting the biological sample with a compound of Formula I.

Provided is a method of treating a GLS1-mediated disorder in a subject in need thereof, comprising the step of administering to the subject a compound of Formula I.

Provided is a method of treating a GLS1-mediated disorder in a subject in need thereof, comprising the sequential or co-administration of a compound of Formula I or a pharmaceutically acceptable salt thereof, and another therapeutic agent.

Provided is a compound of any of Formula I for use in human therapy.

Provided is a compound of any of Formula I for use in treating a GLS1-mediated disease.

Provided is a use of a compound of Formula I for the manufacture of a medicament to treat a GLS1-mediated disease.

### DETAILED DESCRIPTION

### Abbreviations and Definitions

To facilitate understanding of the disclosure, a number of terms and abbreviations as used herein are defined below as follows:

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values that it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20 carbon atoms. In certain embodiments, the alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to 20 carbon atoms. In certain embodiments, the alkyl will comprise from 1 to 10 carbon atoms. In further embodiments, the alkyl will comprise from 1 to 6 carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20 carbon atoms. In certain embodiments, the alkynyl comprises from 2 to 6 carbon atoms. In further embodiments, the alkynyl comprises from 2 to 4 carbon atoms. The term “alkynylene” refers to a carbon-carbon triple bond attached at two positions such as ethynylene (—C:::C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The terms “amido” and “carbamoyl” as used herein, alone or in combination, refer to an amino group as described below attached to the parent molecular moiety through a carbonyl group, or vice versa. The term “C-amido” as used herein, alone or in combination, refers to a —C(O)N(RR′) group with R and R′ as defined herein or as defined by the specifically enumerated “R” groups designated. The term “N-amido” as used herein, alone or in combination, refers to a RC(O)N(R′)— group, with R and R′ as defined herein or as defined by the specifically enumerated “R” groups designated. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted. Additionally, R and R′ may combine to form heterocycloalkyl, either of which may be optionally substituted.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄=derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxyl” or “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In certain embodiments, the cycloalkyl will comprise from 5 to 7 carbon atoms. Examples of such cycloalkyl groups include cyclopropyl, cyclobutyl, cyclopentyl, cyclohexyl, cycloheptyl, tetrahydronapthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom selected from the group consisting of O, S, and N. In certain embodiments, the heteroaryl will comprise from 5 to 7 carbon atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic group containing at least one heteroatom as a ring member, wherein each the heteroatom may be independently selected from the group consisting of nitrogen, oxygen, and sulfur In certain embodiments, the heterocycloalkyl will comprise from 1 to 4 heteroatoms as ring members. In further embodiments, the heterocycloalkyl will comprise from 1 to 2 heteroatoms as ring members. In certain embodiments, the heterocycloalkyl will comprise from 3 to 8 ring members in each ring. In further embodiments, the heterocycloalkyl will comprise from 3 to 7 ring members in each ring. In yet further embodiments, the heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of any one of the formulas disclosed herein.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in a combination, where not otherwise specifically defined, means containing from 1 to and including 6 carbon atoms.

The term “lower aryl,” as used herein, alone or in combination, means phenyl or naphthyl, either of which may be optionally substituted as provided.

The term “lower heteroaryl,” as used herein, alone or in combination, means either 1) monocyclic heteroaryl comprising five or six ring members, of which between one and four the members may be heteroatoms selected from the group consisting of O, S, and N, or 2) bicyclic heteroaryl, wherein each of the fused rings comprises five or six ring members, comprising between them one to four heteroatoms selected from the group consisting of O, S, and N.

The term “lower cycloalkyl,” as used herein, alone or in combination, means a monocyclic cycloalkyl having between three and six ring members. Lower cycloalkyls may be unsaturated. Examples of lower cycloalkyl include cyclopropyl, cyclobutyl, cyclopentyl, and cyclohexyl.

The term “lower heterocycloalkyl,” as used herein, alone or in combination, means a monocyclic heterocycloalkyl having between three and six ring members, of which between one and four may be heteroatoms selected from the group consisting of O, S, and N. Examples of lower heterocycloalkyls include pyrrolidinyl, imidazolidinyl, pyrazolidinyl, piperidinyl, piperazinyl, and morpholinyl. Lower heterocycloalkyls may be unsaturated.

The term “lower amino,” as used herein, alone or in combination, refers to —NRR′, wherein R and R′ are independently selected from the group consisting of hydrogen, lower alkyl, and lower heteroalkyl, any of which may be optionally substituted. Additionally, the R and R′ of a lower amino group may combine to form a five- or six-membered heterocycloalkyl, either of which may be optionally substituted.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′ as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that the group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, lower haloalkylthio, lower perhaloalkylthio, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and Rn where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

GLS1 inhibitor is used herein to refer to a compound that exhibits an IC50 with respect to GLS1 activity of no more than about 100 μM and more typically not more than about 50 μM, as measured in the GLS1 enzyme assay described generally herein below. IC50 is that concentration of inhibitor that reduces the activity of an enzyme (e.g., GLS1) to half-maximal level. Certain compounds disclosed herein have been discovered to exhibit inhibition against GLS1. In certain embodiments, compounds will exhibit an IC50 with respect to GLS1 of no more than about 10 μM; in further embodiments, compounds will exhibit an IC50 with respect to GLS1 of no more than about 5 μM; in yet further embodiments, compounds will exhibit an IC50 with respect to GLS1 of not more than about 1 μM; in yet further embodiments, compounds will exhibit an IC50 with respect to GLS1 of not more than about 200 nM, as measured in the GLS1 binding assay described herein.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans. Examples of patients include humans, livestock (farm animals) such as cows, goats, sheep, pigs, and rabbits, and companion animals such as dogs, cats, rabbits, and horses. Preferably, the patient is a human.

The term “prodrug” refers to a compound that is made more active in vivo. Certain compounds disclosed herein may also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound.

The compounds disclosed herein can exist as therapeutically acceptable salts. The present disclosure includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

### Compounds

The present disclosure provides a compound of structural Formula I

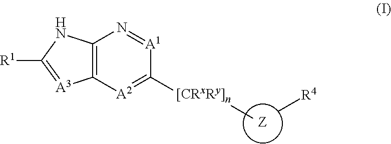

or a salt thereof, wherein: n is chosen from 3, 4, and 5; each R^{x} and R^{y} is independently chosen from alkyl, cyano, H, and halo, wherein two R^{x} groups together with the atoms to which they are attached optionally form a cycloalkyl ring; A¹ and A² are independently chosen from N and CH; A³ is chosen from N and CR²; R¹ is chosen from alkenyl, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, C(O)N(R³)₂, and C(O)C(R³)₃, wherein R¹ may be optionally substituted with between 0 and 3 R^{z} groups; R² is chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, C(O)N(R³)₂, C(O)C(R³)₃, C(O)OH, C(O)OC(R³)₃, wherein R¹ and R² together with the atoms to which they are attached optionally form an form an aryl, cycloalkyl, heteroaryl, or heterocycloalkyl ring, which may be optionally substituted with between 0 and 3 R^{z} groups; each R³ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein each R³ may be optionally substituted with between 0 and 3 R^{z} groups, wherein two R³ groups together with the atoms to which they are attached optionally form an aryl, cycloalkyl, heteroaryl, or heterocycloalkyl ring, which may be optionally substituted with between 0 and 3 R^{z} groups; R⁴ is chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, N(R³)₂, NR³C(O)C(R³)₃, NR³C(O)OC(R³)₃, NR³C(O)N(R³)₂, NR³S(O)C(R³)₃, NR³S(O)₂C(R³)₃, C(O)N(R³)₂, S(O)N(R³)₂, S(O)₂N(R³)₂, C(O)C(R³)₃, SC(R³)₃, S(O)C(R³)₃, and S(O)₂C(R³)₃, wherein R⁴ may be optionally substituted with between 0 and 3 R^{z} groups; each R^{z} group is independently chosen from alkenyl, alkoxy, alkyl, aryl, arylalkyl, cyano, cycloalkyl, cycloalkylalkyl, halo, haloalkyl, heteroaryl, heteroarylalkyl, heterocycloalkyl, heterocycloalkylalkyl, hydroxyl, oxo, N(R⁶)₂, NR⁶C(O)C(R⁶)₃, NR⁶C(O)OC(R⁶)₃, NR⁶C(O)N(R⁶)₂, NR⁶S(O)C(R⁶)₃, NR⁶S(O)₂C(R⁶)₃, C(O)N(R⁶)₂, S(O)N(R⁶)₂, S(O)₂N(R⁶)₂, C(O)C(R⁶)₃, SC(R⁶)₃, S(O)C(R⁶)₃, and S(O)₂C(R⁶)₃; each R⁶ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein two R⁶ groups together with the atoms to which they are attached optionally form an aryl, cycloalkyl, heteroaryl, or heterocycloalkyl ring, which may be optionally substituted with between 0 and 3 R^{x} groups; and Z is heteroaryl, which may be optionally substituted.

In some embodiments, Z is a 5-6 membered monocyclic or 9-10 membered bicyclic heteroaryl, either of which contains one to four heteroatoms chosen from N, O, and S, and either of which may optionally be substituted by one to three substituents chosen from lower alkyl, halogen, CF₃, OCF₃, cyano, and hydroxyl.

In some embodiments, the compound, or a pharmaceutically acceptable salt thereof, has Formula II

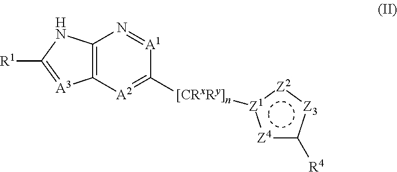

wherein: n is chosen from 3, 4, and 5; each R^{x} and R^{y} is independently chosen from alkyl, cyano, H, and halo, wherein two R^{x} groups together with the atoms to which they are attached optionally form a cycloalkyl ring; A¹ and A² are independently chosen from N and CH; A³ is chosen from N and CR²; Z¹ is chosen from C and N; Z², Z³, and Z⁴ are independently chosen from N, O, S, and CH, wherein at least one of Z¹, Z², Z³, and Z⁴ is chosen from N, O, and S; R¹ is chosen from alkenyl, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, C(O)N(R³)₂, and C(O)C(R³)₃, wherein R¹ may be optionally substituted with between 0 and 3 R^{z} groups; R² is chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, C(O)N(R³)₂, C(O)C(R³)₃, C(O)OH, C(O)OC(R³)₃, wherein R¹ and R² together with the atoms to which they are attached optionally form an form an aryl, cycloalkyl, heteroaryl, or heterocycloalkyl ring, which may be optionally substituted with between 0 and 3 R^{z} groups; each R³ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein each R³ may be optionally substituted with between 0 and 3 R^{z} groups, wherein two R³ groups together with the atoms to which they are attached optionally form an aryl, cycloalkyl, heteroaryl, or heterocycloalkyl ring, which may be optionally substituted with between 0 and 3 R^{z} groups; R⁴ is chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, N(R³)₂, NR³C(O)C(R³)₃, NR³C(O)OC(R³)₃, NR³C(O)N(R³)₂, NR³S(O)C(R³)₃, NR³S(O)₂C(R³)₃, C(O)N(R³)₂, S(O)N(R³)₂, S(O)₂N(R³)₂, C(O)C(R³)₃, SC(R³)₃, S(O)C(R³)₃, and S(O)₂C(R³)₃, wherein R⁴ may be optionally substituted with between 0 and 3 R^{z} groups; each R^{z} group is independently chosen from alkenyl, alkoxy, alkyl, aryl, arylalkyl, cyano, cycloalkyl, cycloalkylalkyl, halo, haloalkyl, heteroaryl, heteroarylalkyl, heterocycloalkyl, heterocycloalkylalkyl, hydroxyl, oxo, N(R⁶)₂, NR⁶C(O)C(R⁶)₃, NR⁶C(O)OC(R⁶)₃, NR⁶C(O)N(R⁶)₂, NR⁶S(O)C(R⁶)₃, NR⁶S(O)₂C(R⁶)₃, C(O)N(R⁶)₂, S(O)N(R⁶)₂, S(O)₂N(R⁶)₂, C(O)C(R⁶)₃, SC(R⁶)₃, S(O)C(R⁶)₃, and S(O)₂C(R⁶)₃; each R⁶ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein two R⁶ groups together with the atoms to which they are attached optionally form an aryl, cycloalkyl, heteroaryl, or heterocycloalkyl ring, which may be optionally substituted with between 0 and 3 R^{x} groups.

In certain embodiments, n is 4; and A¹, A², and A³ are CH.

In certain embodiments, n is 4; A¹ and A³ are N; and A² is CH.

In certain embodiments, n is 4; A¹ and A² are CH; and A³ is N.

In certain embodiments, n is 4; A¹ is N; A² is CH; and A³ is CR².

In certain embodiments, Z₁ is C; Z₂ and Z₃ are N; Z₄ is S; and R⁴ is chosen from N(R³)₂, NR³C(O)C(R³)₃, NR³C(O)OC(R³)₃, and NR³C(O)N(R³)₂.

In certain embodiments, n is 4; A¹ is N; A² is CH; A³ is CR²; Z¹ is C; Z² and Z³ are N; Z⁴ is S; and R⁴ is chosen from N(R³)₂, NR³C(O)C(R³)₃, NR³C(O)OC(R³)₃, and NR³C(O)N(R³)₂.

In certain embodiments, Z¹ is C; Z² and Z³ are N; Z⁴ is S; and R⁴ is C(O)N(R³)₂.

In certain embodiments, n is 4; A¹ is N; A² is CH; A³ is CR²; Z¹ is C; Z² and Z³ are N; Z⁴ is S; and R⁴ is C(O)N(R³)₂.

In certain embodiments, Z¹, Z², and Z³ are N; Z⁴ is CH; and R⁴ is chosen from N(R³)₂, NR³C(O)C(R³)₃, NR³C(O)OC(R³)₃, and NR³C(O)N(R³)₂.

In certain embodiments, n is 4; A¹ is N; A² is CH; A³ is CR²; Z¹, Z², and Z³ are N; Z⁴ is CH; and R⁴ is chosen from N(R³)₂, NR³C(O)C(R³)₃, NR³C(O)OC(R³)₃, and NR³C(O)N(R³)₂.

In certain embodiments, Z¹, Z², and Z³ are N; Z⁴ is CH; and R⁴ is C(O)N(R³)₂.

In certain embodiments, n is 4; A¹ is N; A² is CH; A³ is CR²; Z¹, Z², and Z³ are N; Z⁴ is CH; and R⁴ is C(O)N(R³)₂.

In some embodiments, the compound, or a pharmaceutically acceptable salt thereof, has Formula III

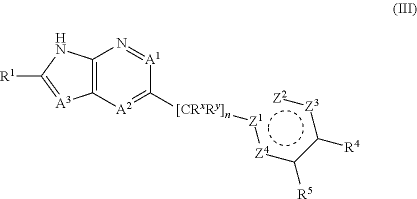

wherein: n is chosen from 3, 4, and 5; each R^{x} and R^{y} is independently chosen from alkyl, cyano, H, and halo, wherein two R^{x} groups together with the atoms to which they are attached optionally form a cycloalkyl ring; A¹ and A² are independently chosen from N and CH; A³ is chosen from N and CR²; Z₁ is chosen from C and N; Z² is chosen from N, CH, and C(O); Z³, and Z⁴ are independently chosen from N and CH, wherein at least one of Z¹, Z², Z³, and Z⁴ is N; R¹ is chosen from alkenyl, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, C(O)N(R³)₂, and C(O)C(R³)₃, wherein R¹ may be optionally substituted with between 0 and 3 R^{z} groups; R² is chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, C(O)N(R³)₂, C(O)C(R³)₃, C(O)OH, C(O)OC(R³)₃, wherein R¹ and R² together with the atoms to which they are attached optionally form an form an aryl, cycloalkyl, heteroaryl, or heterocycloalkyl ring, which may be optionally substituted with between 0 and 3 R^{z} groups; each R³ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein each R³ may be optionally substituted with between 0 and 3 R^{z} groups, wherein two R³ groups together with the atoms to which they are attached optionally form an aryl, cycloalkyl, heteroaryl, or heterocycloalkyl ring, which may be optionally substituted with between 0 and 3 R^{z} groups; R⁴ is chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, N(R³)₂, NR³C(O)C(R³)₃, NR³C(O)OC(R³)₃, NR³C(O)N(R³)₂, NR³S(O)C(R³)₃, NR³S(O)₂C(R³)₃, C(O)N(R³)₂, S(O)N(R³)₂, S(O)₂N(R³)₂, C(O)C(R³)₃, SC(R³)₃, S(O)C(R³)₃, and S(O)₂C(R³)₃, wherein R⁴ may be optionally substituted with between 0 and 3 R^{z} groups; each R^{z} group is independently chosen from alkenyl, alkoxy, alkyl, aryl, arylalkyl, cyano, cycloalkyl, cycloalkylalkyl, halo, haloalkyl, heteroaryl, heteroarylalkyl, heterocycloalkyl, heterocycloalkylalkyl, hydroxyl, oxo, N(R⁶)₂, NR⁶C(O)C(R⁶)₃, NR⁶C(O)OC(R⁶)₃, NR⁶C(O)N(R⁶)₂, NR⁶S(O)C(R⁶)₃, NR⁶S(O)₂C(R⁶)₃, C(O)N(R⁶)₂, S(O)N(R⁶)₂, S(O)₂N(R⁶)₂, C(O)C(R⁶)₃, SC(R⁶)₃, S(O)C(R⁶)₃, and S(O)₂C(R⁶)₃; and each R⁶ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein two R⁶ groups together with the atoms to which they are attached optionally form an aryl, cycloalkyl, heteroaryl, or heterocycloalkyl ring, which may be optionally substituted with between 0 and 3 R^{x} groups.

In certain embodiments, n is 4; and A¹, A², and A³ are CH.

In certain embodiments, n is 4; A¹ and A³ are N; and A² is CH.

In certain embodiments, n is 4; A¹ and A² are CH; and A³ is N.

In certain embodiments, n is 4; A¹ is N; A² is CH; and A³ is CR².

In certain embodiments, Z¹ is C; Z² and Z³ are N; Z⁴ is CH; R⁴ is chosen from N(R³)₂, NR³C(O)C(R³)₃, NR³C(O)OC(R³)₃, and NR³C(O)N(R³)₂; and R⁵ is H.

In certain embodiments, n is 4; A¹ is N; A² is CH; A³ is CR²; Z¹ is C; Z² and Z³ are N; Z⁴ is CH; R⁴ is chosen from N(R³)₂, NR³C(O)C(R³)₃, NR³C(O)OC(R³)₃, and NR³C(O)N(R³)₂; and R⁵ is H.

In certain embodiments, Z¹ is C; Z² and Z³ are N; Z⁴ is CH; R⁴ is C(O)N(R³)₂; and R⁵ is H.

In certain embodiments, n is 4; A¹ is N; A² is CH; A³ is CR²; Z¹ is C; Z² and Z³ are N; Z⁴ is CH; R⁴ is C(O)N(R³)₂; and R⁵ is H.

In certain embodiments, Z¹ is N; Z² is C(O); Z⁴ is CH; R⁴ is chosen from N(R³)₂, NR³C(O)C(R³)₃, NR³C(O)OC(R³)₃, and NR³C(O)N(R³)₂; and R⁵ is H.

In certain embodiments, n is 4; A¹ is N; A² is CH; A³ is CR²; Z¹ is N; Z² is C(O); Z⁴ is CH; R⁴ is chosen from N(R³)₂, NR³C(O)C(R³)₃, NR³C(O)OC(R³)₃, and NR³C(O)N(R³)₂; and R⁵ is H.

In certain embodiments, Z¹ is N; Z² is C(O); Z⁴ is CH; R⁴ is C(O)N(R³)₂; and R⁵ is H.

In certain embodiments, n is 4; A¹ is N; A² is CH; A³ is CR²; Z¹ is N; Z² is C(O); Z⁴ is CH; R⁴ is C(O)N(R³)₂; and R⁵ is H.

Also provided are embodiments wherein any of embodiment above in paragraphs

and [0107]-[0135] above may be combined with any one or more of these embodiments, provided the combination is not mutually exclusive.

### Pharmaceutical Compositions

While it may be possible for the compounds of the subject disclosure to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound of the subject disclosure or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Compounds described herein can be administered as follows:

### Oral Administration

The compounds of the present invention may be administered orally, including swallowing, so the compound enters the gastrointestinal tract, or is absorbed into the blood stream directly from the mouth, including sublingual or buccal administration.

Suitable compositions for oral administration include solid formulations such as tablets, pills, cachets, lozenges and hard or soft capsules, which can contain liquids, gels, powders, or granules.

In a tablet or capsule dosage form the amount of drug present may be from about 0.05% to about 95% by weight, more typically from about 2% to about 50% by weight of the dosage form.

In addition, tablets or capsules may contain a disintegrant, comprising from about 0.5% to about 35% by weight, more typically from about 2% to about 25% of the dosage form. Examples of disintegrants include methyl cellulose, sodium or calcium carboxymethyl cellulose, croscarmellose sodium, polyvinylpyrrolidone, hydroxypropyl cellulose, starch and the like.

Suitable binders, for use in a tablet, include gelatin, polyethylene glycol, sugars, gums, starch, hydroxypropyl cellulose and the like. Suitable diluents, for use in a tablet, include mannitol, xylitol, lactose, dextrose, sucrose, sorbitol and starch.

Suitable surface active agents and glidants, for use in a tablet or capsule, may be present in amounts from about 0.1% to about 3% by weight, and include polysorbate 80, sodium dodecyl sulfate, talc and silicon dioxide.

Suitable lubricants, for use in a tablet or capsule, may be present in amounts from about 0.1% to about 5% by weight, and include calcium, zinc or magnesium stearate, sodium stearyl fumarate and the like.

Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with a liquid diluent. Dyes or pigments may be added to tablets for identification or to characterize different combinations of active compound doses.

Liquid formulations can include emulsions, solutions, syrups, elixirs and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

Compositions for oral administration may be formulated as immediate or modified release, including delayed or sustained release, optionally with enteric coating.

In another embodiment, a pharmaceutical composition comprises a therapeutically effective amount of a compound of Formula (I) or a pharmaceutically acceptable salt thereof, and a pharmaceutically acceptable carrier.

### Parenteral Administration

Compounds of the present invention may be administered directly into the blood stream, muscle, or internal organs by injection, e.g., by bolus injection or continuous infusion. Suitable means for parenteral administration include intravenous, intra-muscular, subcutaneous intraarterial, intraperitoneal, intrathecal, intracranial, and the like. Suitable devices for parenteral administration include injectors (including needle and needle-free injectors) and infusion methods. The formulations may be presented in unit-dose or multi-dose containers, for example sealed ampoules and vials.

Most parenteral formulations are aqueous solutions containing excipients, including salts, buffering, suspending, stabilizing and/or dispersing agents, antioxidants, bacteriostats, preservatives, and solutes which render the formulation isotonic with the blood of the intended recipient, and carbohydrates.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions.

Compositions for parenteral administration may be formulated as immediate or modified release, including delayed or sustained release. Compounds may also be formulated as depot preparations. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

### Topical Administration

Compounds of the present invention may be administered topically (for example to the skin, mucous membranes, ear, nose, or eye) or transdermally. Formulations for topical administration can include, but are not limited to, lotions, solutions, creams, gels, hydrogels, ointments, foams, implants, patches and the like. Carriers that are pharmaceutically acceptable for topical administration formulations can include water, alcohol, mineral oil, glycerin, polyethylene glycol and the like. Topical administration can also be performed by, for example, electroporation, iontophoresis, phonophoresis and the like.

Typically, the active ingredient for topical administration may comprise from 0.001% to 10% w/w (by weight) of the formulation. In certain embodiments, the active ingredient may comprise as much as 10% w/w; less than 5% w/w; from 2% w/w to 5% w/w; or from 0.1% to 1% w/w of the formulation.

Compositions for topical administration may be formulated as immediate or modified release, including delayed or sustained release.

### Rectal, Buccal, and Sublingual Administration

Suppositories for rectal administration of the compounds of the present invention can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

### Administration by Inhalation

For administration by inhalation, compounds may be conveniently delivered from an insufflator, nebulizer pressurized packs or other convenient means of delivering an aerosol spray or powder. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the disclosure may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Other carrier materials and modes of administration known in the pharmaceutical art may also be used. Pharmaceutical compositions of the invention may be prepared by any of the well-known techniques of pharmacy, such as effective formulation and administration procedures. Preferred unit dosage formulations are those containing an effective dose, as herein recited, or an appropriate fraction thereof, of the active ingredient. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. In addition, the route of administration may vary depending on the condition and its severity. The above considerations concerning effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N. Y., 1980; and Kibbe, et al., Eds., Handbook of Pharmaceutical Excipients (3^{rd} Ed.), American Pharmaceutical Association, Washington, 1999.

### Methods of Treatment

The present disclosure provides compounds and pharmaceutical compositions that inhibit glutaminase activity, particularly GLS1 activity and are thus useful in the treatment or prevention of disorders associated with GLS1. Compounds and pharmaceutical compositions of the present disclosure selectively modulate GLS1 and are thus useful in the treatment or prevention of a range of disorders associated with GLS1 and include, but are not limited to, cancer, immunological or neurological diseases associated with GLS1.

### Neurological Disorders

In some embodiments, the compounds and pharmaceutical compositions of the present disclosure may be useful in the treatment or prevention of neurological diseases.

The most common neurotransmitter is glutamate, derived from the enzymatic conversion of glutamine via glutaminase. High levels of glutamate have been shown to be neurotoxic. Following traumatic insult to neuronal cells, there occurs a rise in neurotransmitter release, particularly glutamate. Accordingly, inhibition of glutaminase has been hypothesized as a means of treatment following an ischemic insult, such as stroke.

Huntington's disease is a progressive, fatal neurological condition. In genetic mouse models of Huntington's disease, it was observed that the early manifestation of the disease correlated with dysregulated glutamate release (Raymond et al., Neuroscience, 2011). In HIV-associated dementia, HIV infected macrophages exhibit upregulated glutaminase activity and increased glutamate release, leading to neuronal damage (Huang et al., J. Neurosci., 2011). Similarly, in another neurological disease, the activated microglia in Rett Syndrome release glutamate causing neuronal damage. The release of excess glutamate has been associated with the up-regulation of glutaminase (Maezawa et al., J. Neurosci, 2010). In mice bred to have reduced glutaminase levels, sensitivity to psychotic-stimulating drugs, such as amphetamines, was dramatically reduced, thus suggesting that glutaminase inhibition may be beneficial in the treatment of schizophrenia (Gaisler-Salomon et al., Neuropsychopharmacology, 2009). Bipolar disorder is a devastating illness that is marked by recurrent episodes of mania and depression. This disease is treated with mood stabilizers such as lithium and valproate; however, chronic use of these drugs appear to increase the abundance of glutamate receptors (Nanavati et al., J. Neurochem., 2011), which may lead to a decrease in the drug's effectiveness over time. Thus, an alternative treatment may be to reduce the amount of glutamate by inhibiting glutaminase. This may or may not be in conjunction with the mood stabilizers. Memantine, a partial antagonist of N-methyl-D-aspartate receptor (NMDAR), is an approved therapeutic in the treatment of Alzheimer's disease. Currently, research is being conducted looking at memantine as a means of treating vascular dementia and Parkinson's disease (Oliverares et al., Curr. Alzheimer Res., 2011). Since memantine has been shown to partially block the NMDA glutamate receptor also, it is not unreasonable to speculate that decreasing glutamate levels by inhibiting glutaminase could also treat Alzheimer's disease, vascular dementia and Parkinson's disease. Alzheimer's disease, bipolar disorder, HIV-associated dementia, Huntington's disease, ischemic insult, Parkinson's disease, schizophrenia, stroke, traumatic insult and vascular dementia are but a few of the neurological diseases that have been correlated to increased levels of glutamate. Thus, inhibiting glutaminase with a compound described herein can reduce or prevent neurological diseases. Therefore, in certain embodiments, the compounds may be used for the treatment or prevention of neurological diseases.

### Immunological Disorders

In some embodiments, the compounds and pharmaceutical compositions of the present disclosure may be useful in the treatment or prevention of immunological diseases.

Activation of T lymphocytes induces cell growth, proliferation, and cytokine production, thereby placing energetic and biosynthetic demands on the cell. Glutamine serves as an amine group donor for nucleotide synthesis, and glutamate, the first component in glutamine metabolism, plays a direct role in amino acid and glutathione synthesis, as well as being able to enter the Krebs cycle for energy production (Carr et al., J. Immunol., 2010). Mitogen-induced T cell proliferation and cytokine production require high levels of glutamine metabolism, thus inhibiting glutaminase may serve as a means of immune modulation. In multiple sclerosis, an inflammatory autoimmune disease, the activated microglia exhibit up-regulated glutaminase and release increased levels of extracellular glutamate. Glutamine levels are lowered by sepsis, injury, burns, surgery and endurance exercise (Calder et al., Amino Acids, 1999). These situations put the individual at risk of immunosuppression. In fact, in general, glutaminase gene expression and enzyme activity are both increased during T cell activity. Patients given glutamine following bone marrow transplantation resulted in a lower level of infection and reduced graft v. host disease (Crowther, Proc. Nutr. Soc., 2009). T cell proliferation and activation is involved in many immunological diseases, such as inflammatory bowel disease, Crohn's disease, sepsis, psoriasis, arthritis (including rheumatoid arthritis), multiple sclerosis, graft v. host disease, infections, lupus and diabetes. In an embodiment of the invention, the compounds described herein can be used to treat or prevent immunological diseases.

### Cancer

In some embodiments, the compounds and pharmaceutical compositions of the present disclosure may be useful in the treatment or prevention of cancer.

In addition to serving as the basic building blocks of protein synthesis, amino acids have been shown to contribute to many processes critical for growing and dividing cells, and this is particularly true for cancer cells. Nearly all definitions of cancer include reference to dysregulated proliferation. Numerous studies on glutamine metabolism in cancer indicate that many tumors are avid glutamine consumers (Souba, Ann. Surg., 1993; Collins et al., J. Cell. Physiol., 1998; Medina, J. Nutr., 2001; Shanware et al., J. Mol. Med., 2011). An embodiment of the invention is the use of the compounds described herein for the treatment of cancer.

In some embodiments, the compounds of the present disclosure may be used to prevent or treat cancer, wherein the cancer is one or a variant of Acute Lymphoblastic Leukemia (ALL), Acute Myeloid Leukemia (AML), Adrenocortical Carcinoma, AIDS-Related Cancers (Kaposi Sarcoma and Lymphoma), Anal Cancer, Appendix Cancer, Atypical Teratoid/Rhabdoid Tumor, Basal Cell Carcinoma, Bile Duct Cancer (including Extrahepatic), Bladder Cancer, Bone Cancer (including Osteosarcoma and Malignant Fibrous Histiocytoma), Brain Tumor (such as Astrocytomas, Brain and Spinal Cord Tumors, Brain Stem Glioma, Central Nervous System Atypical Teratoid/Rhabdoid Tumor, Central Nervous System Embryonal Tumors, Craniopharyngioma, Ependymoblastoma, Ependymoma, Medulloblastoma, Medulloepithelioma, Pineal Parenchymal Tumors of Intermediate Differentiation, Supratentorial Primitive Neuroectodermal Tumors and Pineoblastoma), Breast Cancer, Bronchial Tumors, Burkitt Lymphoma, Basal Cell Carcinoma, Bile Duct Cancer (including Extrahepatic), Bladder Cancer, Bone Cancer (including Osteosarcoma and Malignant Fibrous Histiocytoma), Carcinoid Tumor, Carcinoma of Unknown Primary, Central Nervous System (such as Atypical Teratoid/Rhabdoid Tumor, Embryonal Tumors and Lymphoma), Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma (Mycosis Fungoides and Sézary Syndrome), Duct, Bile (Extrahepatic), Ductal Carcinoma In Situ (DCIS), Embryonal Tumors (Central Nervous System), Endometrial Cancer, Ependymoblastoma, Ependymoma, Esophageal Cancer, Esthesioneuroblastoma, Ewing Sarcoma Family of Tumors, Extracranial Germ Cell Tumor, Extragonadal Germ Cell Tumor, Extrahepatic Bile Duct Cancer, Eye Cancer (like Intraocular Melanoma, Retinoblastoma), Fibrous Histiocytoma of Bone (including Malignant and Osteosarcoma) Gallbladder Cancer, Gastric (Stomach) Cancer, Gastrointestinal Carcinoid Tumor, Gastrointestinal Stromal Tumors (GIST), Germ Cell Tumor (Extracranial, Extragonadal, Ovarian), Gestational Trophoblastic Tumor, Glioma, Hairy Cell Leukemia, Head and Neck Cancer, Heart Cancer, Hepatocellular (Liver) Cancer, Histiocytosis, Langerhans Cell, Hodgkin Lymphoma, Hypopharyngeal Cancer, Intraocular Melanoma, Islet Cell Tumors (Endocrine, Pancreas), Kaposi Sarcoma, Kidney (including Renal Cell), Langerhans Cell Histiocytosis, Laryngeal Cancer, Leukemia (including Acute Lymphoblastic (ALL), Acute Myeloid (AML), Chronic Lymphocytic (CLL), Chronic Myelogenous (CML), Hairy Cell), Lip and Oral Cavity Cancer, Liver Cancer (Primary), Lobular Carcinoma In Situ (LCIS), Lung Cancer (Non-Small Cell and Small Cell), Lymphoma (AIDS-Related, Burkitt, Cutaneous T-Cell (Mycosis Fungoides and Sézary Syndrome), Hodgkin, Non-Hodgkin, Primary Central Nervous System (CNS), Macroglobulinemia, Waldenström, Male Breast Cancer, Malignant Fibrous Histiocytoma of Bone and Osteosarcoma, Medulloblastoma, Medulloepithelioma, Melanoma (including Intraocular (Eye)), Merkel Cell Carcinoma, Mesothelioma (Malignant), Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic (CML), Myeloid Leukemia, Acute (AML), Myeloma and Multiple Myeloma, Myeloproliferative Disorders (Chronic), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and, Oropharyngeal Cancer, Osteosarcoma and Malignant Fibrous Histiocytoma of Bone, Ovarian Cancer (such as Epithelial, Germ Cell Tumor, and Low Malignant Potential Tumor), Pancreatic Cancer (including Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pineal Parenchymal Tumors of Intermediate Differentiation, Pineoblastoma and Supratentorial Primitive Neuroectodermal Tumors, Pituitary Tumor, Plasma Cell Neoplasm/Multiple Myeloma, Pleuropulmonary Blastoma, Pregnancy and Breast Cancer, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (like Ewing Sarcoma Family of Tumors, Kaposi, Soft Tissue, Uterine), Sézary Syndrome, Skin Cancer (such as Melanoma, Merkel Cell Carcinoma, Nonmelanoma), Small Cell Lung Cancer, Small Intestine Cancer, Soft Tissue Sarcoma, Squamous Cell Carcinoma, Squamous Neck Cancer with Occult Primary, Metastatic, Stomach (Gastric) Cancer, Supratentorial Primitive Neuroectodermal Tumors, T-Cell Lymphoma (Cutaneous, Mycosis Fungoides and Sézary Syndrome), Testicular Cancer, Throat Cancer, Thymoma and Thymic Carcinoma, Thyroid Cancer, Transitional Cell Cancer of the Renal Pelvis and Ureter, Trophoblastic Tumor (Gestational), Unknown Primary, Unusual Cancers of Childhood, Ureter and Renal Pelvis, Transitional Cell Cancer, Urethral Cancer, Uterine Cancer, Endometrial, Uterine Sarcoma, Waldenström Macroglobulinemia or Wilms Tumor.

In certain embodiments, the cancer to be treated is one specific to T-cells such as T-cell lymphomia and lymphoblastic T-cell leukemia.

In some embodiments, methods described herein are used to treat a disease condition comprising administering to a subject in need thereof a therapeutically effective amount of a compound of Formula I or pharmaceutically acceptable salt thereof, wherein the condition is cancer which has developed resistance to chemotherapeutic drugs and/or ionizing radiation.

### Combinations and Combination Therapy

The compounds of the present invention can be used, alone or in combination with other pharmaceutically active compounds, to treat conditions such as those previously described hereinabove. The compound(s) of the present invention and other pharmaceutically active compound(s) can be administered simultaneously (either in the same dosage form or in separate dosage forms) or sequentially. Accordingly, in one embodiment, the present invention comprises methods for treating a condition by administering to the subject a therapeutically-effective amount of one or more compounds of the present invention and one or more additional pharmaceutically active compounds.

In another embodiment, there is provided a pharmaceutical composition comprising one or more compounds of the present invention, one or more additional pharmaceutically active compounds, and a pharmaceutically acceptable carrier.

In another embodiment, the one or more additional pharmaceutically active compounds is selected from the group consisting of anti-cancer drugs, anti-proliferative drugs, and anti-inflammatory drugs.

GLS1 inhibitor compositions described herein are also optionally used in combination with other therapeutic reagents that are selected for their therapeutic value for the condition to be treated. In general, the compounds described herein and, in embodiments where combination therapy is employed, other agents do not have to be administered in the same pharmaceutical composition and, because of different physical and chemical characteristics, are optionally administered by different routes. The initial administration is generally made according to established protocols and then, based upon the observed effects, the dosage, modes of administration and times of administration subsequently modified. In certain instances, it is appropriate to administer a GLS1 inhibitor compound, as described herein, in combination with another therapeutic agent. By way of example only, the therapeutic effectiveness of a GLS1 inhibitor is enhanced by administration of another therapeutic agent (which also includes a therapeutic regimen) that also has therapeutic benefit. Regardless of the disease, disorder or condition being treated, the overall benefit experienced by the patient is either simply additive of the two therapeutic agents or the patient experiences an enhanced (i.e., synergistic) benefit. Alternatively, if a compound disclosed herein has a side effect, it may be appropriate to administer an agent to reduce the side effect; or the therapeutic effectiveness of a compound described herein may be enhanced by administration of an adjuvant.

Therapeutically effective dosages vary when the drugs are used in treatment combinations. Methods for experimentally determining therapeutically effective dosages of drugs and other agents for use in combination treatment regimens are documented methodologies. Combination treatment further includes periodic treatments that start and stop at various times to assist with the clinical management of the patient. In any case, the multiple therapeutic agents (one of which is a GLS1 inhibitor as described herein) may be administered in any order, or simultaneously. If simultaneously, the multiple therapeutic agents are optionally provided in a single, unified form, or in multiple forms (by way of example only, either as a single pill or as two separate pills).

In some embodiments, one of the therapeutic agents is given in multiple doses, or both are given as multiple doses. If not simultaneous, the timing between the multiple doses optionally varies from more than zero weeks to less than twelve weeks.

In addition, the combination methods, compositions and formulations are not to be limited to the use of only two agents, the use of multiple therapeutic combinations are also envisioned. It is understood that the dosage regimen to treat, prevent, or ameliorate the condition(s) for which relief is sought, is optionally modified in accordance with a variety of factors. These factors include the disorder from which the subject suffers, as well as the age, weight, sex, diet, and medical condition of the subject. Thus, the dosage regimen actually employed varies widely, in some embodiments, and therefore deviates from the dosage regimens set forth herein.

The pharmaceutical agents which make up the combination therapy disclosed herein are optionally a combined dosage form or in separate dosage forms intended for substantially simultaneous administration. The pharmaceutical agents that make up the combination therapy are optionally also administered sequentially, with either agent being administered by a regimen calling for two-step administration. The two-step administration regimen optionally calls for sequential administration of the active agents or spaced-apart administration of the separate active agents. The time between the multiple administration steps ranges from a few minutes to several hours, depending upon the properties of each pharmaceutical agent, such as potency, solubility, bioavailability, plasma half-life and kinetic profile of the pharmaceutical agent.

In another embodiment, a GLS1 inhibitor is optionally used in combination with procedures that provide additional benefit to the patient. A GLS1 inhibitor and any additional therapies are optionally administered before, during or after the occurrence of a disease or condition, and the timing of administering the composition containing a GLS1 inhibitor varies in some embodiments. Thus, for example, a GLS1 inhibitor is used as a prophylactic and is administered continuously to subjects with a propensity to develop conditions or diseases in order to prevent the occurrence of the disease or condition. A GLS1 inhibitor and compositions are optionally administered to a subject during or as soon as possible after the onset of the symptoms. While embodiments of the present invention have been shown and described herein, it will be obvious to those skilled in the art that such embodiments are provided by way of example only. Numerous variations, changes, and substitutions will now occur to those skilled in the art without departing from the invention. It should be understood that in some embodiments of the invention various alternatives to the embodiments described herein are employed in practicing the invention.

A GLS1 inhibitor can be used in combination with anti-cancer drugs, including but not limited to the following classes: alkylating agents, anti-metabolites, plant alkaloids and terpenoids, topoisomerase inhibitors, cytotoxic antibiotics, angiogenesis inhibitors and tyrosine kinase inhibitors.

For use in cancer and neoplastic diseases a GLS1 inhibitor may be optimally used together with one or more of the following non-limiting examples of anti-cancer agents: (1) alkylating agents, including but not limited to cisplatin (PLATIN), carboplatin (PARAPLATIN), oxaliplatin (ELOXATIN), streptozocin (ZANOSAR), busulfan (MYLERAN) and cyclophosphamide (ENDOXAN); (2) anti-metabolites, including but not limited to mercaptopurine (PURINETHOL), thioguanine, pentostatin (NIPENT), cytosine arabinoside (ARA-C), gemcitabine (GEMZAR), fluorouracil (CARAC), leucovorin (FUSILEV) and methotrexate (RHEUMATREX); (3) plant alkaloids and terpenoids, including but not limited to vincristine (ONCOVIN), vinblastine and paclitaxel (TAXOL); (4) topoisomerase inhibitors, including but not limited to irinotecan (CAMPTOSAR), topotecan (HYCAMTIN) and etoposide (EPOSIN); (5) cytotoxic antibiotics, including but not limited to actinomycin D (COSMEGEN), doxorubicin (ADRIAMYCIN), bleomycin (BLENOXANE) and mitomycin (MITOSOL); (6) angiogenesis inhibitors, including but not limited to sunitinib (SUTENT) and bevacizumab (AVASTIN); and (7) tyrosine kinase inhibitors, including but not limited to imatinib (GLEEVEC), erlotinib (TARCEVA), lapatininb (TYKERB) and axitinib (INLYTA).

Where a subject is suffering from or at risk of suffering from an inflammatory condition, a GLS1 inhibitor compound described herein is optionally used together with one or more agents or methods for treating an inflammatory condition in any combination. Therapeutic agents/treatments for treating an autoimmune and/or inflammatory condition include, but are not limited to any of the following examples: (1) corticosteroids, including but not limited to cortisone, dexamethasone, and methylprednisolone; (2) nonsteroidal anti-inflammatory drugs (NSAIDs), including but not limited to ibuprofen, naproxen, acetaminophen, aspirin, fenoprofen (NALFON), flurbiprofen (ANSAID), ketoprofen, oxaprozin (DAYPRO), diclofenac sodium (VOLTAREN), diclofenac potassium (CATAFLAM), etodolac (LODINE), indomethacin (INDOCIN), ketorolac (TORADOL), sulindac (CLINORIL), tolmetin (TOLECTIN), meclofenamate (MECLOMEN), mefenamic acid (PONSTEL), nabumetone (RELAFEN) and piroxicam (FELDENE); (3) immunosuppressants, including but not limited to methotrexate (RHEUMATREX), leflunomide (ARAVA), azathioprine (IMURAN), cyclosporine (NEORAL, SANDIMMUNE), tacrolimus and cyclophosphamide (CYTOXAN); (4) CD20 blockers, including but not limited to rituximab (RITUXAN); (5) Tumor Necrosis Factor (TNF) blockers, including but not limited to etanercept (ENBREL), infliximab (REMICADE) and adalimumab (HUMIRA); (6) interleukin-1 receptor antagonists, including but not limited to anakinra (KINERET); (7) interleukin-6 inhibitors, including but not limited to tocilizumab (ACTEMRA); (8) interleukin-17 inhibitors, including but not limited to AIN457; (9) Janus kinase inhibitors, including but not limited to tasocitinib; and (10) syk inhibitors, including but not limited to fostamatinib.

### Compound Synthesis

Compounds of the present invention can be prepared using methods illustrated in general synthetic schemes and experimental procedures detailed below. General synthetic schemes and experimental procedures are presented for purposes of illustration and are not intended to be limiting. Starting materials used to prepare compounds of the present invention are commercially available or can be prepared using routine methods known in the art.

### LIST OF ABBREVIATIONS

Ac₂O=acetic anhydride; AcCl=acetyl chloride; AcOH=acetic acid; AIBN=azobisisobutyronitrile; aq.=aqueous; Bu₃SnH=tributyltin hydride; CD₃OD=deuterated methanol; CDCl₃=deuterated chloroform; CDI=1,1′-Carbonyldiimidazole; DBU=1,8-diazabicyclo[5.4.0]undec-7-ene; DCM=dichloromethane; DEAD=diethyl azodicarboxylate; DIBAL-H=di-iso-butyl aluminium hydride; DIEA=DIPEA=N,N-diisopropylethylamine; DMAP=4-dimethylaminopyridine; DMF=N,N-dimethylformamide; DMSO-d₆=deuterated dimethyl sulfoxide; DMSO=dimethyl sulfoxide; DPPA=diphenylphosphoryl azide; EDC.HCl=EDCI.HCl=1-ethyl-3-(3-dimethylaminopropyl)carbodiimide hydrochloride; Et₂O=diethyl ether; EtOAc=ethyl acetate; EtOH=ethanol; h=hour; HATU=2-(1H-7-azabenzotriazol-1-yl)-1,1,3,3-tetramethyl uronium hexafluorophosphate methanaminium; HMDS=hexamethyldisilazane; HOBT=1-hydroxybenzotriazole; i-PrOH=isopropanol; LAH=lithium aluminiumhydride; LiHMDS=Lithium bis(trimethylsilyl)amide; MeCN=acetonitrile; MeOH=methanol; MP-carbonate resin=macroporous triethylammonium methylpolystyrene carbonate resin; MsCl=mesyl chloride; MTBE=methyl tertiary butyl ether; n-BuLi=n-butyllithium; NaHMDS=Sodium bis(trimethylsilyl)amide; NaOMe=sodium methoxide; NaOtBu=sodium t-butoxide; NBS=N-bromosuccinimide; NCS=N-chlorosuccinimide; NMP=N-Methyl-2-pyrrolidone; Pd(Ph₃)₄=tetrakis(triphenylphosphine)palladium(0); Pd₂(dba)₃=tris(dibenzylideneacetone)dipalladium(0); PdCl₂(PPh₃)₂=bis(triphenylphosphine)palladium(II) dichloride; PG=protecting group; prep-HPLC=preparative high-performance liquid chromatography; PyBop=(benzotriazol-1-yloxy)tripyrrolidinophosphonium hexafluorophosphate; Pyr=pyridine; RT=room temperature; RuPhos=2-dicyclohexylphosphino-2′,6′-diisopropoxybiphenyl; sat.=saturated; ss=saturated solution; t-BuOH=tert-butanol; T3P=Propylphosphonic Anhydride; TEA=Et₃N=triethylamine; TFA=trifluoroacetic acid; TFAA=trifluoroacetic anhydride; THF=tetrahydrofuran; Tol=toluene; TsCl=tosyl chloride; XPhos=2-dicyclohexylphosphino-2′,4′,6′-triisopropylbiphenyl.

### General Methods for Preparing Compounds

The following schemes can be used to practice the present invention. Additional structural groups, including but not limited to those defined elsewhere in the specification and not shown in the compounds described in the schemes can be incorporated to give various compounds disclosed herein, or intermediate compounds which can, after further manipulations using techniques known to those skilled in the art, be converted to compounds of the present invention. For example in certain embodiments the A-ring in the structures described in the schemes—wherein A is a heteroaromatic ring—can be substituted with various groups as defined herein.

One route for preparation of compounds of the present invention is described in Scheme 1. A suitably functionalized amino-heteroaromatic ring containing two halogen atoms in the ortho- and para-position in respect to the amino group can be functionalized by reaction with a suitable alkyne via Sonogashira cross-coupling reaction (Tetrahedron Lett. 16: 4467-4470). Typically, the above transformation is performed in the presence of a suitable Pd catalyst such as PdCl₂(PPh₃)₂ or Pd(PPh₃)₄, of a copper co-catalyst, typically a halide salt of copper(I), such as CuI or CuBr, and a base such as DIEA or TEA, with mild heating in a variety of solvents, including DMF, toluene and EtOAc. Regio-isomeric mixtures of products derived from ortho- and para-functionalization are typically obtained from this transformation, and the desired ortho-derivatized alkynes can be isolated by means of SiO₂ gel or reverse phase chromatography. This intermediate can then be cyclized to a bicyclic heteroaromatic for example by treatment of the alkyne products with a suitable base such as K₂CO₃, or tBuOK heating in a solvent such as MeOH or THF. The latter can be further functionalized, for example by Negishi cross coupling reaction at the remaining halogen with a functionalized alkyl-zinc reagent (Negishi E., et al., Chem. Comm. 1977, 19, 683). The transformation is typically catalyzed by either a Pd or a Ni complex, such as PdCl₂(PPh₃)₂, Pd₂(dba)₃, Pd(PPh₃)₄, or Ni(dppp)Cl₂, heating in a solvent such as THF, DMF or NMP. Functional group manipulations of the moieties thus installed can then be employed to complete the synthesis of the compounds of this invention. For example, when the functionalized alkyl-zinc reagent used in the above Negishi cross-coupling bears a nitrile group, this can be converted to the corresponding 5-alkyl-2-amino thiadiazole by heating in the presence of TFA and hydrazinecarbothioamide. Functionalization of the 2-amino group is then possible by employing standard methodologies known to those skilled in the art. For example, acylation with an acyl chloride in the presence of a base such as DIEA, pyridine, TEA and in a solvent such as DCM can afford the corresponding 2-carboxamides. Alternatively, the same transformation can be achieved by employing a carboxylic acid and coupling reagents such as EDC.HCl/HOBT, PyBOP, HATU or T3P in the presence of a base such as TEA or DIPEA, in a solvent such as DCM or DMF, with or without heating.

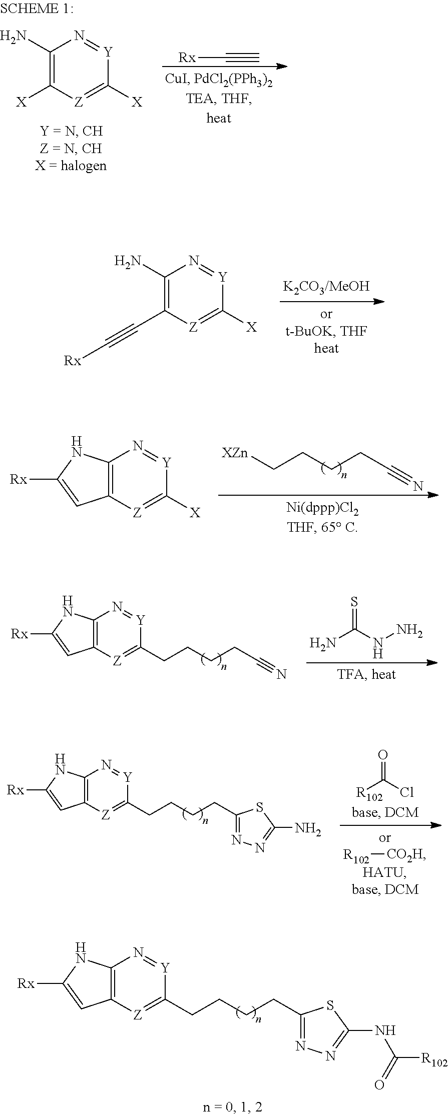

Functionalization of the 5/6 bicyclic heteroaromatic compounds described in Scheme 1 can also be achieved by cross coupling reaction with a suitable alkyne already functionalized with a desired heteroaromatic core. Such transformation can be performed using the Sonogashira cross-coupling conditions, in analogous manner to what has been already described for Scheme 1. The compounds of this invention can then be obtained by hydrogenation of the resulting heteroaromatic alkyne derivative in the presence of a suitable Pd catalyst (such as Pd/C or Pd(OH₂)) in a solvent such as EtOH. Further functional group manipulations of the Rx and/or Ry substituents might be required for the completion of the synthesis, and a non-limiting number of such transformations is represented in Schemes 4 and 5.

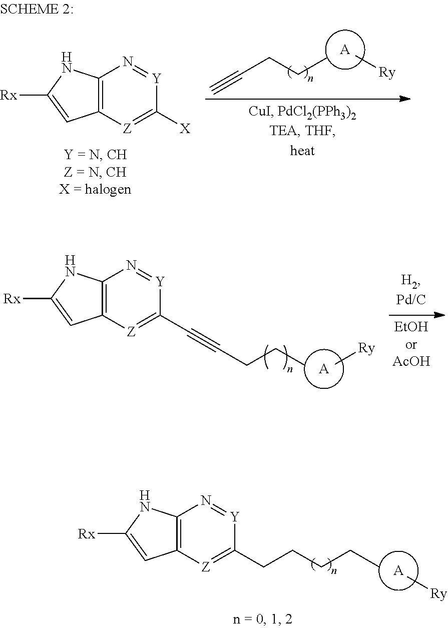

A further route of preparation of the compounds described in this invention is depicted in Scheme 3. A suitably functionalized 1,2-bis-amino-heteroaromatic ring containing a halogen atom can be mono-acylated employing one equivalent of carboxylic acid and coupling reagents such as CDI, EDC.HCl/HOBT, PyBOP or HATU, in the presence of a base such as TEA or DIPEA, in a solvent such as DCM or DMF. Heating the resulting region-isomeric mixture of mono-acylated products in the presence of an acid, for example AcOH, can then result in the formation of the corresponding 5/6-bicyclic heteroaromatic system by cyclo-condensation reaction. The bicyclic heterocyclic core thus obtained can be further functionalized, for example by reaction with a suitable alkyne via Sonogashira cross coupling, followed by hydrogenation of the resulting heteroaromatic alkyne derivative, in analogous conditions to those described for Schemes 1 and 2, and if necessary further transformed to the compounds of this invention.

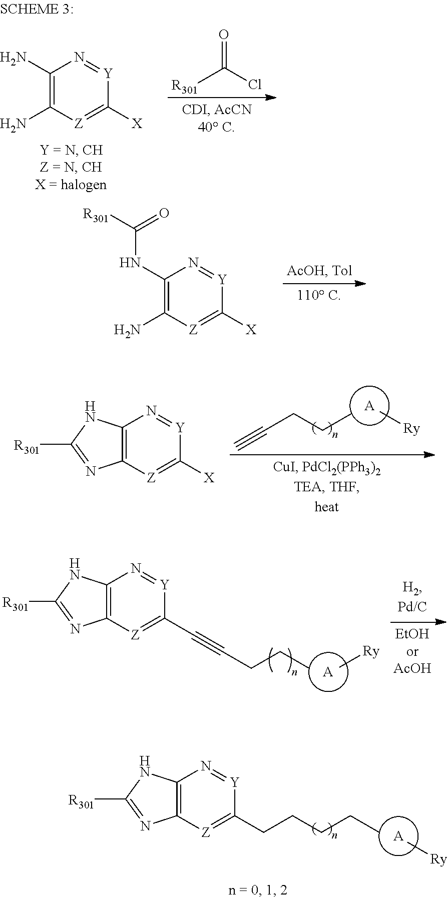

The compounds obtained as described in the previous Schemes 1-3 may be further functionalized as depicted in Schemes 4 and 5. Halogenation of the 5/6-bicyclic core in the 3-position (indole-like numbering) can be achieved by treatment with a suitable agent like Br₂, NCS or ICl in a solvent such as DCM. Further functionalization of the resulting heteroaryl halide can then be achieved via cross-coupling reaction, for example employing a suitable alkyl zinc reagent in Negishi conditions, in an analogous manner to the transformations described in Scheme 1. Alternatively, the hetero-halide can be functionalized via Suzuki cross-coupling, by employing a suitable boronic acid in the presence of a Pd catalyst such as PdCl₂(PPh₃)₂, Pd₂(dba)₃ or Pd(PPh₃)₄, and base such as K₂CO₃, Cs₂CO₃ or NaOH, heating in a solvent like DMF, THF or dioxane (Miyaura N., Suzuki A., et al. Tetrahedron Lett. 1979, 20 (36), 3437-3440). The presence of a suitable N-protecting group might be required in order to accomplish these transformations. Such group can be chosen for example amongst a substituted carbamate, a sulfonamide or other suitable functional groups known to those skilled in the art (see also: P. G. M. Wutz, T. W. Greene, “Greene's protective Groups in Organic Synthesis”, Fourth Edition, John Wiley & Sons).

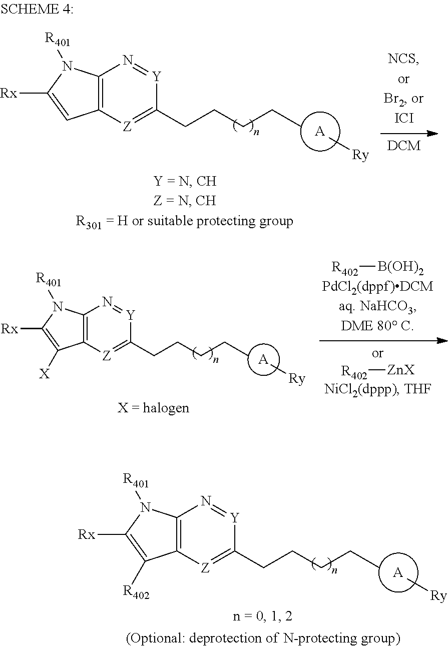

Standard functional group manipulations known to those skilled in the art can be performed with the R^{x} and R^{y} substituents groups of the structures reported in Schemes 1-4. A non-limiting number of examples of such transformations are exemplified in Scheme 5. For simplicity these transformations are described for the R^{y} group only, although they can be applied independently to one or both of the R^{x} and R^{y} groups.

In Scheme 5a, where R^{y}=carboxylic ester, the corresponding carboxamides can be obtained either by direct displacement with a suitable amine heating in a polar solvent such as DMF, or with a two-step sequence involving the base-mediated hydrolysis of the carboxylic ester followed by coupling of the resulting carboxylic acid with an amine. The coupling reaction can be performed using standard coupling reagents such as HATU, PyBOP or EDCI.HCl/HOBT or T3P in the presence of a suitable base such as TEA or DIEA, in a polar solvent such as DMF, with or without heating.

In Scheme 5b the R^{y} group is a protected amine group. Suitable protecting groups for the amine moiety can be chosen amongst substituted carbamates, amides and amines (e.g. benzyl amine, 3,4-dimethoxy-benzylamine, t-butyl carbamate, trifluoroacetamide) or amongst other suitable functional groups known to those skilled in the art (see also: P. G. M. Wutz, T. W. Greene, “Greene's protective Groups in Organic Synthesis”, Fourth Edition, John Wiley & Sons). The free amino group can be obtained by removal of the amine protecting group with techniques known to those skilled in the art (for example: reductive removal of a benzyl group; acid-mediated removal of the tert-butyl carbamoyl group and other conditions reported by P. G. M. Wutz, T. W. Greene in the reference cited above). The obtained amino derivatives can be further functionalized according to the non-limiting reaction examples reported in Scheme 5. Thus, the corresponding carboxamides can be prepared by acylation with a carboxylic chloride in the presence of a base such as DIEA, pyridine or TEA in a solvent such as DCM, or employing a carboxylic acid and coupling reagents such as EDC.HCl/HOBT, PyBOP, HATU or T3P in the presence of a base such as TEA or DIPEA, in a solvent such as DCM or DMF, with or without heating. Similarly, carbamate and sulfamides derivatives can be obtained by reaction of the amine compounds with a suitable carbamoyl- or sulfonyl chloride respectively, in the presence of a base such as TEA or DIPEA in a solvent such as DCM. Urea derivatives can be prepared by reaction of the amine moiety with a suitable isocyanate, in a solvent such as THF or DCM. Alkylation of the amino group can be achieved by treatment with a suitable alkylating agent, for example an alkyl, benzyl or heterobenzyl bromide in the presence of a base of a suitable strength, for example NaH, in a solvent such as THF.

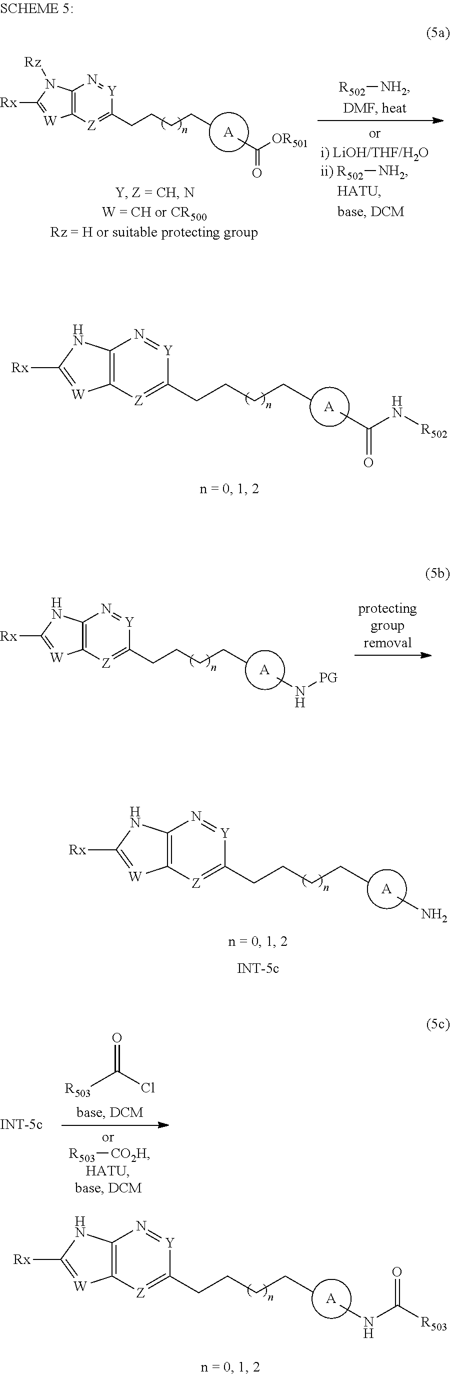

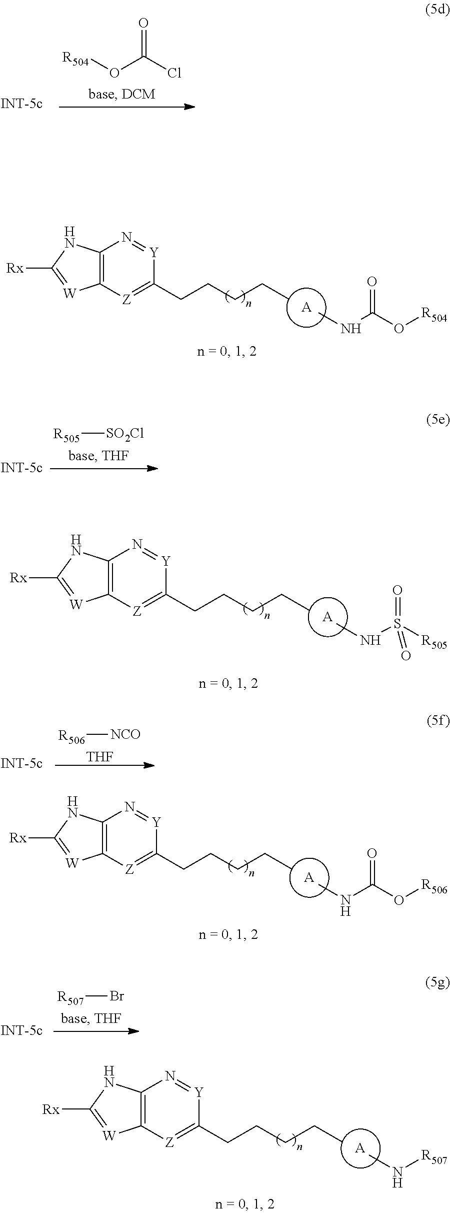

### EXAMPLES

Non-limiting examples include the following compounds and pharmaceutically acceptable salts thereof.

### Example 1

### 5-(4-(7H-pyrrolo[2,3-c]pyridazin-3-yl)butyl)-1,3,4-thiadiazol-2-amine

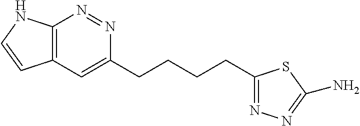 [SMILES: Nc1nnc(CCCCc2cc3cc[nH]c3nn2)s1]

### Steps 1 to 6

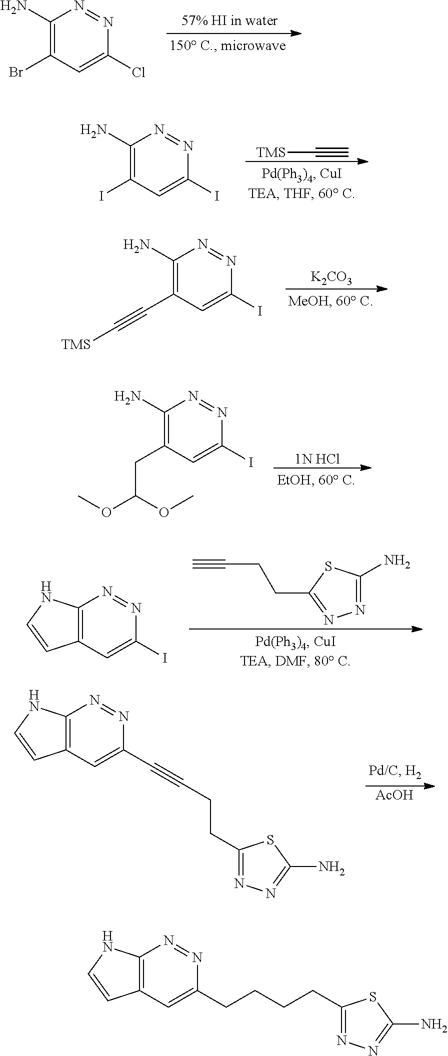

### Step 1: 4,6-diiodopyridazin-3-amine

A microwave vial was charged with 4-bromo-6-chloropyridazin-3-amine (4.0 g, 19 mmol), and HI in water (57% wt, 40 ml) was added. The vial was sealed and the reaction mixture was heated to 150° C. in the microwave reactor for 16 minutes, then diluted with EtOAc, sonicated, filtered and the solid was collected to give the title compound as an HI salt. After drying under reduced pressure, the solid was taken up in 30 ml water and sat. NaHCO₃ was added until pH=8 was reached. The solid was collected by filtration and dried under reduced pressure to give the title compound as a tan solid (4.35 g, 65% yield). MS (ES⁺) C₄H₃I₂N₃ requires: 346, found: 347 [M+H]⁺.

### Step 2: 6-iodo-4-((trimethylsilyl)ethynyl)pyridazin-3-amine

A suspension of 4,6-diiodopyridazin-3-amine (1.0 g, 2.9 mmol) in THF (10 ml) was degassed under a stream of N₂ and ethynyltrimethylsilane (0.283 g, 2.88 mmol), palladium tetrakis (0.666 g, 0.577 mmol), CuI (0.110 g, 0.577 mmol) and Et₃N (4.02 ml, 28.8 mmol) were added. The resulting mixture was stirred at 60° C. for 4 h, cooled to RT and the volatiles were removed under reduced pressure. The residue was taken up in DCM, filtered, the filtrate was concentrated under reduced pressure and the residue was purified via silica gel chromatography (0-30% EtOAc in hexanes to give the title compound as an off-white solid (422 mg, 46% yield). MS (ES⁺) C₉H₁₂IN₃Si requires: 317, found: 318 [M+H]⁺.

### Step 3: 4-(2,2-dimethoxyethyl)-6-iodopyridazin-3-amine

To a suspension of 6-iodo-4-((trimethylsilyl)ethynyl)pyridazin-3-amine (100 mg, 0.315 mmol) in MeOH (3 ml) was added K₂CO₃ (218 mg, 1.58 mmol) and the resulting mixture was stirred at 60° C. for 3 h. The volatiles were removed under reduced pressure, the residue was taken up in DCM, filtered through Buchner funnel, and the filtrate was concentrated under reduced pressure. The residue was purified via silica gel chromatography (0-5% MeOH in DCM to give the title compound as an off-white solid (72 mg, 74% yield). MS (ES⁺) C₈H₁₂IN₃O₂ requires: 309, found: 310 [M+H]⁺.

### Step 4: 3-iodo-7H-pyrrolo[2,3-c]pyridazine

To a solution of 4-(2,2-dimethoxyethyl)-6-iodopyridazin-3-amine (72 mg, 0.23 mmol) in ethanol (2 ml) was added 1N aq. HCl (0.5 ml) and the resulting mixture was stirred at 60° C. for 2 h. The volatiles were removed under reduced pressure and the residue was triturated with DCM to give the title compound as a yellow solid (51 mg, 89% yield). MS (ES⁺) C₆H₄IN₃ requires: 245, found: 246 [M+H]⁺.

### Step 5: 5-(4-(7H-pyrrolo[2,3-c]pyridazin-3-yl)but-3-yn-1-yl)-1,3,4-thiadiazol-2-amine

A mixture of 3-iodo-7H-pyrrolo[2,3-c]pyridazine (50 mg, 0.204 mmol), 5-(but-3-yn-1-yl)-1,3,4-thiadiazol-2-amine (31.3 mg, 0.204 mmol), Pd(PPh₃)₂Cl₂ (14.3 mg, 0.0200 mmol), CuI (3.89 mg, 0.0200 mmol), Et₃N (206 mg, 2.04 mmol) in DMF (2 ml) was degassed under a stream of N₂ and stirred at 80° C. for 2 h. The volatiles were removed under reduced pressure, the residue was taken up in MeOH and purified by mass-triggered preparative HPLC (Mobile phase: A=0.1% TFA/H₂O, B=0.1% TFA/MeCN; Gradient: B=10-50%; 12 min; Column: C18) to give the title compound as an off-white solid (35 mg, 64% yield). MS (ES⁺) Cl₂H₁₀N₆S requires: 270, found: 271 [M+H]⁺.

### Step 6: 5-(4-(7H-pyrrolo[2,3-c]pyridazin-3-yl)butyl)-1,3,4-thiadiazol-2-amine

A reaction vessel was charged with 5-(4-(7H-pyrrolo[2,3-c]pyridazin-3-yl)but-3-yn-1-yl)-1,3,4-thiadiazol-2-amine (30 mg, 0.11 mmol), 10% Pd—C (30 mg, 0.28 mmol) and AcOH (2 ml) under an atmosphere of N₂. The suspension was degassed under a stream of N₂ and purged with H₂. The reaction mixture was then stirred under an atmosphere of H₂ at RT for 2 h, purged with N₂, filtered through a pad of Celite®, and concentrated under reduced pressure to give the title compound as a yellow solid (28 mg, 92%). MS (ES⁺) C₁₉H₁₉N₇OS requires: 274, found: 275 [M+H]⁺. ¹H NMR (600 MHz, DMSO-d₆) δ 13.46 (bs, 1H), 8.56 (bs, 1H), 8.42 (bs, 1H), 7.10 (bs, 2H), 6.92 (s, 1H), 3.14 (t, J=7.2 Hz, 2H), 2.85 (t, J=7.2 Hz, 2H), 1.86-1.81 (m, 2H), 1.72-1.65 (m, 2H).

### Example 2

### N-(5-(4-(7H-pyrrolo[2,3-c]pyridazin-3-yl)butyl)-1,3,4-thiadiazol-2-yl)-2-(pyridin-2-yl)acetamide

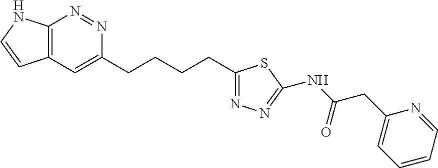 [SMILES: O=C(Cc1ccccn1)Nc1nnc(CCCCc2cc3cc[nH]c3nn2)s1]

To a solution of 2-(pyridin-2-yl)acetic acid hydrochloride (15.8 mg, 0.0910 mmol) in DMF (0.5 ml) were added HATU (38.1 mg, 0.100 mmol), 5-(4-(7H-pyrrolo[2,3-c]pyridazin-3-yl)butyl)-1,3,4-thiadiazol-2-amine (Example 1, 25 mg, 0.091 mmol) and DIEA (0.040 ml, 0.23 mmol) and the resulting mixture was stirred at RT for 16 h. The volatiles were removed under reduced pressure, the residue was taken up in MeOH and purified by mass-triggered preparative HPLC (Mobile phase: A=0.1% TFA/H₂O, B=0.1% TFA/MeCN; Gradient: B=0-30%; 12 min; Column: C18) to give the title compound as a white solid (20 mg, 56% yield). MS (ES⁺) C₁₉H₁₉N₇OS requires: 393, found: 394 [M+H]⁺. ¹H NMR (600 MHz, DMSO-d₆) δ 13.54 (bs, 1H), 12.70 (s, 1H), 8.61 (s, 1H), 8.52 (d, J=4.2 Hz, 1H), 8.47 (s, 1H), 7.83 (m, 1H), 7.43 (d, J=7.8 Hz, 1H), 7.34 (m, 1H), 6.94 (s, 1H), 4.03 (s, 2H), 3.15 (t, J=7.2 Hz, 2H), 3.03 (t, J=7.2 Hz, 2H), 1.90-1.82 (m, 2H), 1.80-1.72 (m, 2H).

### Example 3

### 5-(4-(6-benzyl-7H-pyrrolo[2,3-c]pyridazin-3-yl)butyl)-1,3,4-thiadiazol-2-amine

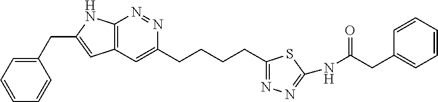 [SMILES: O=C(Cc1ccccc1)Nc1nnc(CCCCc2cc3cc(Cc4ccccc4)[nH]c3nn2)s1]

### Steps 1 to 3

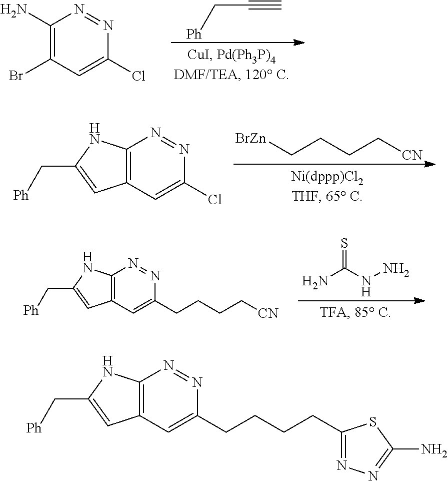

### Step 1: 6-benzyl-3-chloro-7H-pyrrolo[2,3-c]pyridazine

A solution of 4-bromo-6-chloropyridazin-3-amine (3.0 g, 14 mmol) in DMF (19 ml) and Et₃N (9.6 ml) was degassed with Ar for 1 minute. Copper(I) iodide (137 mg, 0.720 mmol), Pd(Ph₃P)₄ (832 mg, 0.720 mmol) and prop-2-yn-1-ylbenzene (1.97 ml, 15.8 mmol) were added and the mixture was degassed with Ar for an additional 1 minute. The reaction mixture was heated at 120° C. for 1 h, and then cooled to RT, filtered and concentrated under reduced pressure. The residue was purified via silica gel chromatography (0-8% MeOH in DCM to give the title compound as a brown solid (2.8 g, 80%). MS (ES⁺) C₁₃H₁₀ClN₃ requires: 243, found: 244 [M+H]⁺.

### Step 2: 5-(6-benzyl-7H-pyrrolo[2,3-c]pyridazin-3-yl)pentanenitrile

A solution of (4-cyanobutyl)zinc(II) bromide (16.4 mL, 8.20 mmol) was degassed with N₂ for 2 minutes. 6-benzyl-3-chloro-7H-pyrrolo[2,3-c]pyridazine (500 mg, 2.05 mmol) and [1,3-bis(diphenylphophino)propane]dichloronickel (II) (167 mg, 0.308 mmol) were added, and the mixture was heated to 65° C. for 12 h, then cooled to RT. The mixture was diluted with MeOH (5 mL) and concentrated under reduced pressure. The residue was purified via silica gel chromatography (0-10% MeOH in DCM to give the title compound as a brown solid. MS (ES⁺) C₁₈H₁₈N₄ requires: 290, found: 291 [M+H]⁺.

### Step 3: 5-(4-(6-benzyl-7H-pyrrolo[2,3-c]pyridazin-3-yl)butyl)-1,3,4-thiadiazol-2-amine

To a suspension of 5-(6-benzyl-7H-pyrrolo[2,3-c]pyridazin-3-yl)pentanenitrile (595 mg, 2.05 mmol) in TFA (4 mL) was added hydrazinecarbothioamide (205 mg, 2.25 mmol). The mixture was heated to 85° C. for 12 h, cooled to RT and concentrated under reduced pressure. The residue was purified by mass-triggered preparative HPLC (Mobile phase: A=0.1% TFA/H₂O, B=0.1% TFA/MeCN; Gradient: B=20%-50%; 16 min; Column: C18). The pulled fractions were neutralized with Biotage MP-Carbonate resin and concentrated under reduced pressure to give the title compound as an off-white solid (400 mg, 54%). MS (ES⁺) C₁₉H₂₀N₆S requires: 364, found: 365 [M+H]⁺; ¹H NMR (600 MHz, Methanol-d₄) δ 8.20 (s, 1H), 7.40-7.32 (m, 4H), 7.30 (m, 1H), 6.65 (s, 1H), 4.36 (s, 2H), 3.19-3.15 (m, 2H), 2.98 (t, J=7.3 Hz, 2H), 1.96-1.90 (m, 2H), 1.89-1.82 (m, 2H).

### Example 4

### N-(5-(4-(6-benzyl-7H-pyrrolo[2,3-c]pyridazin-3-yl)butyl)-1,3,4-thiadiazol-2-yl)-2-phenylacetamide

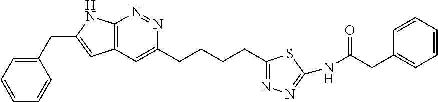 [SMILES: O=C(Cc1ccccc1)Nc1nnc(CCCCc2cc3cc(Cc4ccccc4)[nH]c3nn2)s1]

To a solution of 5-(4-(6-benzyl-7H-pyrrolo[2,3-c]pyridazin-3-yl)butyl)-1,3,4-thiadiazol-2-amine (Example 3, 20 mg, 0.055 mmol) in pyridine (1.1 mL) was added 2-phenylacetyl chloride (8.7 μL, 0.066 mmol) and the resulting mixture was stirred at RT for 12 h. The mixture was concentrated under reduced pressure and the residue was purified by mass-triggered preparative HPLC (Mobile phase: A=0.1% TFA/H₂O, B=0.1% TFA/MeCN; Gradient: B=20-50%; 12 min; Column: C18) to give the title compound as a white solid (7.6 mg, 23%). MS (ES⁺) C₂₇H₂₆N₆OS requires: 482, found: 483 [M+H]⁺; ¹H NMR (600 MHz, Methanol-d₄) δ 8.18 (s, 1H), 7.38-7.31 (m, 8H), 7.31-7.25 (m, 2H), 6.63 (s, 1H), 4.35 (s, 2H), 3.81 (s, 2H), 3.15 (t, J=7.1 Hz, 2H), 3.09 (t, J=6.7 Hz, 2H), 1.95-1.83 (m, 4H).

### Example 5

### (S)—N-(5-(4-(6-benzyl-7H-pyrrolo[2,3-c]pyridazin-3-yl)butyl)-1,3,4-thiadiazol-2-yl)-2-hydroxy-2-phenylacetamide

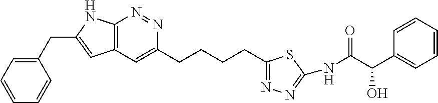 [SMILES: O=C(Nc1nnc(CCCCc2cc3cc(Cc4ccccc4)[nH]c3nn2)s1)[C@@H](O)c1ccccc1]

To a solution of 5-(4-(6-benzyl-7H-pyrrolo[2,3-c]pyridazin-3-yl)butyl)-1,3,4-thiadiazol-2-amine (Example 3; 10 mg, 0.027 mmol) in DMF (549 μl) was added (S)-2-hydroxy-2-phenylacetic acid (5.0 mg, 0.033 mmol) and the resulting mixture was stirred at RT for 12 h. The mixture was concentrated under reduced pressure and the residue was purified by mass-triggered preparative HPLC (Mobile phase: A=0.1% TFA/H₂O, B=0.1% TFA/MeCN; Gradient: B=30%-70%; 12 min; Column: C18) to give the title compound (3.7 mg, 18%). MS (ES⁺) C₂₇H₂₆N₆O₂S requires: 498, found: 499 [M+H]⁺; ¹H NMR (500 MHz, DMSO-d₆) δ 13.61 (s, 1H), 12.43 (s, 1H), 8.27 (s, 1H), 7.54-7.46 (m, 2H), 7.40-7.32 (m, 6H), 7.34-7.25 (m, 2H), 6.65 (s, 1H), 5.32 (bs, 1H), 4.33 (s, 2H), 3.17 (bs, 1H), 3.08 (t, J=7.3 Hz, 2H), 3.00 (t, J=7.2 Hz, 2H), 1.85-1.66 (m, 4H).

### Example 6

### 2-(3-(aminomethyl)phenyl)-N-(5-(4-(6-benzyl-7H-pyrrolo[2,3-c]pyridazin-3-yl)butyl)-1,3,4-thiadiazol-2-yl)acetamide

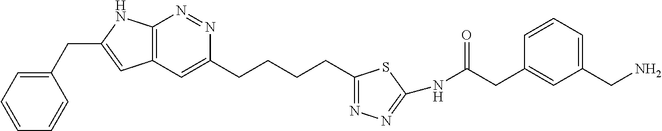 [SMILES: NCc1cccc(CC(=O)Nc2nnc(CCCCc3cc4cc(Cc5ccccc5)[nH]c4nn3)s2)c1]

To a solution of tert-butyl (3-(2-((5-(4-(6-benzyl-7H-pyrrolo[2,3-c]pyridazin-3-yl)butyl)-1,3,4-thiadiazol-2-yl)amino)-2-oxoethyl)benzyl)carbamate (prepared as described for Example 5; 10 mg, 0.016 mmol) in MeCN (5 mL) and water (5 mL) was added TFA (1.00 mL, 5.89 mmol). The mixture was heated at 40° C. for 12 h, then cooled to RT and concentrated under reduced pressure. The residue was purified by mass-triggered preparative HPLC (Mobile phase: A=0.1% TFA/H₂O, B=0.1% TFA/MeCN; Gradient: B=30-70%; 16 min; Column: C18) to give the title compound as a white solid (5 mg, 6%). MS (ES⁺) C₂₈H₂₉N₇OS requires: 511. found: 512 [M+H]⁺; ¹H NMR (600 MHz, Methanol-d₄) δ 8.19 (s, 1H), 7.47-7.32 (m, 8H), 7.33-7.26 (m, 1H), 6.64 (s, 1H), 4.36 (s, 2H), 4.11 (s, 2H), 3.88 (s, 2H), 3.16 (t, J=7.2 Hz, 2H), 3.09 (t, J=6.9 Hz, 2H), 1.98-1.85 (m, 4H).

### Example 7

### N-(5-(5-(6-benzyl-7H-pyrrolo[2,3-c]pyridazin-3-yl)pentyl)-1,3,4-thiadiazol-2-yl)-2-phenylacetamide

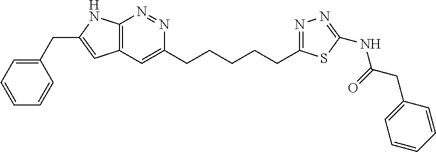 [SMILES: O=C(Cc1ccccc1)Nc1nnc(CCCCCc2cc3cc(Cc4ccccc4)[nH]c3nn2)s1]

### Steps 1 to 4

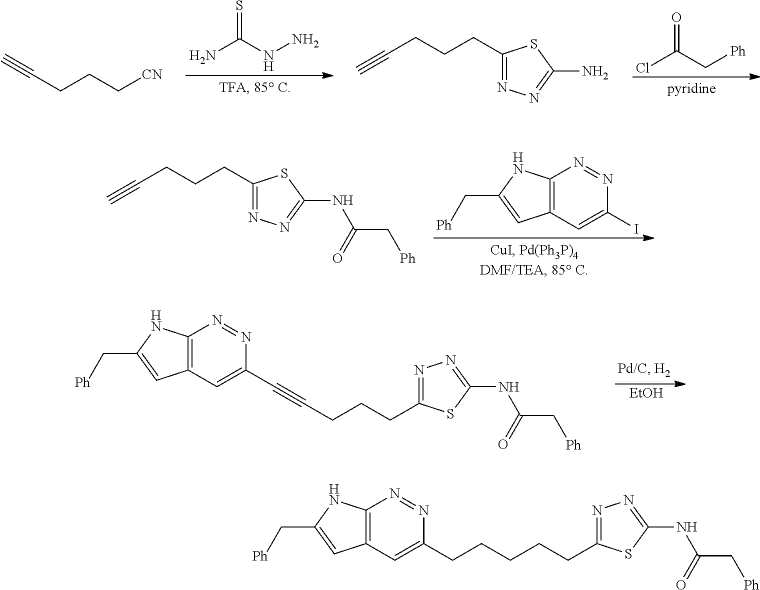

### Step 1: 5-(pent-4-yn-1-yl)-1,3,4-thiadiazol-2-amine

To a suspension of hydrazinecarbothioamide (538 mg, 5.91 mmol) in TFA (2.48 ml, 32.2 mmol) was added hex-5-ynenitrile (562 μl, 5.37 mmol) and the mixture was heated at 85° C. for 6 h. The mixture was cooled to RT, poured onto an ice-water mixture and brought to pH=14 by stirring with NaOH (1.3 g, 32 mmol). The mixture was concentrated under reduced pressure, the residue was adsorbed onto a pad of Celite®, and purified via SiO₂ gel chromatography (0 to 15% MeOH in DCM) to give the title compound as a white solid (680 mg, 76%). MS (ES⁺) C₇H₉N₃S requires: 167, found: 168 [M+H]⁺.

### Step 2: N-(5-(pent-4-yn-1-yl)-1,3,4-thiadiazol-2-yl)-2-phenylacetamide

To a suspension of 5-(pent-4-yn-1-yl)-1,3,4-thiadiazol-2-amine (100 mg, 0.598 mmol) in pyridine (3.0 ml) was added 2-phenylacetyl chloride (95 μl, 0.72 mmol) and the resulting mixture was stirred at RT for 1 h. 2-phenylacetyl chloride (95 μl, 0.72 mmol) was added and the mixture was stirred for an additional 1 h then concentrated under reduced pressure. The residue was purified via silica gel chromatography (0-5% MeOH in DCM) to give the title compound as an off-white solid (170 mg 100%). MS (ES⁺) C₁₅H₁₅N₃OS requires: 285, found: 286 [M+H]⁺.

### Step 3: N-(5-(5-(6-benzyl-7H-pyrrolo[2,3-c]pyridazin-3-yl)pent-4-yn-1-yl)-1,3,4-thiadiazol-2-yl)-2-phenylacetamide

A mixture of 6-benzyl-3-iodo-7H-pyrrolo[2,3-c]pyridazine (Example 10, Step 3; 30 mg, 0.090 mmol), N-(5-(pent-4-yn-1-yl)-1,3,4-thiadiazol-2-yl)-2-phenylacetamide (28 mg, 0.098 mmol) and copper(I) iodide (0.85 mg, 4.5 μmol) in DMF (597 μl) and Et₃N (298 μl) was degassed with N₂ for 2 minutes. Pd(Ph₃P)₄ (3.14 mg, 4.48 μmol) was added and the mixture was degassed with N₂ for an additional 2 minutes. The mixture was heated at 65° C. for 12 h, cooled to RT and concentrated under reduced pressure. The residue was purified via silica gel chromatography (0-10% MeOH in DCM) to give the title compound as a red solid (5 mg, 11%). MS (ES⁺) C₂₈H₂₄N₆OS requires: 492, found: 493 [M+H]⁺.

### Step 4: N-(5-(5-(6-benzyl-7H-pyrrolo[2,3-c]pyridazin-3-yl)pentyl)-1,3,4-thiadiazol-2-yl)-2-phenylacetamide

A reaction vessel was charged with N-(5-(5-(6-benzyl-7H-pyrrolo[2,3-c]pyridazin-3-yl)pent-4-yn-1-yl)-1,3,4-thiadiazol-2-yl)-2-phenylacetamide (5 mg, 10 μmol), 10% Pd—C (1.0 mg, 1.0 μmol) and EtOH (203 μl) under an atmosphere of N₂. The suspension was degassed with N₂ for 2 minutes, purged with H₂ for 2 minutes and stirred under an atmosphere of H₂ at 1 atm for 3 h. The reaction mixture was purged with N₂, filtered through a pad of Celite®, and concentrated under reduced pressure. The residue was purified by mass-triggered preparative HPLC (Mobile phase: A=0.1% TFA/H₂O, B=0.1% TFA/MeCN; Gradient: B=30-70%; 20 min; Column: C18) to give the title compound as a white solid (1.1 mg, 18%). MS (ES⁺) C₂₈H₂₈N₆O requires: 496, found: 497 [M+H]⁺; ¹H NMR (600 MHz, Methanol-d₄) δ 8.18 (s, 1H), 7.39-7.23 (m, 10H), 6.63 (s, 1H), 4.26 (s, 2H), 3.81 (s, 2H), 3.12-3.08 (m, 2H), 3.03 (t, J=7.4 Hz, 2H), 1.92-1.78 (m, 4H), 1.54-1.44 (m, 2H).

### Example 8

### (S)—N-(5-(4-(6-Cyclopropyl-7H-pyrrolo[2,3-c]pyridazin-3-yl)butyl)-1,3,4-thiadiazol-2-yl)-2-hydroxy-2-phenylacetamide

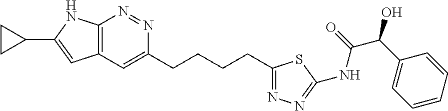 [SMILES: O=C(Nc1nnc(CCCCc2cc3cc(C4CC4)[nH]c3nn2)s1)[C@@H](O)c1ccccc1]

### Steps 1 to 4

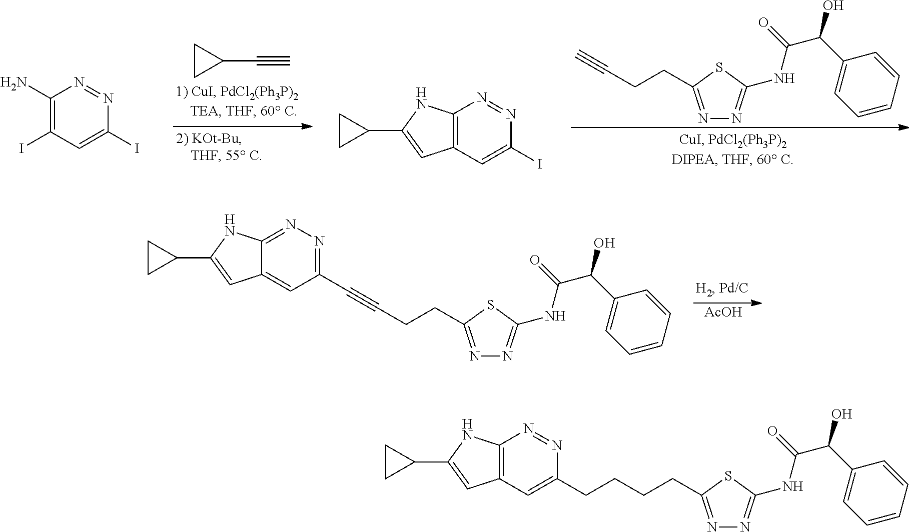

### Step 1: 4-(cyclopropylethynyl)-6-iodopyridazin-3-amine

To a solution of 4,6-diiodopyridazin-3-amine (Example 1, Step 1; 2.00 g, 5.76 mmol) in DMF (29 ml) were added ethynylcyclopropane (0.488 ml, 5.76 mmol), Et₃N (4.8 ml, 34 mmol), copper(I) iodide (113 mg, 0.594 mmol) and Pd(PPh₃)₂Cl₂ (200 mg, 0.285 mmol). The flask was briefly evacuated and filled with N₂, and the resulting dark orange solution was stirred at 40° C. for 17.5 h. An additional 0.1 mL of ethynylcyclopropane was added, and the solution was stirred at 40° C. for an additional 2 h, then cooled to RT and concentrated under reduced pressure. The residue was taken up in acetone, loaded onto silica gel and concentrated to an orange powder, which was purified via silica gel chromatography (0-33% EtOAc in hexanes) to give the 4-(cyclopropylethynyl)-6-iodopyridazin-3-amine as an orange-yellow solid (1.25 g, 4.39 mmol, 76% crude yield). MS (ES⁺) C₉H₈IN₃ requires: 285, found: 286 [M+H]⁺.

### Step 2: 6-Cyclopropyl-3-iodo-7H-pyrrolo[2,3-c]pyridazine

To a suspension of 4-(cyclopropylethynyl)-6-iodopyridazin-3-amine (1.25 g, 4.38 mmol) in THF (44 ml) was added potassium 2-methylpropan-2-olate (984 mg, 8.77 mmol), and the resulting blood-orange solution was stirred at 55° C. for 18 h. Silica gel (12 g) was added, and the mixture was concentrated under reduced pressure to a brown powder. The residue was purified via silica gel chromatography (0-50% EtOAc in hexanes) to give the title compound as a yellow solid (656 mg, 2.30 mmol, 53% yield). MS (ES⁺) C₉H₈IN₃ requires: 285, found: 286 [M+H]⁺.

### Step 3: (S)—N-(5-(4-(6-Cyclopropyl-7H-pyrrolo[2,3-c]pyridazin-3-yl)but-3-yn-1-yl)-1,3,4-thiadiazol-2-yl)-2-hydroxy-2-phenylacetamide

To a solution of 6-cyclopropyl-3-iodo-7H-pyrrolo[2,3-c]pyridazine (74.2 mg, 0.260 mmol) and (S)—N-(5-(but-3-yn-1-yl)-1,3,4-thiadiazol-2-yl)-2-hydroxy-2-phenylacetamide (prepared as described for Example 7, Step 2; 91.3 mg, 0.318 mmol) in THF (5 ml) were added Et₃N (0.220 ml, 1.58 mmol), copper(I) iodide (5.8 mg, 0.030 mmol) and Pd(PPh₃)₂Cl₂ (9.5 mg, 0.014 mmol). The resulting solution was stirred at 60° C. for 17 h, then cooled to RT. The mixture was concentrated under reduced pressure and the residue was purified via silica gel chromatography (0-5% MeOH in DCM) to give the title compound as a pale yellow solid (80.6 mg, 0.181 mmol, 70% yield). MS (ES⁺) C₂₃H₂₀N₆O₂S requires: 444, found: 445 [M+H]⁺.

### Step 4: (S)—N-(5-(4-(6-Cyclopropyl-7H-pyrrolo[2,3-c]pyridazin-3-yl)butyl)-1,3,4-thiadiazol-2-yl)-2-hydroxy-2-phenylacetamide

To a flask containing 10% Pd—C (50.4 mg, 0.474 mmol) under N₂ was added AcOH (2 ml) followed by (S)—N-(5-(4-(6-cyclopropyl-7H-pyrrolo[2,3-c]pyridazin-3-yl)but-3-yn-1-yl)-1,3,4-thiadiazol-2-yl)-2-hydroxy-2-phenylacetamide (51.6 mg, 0.116 mmol). The flask was evacuated and filled with H₂, and the mixture stirred at RT under an atmosphere of H₂ for 42 h. The flask was evacuated and filled with N₂, the suspension was filtered through a pad of Celite®, rinsed with AcOH, and the filtrate concentrated under reduced pressure. The residue was adsorbed onto silica gel and purified via SiO₂ gel chromatography (0-5% MeOH in DCM, then 10% MeOH in DCM) to give the title compound as a yellow solid (7.7 mg, 0.017 mmol, 15% yield). MS (ES⁺) C₂₃H₂₄N₆O₂S requires: 448, found: 449 [M+H]⁺; ¹H NMR (600 MHz, DMSO-d₆) δ 12.38 (bs, 1H), 11.94 (bs, 1H), 7.50 (d, J=7.55 Hz, 2H), 7.41 (s, 1H), 7.32-7.38 (m, 2H), 7.32-7.26 (m, 1H), 6.29 (bs, 1H), 6.09 (s, 1H), 5.30 (s, 1H), 3.00 (t, J=7.18 Hz, 2H), 2.91 (t, J=7.37 Hz, 2H), 2.13-2.04 (m, 1H), 1.83-1.63 (m, 4H), 1.12-1.02 (m, 2H), 0.99-0.90 (m, 2H).

### Example 9

### N-(1-(4-(6-Benzyl-7H-pyrrolo[2,3-c]pyridazin-3-yl)butyl)-2-oxo-1,2-dihydropyridin-4-yl)-2-phenylacetamide

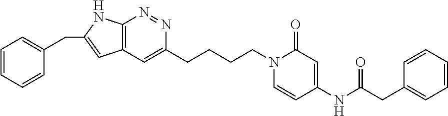 [SMILES: O=C(Cc1ccccc1)Nc1ccn(CCCCc2cc3cc(Cc4ccccc4)[nH]c3nn2)c(=O)c1]

### Steps 1 to 4

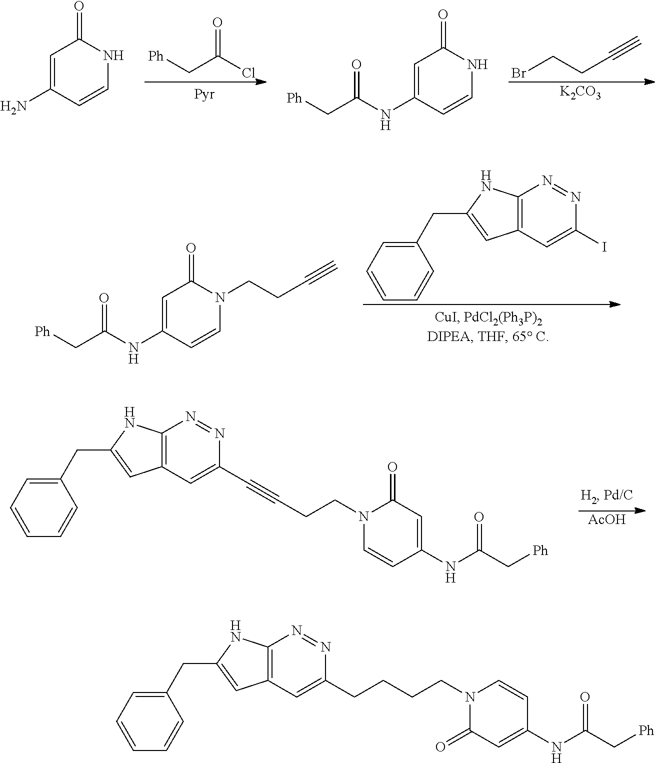

### Step 1: N-(2-oxo-1,2-dihydropyridin-4-yl)-2-phenylacetamide

To a mixture of pyridine (0.220 ml, 2.72 mmol) and 4-aminopyridin-2(1H)-one (0.20 g, 1.8 mmol) in DMF (2 ml) was added 2-phenylacetyl chloride (0.290 ml, 2.36 mmol). The resulting mixture was stirred at RT for 24 h, diluted with water (20 mL), stirred at 0° C. for 10 minutes and filtered. The solid was washed with cold water (5 mL) followed by Et₂O (5 mL) to give the title compound as a white solid (261 mg, 63%). MS (ES⁺) Cl₃H₁₂N₂O₂ requires: 228. found: 229 [M+H]⁺.

### Step 2: N-(1-(but-3-yn-1-yl)-2-oxo-1,2-dihydropyridin-4-yl)-2-phenylacetamide

To a mixture of N-(2-oxo-1,2-dihydropyridin-4-yl)-2-phenylacetamide (1.2 g, 5.26 mmol) and K₂CO₃ (1.45 g, 10.5 mmol) in DMSO (10.5 ml) at 60° C. was added 4-bromobut-1-yne (0.987 ml, 10.5 mmol) in four equal portions over 4 h. The reaction mixture was then diluted with EtOAc (100 mL) and washed with water (3×10 mL) and brine (2×10 mL). The combined organic layers were concentrated under reduced pressure and the residue was adsorbed onto SiO₂ and purified via SiO₂ gel chromatography (0-20% MeOH in DCM) to give the title compound as a brown solid (407 mg, 17%). MS (ES⁺) C₁₇H₁₆N₂O₂ requires: 280, found: 281 [M+H]⁺; ¹H NMR (600 MHz, DMSO-d₆) δ 10.30 (s, 1H), 7.59 (d, J=7.4 Hz, 1H), 7.35-7.28 (m, 4H), 7.25 (m, 1H), 6.75 (d, J=2.3 Hz, 1H), 6.36 (dd, J=7.4, 2.4 Hz, 1H), 3.90 (t, J=6.7 Hz, 2H), 3.66 (s, 2H), 2.88 (t, J=2.6 Hz, 1H), 2.53 (td, J=6.8, 2.7 Hz, 2H).

### Step 3: N-(1-(4-(6-Benzyl-7H-pyrrolo[2,3-c]pyridazin-3-yl)but-3-yn-1-yl)-2-oxo-1,2-dihydropyridin-4-yl)-2-phenylacetamide

To a suspension of 6-benzyl-3-iodo-7H-pyrrolo[2,3-c]pyridazine (Example 10, Step 3; 50.4 mg, 0.150 mmol) in THF (1.5 ml) were added N-(1-(but-3-yn-1-yl)-2-oxo-1,2-dihydropyridin-4-yl)-2-phenylacetamide (52.6 mg, 0.188 mmol), N-ethyl-N-isopropylpropan-2-amine (261 μl, 1.49 mmol), copper(I) iodide (3.5 mg, 0.018 mmol) and Pd(PPh₃)₂Cl₂ (7.5 mg, 10.7 μmol), and the resulting solution was stirred at 60° C. for 20 h. The mixture was concentrated under reduced pressure to give an orange residue, which was taken up in acetone, loaded onto silica gel and concentrated to give a yellow powder. The residue was purified via SiO₂ gel chromatography (0-10% MeOH in DCM) to give the title compound as an orange solid (52.2 mg, 0.107 mmol, 71% yield). MS (ES⁺) C₃₀H₂₅N₅O₂ requires: 487, found: 488 [M+H]⁺.

### Step 4: N-(1-(4-(6-Benzyl-7H-pyrrolo[2,3-c]pyridazin-3-yl)butyl)-2-oxo-1,2-dihydropyridin-4-yl)-2-phenylacetamide

To a solution of N-(1-(4-(6-benzyl-7H-pyrrolo[2,3-c]pyridazin-3-yl)but-3-yn-1-yl)-2-oxo-1,2-dihydropyridin-4-yl)-2-phenylacetamide (51.6 mg, 0.106 mmol) in AcOH (2 ml) under N₂ was added 10% Pd—C (49.8 mg, 0.0470 mmol). The flask was fitted with a two-neck adaptor connected to a vacuum line and a balloon filled with H₂. The flask was evacuated, filled with H₂, and the dark suspension was stirred under a H₂ balloon atmosphere for 16 h then filtered through a pad of Celite® and rinsed with AcOH. The filtrate was concentrated under reduced pressure at 70° C. to ca. 5 mL, and the remaining solution was put under N₂ and treated with 10% Pd—C (52.4 mg) as described above and stirred under a hydrogen balloon atmosphere for 21 h, then concentrated to give an orange residue. The residue was purified by mass-triggered preparative HPLC (Mobile phase: A=0.1% TFA/H₂O, B=0.1% TFA/MeCN; Gradient: B=30-70%; 12 min; Column: C18), and then by SiO₂ gel chromatography (0-10% MeOH in DCM), to give the title compound as an off-white solid (1.1 mg, 2.238 μmol, 2.1% yield). MS (ES⁺) C₃₀H₂₉N₅O₂ requires: 491, found: 492 [M+H]⁺; ¹H NMR (600 MHz, CD₃OD) δ 7.66 (s, 1H), 7.60 (d, J=7.18 Hz, 1H), 7.41-7.27 (m, 10H), 6.95 (bs, 1H), 6.71 (app d, J=5.67 Hz, 1H), 6.26 (s, 1H), 4.26 (s, 2H), 4.03 (t, J=6.42 Hz, 2H), 3.74 (s, 2H), 3.08 (t, J=6.61 Hz, 2H), 1.89-1.79 (m, 4H).

### Example 10

### 5-(4-(6-Benzyl-7H-pyrrolo[2,3-c]pyridazin-3-yl)butyl)-N-isobutyl-1,3,4-thiadiazole-2-carboxamide

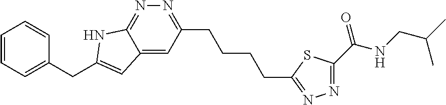 [SMILES: CC(C)CNC(=O)c1nnc(CCCCc2cc3cc(Cc4ccccc4)[nH]c3nn2)s1]

### Steps 1 to 2

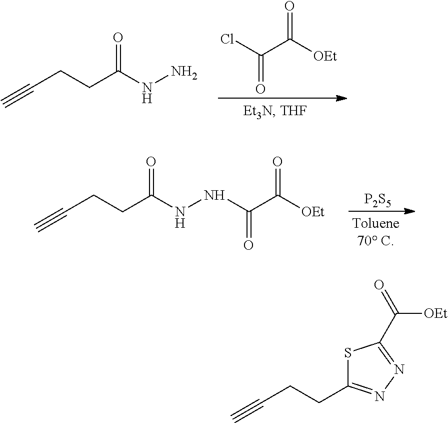

### Step 1: Ethyl 2-oxo-2-(2-pent-4-ynoylhydrazinyl)acetate

To a solution of pent-4-ynehydrazide (560 mg, 5.00 mmol) and TEA (530 mg, 5.25 mmol) in DCM/THF (25 mL/5 mL) at 0° C. was slowly added ethyl 2-chloro-2-oxoacetate (717 mg, 5.25 mmol). The resulting mixture was stirred for 30 minutes while warming to RT, then concentrated under reduced pressure. The residue was triturated with EtOAc (20 mL), filtered, and the filtrate was concentrated under reduced pressure to give the title compound. MS (ES⁺) C₉H₁₂N₂O₄, requires: 212.1, found: 213 [M+H]⁺.

### Step 2: Ethyl 5-(but-3-yn-1-yl)-1,3,4-thiadiazole-2-carboxylate

To a solution of ethyl 2-oxo-2-(2-pent-4-ynoylhydrazinyl)acetate (1.0 g, 5.0 mmol) in toluene (50 mL) at 70° C. was added portionwise P₂S₅ (1.11 g, 5.00 mmol), and the resulting mixture was stirred for 30 minutes at 70° C. The mixture was cooled to RT, filtered, and the filter cake was washed with DCM (20 mL). The filtrate was concentrated under reduced pressure, and the residue was purified via SiO₂ gel chromatography (20% EtOAc in hexanes) to give the title compound as a yellow solid (532 mg, 50%). MS (ES⁺) C₉H₁₀N₂O₂S, requires: 210.05, found: 211 [M+H]+. ¹H NMR (500 MHz, CDCl3) δ 4.54 (m, 2H), 3.44 (t, J=6.9 Hz, 2H), 2.75 (td, J=7.0, 2.7 Hz, 2H), 2.13 (t, J=2.6 Hz, 1H), 1.49 (m, 3H).

### Steps 3 to 6

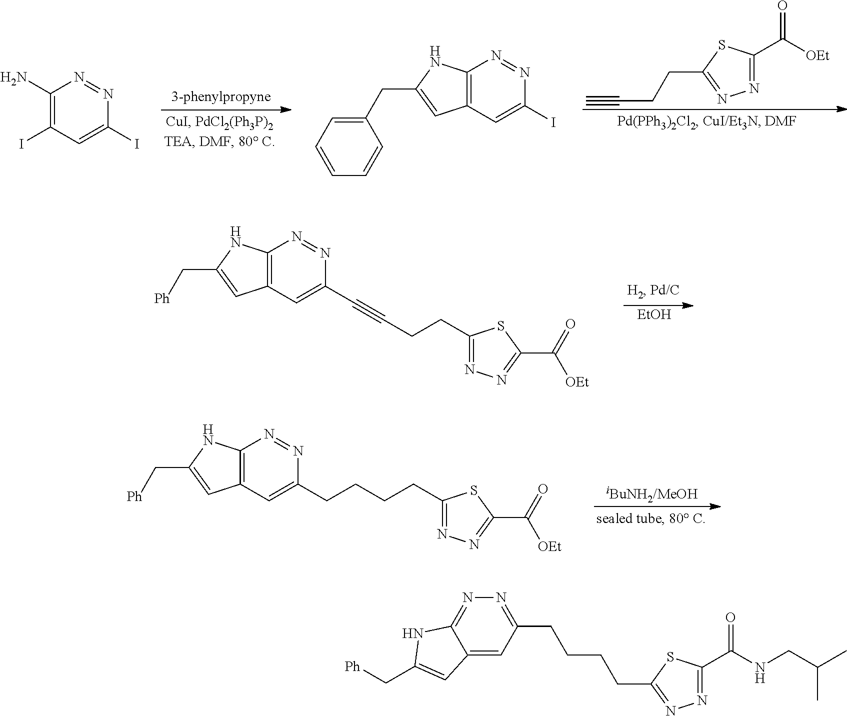

### Step 3: 6-Benzyl-3-iodo-7H-pyrrolo[2,3-c]pyridazine

To a solution of 4,6-diiodopyridazin-3-amine (Example 1, Step 1, 486 mg, 1.40 mmol) in DMF (14 ml) were added Et₃N (1.2 ml, 8.6 mmol), prop-2-yn-1-ylbenzene (0.174 ml, 1.40 mmol), copper(I) iodide (8 mg, 0.04 mmol) and Pd(PPh₃)₂Cl₂ (31 mg, 0.044 mmol). The vial was evacuated and filled with N₂ and the resulting dark orange solution was heated at 80° C. for 15 h, then concentrated under reduced pressure. The residue was purified via SiO₂ gel chromatography (0-33% EtOAc in hexanes) to give the title compound as a tan solid (194 mg, 0.578 mmol, 41% yield). MS (ES⁺) C₁₃H₁₀IN₃ requires: 335, found: 336 [M+H]⁺.

### Step 4: Ethyl 5-(4-(6-benzyl-7H-pyrrolo[2,3-c]pyridazin-3-yl)but-3-ynyl)-1,3,4-thiadiazole-2-carboxylate

To a mixture of 6-benzyl-3-iodo-7H-pyrrolo[2,3-c]pyridazine (1.00 g, 2.98 mmol), ethyl 5-(but-3-ynyl)-1,3,4-thiadiazole-2-carboxylate (630 mg, 2.98 mmol) and Et₃N (2.0 mL, 15 mmol) in DMF (20 mL) was added copper(I) iodide (60 mg, 0.30 mmol) and Pd(PPh₃)₂Cl₂ (105 mg, 0.150 mmol). The mixture was stirred under argon at 60° C. for 16 h, cooled to RT and concentrated under reduced pressure. The residue was diluted with acetone (50 mL), loaded onto silica gel (3.0 g), concentrated under reduced pressure and purified by SiO₂ gel column chromatography (2% MeOH in DCM) to afford the title compound as a tan solid (650 mg, 52%). MS (ES⁺) C₂₂H₁₉N₅O₂S requires: 417, found: 418 [M+H]+.

### Step 5: Ethyl 5-(4-(6-benzyl-7H-pyrrolo[2,3-c]pyridazin-3-yl)butyl)-1,3,4-thiadiazole-2-carboxylate

A reaction vessel was charged with ethyl 5-(4-(6-benzyl-7H-pyrrolo[2,3-c]pyridazin-3-yl)but-3-ynyl)-1,3,4-thiadiazole-2-carboxylate (200 mg, 0.480 mmol), AcOH (0.2 mL) and THF/MeOH (5 mL/3 mL) and 10% Pd—C (40 mg) under an atmosphere of N₂. The suspension was degassed with N₂ for 5 minutes, purged with H₂ and shaken under an atmosphere of H₂ (3 bar) at 60° C. for 16 h in the Parr apparatus. The mixture was then purged with N₂, filtered through a pad of Celite® and washed with MeOH. The filtrate was concentrated under reduced pressure to give the title compound as a yellow solid (200 mg, 99%). MS (ES⁺) C₂₂H₂₃N₅O₂S, requires: 421, found: 422 [M+H]⁺.

### Step 6: 5-(4-(6-Benzyl-7H-pyrrolo[2,3-c]pyridazin-3-yl)butyl)-N-isobutyl-1,3,4-thiadiazole-2-carboxamide

To a solution of ethyl 5-(4-(6-benzyl-7H-pyrrolo[2,3-c]pyridazin-3-yl)butyl)-1,3,4-thiadiazole-2-carboxylate (20 mg, 0.048 mmol) in MeOH (0.5 mL) was added 2-methylpropan-1-amine (25 μL), and the mixture was stirred at 80° C. for 2 h in a sealed tube. The mixture was then filtered and washed with MeOH (0.5 mL) to afford the title compound as a light yellow solid (5 mg, 24%). MS (ES⁺) C₂₄H₂₈N₆OS requires: 448, found: 449 [M+H]+; ¹H NMR (500 MHz, MeOD) δ 8.21 (s, 1H), 7.45-7.31 (m, 4H), 7.32 (m, 1H), 6.66 (s, 1H), 4.38 (s, 2H), 3.34-3.18 (m, 6H), 2.01-1.94 (m, 5H), 0.98 (d, J=7.0 Hz, 6H).

### Example 11

### 1-(4-(7H-pyrrolo[2,3-c]pyridazin-3-yl)butyl)-N-benzyl-1H-1,2,3-triazole-4-carboxamide

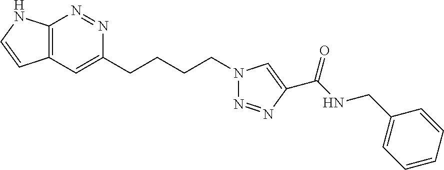 [SMILES: O=C(NCc1ccccc1)c1cn(CCCCc2cc3cc[nH]c3nn2)nn1]

### Steps 1 to 3

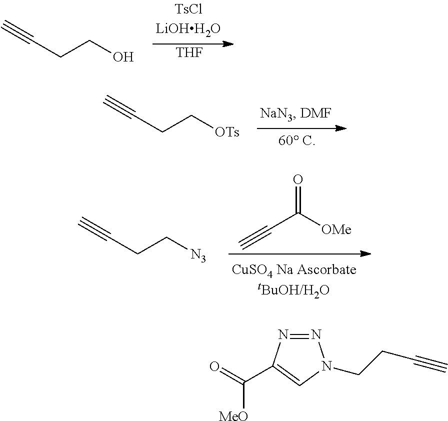

### Step 1: But-3-ynyl 4-methylbenzenesulfonate

To a solution of but-3-yn-1-ol (100 g, 1.42 mol) in THF (500 mL) was added LiOH.H₂O (89.5 g, 2.13 mol) in four portions, followed by the portionwise addition of 4-methylbenzene-1-sulfonyl chloride (270 g, 1.42 mol) while maintaining a temperature between 20-30° C. The resulting mixture was stirred at RT for 16 h, diluted with water (300 mL) and extracted with EtOAc (2×300 mL). The combined organic layers were washed with brine (100 ml), dried (Na₂SO₄), filtered and concentrated under reduced pressure to give the title compound as an oil (286 g, 90%).

### Step 2: 4-Azidobut-1-yne

To a suspension of NaN₃ (49 g, 0.75 mol) in DMF (200 mL) at 60° C. was slowly added but-3-ynyl 4-methylbenzenesulfonate (112 g, 0.500 mol). The mixture was stirred at 60° C. for 16 h, cooled to RT, diluted with water (300 mL) and extracted with Et₂O (3×500 mL). The combined organic layers were washed with water (2×1000 mL), brine (1000 mL), dried (Na₂SO₄), filtered and concentrated under reduced pressure to give the title compound as a yellow oil (48 g, 100%).

### Step 3: methyl 1-(but-3-ynyl)-1H-1,2,3-triazole-4-carboxylate

A mixture of 4-azidobut-1-yne (48 g, 0.50 mol), methyl propiolate (46.2 g, 0.55 mol), CuSO₄ (9.6 g, 60 mmol) and sodium ascorbate (19.2 g, 96.9 mmol) in ^{t}BuOH/H₂O (500 mL, 1/1 v/v) was stirred at RT for 16 h. The reaction mixture was diluted with water (250 mL), extracted with EtOAc (3×500 mL), and the combined organic layers were concentrated under reduced pressure. The residue was triturated with Et₂O (1000 mL) and dried under reduced pressure to give the title compound as a white solid (32 g, 35%). MS (ES⁺) C₈H₉N₃O₂ requires: 179, found: 180 [M+H]⁺. ¹H NMR (400 MHz, DMSO-d₆) δ 8.83 (s, 1H), 4.56 (t, J=6.6 Hz, 2H), 3.84 (s, 3H), 2.92 (t, J=2.6 Hz, 1H), 2.84 (td, J=6.6, 2.6 Hz, 2H).

### Steps 4 to 7

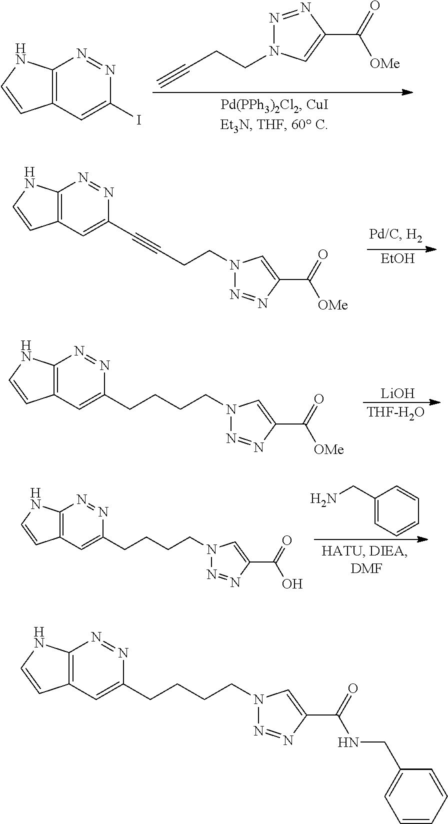

### Step 4: Methyl 1-(4-(7H-pyrrolo[2,3-c]pyridazin-3-yl)but-3-yn-1-yl)-1H-1,2,3-triazole-4-carboxylate

A mixture of 3-iodo-7H-pyrrolo[2,3-c]pyridazine (Example 1, Step 4; 400 mg, 1.63 mmol), methyl 1-(but-3-yn-1-yl)-1H-1,2,3-triazole-4-carboxylate (Step 3; 293 mg, 1.63 mmol), Pd(PPh₃)₂Cl₂ (115 mg, 0.163 mmol), copper (I) iodide (31.1 mg, 0.163 mmol) and Et₃N (2.28 ml, 16.3 mmol) in THF (10 ml) was degassed with N₂ and stirred at 60° C. for 2 h. The mixture was cooled to RT and the volatiles were removed under reduced pressure. The residue was purified by mass-triggered preparative HPLC (Mobile phase: A=0.1% TFA/H₂O, B=0.1% TFA/MeCN; Gradient: B=30-70%; 12 min; Column: C18) to give methyl the title compound as an off-white solid (410 mg, 85% yield). MS (ES⁺) C₁₄H₁₂N₆O₂ requires: 296, found: 297 [M+H]⁺.

### Step 5: Methyl 1-(4-(7H-pyrrolo[2,3-c]pyridazin-3-yl)butyl)-1H-1,2,3-triazole-4-carboxylate

A reaction vessel was charged with methyl 1-(4-(7H-pyrrolo[2,3-c]pyridazin-3-yl)but-3-yn-1-yl)-1H-1,2,3-triazole-4-carboxylate (400 mg, 1.35 mmol), EtOH (40 mL) and 10% Pd—C (400 mg, 3.76 mmol) under an atmosphere of N₂. The suspension was degassed with N₂ for 1 minute, purged with H₂ and stirred under an atmosphere of H₂ at RT for 2 h. The reaction mixture was then purged with N₂, filtered through a pad of Celite® and concentrated under reduced pressure to give the title compound as a yellow solid (356 mg, 88% yield). MS (ES⁺) C₁₄H₁₆N₆O₂ requires: 300, found: 301 [M+H]⁺.

### Step 6: 1-(4-(7H-Pyrrolo[2,3-c]pyridazin-3-yl)butyl)-1H-1,2,3-triazole-4-carboxylic acid

To a solution of methyl 1-(4-(7H-pyrrolo[2,3-c]pyridazin-3-yl)butyl)-1H-1,2,3-triazole-4-carboxylate (200 mg, 0.666 mmol) in THF (3 ml) and water (3 ml) was added LiOH (80 mg, 3.33 mmol) and the resulting mixture was stirred at RT for 1 h. The volatiles were removed under reduced pressure, the residue was taken in MeOH and purified by mass-triggered preparative HPLC (Mobile phase: A=0.1% TFA/H₂O, B=0.1% TFA/MeCN; Gradient: B=0-30%; 12 min; Column: C18) to give the title compound as an off-white solid (152 mg, 80% yield). MS (ES⁺) C₁₃H₁₄N₆O₂ requires: 286, found: 287 [M+H]⁺.

### Step 7: 1-(4-(7H-pyrrolo[2,3-c]pyridazin-3-yl)butyl)-N-benzyl-1H-1,2,3-triazole-4-carboxamide

To a solution of 1-(4-(7H-pyrrolo[2,3-c]pyridazin-3-yl)butyl)-1H-1,2,3-triazole-4-carboxylic acid (15 mg, 0.052 mmol) in DMF (0.5 ml) were added HATU (21.9 mg, 0.0580 mmol), benzylamine (5.7 μl, 0.052 mmol) and DIEA (0.014 ml, 0.079 mmol), and the resulting mixture was stirred at RT for 1 h. The volatiles were removed under reduced pressure. The residue was purified by mass-triggered preparative HPLC (Mobile phase: A=0.1% TFA/H₂O, B=0.1% TFA/MeCN; Gradient: B=10-50%; 12 min; Column: C18) to give the title compound as a white solid (14 mg, 71% yield). MS (ES⁺) C₂₀H₂₁N₇O requires: 375, found: 376 [M+H]⁺. ¹H NMR (600 MHz, DMSO-d₆) δ 13.53 (bs, 1H), 9.07 (t, J=6 Hz, 1H), 8.59 (bs, 1H), 8.43 (bs, 1H), 7.36-7.26 (m, 5H), 6.93 (s, 1H), 4.50-4.42 (m, 4H), 3.14 (t, J=7.8 Hz, 2H), 1.95-1.88 (m, 2H), 1.75-1.68 (m, 2H).

### Example 12

### 1-(4-(6-isopropyl-7H-pyrrolo[2,3-c]pyridazin-3-yl)butyl)-N-(pyridin-2-ylmethyl)-1H-1,2,3-triazole-4-carboxamide

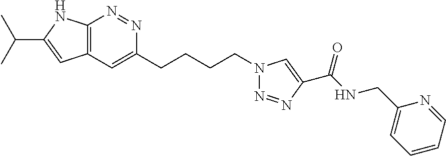 [SMILES: CC(C)c1cc2cc(CCCCn3cc(C(=O)NCc4ccccn4)nn3)nnc2[nH]1]

Prepared as described for Example 11, using 3-iodo-6-isopropyl-7H-pyrrolo[2,3-c]pyridazine (prepared as described in Example 8, Step 2) instead of 3-iodo-7H-pyrrolo[2,3-c]pyridazine. MS (ES⁺) C₂₂H₂₆N₈O requires: 418, found: 419 [M+H]⁺. ¹H NMR (600 MHz, DMSO-d₆) δ 13.48 (s, 1H), 9.09 (t, J=6 Hz, 1H), 8.63 (s, 1H), 8.55 (d, J=4.8 Hz, 1H), 8.30 (s, 1H), 7.84 (m, 1H), 7.40-7.30 (m, 2H), 6.76 (s, 1H), 4.58 (d, J=6.0 Hz, 2H), 4.47 (t, J=7.2 Hz, 2H), 3.28 (m, 1H), 3.12 (t, J=7.2 Hz, 2H), 1.96-1.88 (m, 2H), 1.80-1.70 (m, 2H), 1.38 (d, J=7.2 Hz, 6H).

### Example 13

### N-benzyl-1-(4-(6-isobutyl-7H-pyrrolo[2,3-c]pyridazin-3-yl)butyl)-1H-1,2,3-triazole-4-carboxamide

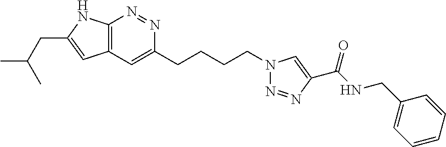 [SMILES: CC(C)Cc1cc2cc(CCCCn3cc(C(=O)NCc4ccccc4)nn3)nnc2[nH]1]

Prepared as described for Example 11, using 3-iodo-6-isobutyl-7H-pyrrolo[2,3-c]pyridazine (prepared as described in Example 8, Step 2) instead of 3-iodo-7H-pyrrolo[2,3-c]pyridazine. MS (ES⁺) C₂₄H₂₉N₇O requires: 431, found: 432 [M+H]⁺. ¹H NMR (600 MHz, DMSO-d₆) δ 13.41 (s, 1H), 9.06 (t, J=7.8 Hz, 1H), 8.59 (s, 1H), 8.25 (bs, 1H), 7.34-7.20 (m, 5H), 6.72 (s, 1H), 4.50-4.40 (m, 4H), 3.09 (t, J=9.0 Hz, 2H), 2.86-2.82 (m, 2H), 2.16 (m, 1H), 1.92 (m, 1H), 1.72 (m, 1H), 0.95 (d, J=7.8 Hz, 6H).

### Example 14

### N-benzyl-1-(4-(6-benzyl-7H-pyrrolo[2,3-c]pyridazin-3-yl)butyl)-1H-1,2,3-triazole-4-carboxamide

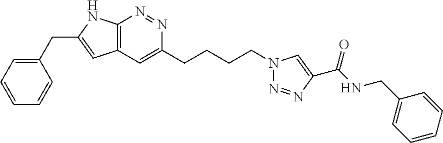 [SMILES: O=C(NCc1ccccc1)c1cn(CCCCc2cc3cc(Cc4ccccc4)[nH]c3nn2)nn1]

Prepared as described for Example 11, using 6-benzyl-3-iodo-7H-pyrrolo[2,3-c]pyridazine (Example 10, Step 3) instead of 3-iodo-7H-pyrrolo[2,3-c]pyridazine. MS (ES⁺) C₂₇H₂₇N₇O requires: 465, found: 466 [M+H]⁺. ¹H NMR (600 MHz, DMSO-d₆) δ 12.22 (s, 1H), 9.00 (t, J=6 Hz, 1H), 8.57 (s, 1H), 7.52 (s, 1H), 7.52-7.21 (m, 10H), 6.14 (s, 1H), 4.48-4.40 (m, 4H), 4.13 (s, 2H), 2.94 (t, J=7.2 Hz, 2H), 1.92-1.84 (m, 2H), 1.72-1.65 (m, 2H).

### Example 15

### N-benzyl-1-(4-(5-iodo-7H-pyrrolo[2,3-c]pyridazin-3-yl)butyl)-1H-1,2,3-triazole-4-carboxamide

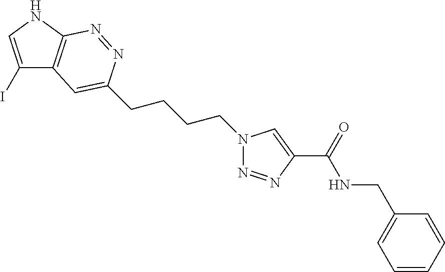 [SMILES: O=C(NCc1ccccc1)c1cn(CCCCc2cc3c(I)c[nH]c3nn2)nn1]

### Steps 1 to 3

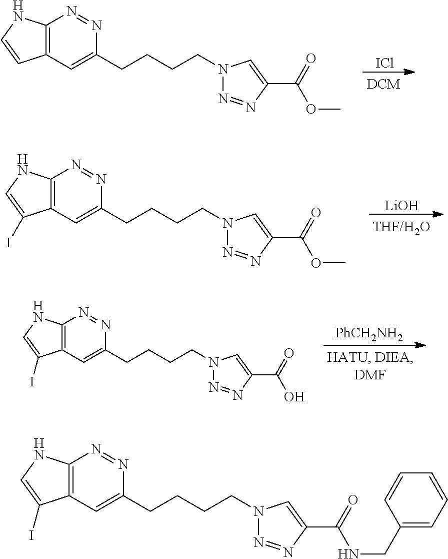

### Step 1: Methyl 1-(4-(5-iodo-7H-pyrrolo[2,3-c]pyridazin-3-yl)butyl)-1H-1,2,3-triazole-4-carboxylate

To a solution of methyl 1-(4-(7H-pyrrolo[2,3-c]pyridazin-3-yl)butyl)-1H-1,2,3-triazole-4-carboxylate (Example 11, Step 5; 30 mg, 0.10 mmol) in DCM (1 ml) was added ICl (24 mg, 0.15 mmol) and the resulting mixture was stirred at RT for 2 h. The resulting suspension was filtered to give the title compound as a yellow solid (34 mg, 80% yield). MS (ES⁺) C₁₄H₁₅IN₆O₂ requires: 426, found: 427 [M+H]⁺.

### Step 2: 1-(4-(5-Iodo-7H-pyrrolo[2,3-c]pyridazin-3-yl)butyl)-1H-1,2,3-triazole-4-carboxylic acid

To a solution of methyl 1-(4-(5-iodo-7H-pyrrolo[2,3-c]pyridazin-3-yl)butyl)-1H-1,2,3-triazole-4-carboxylate (32 mg, 0.075 mmol) in THF (0.5 ml) and water (0.5 ml) was added LiOH (8.99 mg, 0.375 mmol) and the resulting mixture was stirred at RT for 1 h. The volatiles were removed under reduced pressure, and the residue was purified by mass-triggered preparative HPLC (Mobile phase: A=0.1% TFA/H₂O, B=0.1% TFA/MeCN; Gradient: B=10-50%; 12 min; Column: C18) to give the title compound as a yellow solid (22 mg, 71% yield). MS (ES⁺) C₁₃H₁₃IN₆O₂ requires: 412, found: 413 [M+H]⁺.

### Step 3: N-benzyl-1-(4-(5-iodo-7H-pyrrolo[2,3-c]pyridazin-3-yl)butyl)-1H-1,2,3-triazole-4-carboxamide

To a solution of 1-(4-(5-iodo-7H-pyrrolo[2,3-c]pyridazin-3-yl)butyl)-1H-1,2,3-triazole-4-carboxylic acid (10 mg, 0.024 mmol) in DMF (0.5 ml) were added HATU (10 mg, 0.027 mmol), benzylamine (2.9 mg, 0.027 mmol) and DIEA (6.4 μl, 0.036 mmol), and the resulting mixture was stirred at RT for 1 h. The volatiles were removed under reduced pressure and the residue was purified by mass-triggered preparative HPLC (Mobile phase: A=0.1% TFA/H₂O, B=0.1% TFA/MeCN; Gradient: B=40-80%; 12 min; Column: C18) to give the title compound as an off-white solid (8 mg, 66% yield). MS (ES⁺) C₂₀H₂₀IN₇O requires: 501. found: 502 [M+H]⁺. ¹H NMR (600 MHz, DMSO-d₆) δ 12.68 (s, 1H), 9.02 (t, J=6 Hz, 1H), 8.59 (s, 1H), 8.06 (s, 1H), 7.41 (s, 1H), 7.35-7.20 (m, 5H), 4.52-4.40 (m, 4H), 3.05 (t, J=7.2 Hz, 2H), 1.96-1.90 (m, 2H), 1.78-1.70 (m, 2H).

### Example 16

### 1-(4-(6-cyclopropyl-7H-pyrrolo[2,3-c]pyridazin-3-yl)butyl)-N-(pyridin-2-ylmethyl)-1H-1,2,3-triazole-4-carboxamide

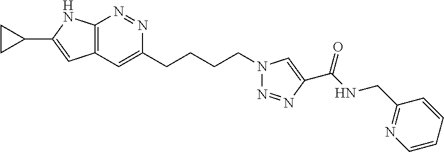 [SMILES: O=C(NCc1ccccn1)c1cn(CCCCc2cc3cc(C4CC4)[nH]c3nn2)nn1]

### Steps 1-4

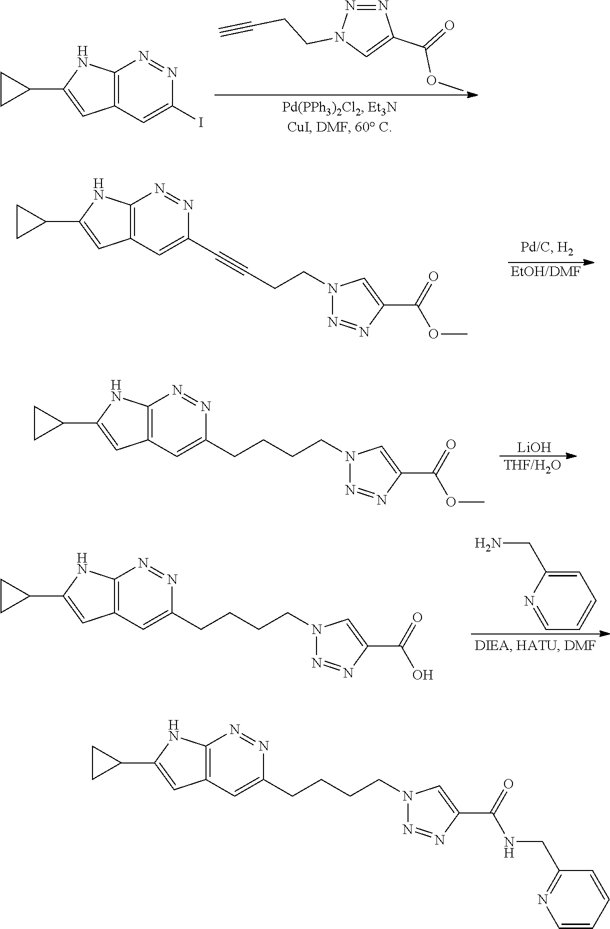

### Step 1: Methyl 1-(4-(6-cyclopropyl-7H-pyrrolo[2,3-c]pyridazin-3-yl)but-3-yn-1-yl)-1H-1,2,3-triazole-4-carboxylate

A mixture of 6-cyclopropyl-3-iodo-7H-pyrrolo[2,3-c]pyridazine (Example 8, Step 2; 350 mg, 1.23 mmol), methyl 1-(but-3-yn-1-yl)-1H-1,2,3-triazole-4-carboxylate (Example 11, Step 3; 220 mg, 1.23 mmol) and Et₃N (1.24 g, 12.3 mmol) in THF (10 ml) was degassed with N₂ for 2 minutes. Pd(PPh₃)₂Cl₂ (86 mg, 0.12 mmol) and copper(I) iodide (23 mg, 0.12 mmol) were added and the mixture was degassed with N₂ for an additional 2 minutes. The reaction mixture was heated at 60° C. for 2 h, then cooled to RT. The volatiles were removed under reduced pressure, the solid residue was sonicated and stirred in water for 10 minutes, filtered, and purified via SiO₂ gel chromatography (1-8% MeOH in DCM) to give the title compound as a yellow solid (330 mg, 80% yield). MS (ES⁺) C₁₈H₂₀N₆O₂ requires: 336, found: 337 [M+H]⁺.

### Step 2: methyl 1-(4-(6-cyclopropyl-7H-pyrrolo[2,3-c]pyridazin-3-yl)butyl)-1H-1,2,3-triazole-4-carboxylate

A reaction vessel was charged with methyl 1-(4-(6-cyclopropyl-7H-pyrrolo[2,3-c]pyridazin-3-yl)but-3-yn-1-yl)-1H-1,2,3-triazole-4-carboxylate (300 mg, 0.892 mmol), 5% Pd—C (300 mg), ethanol (20 ml) and DMF (20 ml) under an atmosphere of N₂. The suspension was degassed with N₂ for 2 minutes, purged with H₂ and stirred under an atmosphere of H₂ at 1 atm for 16 h. The reaction mixture was purged with N₂, filtered through a pad of Celite® and concentrated under reduced pressure to give the title compound as a yellow solid (300 mg, 99% yield). MS (ES⁺) C₁₇H₂₀N₆O₂ requires: 340, found: 341 [M+H]⁺.

### Step 3: 1-(4-(6-cyclopropyl-7H-pyrrolo[2,3-c]pyridazin-3-yl)butyl)-1H-1,2,3-triazole-4-carboxylic acid

To a solution of methyl 1-(4-(6-cyclopropyl-7H-pyrrolo[2,3-c]pyridazin-3-yl)butyl)-1H-1,2,3-triazole-4-carboxylate (300 mg, 0.881 mmol) in THF (5 ml) and water (5 ml) was added LiOH (42 mg, 1.8 mmol) and the resulting mixture was stirred at RT for 1 h. The volatiles were removed under reduced pressure and the residue was taken up in MeOH and purified by mass-triggered preparative HPLC (Mobile phase: A=0.1% TFA/H₂O, B=0.1% TFA/MeCN; Gradient: B=10-40%; 12 min; Column: C18) to give the title compound as a white solid (239 mg, 83% yield). MS (ES⁺) C₁₆H₁₈N₆O₂ requires: 326, found: 327 [M+H]⁺.

### Step 4: 1-(4-(6-cyclopropyl-7H-pyrrolo[2,3-c]pyridazin-3-yl)butyl)-N-(pyridin-2-ylmethyl)-1H-1,2,3-triazole-4-carboxamide

To a solution of 1-(4-(6-cyclopropyl-7H-pyrrolo[2,3-c]pyridazin-3-yl)butyl)-1H-1,2,3-triazole-4-carboxylic acid (34 mg, 0.10 mmol) in DMF (1 ml) were added HATU (43.6 mg, 0.115 mmol), pyridin-2-ylmethanamine (12.4 mg, 0.115 mmol) and DIEA (0.027 ml, 0.16 mmol) and the resulting mixture was stirred at RT for 1 h. The volatiles were removed under reduced pressure and the residue was purified by mass-triggered preparative HPLC (Mobile phase: A=0.1% TFA/H₂O, B=0.1% TFA/MeCN; Gradient: B=10-50%; 12 min; Column: C18) to give the title compound as a white solid (42 mg, 97% yield). MS (ES⁺) C₂₂H₂₄N₈O requires: 416, found: 417 [M+H]⁺. ¹H NMR (600 MHz, DMSO-d₆) δ 13.40 (bs, 1H), 9.05 (t, J=6 Hz, 1H), 8.61 (s, 1H), 8.54 (d, J=4.8 Hz, 1H), 8.15 (s, 1H), 7.83 (t, J=7.2 Hz, 1H), 7.37-7.31 (m, 2H), 6.65 (s, 1H), 4.58 (d, J=6.0 Hz, 2H), 4.47 (t, J=7.2 Hz, 2H), 3.07 (t, J=7.2 Hz, 2H), 2.31 (m, 1H), 1.95-1.88 (m, 2H), 1.77-1.68 (m, 2H), 1.38-1.32 (m, 2H), 1.18-1.12 (m, 2H).

### Example 17

### 1-(4-(6-Cyclopropyl-7H-pyrrolo[2,3-c]pyridazin-3-yl)butyl)-N-((4-(2-hydroxypropan-2-yl)pyridin-2-yl)methyl)-1H-1,2,3-triazole-4-carboxamide 2,2,2-trifluoroacetate

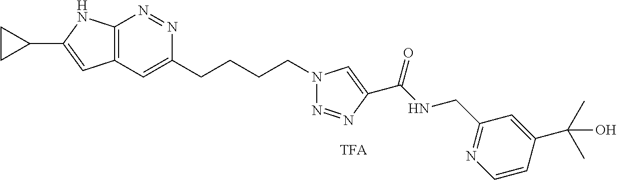 [SMILES: CC(C)(O)c1ccnc(CNC(=O)c2cn(CCCCc3cc4cc(C5CC5)[nH]c4nn3)nn2)c1]

### Steps 1 to 2

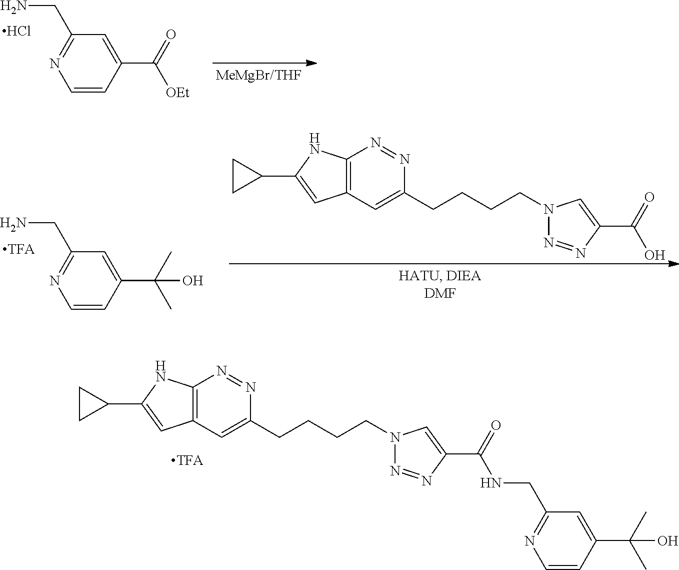 [SMILES: Cl ; CCOC(=O)c1ccnc(CN)c1 ; CC(C)(O)c1ccnc(CN)c1 ; O=C(O)c1cn(CCCCc2cc3cc(C4CC4)[nH]c3nn2)nn1 ; CC(C)(O)c1ccnc(CNC(=O)c2cn(CCCCc3cc4cc(C5CC5)[nH]c4nn3)nn2)c1]

### Step 1: 2-(2-(aminomethyl)pyridin-4-yl)propan-2-ol 2,2,2-trifluoroacetate. To a suspension of ethyl

2-(aminomethyl)isonicotinate hydrochloride (46.7 mg, 0.216 mmol) in THF (2 ml) was added methylmagnesium bromide (3.0 M in ether, 0.40 ml, 1.2 mmol) dropwise. The resulting cloudy blue mixture was stirred at RT for 12.5 h, and then treated dropwise with 1 mL of methanol (abundant gas evolution). The resulting yellow-orange mixture was concentrated under reduced pressure, taken up again in methanol, filtered and the filtrate was concentrated to a reduced volume for loading onto reverse-phase HPLC. The residue was purified by mass-triggered preparative HPLC (Mobile phase: A=0.1% TFA/H₂O, B=0.1% TFA/MeCN; Gradient: B=0-30%; 12 min; Column: C18) to give the title compound. MS (ES⁺) C₉H₁₄N₂O requires: 166, found: 167 [M+H]⁺.

### Step 2: 1-(4-(6-Cyclopropyl-7H-pyrrolo[2,3-c]pyridazin-3-yl)butyl)-N-((4-(2-hydroxypropan-2-yl)pyridin-2-yl)methyl)-1H-1,2,3-triazole-4-carboxamide 2,2,2-trifluoroacetate

A mixture of 2-(2-(aminomethyl)pyridin-4-yl)propan-2-ol 2,2,2-trifluoroacetate (60 mg, 0.22 mmol), 1-(4-(6-cyclopropyl-7H-pyrrolo[2,3-c]pyridazin-3-yl)butyl)-1H-1,2,3-triazole-4-carboxylic acid (Example 16, Step 3; 14.5 mg, 0.0440 mmol), 1-hydroxy benzotriazole hydrate (10.9 mg, 0.0710 mmol), and N-((ethylimino)methylene)-N′,N′-dimethylpropane-1,3-diamine hydrochloride (14.5 mg, 0.0760 mmol) in DMF (1 ml) was stirred at RT for 30 minutes, followed by the addition of Et₃N (65 μl, 0.47 mmol). The resulting yellow mixture was stirred at RT for 39 h then concentrated to a light yellow solid. The residue was purified by mass-triggered preparative HPLC (Mobile phase: A=0.1% TFA/H₂O, B=0.1% TFA/MeCN; Gradient: B=10-50%; 20 min; Column: C18) to give the title as an off-white solid compound (2.2 mg, 3.74 μmol, 8.4% yield). MS (ES⁺) C₂₅H₃₀N₈O₂ requires: 474, found: 475 [M+H]⁺, 238 [(M+2H)/2]⁺; ¹H NMR (600 MHz, METHANOL-d₄) 8.57 (d, J=6.0 Hz, 1H), 8.42 (s, 1H), 8.03 (s, 1H), 7.94 (s, 1H), 7.82 (d, J=5.7 Hz, 1H), 6.58 (s, 1H), 4.54 (t, J=6.7 Hz, 2H), 3.13 (t, J=7.7 Hz, 2H), 2.31 (m, 1H), 2.09-2.01 (m, 2H), 1.85 (t, J=7.55 Hz, 2H), 1.54 (s, 6H), 1.44-1.39 (m, 2H), 1.22-1.17 (m, 2H). Two aliphatic protons are assumed to be coincident with the water peak.

### Example 18

### 1-(4-(5-Bromo-6-cyclopropyl-7H-pyrrolo[2,3-c]pyridazin-3-yl)butyl)-N-((6-methylpyridin-3-yl)methyl)-1H-1,2,3-triazole-4-carboxamide 2,2,2-trifluoroacetate

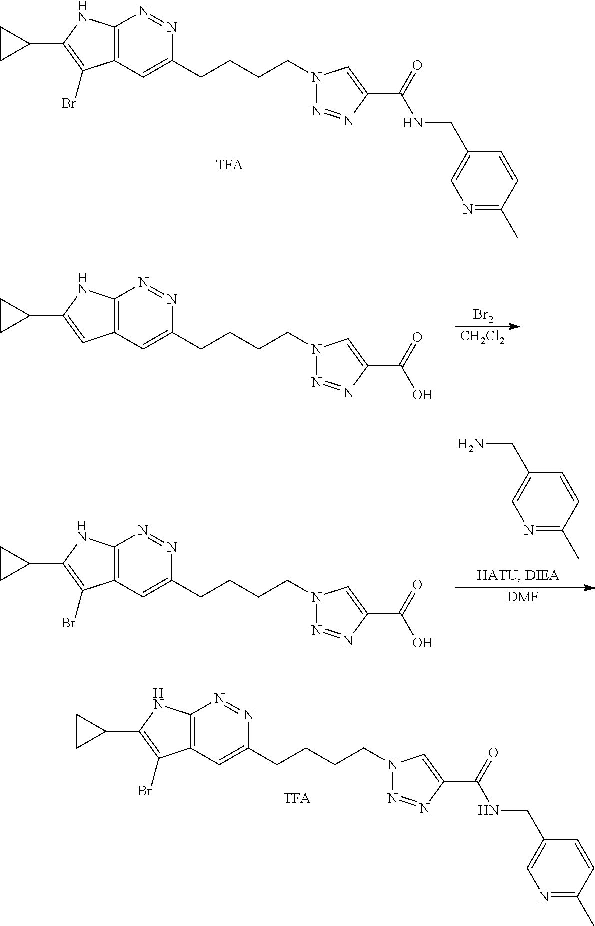

### Step 1: 1-(4-(5-Bromo-6-cyclopropyl-7H-pyrrolo[2,3-c]pyridazin-3-yl)butyl)-1H-1,2,3-triazole-4-carboxylic acid

To a suspension of 1-(4-(6-cyclopropyl-7H-pyrrolo[2,3-c]pyridazin-3-yl)butyl)-1H-1,2,3-triazole-4-carboxylic acid (Example 16, Step 3; 104 mg, 0.318 mmol) in DCM (10 ml) was added Br₂ (0.049 ml, 0.96 mmol) and the resulting mixture was stirred at RT for 30 minutes, diluted with Et₂O (10 mL) and filtered. The solid was rinsed with Et₂O and dried under reduced pressure at 50° C. for 5 h to give the title compound as a yellow solid (131 mg, 0.269 mmol, 84% yield). MS (ES⁺) C₁₆H₁₇BrN₆O₂ requires: 404/405, found: 405/407 [M+H]⁺.

### Step 2: 1-(4-(5-Bromo-6-cyclopropyl-7H-pyrrolo[2,3-c]pyridazin-3-yl)butyl)-N-((6-methylpyridin-3-yl)methyl)-1H-1,2,3-triazole-4-carboxamide 2,2,2-trifluoroacetate

To a solution of 1-(4-(5-bromo-6-cyclopropyl-7H-pyrrolo[2,3-c]pyridazin-3-yl)butyl)-1H-1,2,3-triazole-4-carboxylic acid (17.7 mg, 0.0360 mmol) in DMF (0.36 ml) were added DIPEA (16.0 μl, 0.0920 mmol), HATU (15 mg, 0.040 mmol) and (6-methylpyridin-3-yl)methanamine (5.2 mg, 0.043 mmol). The resulting yellow solution was stirred at RT for 2 h and concentrated under reduced pressure. The residue was purified by mass-triggered preparative HPLC (Mobile phase: A=0.1% TFA/H₂O, B=0.1% TFA/MeCN; Gradient: B=20-50%; 20 min; Column: C18) to give the title compound as a yellow deliquescent solid (1.1 mg, 1.8 μmol, 4.9% yield). MS (ES⁺) C₂₃H₂₅BrN₈O requires: 508/510, found: 509/511 [M+H]⁺, 255/256 [(M+2)/2)]⁺; ¹H NMR (600 MHz, METHANOL-d₄) 8.63 (s, 1H), 8.40 (s, 1H), 8.34 (d, J=7.5 Hz, 1H), 8.02 (s, 1H), 7.78 (d, J=8.3 Hz, 1H), 4.68 (s, 2H), 4.54 (t, J=6.7 Hz, 2H), 3.13-3.20 (m, 2H), 2.72 (s, 3H), 2.50 (m, 1H), 2.01-2.10 (m, 2H), 1.82-1.90 (m, 2H), 1.47-1.53 (m, 2H), 1.31-1.39 (m, 2H).

### Example 19

### 1-(4-(5-Chloro-6-cyclopropyl-7H-pyrrolo[2,3-c]pyridazin-3-yl)butyl)-N-((6-methylpyridin-3-yl)methyl)-1H-1,2,3-triazole-4-carboxamide 2,2,2-trifluoroacetate

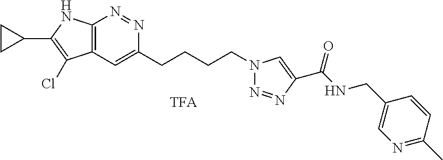 [SMILES: Cc1ccc(CNC(=O)c2cn(CCCCc3cc4c(Cl)c(C5CC5)[nH]c4nn3)nn2)cn1]

### Steps 1 to 2

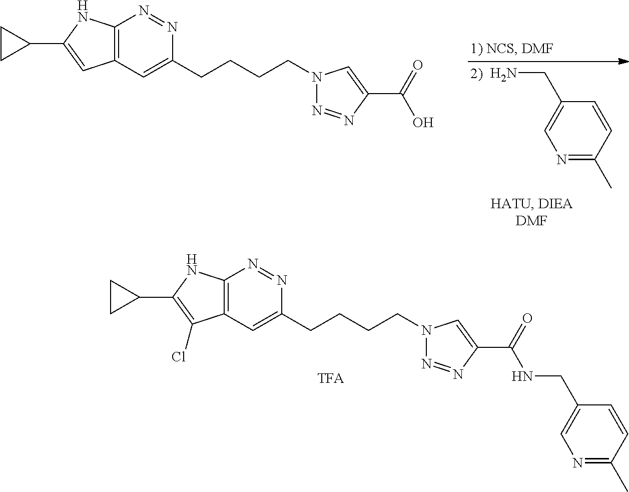

### Step 1: 1-(4-(5-Chloro-6-cyclopropyl-7H-pyrrolo[2,3-c]pyridazin-3-yl)butyl)-1H-1,2,3-triazole-4-carboxylic acid

To a solution of 1-(4-(6-cyclopropyl-7H-pyrrolo[2,3-c]pyridazin-3-yl)butyl)-1H-1,2,3-triazole-4-carboxylic acid (Example 16, Step 3; 21 mg, 0.063 mmol) in DMF (0.20 ml) was added N-chlorosuccinimide (8.7 mg, 0.065 mmol) and the resulting mixture was stirred at RT for 42 h. The volatiles were removed under reduced pressure to give the title compound. MS (ES⁺) C₁₆H₁₇ClN₆O₂ requires: 360, found: 361 [M+H]⁺.

### Step 2: 1-(4-(5-Chloro-6-cyclopropyl-7H-pyrrolo[2,3-c]pyridazin-3-yl)butyl)-N-((6-methylpyridin-3-yl)methyl)-1H-1,2,3-triazole-4-carboxamide 2,2,2-trifluoroacetate

To a solution of 1-(4-(5-chloro-6-cyclopropyl-7H-pyrrolo[2,3-c]pyridazin-3-yl)butyl)-1H-1,2,3-triazole-4-carboxylic acid (0.063 mmol) in DMF (0.30 ml) were added DIEA (0.016 ml, 0.092 mmol), HATU (31 mg, 0.082 mmol) and (6-methylpyridin-3-yl)methanamine (9.9 mg, 0.081 mmol). The resulting mixture was stirred at RT for 1 h, the volatiles were removed under reduced pressure and the residue was purified by mass-triggered preparative HPLC (Mobile phase: A=0.1% TFA/H₂O, B=0.1% TFA/MeCN; Gradient: B=20-50%; 20 min; Column: C18) to give the title compound as a yellow deliquescent solid (2.5 mg, 4.3 μmol, 6.8% yield). MS (ES⁺) C₂₃H₂₅ClN₈O requires: 464, found: 465 [M+H]⁺, 233 [(M+2)/2)]⁺; ¹H NMR (600 MHz, METHANOL-d₄) 8.62 (s, 1H), 8.39 (s, 1H), 8.32 (d, J=8.3 Hz, 1H), 8.09 (s, 1H), 7.77 (d, J=8.3 Hz, 1H), 4.68 (s, 2H), 4.54 (t, J=6.7 Hz, 2H), 3.20-3.14 (m, 2H), 2.71 (s, 3H), 2.51 (m, 1H), 2.09-2.02 (m, 2H), 1.86 (t, J=7.7 Hz, 2H), 1.53-1.48 (m, 2H), 1.39-1.33 (m, 2H).

### Example 20

### 1-(4-(5-phenyl-7H-pyrrolo[2,3-c]pyridazin-3-yl)butyl)-N-(pyridin-2-ylmethyl)-1H-1,2,3-triazole-4-carboxamide

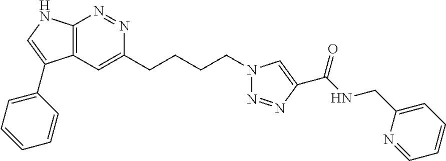 [SMILES: O=C(NCc1ccccn1)c1cn(CCCCc2cc3c(-c4ccccc4)c[nH]c3nn2)nn1]

### Steps 1 to 7

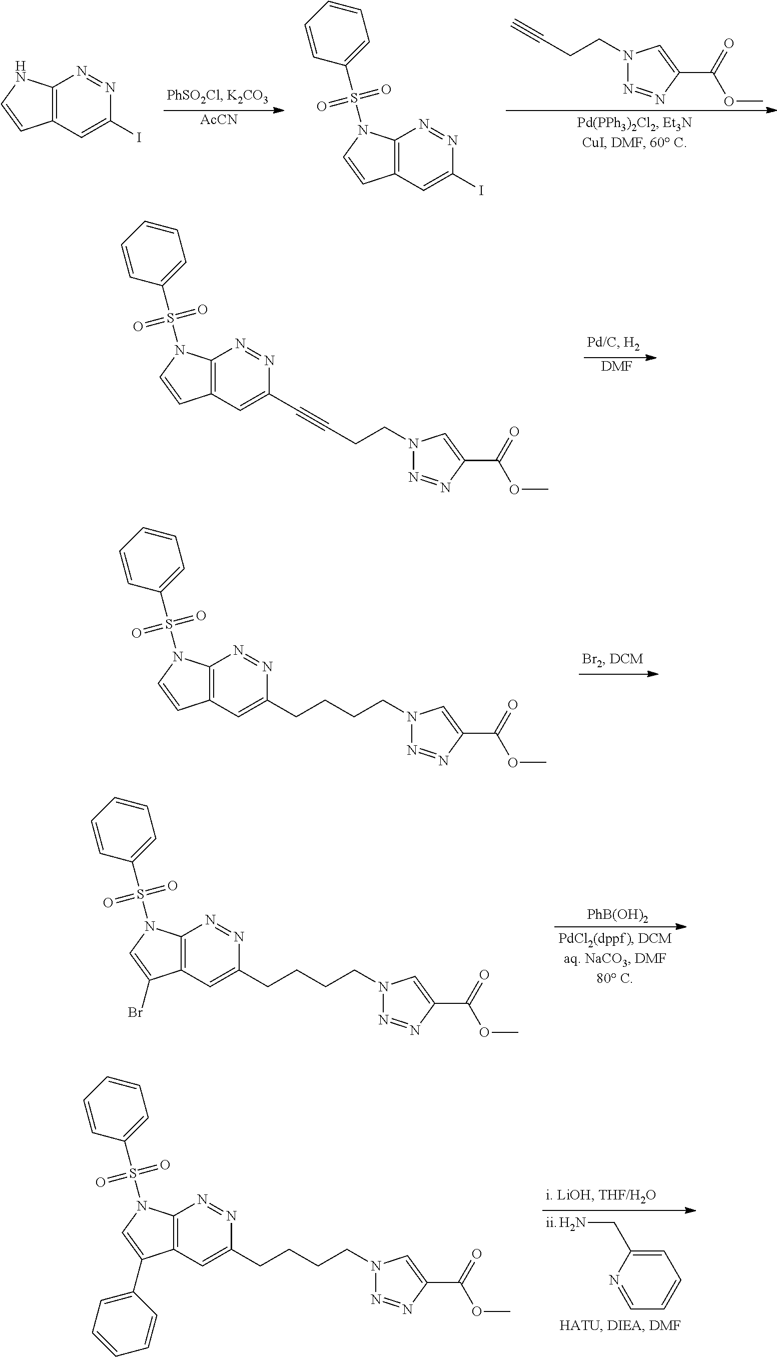

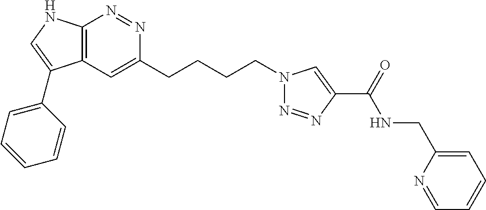 [SMILES: O=C(NCc1ccccn1)c1cn(CCCCc2cc3c(-c4ccccc4)c[nH]c3nn2)nn1]

### Step 1: 3-iodo-7-(phenylsulfonyl)-7H-pyrrolo[2,3-c]pyridazine

To a solution of 3-iodo-7H-pyrrolo[2,3-c]pyridazine (Example 1, Step 4; 300 mg, 1.22 mmol) in MeCN (10 ml) were added K₂CO₃ (169 mg, 1.22 mmol) and benzenesulfonyl chloride (260 mg, 1.47 mmol) and the resulting mixture was stirred at RT for 2 h. The reaction mixture was filtered through a pad of Celite® and concentrated under reduced pressure. The residue was purified via silica gel chromatography (0-40% EtOAc in hexanes) to give the title compound as a yellow solid (354 mg, 75% yield). MS(ES⁺) Cl₂H₈IN₃O₂S requires: 385, found: 386 [M+H]⁺.

### Step 2: methyl 1-(4-(7-(phenylsulfonyl)-7H-pyrrolo[2,3-c]pyridazin-3-yl)but-3-yn-1-yl)-1H-1,2,3-triazole-4-carboxylate

A mixture of 3-iodo-7-(phenylsulfonyl)-7H-pyrrolo[2,3-c]pyridazine (300 mg, 0.779 mmol), methyl 1-(but-3-yn-1-yl)-1H-1,2,3-triazole-4-carboxylate (Example 11, Step 3; 140 mg, 0.779 mmol), Pd(PPh₃)₂Cl₂ (54.7 mg, 0.0780 mmol), copper(I) iodide (14.8 mg, 0.0780 mmol) and Et₃N (788 mg, 7.79 mmol) was degassed with a stream of N₂ and stirred at 60° C. for 2 h. The mixture was cooled to RT, the volatiles were removed under reduced pressure and the residue was purified via SiO₂ gel chromatography (0-5% MeOH in DCM) to give the title compound as a yellow solid (285 mg, 84% yield). MS (ES⁺) C₂₀H₁₆N₆O₄S requires: 436, found: 437 [M+H]⁺.

### Step 3: methyl 1-(4-(7-(phenylsulfonyl)-7H-pyrrolo[2,3-c]pyridazin-3-yl)butyl)-1H-1,2,3-triazole-4-carboxylate

A reaction vessel was charged with methyl 1-(4-(7-(phenylsulfonyl)-7H-pyrrolo[2,3-c]pyridazin-3-yl)but-3-yn-1-yl)-1H-1,2,3-triazole-4-carboxylate (260 mg, 0.596 mmol), 5% Pd—C (250 mg, 2.349 mmol), DMF (15 ml) and ethanol (15 ml) under an atmosphere of N₂. The suspension was degassed with a steam of N₂, purged with H₂ and stirred under an atmosphere of H₂ at 1 atm for 4 h. The reaction mixture was then purged with N₂, filtered through a pad of Celite® and concentrated under reduced pressure. The residue was purified by mass-triggered preparative HPLC (Mobile phase: A=0.1% TFA/H₂O, B=0.1% TFA/MeCN; Gradient: B=20-60%; 12 min; Column: C18) to give the title compound as a yellow solid (90 mg, 34% yield). MS (ES⁺) C₂₀H₂₀N₆O₄S requires: 440, found: 441 [M+H]⁺.

### Step 4: methyl 1-(4-(5-bromo-7-(phenylsulfonyl)-7H-pyrrolo[2,3-c]pyridazin-3-yl)butyl)-1H-1,2,3-triazole-4-carboxylate

To a solution of methyl 1-(4-(7-(phenylsulfonyl)-7H-pyrrolo[2,3-c]pyridazin-3-yl)butyl)-1H-1,2,3-triazole-4-carboxylate (90 mg, 0.20 mmol) in DCM (2 ml) was added Br₂ (0.032 ml, 0.61 mmol) and the resulting mixture was stirred at RT for 16 h. The reaction mixture was filtered to give the title compound as a yellow solid (77 mg, 73% yield). MS (ES⁺) C₂₀H₁₉BrN₆O₄S requires: 518, found: 519 [M+H]⁺.

### Step 5: methyl 1-(4-(5-phenyl-7-(phenylsulfonyl)-7H-pyrrolo[2,3-c]pyridazin-3-yl)butyl)-1H-1,2,3-triazole-4-carboxylate

A mixture of methyl 1-(4-(5-bromo-7-(phenylsulfonyl)-7H-pyrrolo[2,3-c]pyridazin-3-yl)butyl)-1H-1,2,3-triazole-4-carboxylate (45 mg, 0.087 mmol) and phenylboronic acid (21.1 mg, 0.173 mmol) in DME (1 ml) was degassed with a stream of N₂ before adding PdCl₂(dppf)-CH₂Cl₂ adduct (7.08 mg, 8.66 μmol) and 2N aq. Na₂CO₃ (0.087 ml, 0.17 mmol). The mixture was heated at 80° C. and stirred for 16 h. The volatiles were removed under reduced pressure, the residue was taken up in DCM, filtered through a pad of Celite®, and the filtrate was concentrated under reduced pressure. The residue was purified via SiO₂ gel chromatography (0-5% MeOH in DCM) to give the title compound as a brown oil (32 mg, 72% yield). MS (ES⁺) C₂₆H₂₄N₆O₄S requires: 516, found: 517 [M+H]⁺.

### Step 6: 1-(4-(5-phenyl-7H-pyrrolo[2,3-c]pyridazin-3-yl)butyl)-1H-1,2,3-triazole-4-carboxylic acid

To a solution of methyl 1-(4-(5-phenyl-7-(phenylsulfonyl)-7H-pyrrolo[2,3-c]pyridazin-3-yl)butyl)-1H-1,2,3-triazole-4-carboxylate (30 mg, 0.058 mmol) in THF (0.5 ml) and water (0.5 ml) was added LiOH (7.0 mg, 0.29 mmol) and the resulting mixture was stirred at RT for 2 h. The volatiles were removed under reduced pressure and the residue was taken up in MeOH and purified by mass-triggered preparative HPLC (Mobile phase: A=0.1% TFA/H₂O, B=0.1% TFA/MeCN; Gradient: B=10-50%; 12 min; Column: C18) to give the title compound as a yellow solid (13 mg, 62% yield). MS(ES⁺) C₁₉H₈N₆O₂ requires: 362, found: 363 [M+H]⁺.

### Step 7: 1-(4-(5-phenyl-7H-pyrrolo[2,3-c]pyridazin-3-yl)butyl)-N-(pyridin-2-ylmethyl)-1H-1,2,3-triazole-4-carboxamide

To a solution of 1-(4-(5-phenyl-7H-pyrrolo[2,3-c]pyridazin-3-yl)butyl)-1H-1,2,3-triazole-4-carboxylic acid (12 mg, 0.033 mmol) in DMF (0.5 ml) were added HATU (13.9 mg, 0.0360 mmol), pyridin-2-ylmethanamine (3.9 mg, 0.036 mmol) and DIEA (8.9 μl, 0.050 mmol) and the resulting mixture was stirred at RT for 1 h. The volatiles were removed under reduced pressure and the residue was purified by mass-triggered preparative HPLC (Mobile phase: A=0.1% TFA/H₂O, B=0.1% TFA/MeCN; Gradient: B=10-50%; 12 min; Column: C18) to give the title compound as a yellow solid (11 mg, 0.024 mmol, 73% yield). MS (ES⁺) C₂₅H₂₄N₈O requires: 452, found: 453 [M+H]⁺. ¹H NMR (600 MHz, DMSO-d₆) δ 13.17 (bs, 1H), 9.02 (t, J=6.0 Hz, 1H), 8.67 (bs, 1H), 8.62 (s, 1H), 8.51 (d, J=4.8 Hz, 1H), 8.41 (bs, 1H), 8.71 (d, J=7.2 Hz, 2H), 7.76 (m, 1H), 7.50-7.45 (m, 2H), 7.38-7.31 (m, 3H), 4.55 (d, J=6.0 Hz, 2H), 4.49 (t, J=7.2 Hz, 2H), 3.13 (t, J=7.2 Hz, 2H), 1.98-1.90 (m, 2H), 1.80-1.74 (m, 2H).

### Example 21

### 1-(4-(6-(3-hydroxyoxetan-3-yl)-7H-pyrrolo[2,3-c]pyridazin-3-yl)butyl)-N-(pyridin-2-ylmethyl)-1H-1,2,3-triazole-4-carboxamide

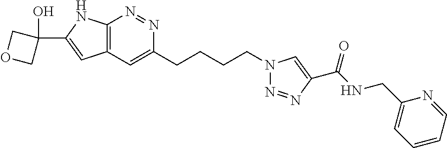 [SMILES: O=C(NCc1ccccn1)c1cn(CCCCc2cc3cc(C4(O)COC4)[nH]c3nn2)nn1]

### Steps 1 to 3

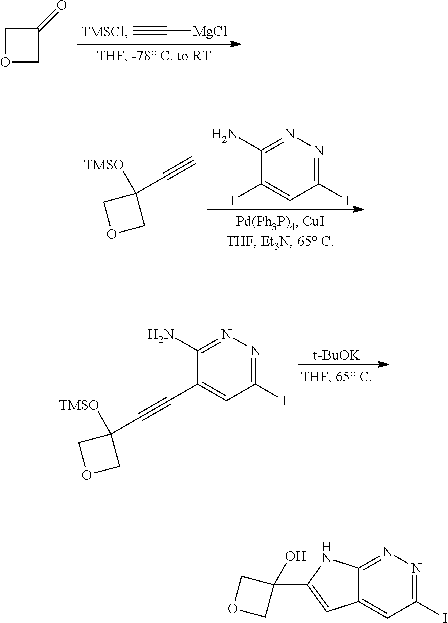

### Step 1: ((3-ethynyloxetan-3-yl)oxy)trimethylsilane

To a solution of ethynylmagnesium chloride (8.33 mL, 4.16 mmol) in THF (13.9 mL) at −78° C. was added oxetan-3-one (0.178 mL, 2.78 mmol) dropwise, followed by TMS-Cl (0.532 mL, 4.16 mmol). The resulting mixture was stirred at −78° C. for 15 minutes, then allowed to warm to RT and stirred for 1 h. Saturated aq. NaHCO₃ (3 mL) was added, the layers were separated and the aqueous phase was extracted with EtOAc (3×10 mL). The combined organic layers were washed with brine (5 mL), dried over Na₂SO₄, filtered and concentrated under reduced pressure. The residue was purified via SiO₂ gel chromatography (0-10% EtOAc in hexanes) to give the title compound as a colorless liquid (345 mg, 73%). ¹H NMR (600 MHz, Chloroform-d) δ 4.75 (d, J=6.1 Hz, 2H), 4.67 (d, J=6.2 Hz, 2H), 2.67 (s, 1H), 0.17 (s, 9H).

### Step 2: 6-iodo-4-((3-((trimethylsilyl)oxy)oxetan-3-yl)ethynyl)pyridazin-3-amine

To a solution of 4,6-diiodopyridazin-3-amine (Example 1, Step 1; 100 mg, 0.288 mmol) in THF (961 μl) were added Et₃N (442 μl, 3.17 mmol), ((3-ethynyloxetan-3-yl)oxy)trimethylsilane (59 mg, 0.35 mmol), Pd(Ph₃P)₄ (17 mg, 0.014 mmol) and CuI (1.6 mg, 8.7 μmol). The mixture was degassed with a stream of N₂ for 2 minutes and the resulting mixture was stirred at 65° C. for 12 h. The mixture was cooled to RT and concentrated under reduced pressure. The residue was purified via SiO₂ gel chromatography (0-15% MeOH in DCM) to give the title compound (125 mg, 100%). MS (ES+) C₁₂H₁₆IN₃O₂Si requires: 389, found: 390 [M+H]⁺.

### Step 3: 3-(3-iodo-7H-pyrrolo[2,3-c]pyridazin-6-yl)oxetan-3-ol

To a suspension of 6-iodo-4-((3-((trimethylsilyl)oxy)oxetan-3-yl)ethynyl)pyridazin-3-amine (112 mg, 0.288 mmol) in THF (2.9 mL) was added t-BuOK (48 mg, 0.43 mmol) and the resulting mixture was stirred at 65° C. for 12 h. The mixture was cooled to RT and concentrated under reduced pressure. The residue was purified via SiO₂ gel chromatography (0-15% MeOH in DCM to give the title compound as a brown solid (16 mg, 18%). C₉H₈IN₃O₂ requires: 317. found: 318 [M+H]⁺.

### Steps 4 to 6

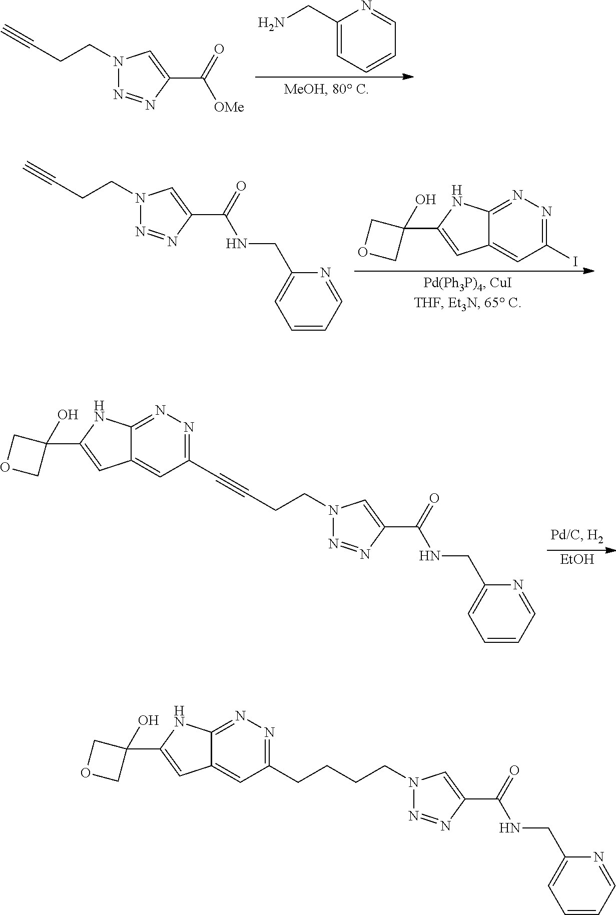

### Step 4: 1-(but-3-yn-1-yl)-N-(pyridin-2-ylmethyl)-1H-1,2,3-triazole-4-carboxamide

To a suspension of methyl 1-(but-3-yn-1-yl)-1H-1,2,3-triazole-4-carboxylate (Example 11 Step 3; 50 mg, 0.28 mmol) in MeOH (1.4 mL) was added pyridin-2-ylmethanamine (144 μl, 1.40 mmol) and the resulting mixture was stirred at 80° C. for 72 h. The mixture was cooled to RT and concentrated under reduced pressure. The residue was purified via SiO₂ gel chromatography (50 to 100% EtOAc in hexanes) to give the title compound as a white solid (40 mg, 56%): MS (ES⁺) C₁₃H₃N₅O requires: 255, found: 256 [M+H]⁺.

### Step 5: 1-(4-(6-(3-hydroxyoxetan-3-yl)-7H-pyrrolo[2,3-c]pyridazin-3-yl)but-3-yn-1-yl)-N-(pyridin-2-ylmethyl)-1H-1,2,3-triazole-4-carboxamide

To a solution of 3-(3-iodo-7H-pyrrolo[2,3-c]pyridazin-6-yl)oxetan-3-ol (Step 3, 16 mg, 0.050 mmol) in THF (252 μl) were added Et₃N (77 μl, 0.55 mmol), 1-(but-3-yn-1-yl)-N-(pyridin-2-ylmethyl)-1H-1,2,3-triazole-4-carboxamide (14 mg, 0,056 mmol), Pd(Ph₃P)₄ (2.9 mg, 2.5 μmol) and CuI (0.48 mg, 2.5 μmol). The mixture was degassed with a stream of N₂ for 2 minutes and the resulting mixture was stirred at 65° C. for 12 h, then cooled to RT and concentrated under reduced pressure. The residue was purified via SiO₂ gel chromatography (0-20% MeOH in DCM) to give the title compound as a brown solid (15 mg, 67% yield). MS (ES⁺) C₂₂H₂₀N₈O₃ requires: 444, found: 445 [M+H]⁺.

### Step 6: 1-(4-(6-(3-hydroxyoxetan-3-yl)-7H-pyrrolo[2,3-c]pyridazin-3-yl)butyl)-N-(pyridin-2-ylmethyl)-1H-1,2,3-triazole-4-carboxamide

A reaction vessel was charged with 1-(4-(6-(3-hydroxyoxetan-3-yl)-7H-pyrrolo[2,3-c]pyridazin-3-yl)but-3-yn-1-yl)-N-(pyridin-2-ylmethyl)-1H-1,2,3-triazole-4-carboxamide (15 mg, 0.034 mmol), 10% Pd/C (3.6 mg) and EtOH (337 μl) under an atmosphere of N₂. The suspension was degassed with N₂ for 1 minute and purged with H₂ for 2 minutes. The reaction mixture was stirred under an atmosphere of H₂ for 1 h, then purged with N₂, filtered through a pad of Celite®, and concentrated under reduced pressure. The residue was purified by mass-triggered preparative HPLC (Mobile phase: A=0.1% TFA/H₂O, B=0.1% TFA/MeCN; Gradient: B=10-40%; 20 min; Column: C18) to give the title compound as a colorless liquid (4.1 mg, 22%). MS (ES⁺) C₂₂H₂₄N₈O₃ requires: 448, found: 449 [M+H]⁺. ¹H NMR (500 MHz, Methanol-d₄) δ 8.81 (m, 1H), 8.71 (d, J=5.4 Hz, 1H), 8.47 (d, J=8.1 Hz, 1H), 8.41 (s, 1H), 8.32 (s, 1H), 7.94 (dd, J=8.1, 5.6 Hz, 1H), 7.10 (s, 1H), 4.97 (d, J=7.0 Hz, 2H), 4.93 (d, J=7.0 Hz, 2H), 4.74 (s, 2H), 4.55 (t, J=6.8 Hz, 2H), 3.20 (t, J=7.8 Hz, 2H), 2.11-2.04 (m, 2H), 1.92-1.81 (m, 2H).

### Example 22

### 5-(4-(2-benzyl-3H-imidazo[4,5-b]pyridin-6-yl)butyl)-1,3,4-thiadiazol-2-amine

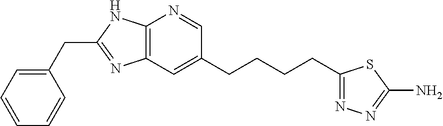 [SMILES: Nc1nnc(CCCCc2cnc3[nH]c(Cc4ccccc4)nc3c2)s1]

### Steps 1 to 5

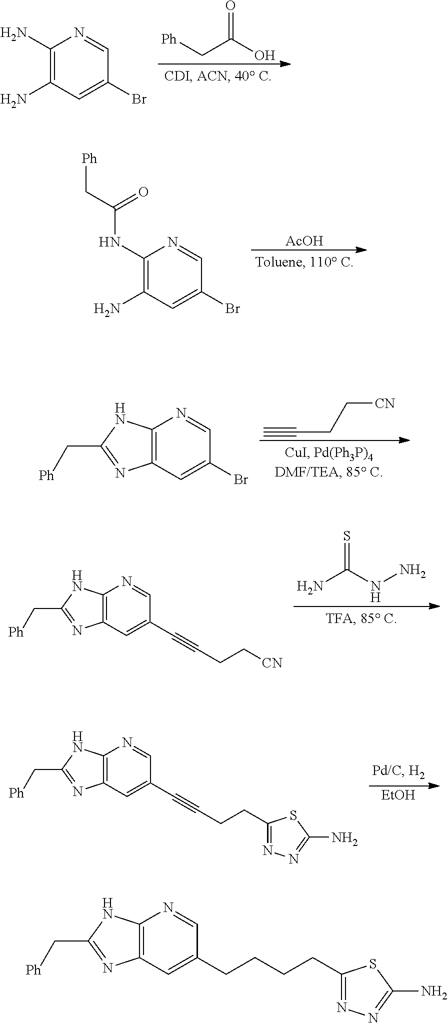

### Step 1: N-(3-amino-5-bromopyridin-2-yl)-2-phenylacetamide

To a solution of 2-phenylacetic acid (0.335 mL, 2.66 mmol) in MeCN (13 mL) was added CDI (431 mg, 2.66 mmol) and the resulting mixture was stirred at 40° C. for 15 minutes before the addition of 5-bromopyridine-2,3-diamine (500 mg, 2.66 mmol). The mixture was heated at 65° C. for 12 h, cooled to RT and collected by filtration. The filtrate was washed with EtOAc (2×3 mL) and dried under reduced pressure to give the title compound (280 mg, 34%). MS (ES⁺) C₁₃H₁₂BrN₃O requires: 305, found: 306 [M+H]⁺.

### Step 2: 2-benzyl-6-bromo-3H-imidazo[4,5-b]pyridine

To a suspension of N-(3-amino-5-bromopyridin-2-yl)-2-phenylacetamide (150 mg, 0.490 mmol) in toluene (4.9 mL) was added AcOH (280 μl, 4.90 mmol) and the resulting mixture was stirred at 110° C. for 12 h. The reaction mixture was then cooled to RT and concentrated under reduced pressure to give the title compound. MS (ES⁺) C₁₃H₁₀BrN₃ requires: 287, found: 288 [M+H]⁺.

### Step 3: 5-(2-benzyl-3H-imidazo[4,5-b]pyridin-6-yl)pent-4-ynenitrile

A solution of 2-benzyl-6-bromo-3H-imidazo[4,5-b]pyridine (50 mg, 0.17 mmol) in DMF (578 μl) was degassed with Ar for 1 minute. Copper(I) iodide (1.7 mg, 8.7 μmol), Et₃N (289 μl), Pd(Ph₃P)₄ (6.1 mg, 8.7 μmol) and pent-4-ynenitrile (30 μL, 0.35 mmol) were added and the mixture was degassed with Ar for an additional 1 minute. The mixture was heated at 85° C. for 4 h, then cooled to RT and concentrated under reduced pressure. The residue was purified via silica gel chromatography (0-10% MeOH in DCM) to give the title compound. MS (ES⁺) C₁₈H₁₄N₄ requires: 286, found: 287 [M+H]⁺.

### Step 4: 5-(4-(2-benzyl-3H-imidazo[4,5-b]pyridin-6-yl)but-3-yn-1-yl)-1,3,4-thiadiazol-2-amine

To a suspension of 5-(2-benzyl-3H-imidazo[4,5-b]pyridin-6-yl)pent-4-ynenitrile (50 mg, 0.18 mmol) in TFA (873 μl) was added hydrazinecarbothioamide (17.5 mg, 0.192 mmol) and the resulting mixture was heated at 85° C. for 12 h. The reaction mixture was cooled to RT and concentrated under reduced pressure. The residue was purified by mass-triggered preparative HPLC (Mobile phase: A=0.1% TFA/H₂O, B=0.1% TFA/MeCN; Gradient: B=20-50%; 12 min; Column: C18) to give the title compound as an off-white solid (21 mg, 44%). MS (ES⁺) C₁₉H₁₆N₆S requires: 360, found: 361 [M+H]⁺.

### Step 5: 5-(4-(2-benzyl-3H-imidazo[4,5-b]pyridin-6-yl)butyl)-1,3,4-thiadiazol-2-amine

A reaction vessel was charged with 5-(4-(2-benzyl-3H-imidazo[4,5-b]pyridin-6-yl)but-3-yn-1-yl)-1,3,4-thiadiazol-2-amine (20 mg, 0.044 mmol), Pd—C (47 mg, 0.044 mmol) and AcOH (437 μL) under an atmosphere of N2. The suspension was degassed with N2 for 2 minutes and purged with H2 for 2 minutes, then stirred under an atmosphere of H2 at 1 atm for 6 h. The reaction mixture was purged with N2, filtered through a pad of Celite®, and concentrated under reduced pressure. The residue was purified by mass-triggered preparative HPLC (Mobile phase: A=0.1% TFA/H2O, B=0.1% TFA/MeCN; Gradient: B=10-40%; 12 min; Column: C18) to give the title compound as a white solid (1.9 mg, 10%). MS (ES+) C19H20N6S requires: 364, found: 365 [M+H]+; 1H NMR (600 MHz, DMSO-d6) δ 8.39 (m, 1H), 8.07 (m, 1H), 7.65 (s, 2H), 7.41-7.32 (m, 4H), 7.29 (m, 1H), 4.42-4.33 (m, 2H), 2.84 (t, J=7.1 Hz, 2H), 2.82-2.75 (m, 2H), 1.75-1.56 (m, 4H).

### Example 23

### N-(5-(4-(2-benzyl-3H-imidazo[4,5-b]pyridin-6-yl)butyl)-1,3,4-thiadiazol-2-yl)-2-phenylacetamide

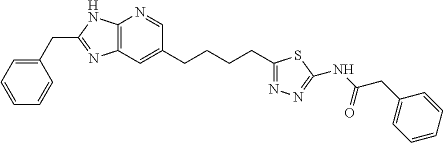 [SMILES: O=C(Cc1ccccc1)Nc1nnc(CCCCc2cnc3[nH]c(Cc4ccccc4)nc3c2)s1]

To a solution of 5-(4-(2-benzyl-3H-imidazo[4,5-b]pyridin-6-yl)butyl)-1,3,4-thiadiazol-2-amine (Example 22; 15 mg, 0.033 mmol) in Pyridine (325 μl) was added 2-phenylacetyl chloride (6.5 μl, 0.049 mmol) and the resulting mixture was stirred at RT for 15 minutes. The mixture was concentrated under reduced pressure and the residue was purified by mass-triggered preparative HPLC (Mobile phase: A=0.1% TFA/H₂O, B=0.1% TFA/MeCN; Gradient: B=30-70%; 12 min; Column: C18) to give the title compound as a white solid (1.4 mg, 7%). MS (ES⁺) C₂₇H₂₆N₆OS requires: 482, found: 483 [M+H]⁺; ¹H NMR (600 MHz, Methanol-d₄) δ 8.42 (s, 1H), 8.09 (s, 1H), 7.42-7.35 (m, 4H), 7.37-7.28 (m, 5H), 7.31-7.24 (m, 1H), 4.44 (s, 2H), 3.81 (s, 2H), 3.07 (t, J=7.2 Hz, 2H), 2.90 (t, J=7.4 Hz, 2H), 1.88-1.72 (m, 4H).

### Example 24

### N-(5-(4-(1H-pyrrolo[2,3-b]pyridin-5-yl)butyl)-1,3,4-thiadiazol-2-yl)-2-phenylacetamide

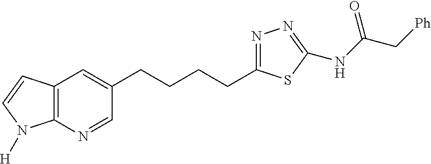 [SMILES: O=C(Cc1ccccc1)Nc1nnc(CCCCc2cnc3[nH]ccc3c2)s1]

To a solution of 5-(4-(1-(phenylsulfonyl)-1H-pyrrolo[2,3-b]pyridin-5-yl)butyl)-1,3,4-thiadiazol-2-amine (prepared as described for Example 23, using 5-(1-(phenylsulfonyl)-1H-pyrrolo[2,3-b]pyridin-5-yl)pent-4-ynenitrile instead of 5-(2-benzyl-3H-imidazo[4,5-b]pyridin-6-yl)pent-4-ynenitrile; 70 mg, 0.17 mmol) in Pyridine (846 μl) was added 2-phenylacetyl chloride (24.6 μl, 0.186 mmol) and the resulting mixture was stirred at 40° C. for 4 h. The reaction mixture was allowed to cool to RT and the volatiles were removed under reduced pressure. The residue was taken up in EtOH (627 μl), aq. NaOH (7.5 μl, 0.094 mmol) was added and the resulting mixture was stirred at 40° C. for 2 h. The reaction mixture was cooled to RT, the volatiles were removed under reduced pressure and the residue was purified by mass-triggered preparative HPLC (Mobile phase: A=0.1% TFA/H₂O, B=0.1% TFA/MeCN; Gradient: B=20-50%; 12 min; Column: C18) to give the title compound as a white solid (4 mg, 6%). MS (ES⁺) C₂₁H₂₁N₅OS requires: 391, found: 392 [M+H]⁺; ¹H NMR (600 MHz, DMSO-d₆) δ 12.64 (s, 1H), 11.62 (s, 1H), 8.09 (d, J=2.0 Hz, 1H), 7.83 (s, 1H), 7.44 (m, 1H), 7.36-7.29 (m, 4H), 7.27 (m, 1H), 6.40 (dd, J=3.3, 1.8 Hz, 1H), 3.79 (s, 2H), 3.00 (t, J=7.1 Hz, 2H), 2.71 (t, J=7.1 Hz, 2H), 1.75-1.63 (m, 4H).

### Example 134

### 1-(4-(6-(2-fluorophenyl)-7H-pyrrolo[2,3-c]pyridazin-3-yl)butyl)-N-(pyridin-2-ylmethyl)-1H-1,2,3-triazole-4-carboxamide

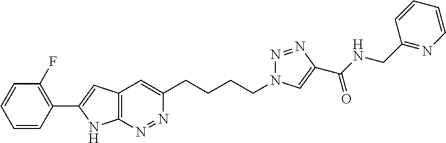 [SMILES: O=C(NCc1ccccn1)c1cn(CCCCc2cc3cc(-c4ccccc4F)[nH]c3nn2)nn1]

### Steps 1 to 8

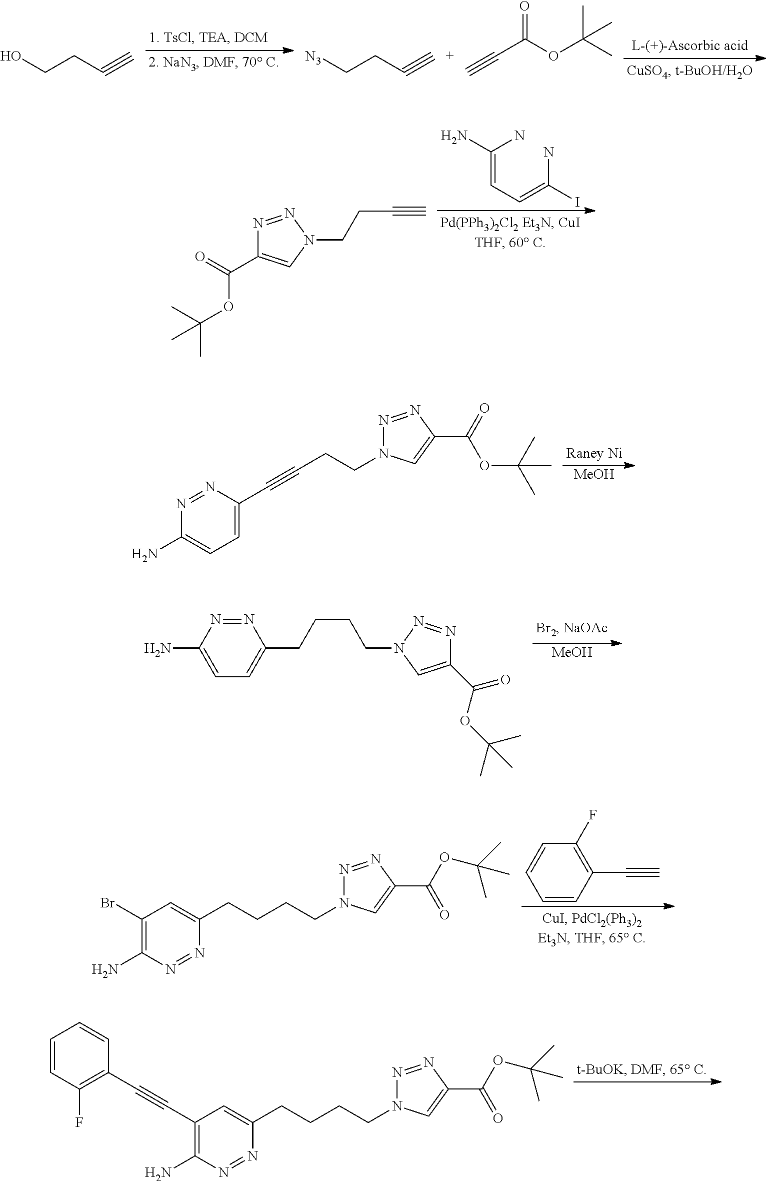

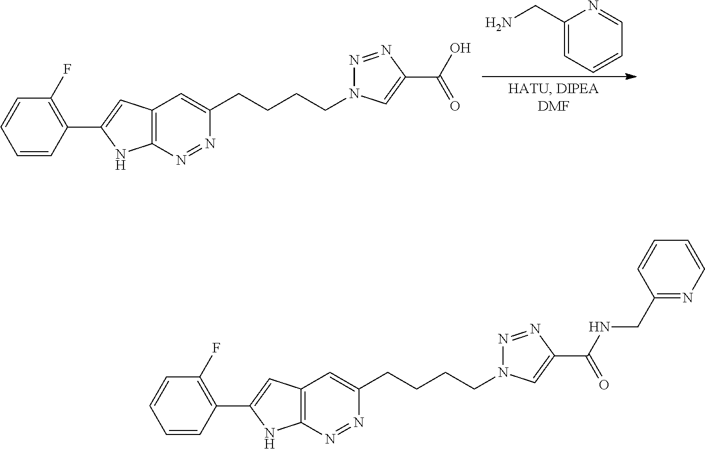 [SMILES: O=C(O)c1cn(CCCCc2cc3cc(-c4ccccc4F)[nH]c3nn2)nn1 ; NCc1ccccn1 ; O=C(NCc1ccccn1)c1cn(CCCCc2cc3cc(-c4ccccc4F)[nH]c3nn2)nn1]

### Step 1: 4-azidobut-1-yne

To a solution of but-3-yn-1-ol (40 g, 0.57 mol), TEA (115 g, 1.14 mol) in DCM (800 ml) at 0° C. was added TsCl (119 g, 0.627 mol) and the mixture was stirred at rt for 16 h. The mixture was diluted with water (500 mL), extracted with DCM (300 mL) and the combined organic layers were concentrated to afford a brown oil. The residue was dissolved in DMF (500 mL), NaN₃ (41 g, 0.63 mol) was added and the mixture was heated at 70° C. for 12 h. The mixture was diluted with water (1 L) and extracted with diethyl ether (800 mL). The combined organic layers were concentrated at rt to give the assumed title compound as a yellow oil and taken on immediately without purification.

### Step 2: tert-butyl 1-(but-3-ynyl)-1H-1,2,3-triazole-4-carboxylate

A mixture of 4-azidobut-1-yne (20 g, 0.21 mol), tert-butyl propiolate (26.5 g, 0.210 mol), L-(+)-Ascorbic acid (8.0 g, 46 mmol) and CuSO₄ (4.0 g, 25 mmol) in t-BuOH/H₂O=1:1 (400 mL) was stirred at rt for 12 h. The mixture was concentrated at reduce pressure and 200 mL of water was added. The aqueous layer was extracted with EtOAc (3×300 mL) and the combined organic layers were concentrated at reduced pressure to give a yellow solid. The solid was washed with petroleum ether to give the title compound as a white solid (25 g, 54%). MS (ES⁺) C₁₁H₁₅N₃O₂ requires: 221, found: 222 [M+H]⁺.

### Step 3: tert-butyl 1-(4-(6-aminopyridazin-3-yl)but-3-ynyl)-1H-1,2,3-triazole-4-carboxylate

A mixture of tert-butyl 1-(but-3-ynyl)-1H-1,2,3-triazole-4-carboxylate (15 g, 68 mmol), 6-iodopyridazin-3-amine (15 g, 68 mmol), Pd(PPh₃)₂Cl₂ (4.77 g, 6.80 mmol), CuI (1.29 g, 6.8 mmol) and TEA (34.2 g, 339 mmol) in 300 mL anhydrous THF was heated at 60° C. under N₂ for 12 h. The mixture was cooled to rt, DCM/MeOH (500 mL, 10:1) was added, filtered off and concentrated under reduced pressure to give a yellow oil. The oil was purified by silica gel column (0 to 9% DCM in MeOH) to give the title compound as a yellow solid (18.0 g, 84%). MS (ES⁺) C₁₅H₁₈N₆O₂ requires: 314, found: 315 [M+H]⁺.

### Step 4: tert-butyl 1-(4-(6-aminopyridazin-3-yl)butyl)-1H-1,2,3-triazole-4-carboxylate

To a solution of tert-butyl 1-(4-(6-aminopyridazin-3-yl)but-3-ynyl)-1H-1,2,3-triazole-4-carboxylate (3.5 g, 11 mmol), Raney Ni (300 mg) in 250 ml MeOH. The system was evacuated and then refilled with hydrogen, stirred at room temperature overnight. Filtered off, the combined organic layers were concentrated to afford a yellow solid. The solid was purified by silica gel column chromatography (DCM: MeOH=0˜9%) afford the tert-butyl 1-(4-(6-aminopyridazin-3-yl)butyl)-1H-1,2,3-triazole-4-carboxylate as a yellow solid (3.17 g, 89%). MS (ES⁺) C₁₅H₂₂N₆O₂ requires: 318, found: 319 [M+H]⁺.

### Step 5: tert-butyl 1-(4-(6-amino-5-bromopyridazin-3-yl)butyl)-1H-1,2,3-triazole-4-carboxylate

To a suspension of methyl 1-(4-(6-aminopyridazin-3-yl)butyl)-1H-1,2,3-triazole-4-carboxylate (400 mg, 1.45 mmol) in MeOH (3.6 mL) were added NaHCO₃ (365 mg, 4.34 mmol) and bromine (112 μL, 2.17 mmol) dropwise and the resulting mixture was stirred at 23° C. for 16 h. The reaction mixture was filtered through celite, and the filtrate was concentrated under reduced pressure. The residue was purified via silica gel chromatography (0-10% MeOH in DCM) to give the title compound as an orange solid (176 mg, 34%). MS (ES⁺) C₁₂H₁₅BrN₆O₂ requires: 354/356, found: 355/357 [M+H]⁺.

### Step 6: tert-butyl 1-(4-(6-amino-5-((2-fluorophenyl)ethynyl)pyridazin-3-yl)butyl)-1H-1,2,3-triazole-4-carboxylate

To a solution of tert-butyl 1-(4-(6-amino-5-bromopyridazin-3-yl)butyl)-1H-1,2,3-triazole-4-carboxylate (200 mg, 0.503 mmol) in THF (1.7 mL) were added triethylamine (772 μL, 5.54 mmol), 1-ethynyl-2-fluorobenzene (73 mg, 0.604 mmol), bis(triphenylphosphine)-palladium(II) chloride (18 mg, 0.025 mmol) and CuI (2.88 mg, 0.015 mmol). The mixture was degassed for 2 min and the resulting mixture was stirred at 65° C. for 16 h. The mixture was cooled to rt and concentrated under reduced pressure. The residue was purified via silica gel chromatography (0-10% MeOH in DCM) to give the title compound as a brown solid (173 mg, 79%). MS (ES⁺) C₂₃H₂₅FN₆O₂ requires: 436.5, found: 437.4 [M+H]⁺.

### Step 7: 1-(4-(6-(2-fluorophenyl)-7H-pyrrolo[2,3-c]pyridazin-3-yl)butyl)-1H-1,2,3-triazole-4-carboxylic acid

To a solution of tert-butyl 1-(4-(6-amino-5-((2-fluorophenyl)ethynyl)pyridazin-3-yl)butyl)-1H-1,2,3-triazole-4-carboxylate (173 mg, 0.396 mmol) in DMF (4.0 mL) was added t-BuOK (133 mg, 1.19 mmol) and the resulting mixture was stirred at 65° C. for 3 h. Additional t-BuOK (110 mg, 0.99 mmol) was added and the mixture was heated at 65° C. for 15 h. The mixture was cooled to rt, neutralized with HCl (330 μl, 1.982 mmol) and concentrated under reduced pressure. The residue was purified by mass-triggered preparative HPLC (Mobile phase: A=0.1% TFA/H₂O, B=0.1% TFA/MeCN; Gradient: B=20-50%; 12 min; Column: C18) to give the title compound as a tan solid (68 mg, 35%). MS (ES⁺) C₁₉H₁₇FN₆O₂ requires: 380.4. found: 381 [M+H]⁺.

### Step 8: 1-(4-(6-(2-fluorophenyl)-7H-pyrrolo[2,3-c]pyridazin-3-yl)butyl)-N-(pyridin-2-ylmethyl)-1H-1,2,3-triazole-4-carboxamide

To a solution of 1-(4-(6-(2-fluorophenyl)-7H-pyrrolo[2,3-c]pyridazin-3-yl)butyl)-1H-1,2,3-triazole-4-carboxylic acid (10 mg, 0.026 mmol) in DMF (526 μL) were added DIPEA (23 μL, 0.13 mmol) and HATU (15 mg, 0.039 mmol) and the resulting mixture was stirred for 1 h. The mixture was concentrated under reduced pressure. The residue was purified by mass-triggered preparative HPLC (Mobile phase: A=0.1% TFA/H₂O, B=0.1% TFA/MeCN; Gradient: B=10-40%; 20 min; Column: C18) to give an orange solid. The residue was re-purified by mass-triggered preparative HPLC (Mobile phase: A=0.1% TFA/H₂O, B=0.1% TFA/MeCN; Gradient: B=10-40%; 30 min; Column: C18) to give the title compound as a yellow solid (3.3 mg, 22%). MS (ES⁺) C₂₅H₂₃FN₈O requires: 470.5, found: 471.5 [M+H]⁺. ¹H NMR (600 MHz, Methanol-d₄) δ 8.60 (d, J=5.1 Hz, 1H), 8.41 (s, 1H), 8.33 (s, 1H), 8.09 (td, J=8.0, 1.7 Hz, 1H), 8.04 (td, J=7.7, 1.7 Hz, 1H), 7.71-7.62 (m, 2H), 7.59-7.53 (m, 1H), 7.46 (t, J=7.6 Hz, 1H), 7.42 (dd, J=11.6, 8.3 Hz, 1H), 7.35 (d, J=2.1 Hz, 1H), 4.77 (s, 2H), 4.57 (t, J=6.9 Hz, 2H), 3.21 (t, J=7.8 Hz, 2H), 2.15-2.01 (m, 2H), 1.93-1.83 (m, 2H).

### Example 111

### tert-butyl 3-(3-(4-(4-((2-fluoro-5-(trifluoromethoxy)benzyl)carbamoyl)-1H-1,2,3-triazol-1-yl)butyl)-7H-pyrrolo[2,3-c]pyridazin-6-yl)azetidine-1-carboxylate

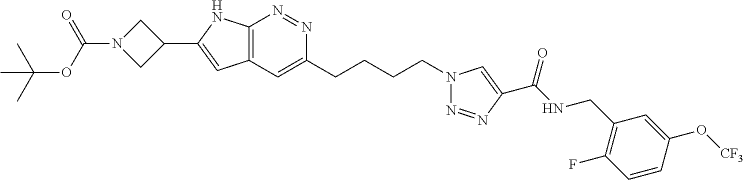 [SMILES: CC(C)(C)OC(=O)N1CC(c2cc3cc(CCCCn4cc(C(=O)NCc5cc(OC(F)(F)F)ccc5F)nn4)nnc3[nH]2)C1]

### Steps 1 to 4

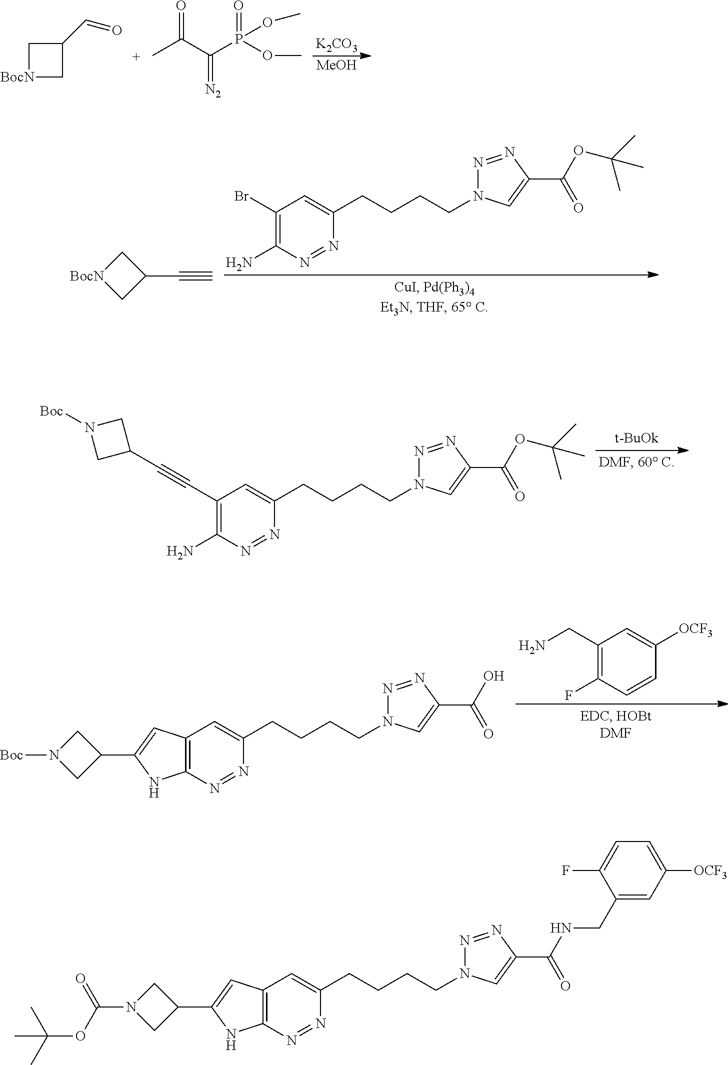

### Step 1: tert-butyl 3-ethynylazetidine-1-carboxylate

To a solution of tert-butyl 3-formylazetidine-1-carboxylate (5.0 g, 27 mmol) in MeOH (70 mL) at 0° C. was added dimethyl (1-diazo-2-oxopropyl)phosphonate (6.0 g, 31 mmol), followed by potassium carbonate (14.9 g, 108 mmol) and the reaction was stirred for 3 hr. The reaction mixture was then diluted with EtOAc (150 mL), filtered and concentrated under reduced pressure. The residue was purified by flash chromatography (0-100% EtOAc in hexanes) to give the title compound (4.6 g, 94%). ¹H NMR (400 MHz, DMSO-d6) δ 4.04 (t, J=8.4 Hz, 2H), 3.68 (t, J=6.8, 2 H), 3.34-3.39 (m, 1H), 3.22-3.23 (m, 1H), 1.34 (s, 9H).

### Step 2: tert-butyl 1-(4-(6-amino-5-((1-(tert-butoxycarbonyl)azetidin-3-yl)ethynyl)pyridazin-3-yl)butyl)-1H-1,2,3-triazole-4-carboxylate

To a solution of tert-butyl 3-ethynylazetidine-1-carboxylate (0.684 g, 3.78 mmol) in THF (12.6 mL) were added triethylamine (0.702 mL, 5.03 mmol), tert-butyl 1-(4-(6-amino-5-bromopyridazin-3-yl)butyl)-1H-1,2,3-triazole-4-carboxylate (Example 134, step 5) (1.00 g, 2.52 mmol), Tetrakis(triphenylphosphine)palladium(0) (0.145 g, 0.126 mmol) and copper(I) iodide (0.024 g, 0.13 mmol). The mixture was degassed for 2 min and the resulting mixture was stirred at 65° C. for 16 h. The mixture was cooled to rt and concentrated under reduced pressure. The residue was purified via silica gel chromatography (0-10% MeOH in DCM) to give the title compound as an orange solid (766 mg, 61%). MS (ES⁺) C₂₅H₃₅N₇O₄ requires: 497, found: 498 [M+H]⁺.

### Step 3: 1-(4-(6-(1-(tert-butoxycarbonyl)azetidin-3-yl)-7H-pyrrolo[2,3-c]pyridazin-3-yl)butyl)-1H-1,2,3-triazole-4-carboxylic acid

To a suspension of tert-butyl 1-(4-(6-amino-5-((1-(tert-butoxycarbonyl)azetidin-3-yl)ethynyl)pyridazin-3-yl)butyl)-1H-1,2,3-triazole-4-carboxylate (0.76 g, 1.5 mmol) in THF (7.6 mL) was added t-BuOK (0.189 g, 1.68 mmol) and the resulting mixture was stirred at 60° C. for 2 h. The mixture was cooled to rt and concentrated under reduced pressure. The residue was purified via silica gel chromatography (10-20% MeOH in DCM with 2% acetic acid) to give the title compound as a brown solid (313 mg, 46%). MS (ES⁺) C₂₁H₂₇N₇O₄ requires: 441, found: 442 [M+H]⁺.

### Step 4: tert-butyl 3-(3-(4-(4-((2-fluoro-5-(trifluoromethoxy)benzyl)carbamoyl)-1H-1,2,3-triazol-1-yl)butyl)-7H-pyrrolo[2,3-c]pyridazin-6-yl)azetidine-1-carboxylate

To a solution of 1-(4-(6-(1-(tert-butoxycarbonyl)azetidin-3-yl)-7H-pyrrolo[2,3-c]pyridazin-3-yl)butyl)-1H-1,2,3-triazole-4-carboxylic acid (300 mg, 0.680 mmol) in DMF (6.8 mL) were added (2-fluoro-5-(trifluoromethoxy)phenyl)methanamine (213 mg, 1.02 mmol), EDC (195 mg, 1.02 mmol), and HOBt (156 mg, 1.02 mmol) and the resulting mixture was stirred at 23° C. for 16 h. The mixture was diluted with DCM, the pH was adjusted to ˜7-8 by using IN NaOH, and the layers were separated. The organic layer was washed with water, dried over Na₂SO₄, filtered, and concentrated under reduced pressure. The residue was purified via silica gel chromatography (0-10% MeOH in DCM) to give the title compound as a brown solid (192 mg, 45%). MS (ES⁺) C₂₉H₃₂F₄N₈O₄ requires: 632, found 633 [M+H]⁺. ¹H NMR (600 MHz, DMSO-d₆) δ 12.30 (s, 1H), 9.16 (t, J=6.1 Hz, 1H), 8.62 (s, 1H), 7.54 (s, 1H), 7.37-7.27 (m, 3H), 6.40 (s, 1H), 4.49 (d, J=6.1 Hz, 2H), 4.46 (t, J=7.0 Hz, 2H), 4.23 (s, 2H), 4.11-3.93 (m, 3H), 2.96 (t, J=7.5 Hz, 2H), 1.94-1.80 (m, 2H), 1.75-1.61 (m, 2H), 1.40 (s, 9H).

### Example 229

### N-(2-fluoro-5-(trifluoromethoxy)benzyl)-1-(4-(6-(1-(3,3,3-trifluoro-2,2-dimethylpropanoyl)azetidin-3-yl)-7H-pyrrolo[2,3-c]pyridazin-3-yl)butyl)-1H-1,2,3-triazole-4-carboxamide

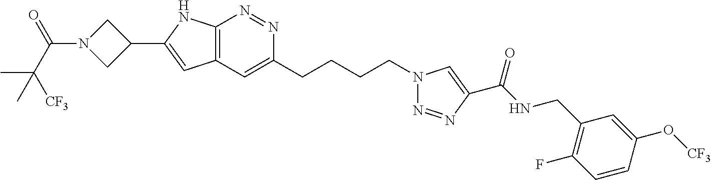 [SMILES: CC(C)(C(=O)N1CC(c2cc3cc(CCCCn4cc(C(=O)NCc5cc(OC(F)(F)F)ccc5F)nn4)nnc3[nH]2)C1)C(F)(F)F]

### Steps 1 to 2

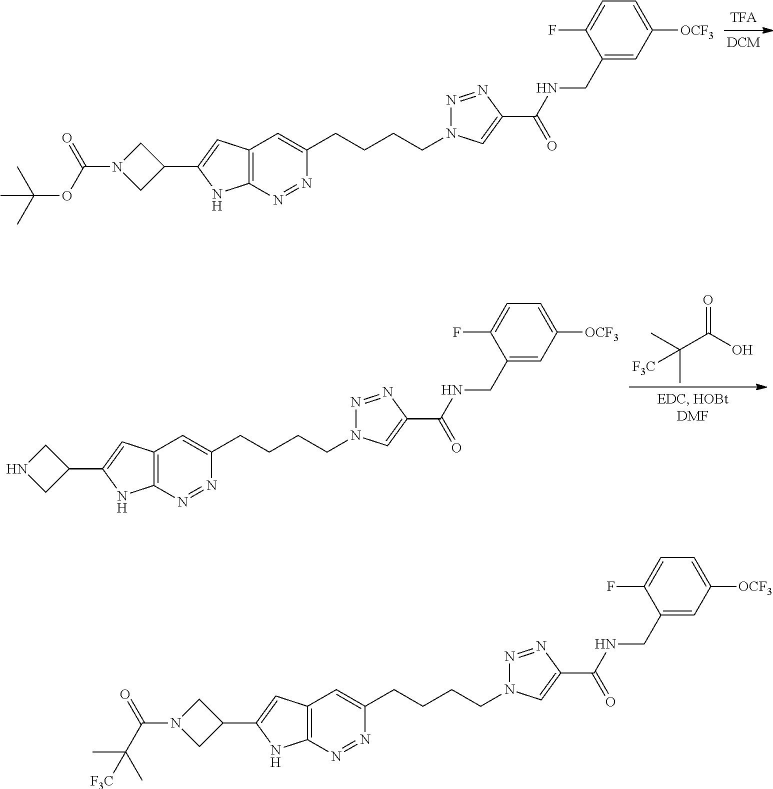

### Step 1: 1-(4-(6-(azetidin-3-yl)-7H-pyrrolo[2,3-c]pyridazin-3-yl)butyl)-N-(2-fluoro-5-(trifluoromethoxy)benzyl)-1H-1,2,3-triazole-4-carboxamide

To a solution of tert-butyl 3-(3-(4-(4-((2-fluoro-5-(trifluoromethoxy)benzyl)carbamoyl)-1H-1,2,3-triazol-1-yl)butyl)-7H-pyrrolo[2,3-c]pyridazin-6-yl)azetidine-1-carboxylate (Example 111, step 3) (112 mg, 0.177 mmol) in DCM (1.77 mL) was added TFA (205 μL, 2.66 mmol) and the resulting mixture was stirred at 23° C. for 3 h. The mixture was concentrated under reduced pressure and the residue was purified via silica gel chromatography (5-25% MeOH in DCM with 2% of NH₄OH) to give the title compound as an off-white solid (75 mg, 80%). MS (ES⁺) C₂₄H₂₄F₄N₈O₂ requires: 532, found 533 [M+H]⁺.

### Step 2: N-(2-fluoro-5-(trifluoromethoxy)benzyl)-1-(4-(6-(1-(3,3,3-trifluoro-2,2-dimethylpropanoyl)azetidin-3-yl)-7H-pyrrolo[2,3-c]pyridazin-3-yl)butyl)-1H-1,2,3-triazole-4-carboxamide

To a solution of 1-(4-(6-(azetidin-3-yl)-7H-pyrrolo[2,3-c]pyridazin-3-yl)butyl)-N-(2-fluoro-5-(trifluoromethoxy)benzyl)-1H-1,2,3-triazole-4-carboxamide (15 mg, 0.028 mmol) in DMF (282 μL) were added 3,3,3-trifluoro-2,2-dimethylpropanoic acid (5.7 mg, 0.037 mmol), EDC (6.5 mg, 0.034 mmol), and HOBt (5.2 mg, 0.034 mmol) and the resulting mixture was stirred at 23° C. for 16 h. The mixture was diluted with DCM (2 mL), pH was adjusted to ˜7-8 by using 1N NaOH, and the two layers were separated. The organic layer was washed with water (1 mL), dried over Na₂SO₄, filtered, and concentrated under reduced pressure. The residue was purified via silica gel chromatography (0-15% MeOH in DCM) to give the title compound as a white solid (4.1 mg, 22%). MS (ES⁺) C₂₉H₂₉F₇N₈O₃ requires: 670, found 671 [M+H]⁺. ¹H NMR (500 MHz, DMSO-d₆) δ 12.34 (s, 1H), 9.15 (t, J=6.2 Hz, 1H), 8.61 (s, 1H), 7.56 (s, 1H), 7.38-7.25 (m, 3H), 6.43 (s, 1H), 4.87-4.75 (m, 1H), 4.56 (s, 1H), 4.53-4.40 (m, 4H), 4.35-4.27 (m, 1H), 4.15-3.99 (m, 2H), 2.96 (t, J=7.5 Hz, 2H), 1.94-1.83 (m, 2H), 1.74-1.65 (m, 2H), 1.39 (d, J=3.8 Hz, 6H).

### Example 96

### 1-(4-(5,6-dicyclopropyl-7H-pyrrolo[2,3-c]pyridazin-3-yl)butyl)-N-((6-methylpyridin-3-yl)methyl)-1H-1,2,3-triazole-4-carboxamide

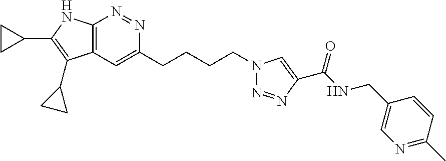 [SMILES: Cc1ccc(CNC(=O)c2cn(CCCCc3cc4c(C5CC5)c(C5CC5)[nH]c4nn3)nn2)cn1]

### Steps 1 to 9

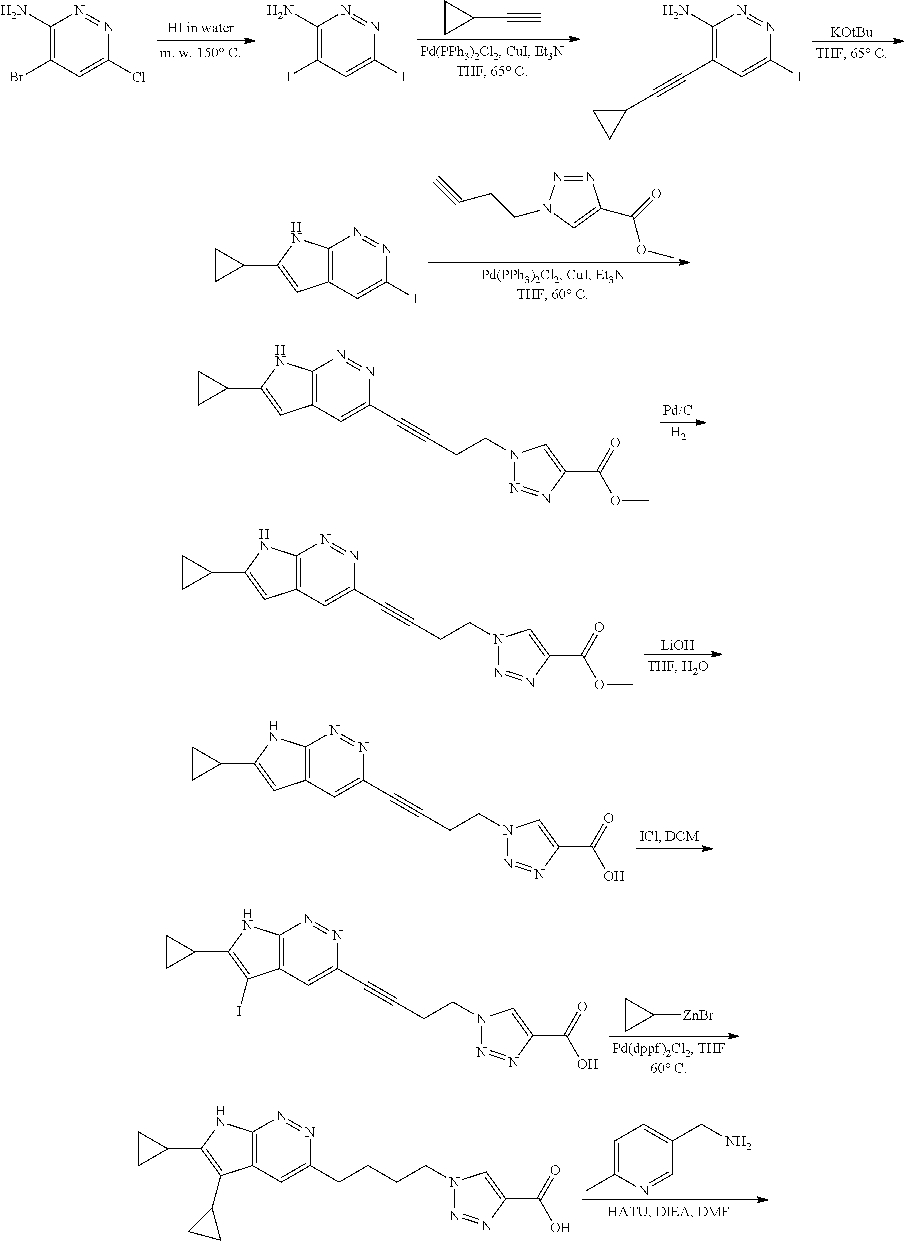

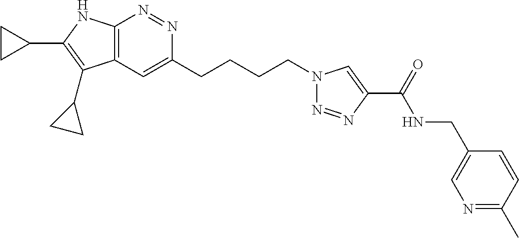 [SMILES: Cc1ccc(CNC(=O)c2cn(CCCCc3cc4c(C5CC5)c(C5CC5)[nH]c4nn3)nn2)cn1]

### Step 1: 4,6-diiodopyridazin-3-amine

A microwave vial was charged with 4-bromo-6-chloropyridazin-3-amine (4 g, 19.19 mmol), and 57% HI in water (40 ml) was added. The vial was sealed and the reaction mixture was heated to 150° C. in the microwave reactor for 16 minutes. The reaction mixture was diluted with EtOAc, sonicated and filtered to give 4,6-diiodopyridazin-3-amine (4.35 g, 65.3%) as a HI salt. After drying, the salt was put in 30 ml water, and sat. NaHCO₃ was added until pH=8. Filter and dry to give the title compound (4.35 g, 65.3%) as a tan solid. MS (ES⁺) C₄H₃I₂N₃ requires: 346, found: 347 [M+H]⁺.

### Step 2: 4-(cyclopropylethynyl)-6-iodopyridazin-3-amine

To a solution of 4,6-diiodopyridazin-3-amine (5 g, 14.41 mmol) in THF (48.0 ml) were added triethylamine (22.10 ml, 159 mmol), ethynylcyclopropane (1.464 ml, 17.30 mmol), Pd(PPh₃)₄ (0.833 g, 0.721 mmol) and CuI (0.082 g, 0.432 mmol). The mixture was degassed for 2 min and the resulting mixture was stirred at 65° C. for 48 h. It was cooled to room temperature, filtered through Celite, washed with DCM (3×30 mL), and concentrated. The residue was purified via silica gel chromatography (0-5% MeOH in DCM) to give the title compound (3.84 g, 93%) as a tan solid. MS (ES⁺) C₉H₈IN₃ requires: 285, found: 286 [M+H]⁺.

### Step 3: 6-cyclopropyl-3-iodo-7H-pyrrolo[2,3-c]pyridazine

To a suspension of 4-(cyclopropylethynyl)-6-iodopyridazin-3-amine (1.5 g, 5.26 mmol) in THF (26.3 ml) was added K-OtBu (0.886 g, 7.89 mmol) and the resulting mixture was stirred at 65° C. for 12 h. The reaction mixture was allowed to cool to RT, filtered through Celite, washed with EtOAc (3×20 mL), and concentrated under reduced pressure. The residue was purified via silica gel chromatography (1-4% MeOH in DCM) to give the title compound (1.04 g, 69.3%) as a tan solid. MS (ES⁺) C₉H₈IN₃ requires: 285, found: 286 [M+H]⁺.

### Step 4: methyl 1-(4-(6-cyclopropyl-7H-pyrrolo[2,3-c]pyridazin-3-yl)but-3-yn-1-yl)-1H-1,2,3-triazole-4-carboxylate

A solution of 6-cyclopropyl-3-iodo-7H-pyrrolo[2,3-c]pyridazine (350 mg, 1.228 mmol), methyl 1-(but-3-yn-1-yl)-1H-1,2,3-triazole-4-carboxylate (220 mg, 1.228 mmol) and triethylamine (1.24 g, 12.28 mmol) in THF (10 ml) was degassed with N₂ for 2 minutes. bis(triphenylphosphine)palladium(II) chloride (86 mg, 0.123 mmol) and copper(I) iodide (23 mg, 0.123 mmol) were added and the mixture was degassed with N₂ for an additional 1 minutes. The reaction mixture was heated to 60° C. and stirred for 2 h. The volatiles were removed under reduced pressure. The solid was sonicated and stirred in water for 10 min and filtered. The residue was purified via silica gel chromatography (1-8% MeOH in DCM) to give the title compound (330 mg, 80%) as an yellow solid. MS (ES⁺) C₁₇H₁₆N₆O₂ requires: 336, found: 337 [M+H]⁺.

### Step 5: methyl 1-(4-(6-cyclopropyl-7H-pyrrolo[2,3-c]pyridazin-3-yl)butyl)-1H-1,2,3-triazole-4-carboxylate

A reaction vessel was charged with methyl 1-(4-(6-cyclopropyl-7H-pyrrolo[2,3-c]pyridazin-3-yl)but-3-yn-1-yl)-1H-1,2,3-triazole-4-carboxylate (300 mg, 0.892 mmol), 5% Pd—C (300 mg), ethanol (20 ml) and DMF (20 ml) under an atmosphere of N₂. The suspension was degassed with N₂ for 2 minutes and purged with H₂ for 2 minutes. The reaction mixture was stirred under an atmosphere of H₂ at 1 atm for 16 h. The reaction mixture was purged with N₂, and filtered through Celite and concentrated under reduced pressure to give the title compound as a yellow solid (282 mg, 93%). It could be carried to next step without further purification. MS (ES⁺) C₁₇H₂₀N₆O₂ requires: 340, found: 341 [M+H]⁺.

### Step 6: 1-(4-(6-cyclopropyl-7H-pyrrolo[2,3-c]pyridazin-3-yl)butyl)-1H-1,2,3-triazole-4-carboxylic acid

To a solution of methyl 1-(4-(6-cyclopropyl-7H-pyrrolo[2,3-c]pyridazin-3-yl)butyl)-1H-1,2,3-triazole-4-carboxylate (300 mg, 0.881 mmol) in THF (5 ml) and Water (5 ml) were added LiOH (42.2 mg, 1.763 mmol) and the resulting mixture was stirred at 20° C. for 1 h. The volatiles were removed under reduced pressure. The residue was taken in MeOH and purified by mass-triggered preparative HPLC (Mobile phase: A=0.1% TFA/H₂O, B=0.1% TFA/MeCN; Gradient: B=10-40%; 12 min; Column: C18) to give the title compound (239 mg, 83%) as a white solid. MS (ES⁺) C₁₆H₁₈N₆O₂ requires: 326, found: 327 [M+H]⁺.

### Step 7: 1-(4-(6-cyclopropyl-5-iodo-7H-pyrrolo[2,3-c]pyridazin-3-yl)butyl)-1H-1,2,3-triazole-4-carboxylic acid

To a suspension of 1-(4-(6-cyclopropyl-7H-pyrrolo[2,3-c]pyridazin-3-yl)butyl)-1H-1,2,3-triazole-4-carboxylic acid (150 mg, 0.460 mmol) in DCM (3 ml) were added ICl (0.035 ml, 0.689 mmol) and the resulting mixture was stirred at 20° C. for 1 h. The reaction mixture was filtered through Buchner funnel to give the title compound (191 mg, 92%) as a yellow solid. MS (ES⁺) C₁₆H₁₇IN₆O₂ requires: 452, found: 453 [M+H]⁺.

### Step 8: 1-(4-(5,6-dicyclopropyl-7H-pyrrolo[2,3-c]pyridazin-3-yl)butyl)-1H-1,2,3-triazole-4-carboxylic acid

A degassed solution of 1-(4-(6-cyclopropyl-5-iodo-7H-pyrrolo[2,3-c]pyridazin-3-yl)butyl)-1H-1,2,3-triazole-4-carboxylic acid (30 mg, 0.066 mmol), PdCl2(dppf)-CH2Cl2Adduct (10.83 mg, 0.013 mmol) and cyclopropylzinc(II) bromide (1500 μl, 0.750 mmol) (0.5 M in THF) was stirred at 60° C. for 2 h. The volatiles were removed under reduced pressure. The residue was purified by mass-triggered preparative HPLC (Mobile phase: A=0.1% TFA/H2O, B=0.1% TFA/MeCN; Gradient: B=10-50%; 12 min; Column: C18) to give the title compound (11 mg, 45.3%) as a yellow solid. MS (ES⁺) C₁₉H₂₂N₆O₂ requires: 366, found: 367 [M+H]⁺.

### Step 9: 1-(4-(5,6-dicyclopropyl-7H-pyrrolo[2,3-c]pyridazin-3-yl)butyl)-N-((6-methylpyridin-3-yl)methyl)-1H-1,2,3-triazole-4-carboxamide 2,2,2-trifluoroacetate

To a solution of 1-(4-(5,6-dicyclopropyl-7H-pyrrolo[2,3-c]pyridazin-3-yl)butyl)-1H-1,2,3-triazole-4-carboxylic acid (10 mg, 0.027 mmol) in DMF (0.5 ml) were added HATU (11.41 mg, 0.030 mmol), (6-methylpyridin-3-yl)methanamine (3.67 mg, 0.030 mmol) and DIEA (7.15 μl, 0.041 mmol) and the resulting mixture was stirred at 20° C. for 1 h. The volatiles were removed under reduced pressure. The residue was purified by mass-triggered preparative HPLC (Mobile phase: A=0.1% TFA/H2O, B=0.1% TFA/MeCN; Gradient: B=10-50%; 20 min; Column: C18) to give the title compound (8 mg, 50.1%) as a white solid. MS (ES⁺) C₂₆H₃₀N₈O requires: 470, found: 471 [M+H]⁺. ¹H NMR (DMSO-d₆) δ: 12.84 (s, 1H), 9.19 (t, J=5.8 Hz, 1H), 8.60 (s, 1H), 8.56 (s, 1H), 8.11 (s, 1H), 7.99 (s, 1H), 7.53 (s, 1H), 4.52-4.43 (m, 4H), 3.07 (t, J=7.6 Hz, 2H), 2.56 (s, 3H), 1.95-1.85 (m, 3H), 2.54-2.50 (m, 1H) 1.76-1.68 (m, 2H), 1.40-1.34 (m, 2H), 1.29-1.24 (m, 2H), 1.04-0.99 (m, 2H), 0.76-0.72 (m, 2H).

### Example 114

### 1-(4-(5-((6-chloropyridin-3-yl)methyl)-6-cyclopropyl-7H-pyrrolo[2,3-c]pyridazin-3-yl)butyl)-N-(pyridin-2-ylmethyl)-1H-1,2,3-triazole-4-carboxamide 2,2,2-trifluoroacetate

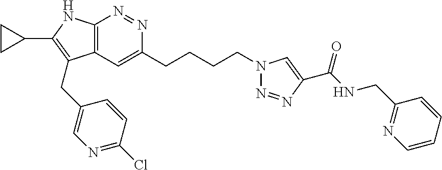 [SMILES: O=C(NCc1ccccn1)c1cn(CCCCc2cc3c(Cc4ccc(Cl)nc4)c(C4CC4)[nH]c3nn2)nn1]

### Steps 1 to 2

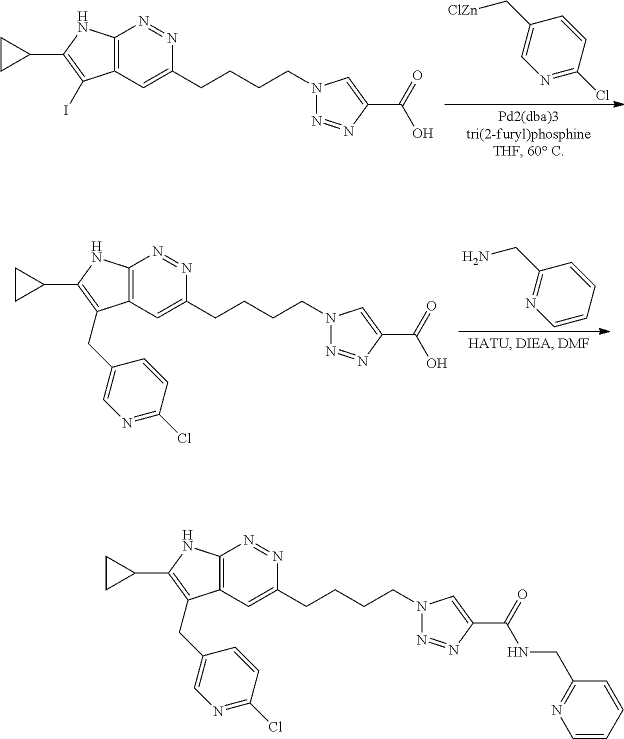

### Step 1: 1-(4-(5-((6-chloropyridin-3-yl)methyl)-6-cyclopropyl-7H-pyrrol[2,3-c]pyridazin-3-yl)butyl)-1H-1,2,3-triazole-4-carboxylic acid

A degassed solution of 1-(4-(6-cyclopropyl-5-iodo-7H-pyrrolo[2,3-c]pyridazin-3-yl)butyl)-1H-1,2,3-triazole-4-carboxylic acid (Example 96, step 7) (35 mg, 0.077 mmol), ((6-chloropyridin-3-yl)methyl)zinc(II) chloride (1548 μl, 0.774 mmol) (0.5M in THF), Pd₂(dba)₃ (14.17 mg, 0.015 mmol), tri(2-furyl)phosphine (3.59 mg, 0.015 mmol) was stirred at 60° C. for 2 h. The volatiles were removed under reduced pressure. The residue was dissolved in MeOH and TFA and purified by mass-triggered preparative HPLC (Mobile phase: A=0.1% TFA/H₂O, B=0.1% TFA/MeCN; Gradient: B=10-50%; 12 min; Column: C18) to give the title compound (8 mg, 22.9%) as a yellow solid. MS (ES⁺) C₂₂H₂₂ClN₇O₂ requires: 451, found: 452 [M+H]⁺.

### Step 2: 1-(4-(5-((6-chloropyridin-3-yl)methyl)-6-cyclopropyl-7H-pyrrol[2,3-c]pyridazin-3-yl)butyl)-N-(pyridin-2-ylmethyl)-1H-1,2,3-triazole-4-carboxamide 2,2,2-trifluoroacetate

To a solution of 1-(4-(5-((6-chloropyridin-3-yl)methyl)-6-cyclopropyl-7H-pyrrolo[2,3-c]pyridazin-3-yl)butyl)-1H-1,2,3-triazole-4-carboxylic acid (8 mg, 0.018 mmol) in DMF (0.5 ml) were added HATU (7.40 mg, 0.019 mmol), pyridin-2-ylmethanamine (2.106 mg, 0.019 mmol) and DIEA (4.64 μl, 0.027 mmol) and the resulting mixture was stirred at 20° C. for 1 h. The volatiles were removed under reduced pressure. The residue was purified by mass-triggered preparative HPLC (Mobile phase: A=0.1% TFA/H₂O, B=0.1% TFA/MeCN; Gradient: B=10-40%; 20 min; Column: C18) to give the title compound (7 mg, 60.3%) as a yellow solid. MS (ES⁺) C₂₈H₂₈ClN₉O requires: 542, found: 543 [M+H]⁺. ¹H NMR (MeOH-d4) δ: 8.74 (d, J=5.7 Hz, 1H), 8.47 (t, J=7.9 Hz, 1H), 8.43 (s, 1H), 8.33 (s, 1H), 8.03 (s, 1H), 7.97 (d, J=8.2 Hz, 1H), 7.89 (t, J=6.7 Hz, 1H), 7.68 (d, J=8.3 Hz, 1H), 7.37 (d, J=8.3 Hz, 1H), 4.54 (t, J=6.8 Hz, 2H), 4.33 (s, 2H), 3.12 (t, J=7.8 Hz, 2H), 2.48-2.42 (m, 1H), 2.06-2.00 (m, 4H), 1.85-1.78 (m, 2H), 1.44-1.38 (m, 2H), 1.29-1.24 (m, 2H).

### Example 217

### 1-(4-(6-cyclopropyl-5-(2,4-difluorophenyl)-7H-pyrrolo[2,3-c]pyridazin-3-yl)butyl)-N-((6-(trifluoromethyl)pyridin-3-yl)methyl)-1H-1,2,3-triazole-4-carboxamide

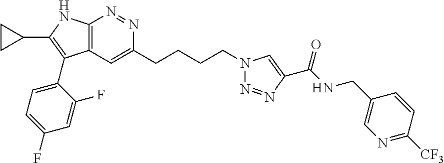 [SMILES: O=C(NCc1ccc(C(F)(F)F)nc1)c1cn(CCCCc2cc3c(-c4ccc(F)cc4F)c(C4CC4)[nH]c3nn2)nn1]

### Steps 1 to 4

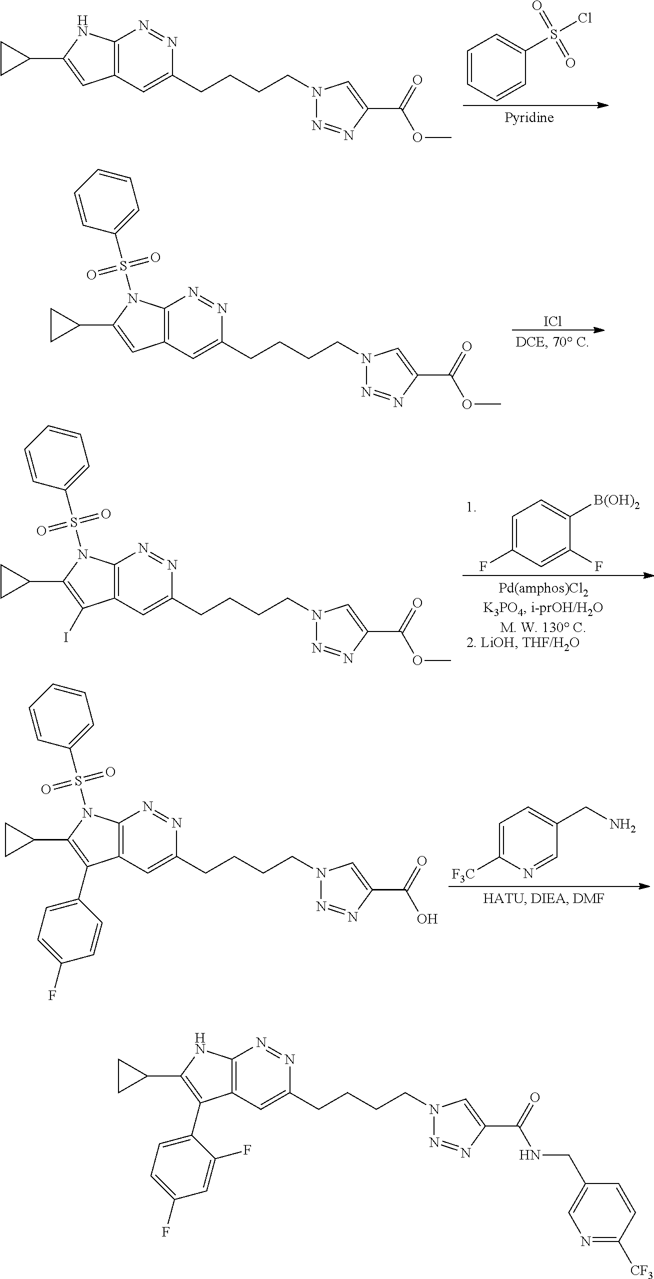

### Step 1: methyl 1-(4-(6-cyclopropyl-7-(phenylsulfonyl)-7H-pyrrolo[2,3-c]pyridazin-3-yl)butyl)-1H-1,2,3-triazole-4-carboxylate

To a solution of methyl 1-(4-(6-cyclopropyl-7H-pyrrolo[2,3-c]pyridazin-3-yl)butyl)-1H-1,2,3-triazole-4-carboxylate (Example 16, step 2) (200 mg, 0.588 mmol) in Pyridine (5 ml) were added benzenesulfonyl chloride (208 mg, 1.175 mmol) and the resulting mixture was stirred at 60° C. for 3 h. LCMS showed 60% complete of the reaction. One more eq of sulfonyl chloride was added and the mixture was heated for another 3 hrs. The volatiles were removed under reduced pressure. Sat. NH₄Cl (30 mL) was added, and the layers were separated. The aqueous phase was extracted with DCM (3×30 mL), the combined organic layers were washed with Sat. NaCl, dried over MgSO₄, filtered and concentrated under reduced pressure. The residue was purified via silica gel chromatography (0-5% MeOH in DCM) to give the title compound (168 mg, 59.5%) as a yellow solid. MS (ES⁺) C₂₃H₂₄N₆O₄S requires: 480, found: 481 [M+H]⁺.

### Step 2: methyl 1-(4-(6-cyclopropyl-5-iodo-7-(phenylsulfonyl)-7H-pyrrolo[2,3-c]pyridazin-3-yl)butyl)-1H-1,2,3-triazole-4-carboxylate

To a solution of methyl 1-(4-(6-cyclopropyl-7-(phenylsulfonyl)-7H-pyrrolo[2,3-c]pyridazin-3-yl)butyl)-1H-1,2,3-triazole-4-carboxylate (160 mg, 0.333 mmol) in 1,2-Dichloroethane (3 ml) were added ICl (0.050 ml, 0.999 mmol) and the resulting mixture was stirred at 70° C. for 8 h. Upon cooling, precipitate formed. The reaction mixture was filtered through Buchner funnel to give the title compound (200 mg, 99%) as a yellow solid. MS (ES⁺) C₂₃H₂₃IN₆O₄S requires: 606, found: 607 [M+H]⁺.

### Step 3: 1-(4-(6-cyclopropyl-5-(2,4-difluorophenyl)-7H-pyrrolo[2,3-c]pyridazin-3-yl)butyl)-1H-1,2,3-triazole-4-carboxylic acid

A degassed solution of methyl 1-(4-(6-cyclopropyl-5-iodo-7-(phenylsulfonyl)-7H-pyrrolo[2,3-c]pyridazin-3-yl)butyl)-1H-1,2,3-triazole-4-carboxylate (400 mg, 0.660 mmol), 2,4-difluorophenyl)boronic acid (208 mg, 1.319 mmol), bis[(dicyclohexyl)(4-dimethylaminophenyl)phosphine]palladium(II) chloride (53.6 mg, 0.066 mmol), tripotassium phosphate (0.989 ml, 1.979 mmol) (2N in water) in 2-Propanol (4 ml) and Water (2 ml) was heated in microwave reactor at 130° C. for 30 min. The volatiles were removed under reduced pressure. Then LiOH (158 mg, 6.60 mmol), THF (5 ml) and water (5 ml) was added. The mixture was stirred at 20° C. for 16 hrs. The volatiles were removed under reduced pressure. The residue was purified by mass-triggered preparative HPLC (Mobile phase: A=0.1% TFA/H₂O, B=0.1% TFA/MeCN; Gradient: B=10-40%; 20 min; Column: C18) to the title compound (88 mg, 30.4%) as a pale yellow solid. MS (ES⁺) C₂₂H₂₀F₂N₆O₂ requires: 438. found: 439 [M+H]⁺.

### Step 4: 1-(4-(6-cyclopropyl-5-(2,4-difluorophenyl)-7H-pyrrolo[2,3-c]pyridazin-3-yl)butyl)-N-((6-(trifluoromethyl)pyridin-3-yl)methyl)-1H-1,2,3-triazole-4-carboxamide 2,2,2-trifluoroacetate

To a solution of 1-(4-(6-cyclopropyl-5-(2,4-difluorophenyl)-7H-pyrrolo[2,3-c]pyridazin-3-yl)butyl)-1H-1,2,3-triazole-4-carboxylic acid (8 mg, 0.018 mmol) in DMF (0.5 ml) were added HATU (7.63 mg, 0.020 mmol), (6-(trifluoromethyl)pyridin-3-yl)methanamine (3.54 mg, 0.020 mmol) and DIEA (4.78 μl, 0.027 mmol) and the resulting mixture was stirred at 20° C. for 1 h. The volatiles were removed under reduced pressure. The residue was purified by mass-triggered preparative HPLC (Mobile phase: A=0.1% TFA/H₂O, B=0.1% TFA/MeCN; Gradient: B=20-60%; 20 min; Column: C18) to give the title compound (8 mg, 61.7%) as a yellow solid. MS (ES⁺) C₂₉H₂₅F₅N₈O requires: 596, found: 597 [M+H]⁺. ¹H NMR (MeOH-d4) δ: 8.7 (s, 1H), 8.36 (s, 1H), 8.01 (dd, J=8.1 Hz, 1.4 Hz, 1H), 7.94 (s, 1H), 7.78 (d, J=8.1 Hz, 1H), 7.65-7.59 (m, 1H), 7.25-7.16 (m, 2H), 4.68 (s, 2H), 4.51 (t, J=6.9 Hz, 2H), 3.13 (t, J=7.8 Hz, 2H), 2.32-2.24 (m, 1H), 2.06-1.98 (m, 2H), 1.85-1.77 (m, 2H), 1.45-1.39 (m, 2H), 1.34-1.28 (m, 2H).

### Example 129

### 1-(4-(6-cyclopropyl-5-(pyrimidin-5-yl)-7H-pyrrolo[2,3-c]pyridazin-3-yl)butyl)-N-(pyridin-2-ylmethyl)-1H-1,2,3-triazole-4-carboxamide

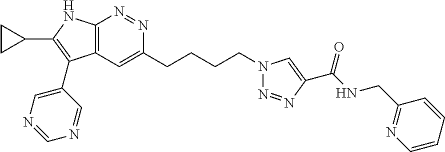 [SMILES: O=C(NCc1ccccn1)c1cn(CCCCc2cc3c(-c4cncnc4)c(C4CC4)[nH]c3nn2)nn1]

### Steps 1 to 2

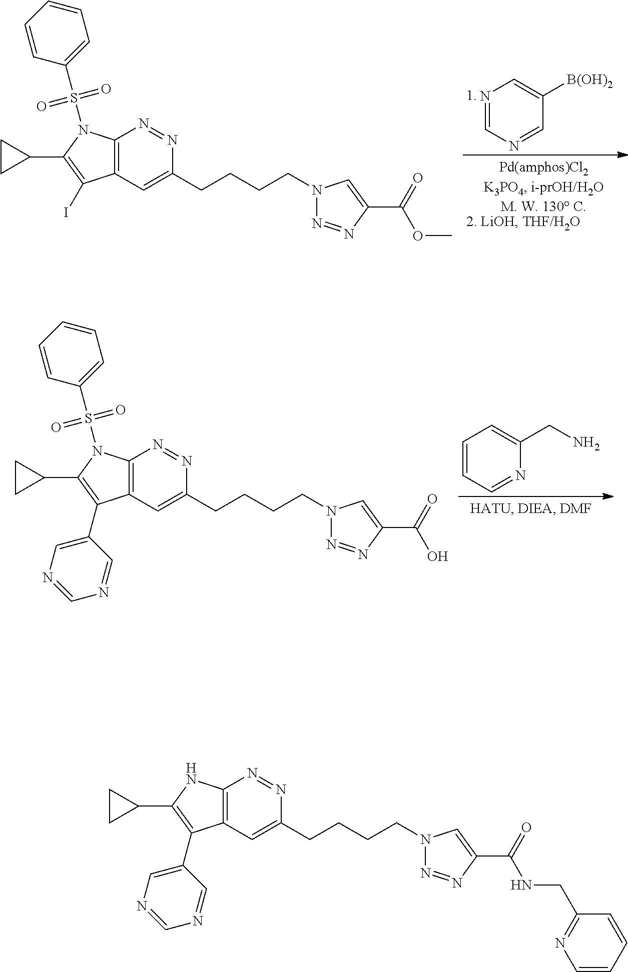

### Step 1: 1-(4-(6-cyclopropyl-5-(pyrimidin-5-yl)-7H-pyrrolo[2,3-c]pyridazin-3-yl)butyl)-1H-1,2,3-triazole-4-carboxylic acid

A degassed solution of methyl 1-(4-(6-cyclopropyl-5-iodo-7-(phenylsulfonyl)-7H-pyrrolo[2,3-c]pyridazin-3-yl)butyl)-1H-1,2,3-triazole-4-carboxylate (30 mg, 0.049 mmol), pyrimidin-5-ylboronic acid (12.26 mg, 0.099 mmol), bis[(dicyclohexyl)(4-dimethylaminophenyl)phosphine]palladium(II) chloride (8.04 mg, 9.89 μmol), tripotassium phosphate (31.5 mg, 0.148 mmol) in 2-Propanol (0.5 ml) and Water (0.5 ml) was stirred at 90° C. for 16 h. LiOH (5.92 mg, 0.247 mmol) was then added and the mixture was stirred at 20° C. for 16 h. The volatiles were removed under reduced pressure. The residue was taken in MeOH and purified by mass-triggered preparative HPLC (Mobile phase: A=0.1% TFA/H₂O, B=0.1% TFA/MeCN; Gradient: B=10-40%; 20 min; Column: C18) to the title compound (8 mg, 40.0%) as a pale yellow solid. MS (ES⁺) C₂₀H₂₀N₈O₂ requires: 404, found: 405 [M+H]⁺.

### Step 2: 1-(4-(6-cyclopropyl-5-(pyrimidin-5-yl)-7H-pyrrolo[2,3-c]pyridazin-3-yl)butyl)-N-(pyridin-2-ylmethyl)-1H-1,2,3-triazole-4-carboxamide 2,2,2-trifluoroacetate

To a solution of 1-(4-(6-cyclopropyl-5-(pyrimidin-5-yl)-7H-pyrrolo[2,3-c]pyridazin-3-yl)butyl)-1H-1,2,3-triazole-4-carboxylic acid (8 mg, 0.020 mmol) in DMF (0.5 ml) were added HATU (8.27 mg, 0.022 mmol), pyridin-2-ylmethanamine (2.353 mg, 0.022 mmol) and DIEA (5.18 μl, 0.030 mmol) and the resulting mixture was stirred at 20° C. for 1 h. The volatiles were removed under reduced pressure. The residue was purified by mass-triggered preparative HPLC (Mobile phase: A=0.1% TFA/H₂O, B=0.1% TFA/MeCN; Gradient: B=10-40%; 20 min; Column: C18) to give the title compound (7 mg, 58.1% yield) as a pale yellow solid. MS (ES⁺) C₂₆H₂₆N₁₀O requires: 494, found: 495 [M+H]⁺. ¹H NMR (DMSO-d₆) δ: 13.42 (s, 1H), 9.29 (s, 1H), 9.12-9.08 (m, 3H), 8.61 (s, 1H), 8.56 (d, J=4.7 Hz, 1H), 8.42 (s, 1H), 7.89 (t, J=7.4 H, 1H), 7.42-7.37 (m, 2H), 4.59 (d, J=6 Hz, 2H), 4.46 (t, J=6.9 Hz, 2H), 3.10 (t, J=7.6 Hz, 2H), 2.48-2.41 (m, 1H), 1.93-1.86 (m, 2H), 1.79-1.72 (m, 2H), 1.41-1.32 (m, 4H).

### Example 177

### 1-(4-(5-cyclopropyl-6-(pyridin-2-yl)-7H-pyrrolo[2,3-c]pyridazin-3-yl)butyl)-N-(3-(trifluoromethoxy)benzyl)-1H-1,2,3-triazole-4-carboxamide 2,2,2-trifluoroacetate

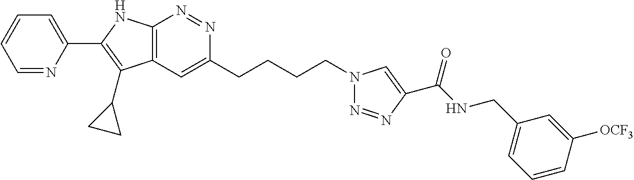 [SMILES: O=C(NCc1cccc(OC(F)(F)F)c1)c1cn(CCCCc2cc3c(C4CC4)c(-c4ccccn4)[nH]c3nn2)nn1]

### Steps 1 to 5

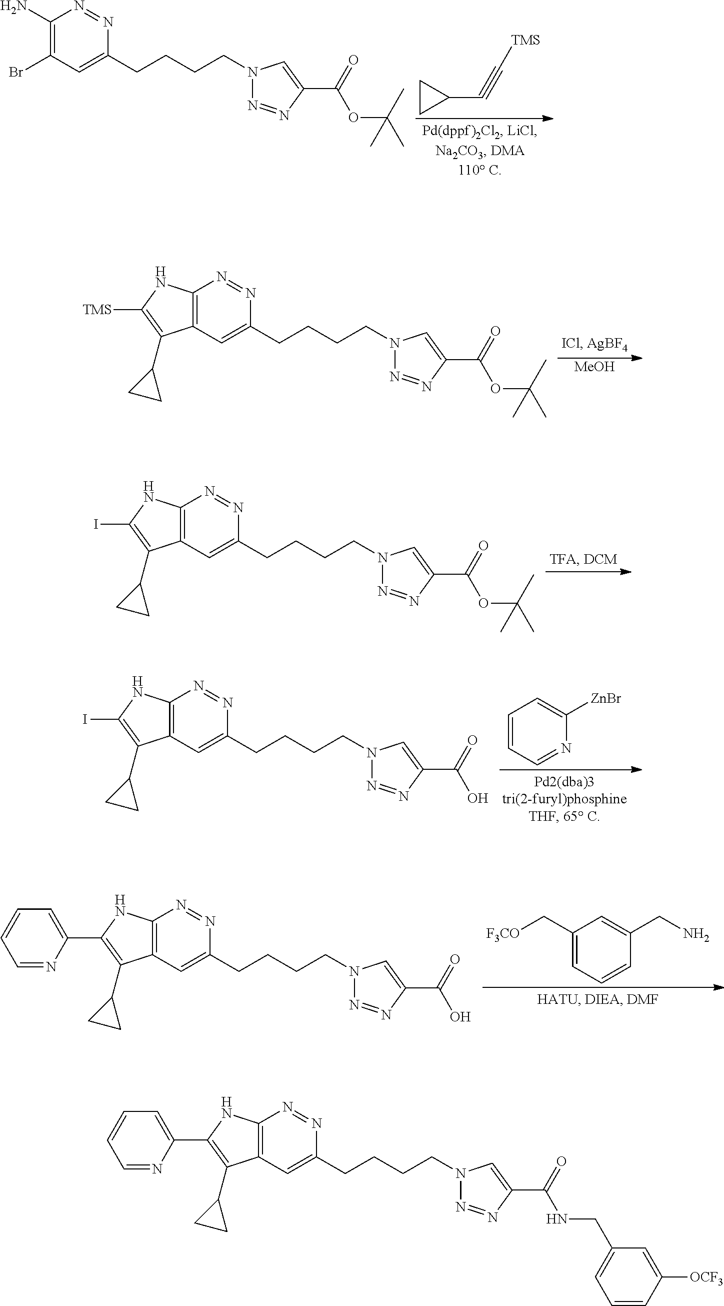

### Step 1: tert-butyl 1-(4-(5-cyclopropyl-6-(trimethylsilyl)-7H-pyrrolo[2,3-c]pyridazin-3-yl)butyl)-1H-1,2,3-triazole-4-carboxylate

A degassed solution of tert-butyl 1-(4-(6-amino-5-bromopyridazin-3-yl)butyl)-1H-1,2,3-triazole-4-carboxylate (Example 134, step 5) (200 mg, 0.503 mmol), (cyclopropylethynyl)trimethylsilane (139 mg, 1.007 mmol), PdCl2(dppf)-CH2Cl2Adduct (82 mg, 0.101 mmol), Na2CO3 (107 mg, 1.007 mmol) and lithium chloride (21.34 mg, 0.503 mmol) in DMA (5 ml) was stirred at 110° C. for 16 h. The volatiles were removed under reduced pressure. The residue was purified by mass-triggered preparative HPLC (Mobile phase: A=0.1% TFA/H₂O, B=0.1% TFA/MeCN; Gradient: B=20-60%; 20 min; Column: C18) to the title compound (54 mg, 23.59%) as a yellow solid. MS (ES⁺) C₂₃H₃₄N₆O₂Si requires: 454. found: 455 [M+H]⁺.

### Step 2: tert-butyl 1-(4-(5-cyclopropyl-6-iodo-7H-pyrrolo[2,3-c]pyridazin-3-yl)butyl)-1H-1,2,3-triazole-4-carboxylate

To a solution of tert-butyl 1-(4-(5-cyclopropyl-6-(trimethylsilyl)-7H-pyrrolo[2,3-c]pyridazin-3-yl)butyl)-1H-1,2,3-triazole-4-carboxylate (100 mg, 0.220 mmol) in MeOH (2 ml) were added silver(I) tetrafluoroborate (47.1 mg, 0.242 mmol) and ICl (0.017 ml, 0.330 mmol) and the resulting mixture was stirred at 20° C. for 4 h. The volatiles were removed under reduced pressure. The residue was purified via silica gel chromatography (0-5% MeOH in DCM to give the title compound (52 mg, 46.5%) as a yellow liquid. MS (ES⁺) C₂₀H₂₅IN₆O₂ requires: 508, found: 509 [M+H]⁺.

### Step 3: 1-(4-(5-cyclopropyl-6-iodo-7H-pyrrolo[2,3-c]pyridazin-3-yl)butyl)-1H-1,2,3-triazole-4-carboxylic acid

A solution of tert-butyl 1-(4-(5-cyclopropyl-6-iodo-7H-pyrrolo[2,3-c]pyridazin-3-yl)butyl)-1H-1,2,3-triazole-4-carboxylate (52 mg, 0.102 mmol) in TFA (0.5 ml) and DCM (1.5 ml) was stirred at 20° C. for 4 h. The volatiles were removed under reduced pressure. The residue was purified by mass-triggered preparative HPLC (Mobile phase: A=0.1% TFA/H₂O, B=0.1% TFA/MeCN; Gradient: B=10-50%; 12 min; Column: C18) to the title compound (26 mg, 56.2%) as a yellow solid. MS (ES⁺) C₁₆H₁₇IN₆O₂ requires: 452, found: 453 [M+H]⁺.

### Step 4: 1-(4-(5-cyclopropyl-6-(pyridin-2-yl)-7H-pyrrolo[2,3-c]pyridazin-3-yl)butyl)-1H-1,2,3-triazole-4-carboxylic acid

A degassed solution of 1-(4-(5-cyclopropyl-6-iodo-7H-pyrrolo[2,3-c]pyridazin-3-yl)butyl)-1H-1,2,3-triazole-4-carboxylic acid (25 mg, 0.055 mmol), tri(furan-2-yl)phosphine (1.283 mg, 5.53 μmol), Pd₂(dba)₃ (5.06 mg, 5.53 μmol) and pyridin-2-ylzinc(II) bromide (1106 μl, 0.553 mmol) (0.5 M THF) was stirred at 65° C. for 2 h. The volatiles were removed under reduced pressure. The residue was taken in TFA and MeOH and purified by mass-triggered preparative HPLC (Mobile phase: A=0.1% TFA/H₂O, B=0.1% TFA/MeCN; Gradient: B=10-40%; 12 min; Column: C18) to the title compound (7 mg, 31.4%) as a yellow solid. MS (ES⁺) C₂₁H₂₁N₇O₂ requires: 403, found: 404 [M+H]⁺.

### Step 5: 1-(4-(5-cyclopropyl-6-(pyridin-2-yl)-7H-pyrrolo[2,3-c]pyridazin-3-yl)butyl)-N-(3-(trifluoromethoxy)benzyl)-1H-1,2,3-triazole-4-carboxamide 2,2,2-trifluoroacetate

To a solution of 1-(4-(5-cyclopropyl-6-(pyridin-2-yl)-7H-pyrrolo[2,3-c]pyridazin-3-yl)butyl)-1H-1,2,3-triazole-4-carboxylic acid (7 mg, 0.017 mmol) in DMF (0.5 ml) were added HATU (9.90 mg, 0.026 mmol), (3-(trifluoromethoxy)phenyl)methanamine (3.65 mg, 0.019 mmol) and DIEA (4.55 μl, 0.026 mmol) and the resulting mixture was stirred at 20° C. for 1 h. The volatiles were removed under reduced pressure. The residue was purified by mass-triggered preparative HPLC (Mobile phase: A=0.1% TFA/H₂O, B=0.1% TFA/MeCN; Gradient: B=10-50%; 20 min; Column: C18) to give the title compound (7 mg, 58.4%) as a yellow solid. MS (ES⁺) C₂₉H₂₇F₃N₈O₂ requires: 576, found: 577 [M+H]⁺. ¹H NMR (MeOH-d4) δ: 8.88-8.85 (m, 1H), 8.56 (d, J=8.0 Hz, 1H), 8.43 (s, 1H), 8.39 (s, 1H), 8.10-8.06 (m, 1H), 7.60-7.57 (m, 1H), 7.42 (t, J=7.9 Hz, 1H), 7.35 (d, J=7.7 Hz, 1H), 7.26 (s, 1H), 7.16 (d, J=8.2 Hz, 1H), 4.59 (s, 2H), 4.56 (t, J=6.8 Hz, 2H), 3.21 (t, J=7.7 Hz, 2H), 2.23-2.17 (m, 1H), 2.12-2.05 (m, 2H), 1.91-1.85 (m, 2H), 1.26-1.22 (m, 2H), 0.73-0.69 (m, 2H).

### Example 90

### 1-(4-(6-cyclopropyl-5-fluoro-7H-pyrrolo[2,3-c]pyridazin-3-yl)butyl)-N-((6-methylpyridin-3-yl)methyl)-1H-1,2,3-triazole-4-carboxamide 2,2,2-trifluoroacetate

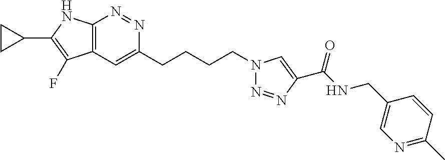 [SMILES: Cc1ccc(CNC(=O)c2cn(CCCCc3cc4c(F)c(C5CC5)[nH]c4nn3)nn2)cn1]

### Steps 1 to 3

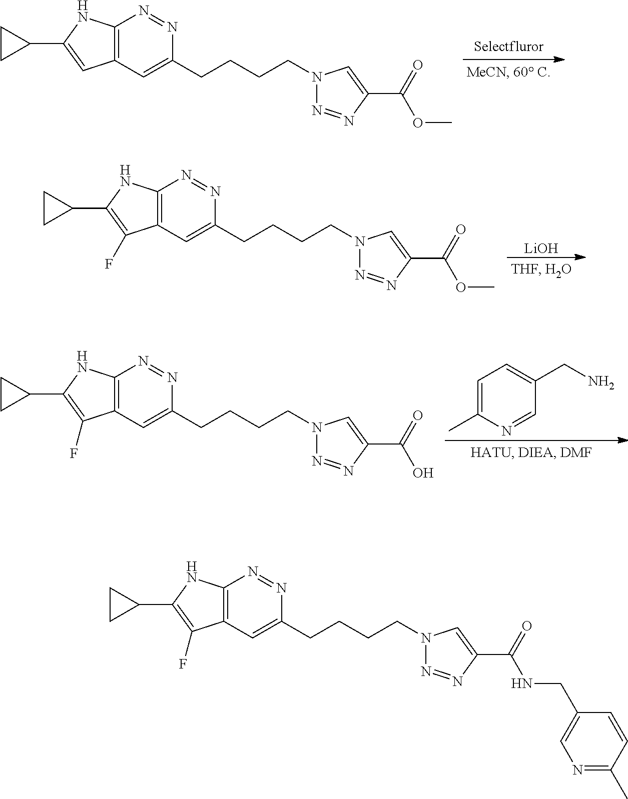

### Step 1: methyl 1-(4-(6-cyclopropyl-5-fluoro-7H-pyrrolo[2,3-c]pyridazin-3-yl)butyl)-1H-1,2,3-triazole-4-carboxylate

To a solution of methyl 1-(4-(6-cyclopropyl-7H-pyrrolo[2,3-c]pyridazin-3-yl)butyl)-1H-1,2,3-triazole-4-carboxylate (Example 16, step 2) (40 mg, 0.118 mmol) in MeCN (1 ml) were added SELECTFLUOR (125 mg, 0.353 mmol) and the resulting mixture was stirred at 60° C. for 16 h. The volatiles were removed under reduced pressure. The residue was purified by mass-triggered preparative HPLC (Mobile phase: A=0.1% TFA/H₂O, B=0.1% TFA/MeCN; Gradient: B=10-40%; 20 min; Column: C18) to give the title compound (8 mg, 19.00%) as a pale yellow solid. MS (ES⁺) C₁₇H₁₉FN₆O₂ requires: 358, found: 359 [M+H]⁺.

### Step 2: 1-(4-(6-cyclopropyl-5-fluoro-7H-pyrrolo[2,3-c]pyridazin-3-yl)butyl)-1H-1,2,3-triazole-4-carboxylic acid

To a solution of methyl 1-(4-(6-cyclopropyl-5-fluoro-7H-pyrrolo[2,3-c]pyridazin-3-yl)butyl)-1H-1,2,3-triazole-4-carboxylate (8 mg, 0.022 mmol) in THF (0.5 ml) and Water (0.5 ml) were added LiOH (1.6 mg, 0.067 mmol) and the resulting mixture was stirred at 20° C. for 1 h. The volatiles were removed under reduced pressure. The residue was purified by mass-triggered preparative HPLC (Mobile phase: A=0.1% TFA/H₂O, B=0.1% TFA/MeCN; Gradient: B=10-40%; 12 min; Column: C18) to give the title compound (6 mg, 78%) as a pale yellow powder. MS (ES⁺) C₁₆H₁₇FN₆O₂ requires: 344, found: 345 [M+H]⁺.

### Step 3: 1-(4-(6-cyclopropyl-5-fluoro-7H-pyrrolo[2,3-c]pyridazin-3-yl)butyl)-N-((6-methylpyridin-3-yl)methyl)-1H-1,2,3-triazole-4-carboxamide 2,2,2-trifluoroacetate

To a solution of 1-(4-(6-cyclopropyl-5-fluoro-7H-pyrrolo[2,3-c]pyridazin-3-yl)butyl)-1H-1,2,3-triazole-4-carboxylic acid (6 mg, 0.017 mmol) in DMF (0.5 ml) were added HATU (7.29 mg, 0.019 mmol), (6-methylpyridin-3-yl)methanamine (2.34 mg, 0.019 mmol) and DIEA (4.56 μl, 0.026 mmol) and the resulting mixture was stirred at 20° C. for 1 h. The volatiles were removed under reduced pressure. The residue was purified by mass-triggered preparative HPLC (Mobile phase: A=0.1% TFA/H₂O, B=0.1% TFA/MeCN; Gradient: B=10-40%; 20 min; Column: C18) to give the title compound (7 mg, 71.4%) as a yellow solid. MS (ES⁺) C₂₃H₂₅FN₈O requires: 448, found: 449 [M+H]⁺. ¹H NMR (MeOH-d4) δ: 8.68 (s, 1H), 8.46 (d, J=8.2 Hz, 1H), 8.40 (s, 1H), 8.13 (s, 1H), 7.88 (d, J=8.2 Hz, 1H), 4.71 (s, 2H), 4.54 (t, J=7.0 hz, 2H), 3.16 (t, J=7.7 Hz, 2H), 2.77 (s, 3H), 2.42-2.37 (m, 1H), 2.08-2.02 (m, 2H), 1.89-1.82 (m, 2H), 1.49-1.45 (m, 2H), 1.35-1.33 (m, 2H).

### Example 109

### 1-(4-(6-cyclopropyl-5-methyl-7H-pyrrolo[2,3-c]pyridazin-3-yl)butyl)-N-(pyridin-2-ylmethyl)-1H-1,2,3-triazole-4-carboxamide

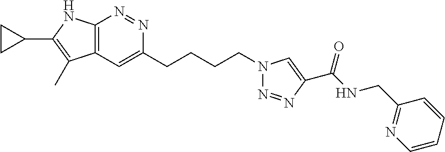 [SMILES: Cc1c(C2CC2)[nH]c2nnc(CCCCn3cc(C(=O)NCc4ccccn4)nn3)cc12]

### Steps 1 to 3

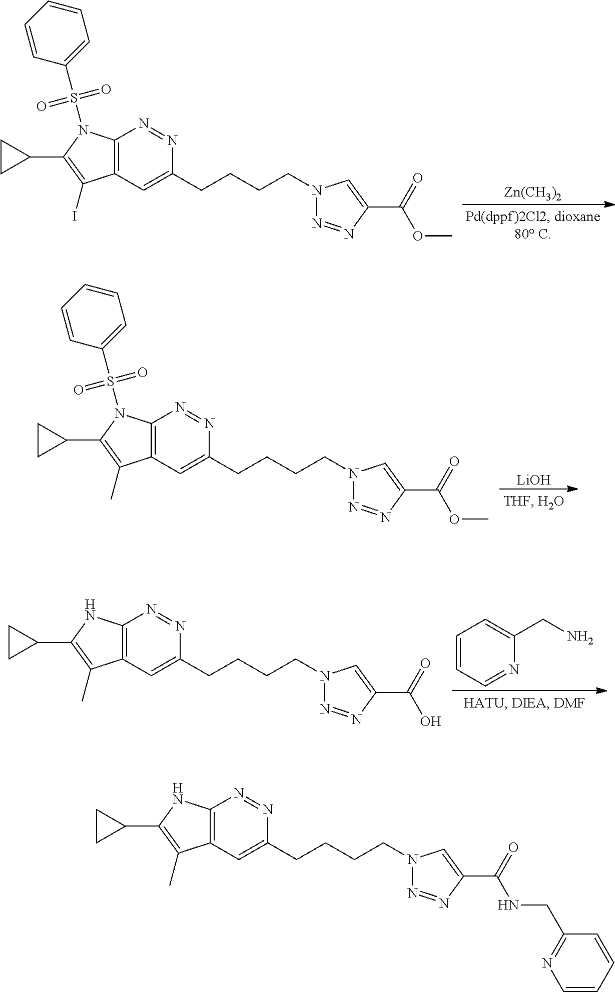

### Step 1: methyl 1-(4-(6-cyclopropyl-5-methyl-7-(phenylsulfonyl)-7H-pyrrolo[2,3-c]pyridazin-3-yl)butyl)-1H-1,2,3-triazole-4-carboxylate

To a degassed solution of methyl 1-(4-(6-cyclopropyl-5-iodo-7-(phenylsulfonyl)-7H-pyrrolo[2,3-c]pyridazin-3-yl)butyl)-1H-1,2,3-triazole-4-carboxylate (Example 217, step 2) (30 mg, 0.049 mmol) and PdCl₂(dppf)-CH2Cl2Adduct (4.04 mg, 4.95 μmol) in Dioxane (1 ml) were added dimethylzinc (0.148 ml, 0.148 mmol) (IM in heptane) and the resulting mixture was stirred at 80° C. for 2 h. The volatiles were removed under reduced pressure. The residue was taken in MeCN and TFA and purified by mass-triggered preparative HPLC (Mobile phase: A=0.1% TFA/H₂O, B=0.1% TFA/MeCN; Gradient: B=30-70%; 20 min; Column: C18) to give the title compound (14 mg, 57.2%) as a yellow solid. MS (ES⁺) C₂₄H₂₆N₆O₄S requires: 494. found: 495 [M+H]⁺.

### Step 2: 1-(4-(6-cyclopropyl-5-methyl-7H-pyrrolo[2,3-c]pyridazin-3-yl)butyl)-1H-1,2,3-triazole-4-carboxylic acid

To a solution of methyl 1-(4-(6-cyclopropyl-5-methyl-7-(phenylsulfonyl)-7H-pyrrolo[2,3-c]pyridazin-3-yl)butyl)-1H-1,2,3-triazole-4-carboxylate (13 mg, 0.026 mmol) in THF (0.5 ml) and Water (0.5 ml) were added LiOH (6.29 mg, 0.263 mmol) and the resulting mixture was stirred at 50° C. for 4 h. The volatiles were removed under reduced pressure. The residue was purified by mass-triggered preparative HPLC (Mobile phase: A=0.1% TFA/H₂O, B=0.1% TFA/MeCN; Gradient: B=10-50%; 12 min; Column: C18) to give the title compound (7 mg, 78%) as a yellow solid. MS (ES⁺) C₁₇H₂₀N₆O₂ requires: 340, found: 341 [M+H]⁺.

### Step 3: 1-(4-(6-cyclopropyl-5-methyl-7H-pyrrolo[2,3-c]pyridazin-3-yl)butyl)-N-(pyridin-2-ylmethyl)-1H-1,2,3-triazole-4-carboxamide 2,2,2-trifluoroacetate

To a solution of 1-(4-(6-cyclopropyl-5-methyl-7H-pyrrolo[2,3-c]pyridazin-3-yl)butyl)-1H-1,2,3-triazole-4-carboxylic acid (7 mg, 0.021 mmol) in DMF (0.5 ml) were added HATU (8.60 mg, 0.023 mmol), pyridin-2-ylmethanamine (2.446 mg, 0.023 mmol) and DIEA (5.39 μl, 0.031 mmol) and the resulting mixture was stirred at 20° C. for 1 h. The volatiles were removed under reduced pressure. The residue was purified by mass-triggered preparative HPLC (Mobile phase: A=0.1% TFA/H₂O, B=0.1% TFA/MeCN; Gradient: B=10-50%; 12 min; Column: C18) to give the title compound (7 mg, 62.5%) as a yellow solid. MS (ES⁺) C₂₃H₂₆N₈O requires: 430, found: 431 [M+H]⁺. ¹H NMR (DMSO-d₆) δ: 12.83 (s, 1H), 9.08 (t, J=5.9 Hz, 1H), 8.63 (s, 1H), 8.53 (d, J=5.0 Hz, 1H), 8.20 (s, 1H), 7.81 (t, J=7.8 Hz, 1H), 7.37-7.31 (m, 2H), 4.57 (d, J=5.9 Hz, 2H), 4.47 (t, J=6.7 Hz, 2H), 3.07 (t, J=7.1 Hz, 2H), 2.44-2.37 (m, 1H), 2.35 (s, 3H), 1.94-1.87 (m, 2H), 1.78-1.71 (m, 2H), 1.36-1.31 (m, 2H), 1.26-1.22 (m, 2H).

### Example 142

### N-(pyridin-2-ylmethyl)-1-(4-(5,6,7,8-tetrahydrocyclopenta[4,5]pyrrolo[2,3-c]pyridazin-3-yl)butyl)-1H-1,2,3-triazole-4-carboxamide

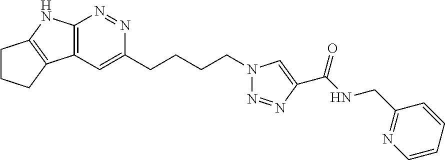 [SMILES: O=C(NCc1ccccn1)c1cn(CCCCc2cc3c4c([nH]c3nn2)CCC4)nn1]

### Steps 1 to 2

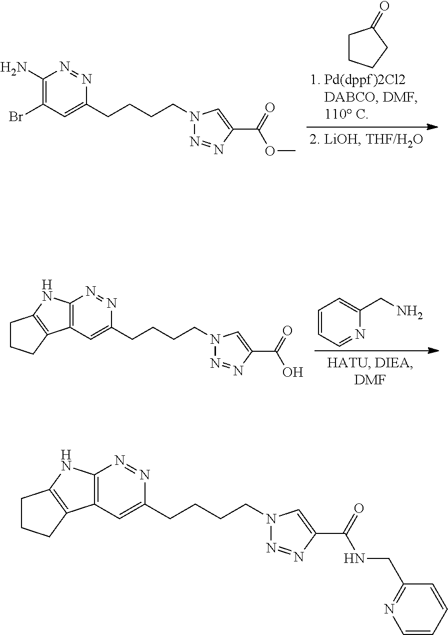

### Step 1: 1-(4-(5,6,7,8-tetrahydrocyclopenta[4,5]pyrrolo[2,3-c]pyridazin-3-yl)butyl)-1H-1,2,3-triazole-4-carboxylic acid

A degassed solution of methyl 1-(4-(6-amino-5-bromopyridazin-3-yl)butyl)-1H-1,2,3-triazole-4-carboxylate (Example 134, step 5) (20 mg, 0.056 mmol), cyclopentanone (9.47 mg, 0.113 mmol), DABCO (18.95 mg, 0.169 mmol) and PdCl₂(dppf)-CH2Cl2Adduct (4.60 mg, 5.63 μmol) in DMF (1 ml) was stirred at 110° C. for 16 h. The volatiles were removed under reduced pressure. THF (0.5 ml), water (0.5 ml) and LiOH (4.05 mg, 0.169 mmol) were added and the mixture was stirred at 20° C. for 1 h. The volatiles were removed under reduced pressure. The residue was purified by mass-triggered preparative HPLC (Mobile phase: A=0.1% TFA/H₂O, B=0.1% TFA/MeCN; Gradient: B=10-40%; 12 min; Column: C18) to give the title compound (3 mg, 16.33%) as a pale yellow solid. MS (ES⁺) C₁₆H₁₈N₆O₂ requires: 326. found: 327 [M+H]⁺.

### Step 2: N-(pyridin-2-ylmethyl)-1-(4-(5,6,7,8-tetrahydrocyclopenta[4,5]pyrrolo[2,3-c]pyridazin-3-yl)butyl)-1H-1,2,3-triazole-4-carboxamide 2,2,2-trifluoroacetate

To a solution of 1-(4-(5,6,7,8-tetrahydrocyclopenta[4,5]pyrrolo[2,3-c]pyridazin-3-yl)butyl)-1H-1,2,3-triazole-4-carboxylic acid (3 mg, 9.19 μmol) in DMF (0.5 ml) were added HATU (3.84 mg, 10.11 μmol), pyridin-2-ylmethanamine (1.093 mg, 10.11 μmol) and DIEA (2.408 μl, 0.014 mmol) and the resulting mixture was stirred at 20° C. for 1 h. The volatiles were removed under reduced pressure. The residue was purified by mass-triggered preparative HPLC (Mobile phase: A=0.1% TFA/H₂O, B=0.1% TFA/MeCN; Gradient: B=0-30%; 20 min; Column: C18) to give the title compound (3 mg, 61.5%) as a pale yellow solid. MS (ES⁺) C₂₂H₂₄N₈O requires: 416, found: 417 [M+H]⁺. ¹H NMR (MeOH-d4) δ: 8.60 (d, J=4.9 Hz, 1H), 8.40 (s, 1H), 8.09 (t, J=7.8 Hz, 1H), 8.02 (s, 1H), 7.66 (d, J=8.0 Hz, 1H), 7.56 (t, J=6.4 Hz, 1H), 4.78 (s, 2H), 4.55 (t, J=6.9 Hz, 2H), 3.20-3.12 (m, 4H), 2.95 (t, J=7.0 Hz, 2H), 2.68-2.62 (m, 2H), 2.10-2.02 (m, 2H), 1.89-1.83 (m, 2H).

### Example 241

### 1-(2-fluoro-4-(6-(2-fluorophenyl)-7H-pyrrolo[2,3-c]pyridazin-3-yl)butyl)-N-((6-methylpyridin-3-yl)methyl)-1H-1,2,3-triazole-4-carboxamide

### Steps 1 to 10

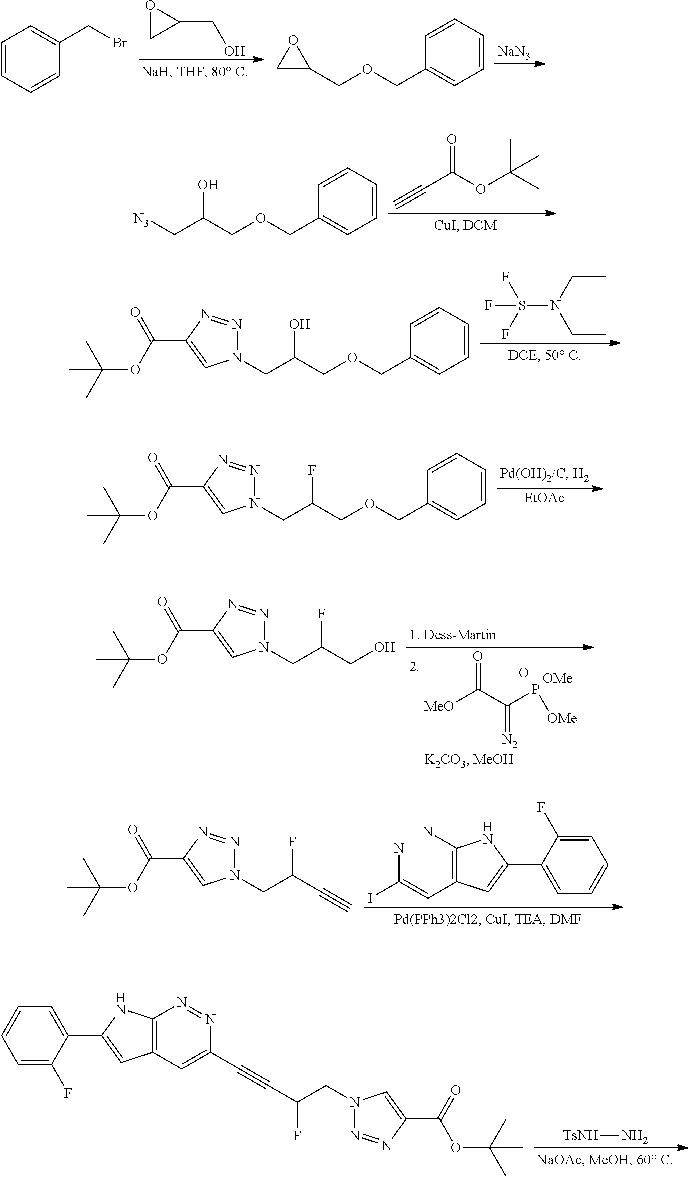

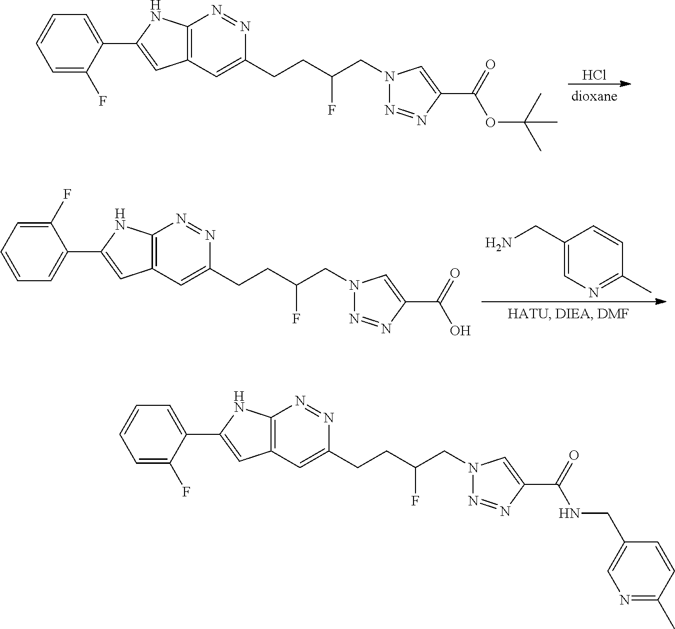

### Step 1: 2-(benzyloxymethyl)oxirane

To a solution of (bromomethyl)benzene (30 g, 175 mmol) in THF (200 mL) were added oxiran-2-ylmethanol (10 g, 135 mmol) and sodium hydride (5.4 g, 135 mmol). The resulting mixture was stirred at 80° C. for 15 h. The volatiles were removed under reduced pressure. The residue was purified via silica gel chromatography (10%-100% Pet ether in EtOAc) to give the title compound as a white solid (4 g, 18%). MS (ES⁺) C₁₀H₁₂O₂ requires: 163, found: 164 [M+H]⁺.

### Step 2: 1-azido-3-(benzyloxy)propan-2-ol

To a mixture of 2-((benzyloxy)methyl)oxirane (2.0 ml, 13.12 mmol) in Methanol (40 ml) and Water (6 ml) were added NH₄Cl (1.491 g, 27.9 mmol) and sodium azide (6.804 g, 105 mmol) and the resulting mixture was stirred at RT for 3 days. The volatiles were removed under reduced pressure. The residue was partitioned between 40 mL of water and 40 mL of ethyl acetate, and the aqueous layer was extracted with EtOAc (2×40 mL). The combined organic layers were dried over Na₂SO₄, filtered and concentrated to give the title compound (2.53 g, 93%) as a colorless oil. It could be carried to next step without further purification. The compound did not ionize well. It was characterized by proton NMR. ¹H NMR (600 MHz, CDCl₃) d 7.27-7.40 (m, 5H), 4.56 (s, 2H), 3.97 (quin, J=5.19 Hz, 1H), 3.47-3.56 (m, 2H), 3.33-3.43 (m, 2H), 2.18-2.57 (br s, 1H).

### Step 3: tert-butyl 1-(3-(benzyloxy)-2-hydroxypropyl)-1H-1,2,3-triazole-4-carboxylate

To a solution of 1-azido-3-(benzyloxy)propan-2-ol (crude) (2.53 g, 12.21 mmol) in CH₂Cl₂ (60 ml) were added N-ethyl-N-isopropylpropan-2-amine (211.0 μL, 1.220 mmol), acetic acid (70.0 μL, 1.223 mmol), tert-butyl propiolate (2000 μL, 14.59 mmol) and copper(I) iodide (114.6 mg, 0.602 mmol) and the resulting mixture was stirred at RT for 4 h. 20 g of silica gel was added and the resulting mixture was concentrated to a pale yellow powder. The residue was purified via silica gel chromatography (0-50% EtOAc in hexanes) to give the title compound (3.18 g, 78%) as a white solid. MS (ES⁺) C₁₇H₂₃N₃O₄ requires: 333, found: 334 [M+H]⁺.

### Step 4: tert-butyl 1-(3-(benzyloxy)-2-fluoropropyl)-1H-1,2,3-triazole-4-carboxylate

To a solution of tert-butyl 1-(3-(benzyloxy)-2-hydroxypropyl)-1H-1,2,3-triazole-4-carboxylate (997.7 mg, 2.99 mmol) in DCE (anhydrous) (15 ml) was added DAST (0.967 ml, 7.32 mmol) and the resulting mixture was stirred at 50° C. for 3 h. The resulting cloudy yellow solution was allowed to cool to RT, treated with additional DAST (0.396 ml, 3.00 mmol) and then stirred at 50° C. for additional 15 h. The cloudy orange solution was chilled in an ice/water bath, treated with 15 g of silica gel, and concentrated to a pale yellow powder. The residue was purified via silica gel chromatography (0-25% EtOAc in hexanes) to give the title compound (590.0 mg, 58.8%) as a white solid. MS (ES⁺) C₁₇H₂₂FN₃O₃ requires: 335, found: 336 [M+H]⁺, 358 [M+Na]⁺, 280 [M-tBu+H]⁺.

### Step 5: tert-butyl 1-(2-fluoro-3-hydroxypropyl)-1H-1,2,3-triazole-4-carboxylate

A reaction vessel was charged with tert-butyl 1-(3-(benzyloxy)-2-fluoropropyl)-1H-1,2,3-triazole-4-carboxylate (584.2 mg, 1.742 mmol) and EtOAc (18 ml) under an atmosphere of N₂. The flask was twice evacuated and filled with nitrogen. Palladium hydroxide on carbon (300 mg, 0.214 mmol) was added. The flask was evacuated and filled with hydrogen, and the dark suspension was stirred under an hydrogen balloon atmosphere for 19 h. The suspension was filtered through Celite 545 and concentrated under reduced pressure to give the title compound (438.4 mg, 103%) as a white solid. MS (ES⁺) C₁₀H₁₆FN₃O₃ requires: 245, found: 246 [M+H]⁺, 268 [M+Na]⁺, 190 [M-tBu+H]⁺.

### Step 6: tert-butyl 1-(2-fluorobut-3-ynyl)-1H-1,2,3-triazole-4-carboxylate

To a solution of tert-butyl 1-(2-fluoro-3-hydroxypropyl)-1H-1,2,3-triazole-4-carboxylate (49.5 mg, 0.202 mmol) in DCM (anhydrous) (2 ml) was added DMP (135.1 mg, 0.319 mmol) and the cloudy white mixture was stirred at 25° C. for 1 hour. MeOH (0.082 ml, 2.027 mmol) was added. The resulting mixture was stirred for 5 minutes, and concentrated. The remaining white residue was taken up in Methanol (2 ml), then potassium carbonate (371.0 mg, 2.68 mmol), dimethyl (1-diazo-2-oxopropyl)phosphonate (10% in acetonitrile) (1.475 ml, 0.614 mmol) were added. The resulting white mixture was stirred at 25° C. for 3.5 h. The volatiles were removed under reduced pressure. The residue was purified via silica gel chromatography (0-33% EtOAc in hexanes) to give the title compound (22.0 mg, 45.6%) as a white solid. MS (ES⁺) C₁₁H₁₄FN₃O₂ requires: 239, found: 240 [M+H]⁺, 262 [M+Na]⁺, 184 [M-tBu+H]⁺.

### Step 7: tert-butyl 1-(2-fluoro-4-(6-(2-fluorophenyl)-7H-pyrrolo[2,3-c]pyridazin-3-yl)but-3-ynyl)-1H-1,2,3-triazole-4-carboxylate

To a solution of tert-butyl 1-(2-fluorobut-3-ynyl)-1H-1,2,3-triazole-4-carboxylate (175 mg, 0.7 mmol) in DMF (5 mL) were added 6-(2-fluorophenyl)-3-iodo-7H-pyrrolo[2,3-c]pyridazine (190 mg, 0.56 mmol), Pd(PPh₃)₂Cl₂ (40 mg, 0.056 mmol), CuI (10.7 mg, 0.056 mmol) and TEA (282 mg, 2.8 mmol) and the resulting mixture was stirred at RT for 15 h. The volatiles were removed under reduced pressure. The residue was purified with silica gel chromatography (10%-100% PE in EtOAc) to give the title compound as a white solid (50 mg, 19%). MS (ES⁺) C₂₃H₂₀F₂N₆O₂ requires: 449, found: 450 [M+H]⁺.

### Step 8: tert-butyl 1-(2-fluoro-4-(6-(2-fluorophenyl)-7H-pyrrolo[2,3-c]pyridazin-3-yl)butyl)-1H-1,2,3-triazole-4-carboxylate

To a solution of tert-butyl 1-(2-fluoro-4-(6-(2-fluorophenyl)-7H-pyrrolo[2,3-c]pyridazin-3-yl)but-3-ynyl)-1H-1,2,3-triazole-4-carboxylate (40 mg, 0.08 mmol) in MeOH (5 mL) were added 4-methylbenzenesulfonohydrazide (248 mg, 1.33 mmol) and sodium acetate (54 mg, 0.66 mmol) and the resulting mixture was stirred at 60° C. for 15 h. The volatiles were removed under reduced pressure. The residue was purified by preparative HPLC to give the title compound (10 mg, 11%) as a white solid. MS (ES⁺) C₂₃H₂₄F₂N₆O₂ requires: 453, found: 454 [M+H]⁺.

### Step 9: 1-(2-fluoro-4-(6-(2-fluorophenyl)-7H-pyrrolo[2,3-c]pyridazin-3-yl)butyl)-1H-1,2,3-triazole-4-carboxylic acid

A solution of tert-butyl 1-(2-fluoro-4-(6-(2-fluorophenyl)-7H-pyrrolo[2,3-c]pyridazin-3-yl)butyl)-1H-1,2,3-triazole-4-carboxylate (15 mg, 0.03 mmol) in HCl in dioxane (15 mL, 4M) was stirred at RT for 8 h. The volatiles were removed under reduced pressure to give the title compound (14 mg, 98%) as a white solid. MS (ES⁺) C₁₉H₁₆F₂N₆O₂ requires: 398. found: 399 [M+H]⁺.

### Step 10: 1-(2-fluoro-4-(6-(2-fluorophenyl)-7H-pyrrolo[2,3-c]pyridazin-3-yl)butyl)-N-((6-methylpyridin-3-yl)methyl)-1H-1,2,3-triazole-4-carboxamide

To a solution of 1-(2-fluoro-4-(6-(2-fluorophenyl)-7H-pyrrolo[2,3-c]pyridazin-3-yl)butyl)-1H-1,2,3-triazole-4-carboxylic acid (20 mg, 0.05 mmol) in DMF (3 mL) were added (6-methylpyridin-3-yl)methanamine (9.2 mg, 0.075 mmol), HATU (28 mg, 0.75 mmol) and DIEA (20 mg, 0.15 mmol) and the resulting mixture was stirred at 25° C. for 2 h. The volatiles were removed under reduced pressure. The residue was purified by preparative HPLC to give the title compound (10 mg, 39%) as white solid. MS (ES⁺) C₂₆H₂₄F₂N₈O requires: 501, found: 502 [M+H]⁺. ¹H NMR (500 MHz, DMSO-d₆) δ 9.19 (d, J=6.1 Hz, 1H), 8.59 (s, 1H), 8.39 (s, 1H), 8.08 (t, J=7.4 Hz, 1H), 7.74 (s, 1H), 7.61-7.39 (m, 4H), 7.19 (d, J=8.1 Hz, 1H), 6.91 (d, J=3.0 Hz, 1H), 5.12-4.97 (m, 1H), 4.89-4.71 (m, 2H), 4.41 (d, J=6.1 Hz, 2H), 3.20-3.08 (m, 2H), 2.42 (s, 3H), 2.28-2.07 (m, 2H).

Non-limiting examples include the following compounds and pharmaceutically acceptable salts thereof:

TABLE 1
Synthesized Examples

| Ex. | | |
|---|---|---|
| No. | Structure | IUPAC Name |
| 1 | 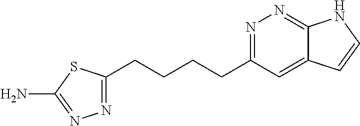 [SMILES: Nc1nnc(CCCCc2cc3cc[nH]c3nn2)s1] | 5-(4-(7H-pyrrolo[2,3- c]pyridazin-3-yl)butyl)-1,3,4- thiadiazol-2-amine |
| 2 | 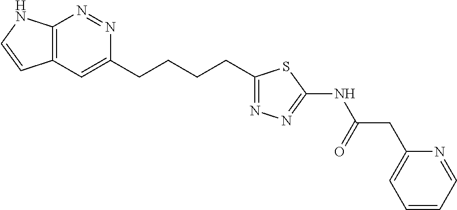 [SMILES: O=C(Cc1ccccn1)Nc1nnc(CCCCc2cc3cc[nH]c3nn2)s1] | N-(5-(4-(7H-pyrrolo[2,3- c]pyridazin-3-yl)butyl)-1,3,4- thiadiazol-2-yl)-2-(pyridin-2- yl)acetamide |
| 3 | 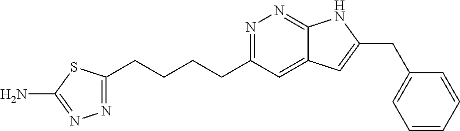 [SMILES: Nc1nnc(CCCCc2cc3cc(Cc4ccccc4)[nH]c3nn2)s1] | 5-(4-(6-benzyl-7H- pyrrolo[2,3-c]pyridazin-3- yl)butyl)-1,3,4-thiadiazol-2- amine |
| 4 | 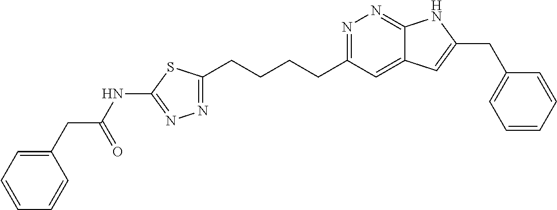 [SMILES: O=C(Cc1ccccc1)Nc1nnc(CCCCc2cc3cc(Cc4ccccc4)[nH]c3nn2)s1] | N-(5-(4-(6-benzyl-7H- pyrrolo[2,3-c]pyridazin-3- yl)butyl)-1,3,4-thiadiazol-2- yl)-2-phenylacetamide |
| 5 | 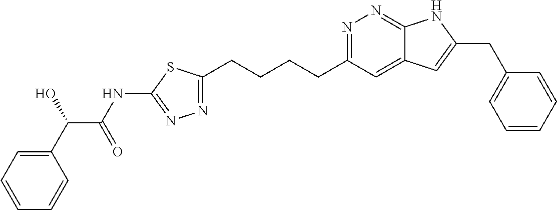 [SMILES: O=C(Nc1nnc(CCCCc2cc3cc(Cc4ccccc4)[nH]c3nn2)s1)[C@@H](O)c1ccccc1] | (S)-N-(5-(4-(6-benzyl-7H- pyrrolo[2,3-c]pyridazin-3- yl)butyl)-1,3,4-thiadiazol-2- yl)-2-hydroxy-2- phenylacetamide |
| 6 | 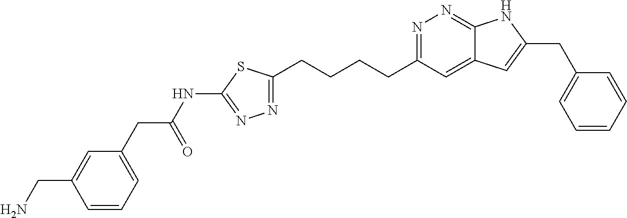 [SMILES: NCc1cccc(CC(=O)Nc2nnc(CCCCc3cc4cc(Cc5ccccc5)[nH]c4nn3)s2)c1] | 2-(3-(aminomethyl)phenyl)- N-(5-(4-(6-benzyl-7H- pyrrolo[2,3-c]pyridazin-3- yl)butyl)-1,3,4-thiadiazol-2- yl)acetamide |
| 7 | 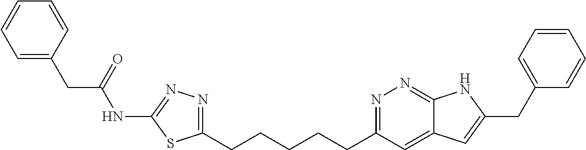 [SMILES: O=C(Cc1ccccc1)Nc1nnc(CCCCCc2cc3cc(Cc4ccccc4)[nH]c3nn2)s1] | N-(5-(5-(6-benzyl-7H- pyrrolo[2,3-c]pyridazin-3- yl)pentyl)-1,3,4-thiadiazol-2- yl)-2-phenylacetamide |
| 8 | 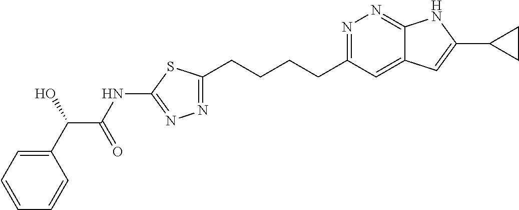 [SMILES: O=C(Nc1nnc(CCCCc2cc3cc(C4CC4)[nH]c3nn2)s1)[C@@H](O)c1ccccc1] | (S)-N-(5-(4-(6-Cyclopropyl- 7H-pyrrolo[2,3-c]pyridazin-3- yl)butyl)-1,3,4-thiadiazol-2- yl)-2-hydroxy-2- phenylacetamide |
| 9 | 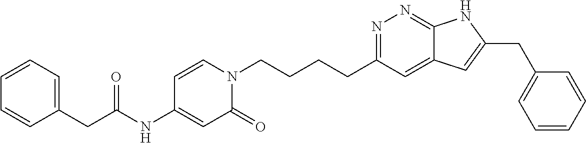 [SMILES: O=C(Cc1ccccc1)Nc1ccn(CCCCc2cc3cc(Cc4ccccc4)[nH]c3nn2)c(=O)c1] | N-(1-(4-(6-Benzyl-7H- pyrrolo[2,3-c]pyridazin-3- yl)butyl)-2-oxo-1,2- dihydropyridin-4-yl)-2- phenylacetamide |
| 10 | 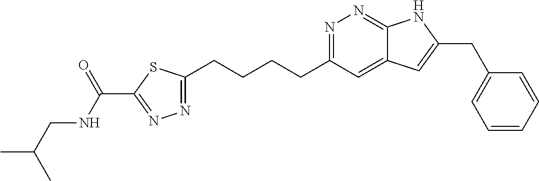 [SMILES: CC(C)CNC(=O)c1nnc(CCCCc2cc3cc(Cc4ccccc4)[nH]c3nn2)s1] | 5-(4-(6-Benzyl-7H- pyrrolo[2,3-c]pyridazin-3- yl)butyl)-N-isobutyl-1,3,4- thiadiazole-2-carboxamide |
| 11 | 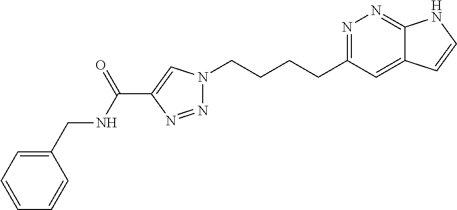 [SMILES: O=C(NCc1ccccc1)c1cn(CCCCc2cc3cc[nH]c3nn2)nn1] | 1-(4-(7H-pyrrolo[2,3- c]pyridazin-3-yl)butyl)-N- benzyl-1H-1,2,3-triazole-4- carboxamide |
| 12 | 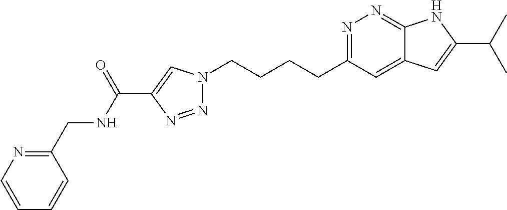 [SMILES: CC(C)c1cc2cc(CCCCn3cc(C(=O)NCc4ccccn4)nn3)nnc2[nH]1] | 1-(4-(6-isopropyl-7H- pyrrolo[2,3-c]pyridazin-3- yl)butyl)-N-(pyridin-2- ylmethyl)-1H-1,2,3-triazole- 4-carboxamide |
| 13 | 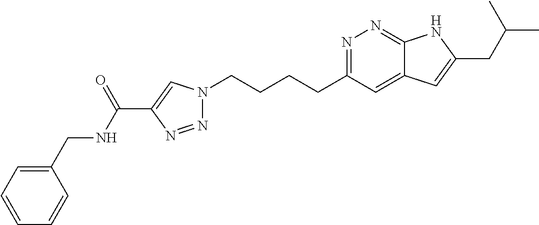 [SMILES: CC(C)Cc1cc2cc(CCCCn3cc(C(=O)NCc4ccccc4)nn3)nnc2[nH]1] | N-benzyl-1-(4-(6-isobutyl- 7H-pyrrolo[2,3-c]pyridazin-3- yl)butyl)-1H-1,2,3-triazole-4- carboxamide |
| 14 | 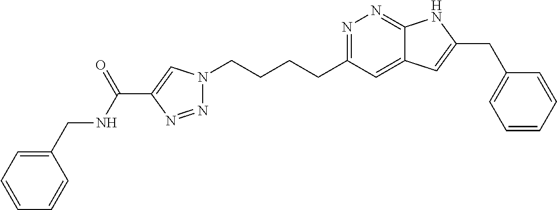 [SMILES: O=C(NCc1ccccc1)c1cn(CCCCc2cc3cc(Cc4ccccc4)[nH]c3nn2)nn1] | N-benzyl-1-(4-(6-benzyl-7H- pyrrolo[2,3-c]pyridazin-3- yl)butyl)-1H-1,2,3-triazole-4- carboxamide |
| 15 | 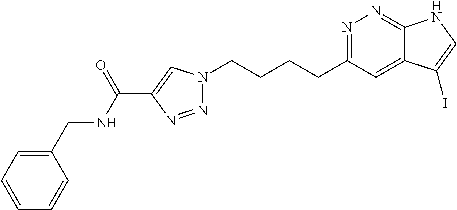 [SMILES: O=C(NCc1ccccc1)c1cn(CCCCc2cc3c(I)c[nH]c3nn2)nn1] | N-benzyl-1-(4-(5-iodo-7H- pyrrolo[2,3-c]pyridazin-3- yl)butyl)-1H-1,2,3-triazole-4- carboxamide |
| 16 | 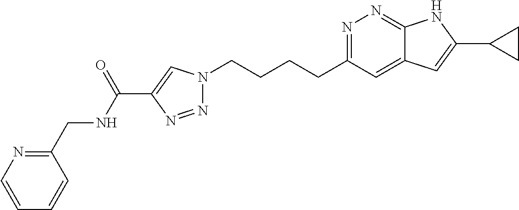 [SMILES: O=C(NCc1ccccn1)c1cn(CCCCc2cc3cc(C4CC4)[nH]c3nn2)nn1] | 1-(4-(6-cyclopropyl-7H- pyrrolo[2,3-c]pyridazin-3- yl)butyl)-N-(pyridin-2- ylmethyl)-1H-1,2,3-triazole- 4-carboxamide |
| 17 | 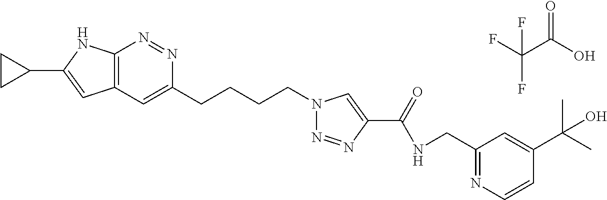 | 1-(4-(6-cyclopropyl-7H- pyrrolo[2,3-c]pyridazin-3- yl)butyl)-N-((4-(2- hydroxypropan-2-yl)pyridin- 2-yl)methyl)-1H-1,2,3- triazole-4-carboxamide 2,2,2- trifluoroacetate |
| 18 | 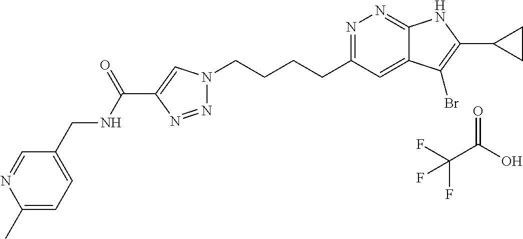 | 1-(4-(5-Bromo-6- cyclopropyl-7H-pyrrolo[2,3- c]pyridazin-3-yl)butyl)-N-((6- methylpyridin-3-yl)methyl)- 1H-1,2,3-triazole-4- carboxamide 2,2,2- trifluoroacetate |
| 19 | 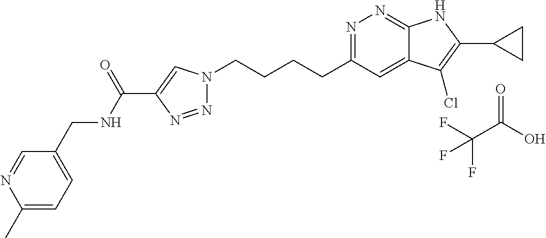 | 1-(4-(5-Chloro-6- cyclopropyl-7H-pyrrolo[2,3- c]pyridazin-3-yl)butyl)-N-((6- methylpyridin-3-yl)methyl)- 1H-1,2,3-triazole-4- carboxamide 2,2,2- trifluoroacetate |
| 20 | 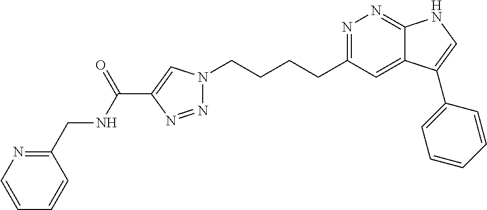 [SMILES: O=C(NCc1ccccn1)c1cn(CCCCc2cc3c(-c4ccccc4)c[nH]c3nn2)nn1] | 1-(4-(5-phenyl-7H- pyrrolo[2,3-c]pyridazin-3- yl)butyl)-N-(pyridin-2- ylmethyl)-1H-1,2,3-triazole- 4-carboxamide |
| 21 | 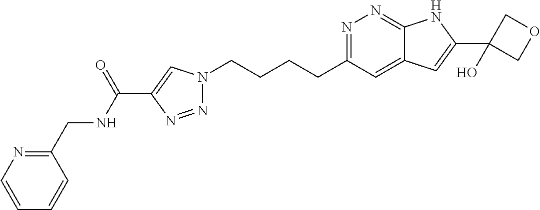 [SMILES: O=C(NCc1ccccn1)c1cn(CCCCc2cc3cc(C4(O)COC4)[nH]c3nn2)nn1] | 1-(4-(6-(3-hydroxyoxetan-3- yl)-7H-pyrrolo[2,3- c]pyridazin-3-yl)butyl)-N- (pyridin-2-ylmethyl)-1H- 1,2,3-triazole-4-carboxamide |
| 22 | 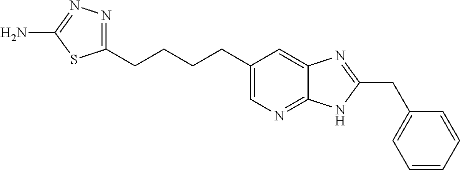 [SMILES: Nc1nnc(CCCCc2cnc3[nH]c(Cc4ccccc4)nc3c2)s1] | 5-(4-(2-benzyl-3H- imidazo[4,5-b]pyridin-6- yl)butyl)-1,3,4-thiadiazol-2- amine |
| 23 | 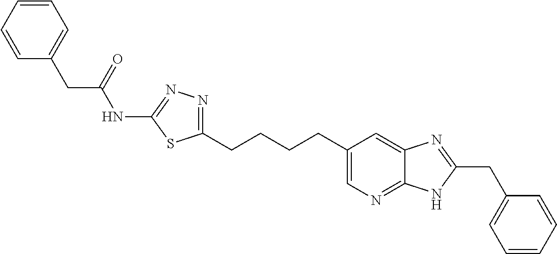 [SMILES: O=C(Cc1ccccc1)Nc1nnc(CCCCc2cnc3[nH]c(Cc4ccccc4)nc3c2)s1] | N-(5-(4-(2-benzyl-3H- imidazo[4,5-b]pyridin-6- yl)butyl)-1,3,4-thiadiazol-2- yl)-2-phenylacetamide |
| 24 | 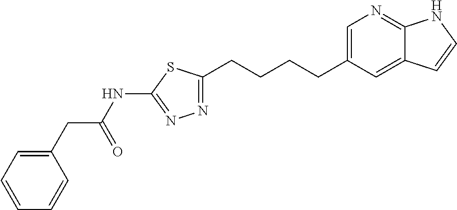 [SMILES: O=C(Cc1ccccc1)Nc1nnc(CCCCc2cnc3[nH]ccc3c2)s1] | N-(5-(4-(1H-pyrrolo[2,3- b]pyridin-5-yl)butyl)-1,3,4- thiadiazol-2-yl)-2- phenylacetamide |
| 25 | 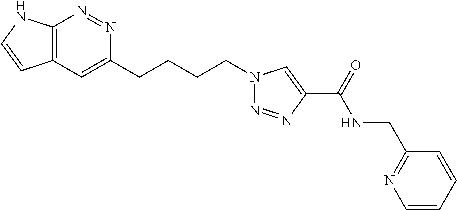 [SMILES: O=C(NCc1ccccn1)c1cn(CCCCc2cc3cc[nH]c3nn2)nn1] | 1-(4-(7H-pyrrolo[2,3- c]pyridazin-3-yl)butyl)-N- (pyridin-2-ylmethyl)-1H- 1,2,3-triazole-4-carboxamide |
| 26 | 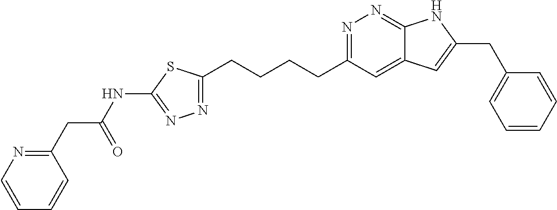 [SMILES: O=C(Cc1ccccn1)Nc1nnc(CCCCc2cc3cc(Cc4ccccc4)[nH]c3nn2)s1] | N-[5-(4-{6-benzyl-7H- pyrrolo[2,3-c]pyridazin-3- yl}butyl)-1,3,4-thiadiazol-2- yl]-2-(pyridin-2-yl)acetamide |
| 27 | 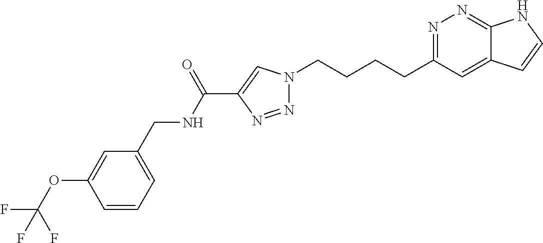 [SMILES: O=C(NCc1cccc(OC(F)(F)F)c1)c1cn(CCCCc2cc3cc[nH]c3nn2)nn1] | 1-(4-{7H-pyrrolo[2,3- c]pyridazin-3-yl}butyl)-N- {[3- (trifluoromethoxy)phenyl] methyl}-1H-1,2,3-triazole-4- carboxamide |
| 28 | 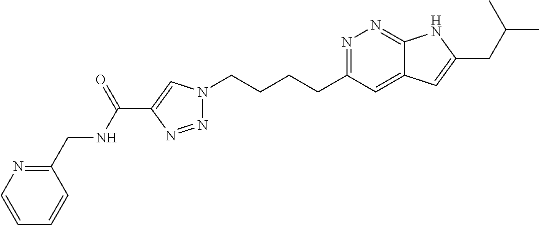 [SMILES: CC(C)Cc1cc2cc(CCCCn3cc(C(=O)NCc4ccccn4)nn3)nnc2[nH]1] | 1-{4-[6-(2-methylpropyl)-7H- pyrrolo[2,3-c]pyridazin-3- yl]butyl}-N-(pyridin-2- ylmethyl)-1H-1,2,3-triazole- 4-carboxamide |
| 29 | 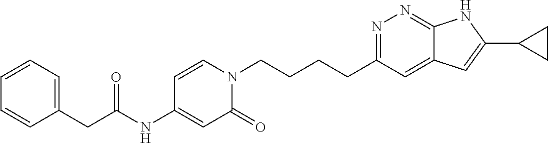 [SMILES: O=C(Cc1ccccc1)Nc1ccn(CCCCc2cc3cc(C4CC4)[nH]c3nn2)c(=O)c1] | N-[1-(4-{6-cyclopropyl-7H- pyrrolo[2,3-c]pyridazin-3- yl}butyl)-2-oxo-1,2- dihydropyridin-4-yl]-2- phenylacetamide |
| 30 | 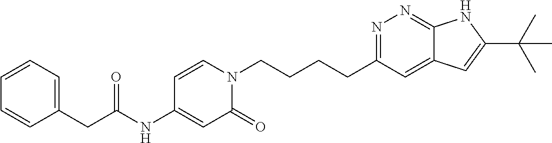 [SMILES: CC(C)(C)c1cc2cc(CCCCn3ccc(NC(=O)Cc4ccccc4)cc3=O)nnc2[nH]1] | N-[1-(4-{6-tert-butyl-7H- pynolo[2,3-c]pyridazin-3- yl}butyl)-2-oxo-1,2- dihydropyridin-4-yl]-2- phenylacetamide |
| 31 | 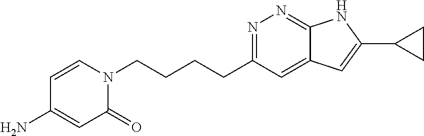 [SMILES: Nc1ccn(CCCCc2cc3cc(C4CC4)[nH]c3nn2)c(=O)c1] | 4-amino-1-(4-{6-cyclopropyl- 7H-pyrrolo[2,3-c]pyridazin-3- yl}butyl)-1,2-dihydropyridin- 2-one |
| 32 | 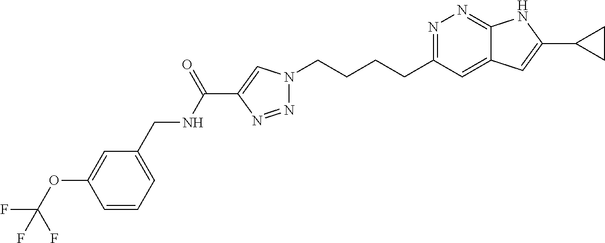 [SMILES: O=C(NCc1cccc(OC(F)(F)F)c1)c1cn(CCCCc2cc3cc(C4CC4)[nH]c3nn2)nn1] | 1-(4-{6-cyclopropyl-7H- pyrrolo[2,3-c]pyridazin-3- yl}butyl)-N-{[3- (trifluoromethoxy)phenyl] methyl}-1H-1,2,3-triazole-4- carboxamide |
| 33 | 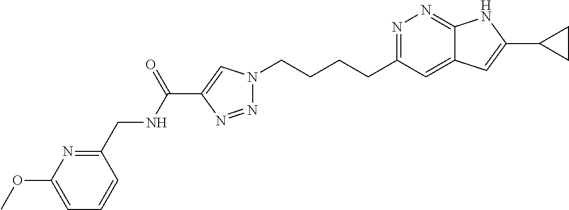 [SMILES: COc1cccc(CNC(=O)c2cn(CCCCc3cc4cc(C5CC5)[nH]c4nn3)nn2)n1] | 1-(4-{6-cyclopropyl-7H- pyrrolo[2,3-c]pyridazin-3- yl}butyl)-N-[(6- methoxypyridin-2-yl)methyl]- 1H-1,2,3-triazole-4- carboxamide |
| 34 | 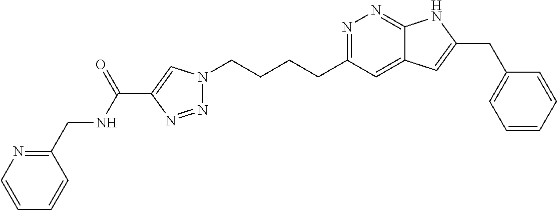 [SMILES: O=C(NCc1ccccn1)c1cn(CCCCc2cc3cc(Cc4ccccc4)[nH]c3nn2)nn1] | 1-(4-{6-benzyl-7H- pyrrolo[2,3-c]pyridazin-3- yl}butyl)-N-(pyridin-2- ylmethyl)-1H-1,2,3-triazole- 4-carboxamide |
| 35 | 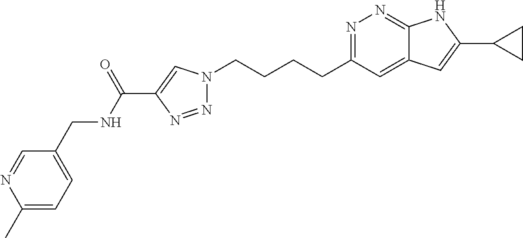 [SMILES: Cc1ccc(CNC(=O)c2cn(CCCCc3cc4cc(C5CC5)[nH]c4nn3)nn2)cn1] | 1-(4-{6-cyclopropyl-7H- pyrrolo[2,3-c]pyridazin-3- yl}butyl)-N-[(6- methylpyridin-3-yl)methyl]- 1H-1,2,3-triazole-4- carboxamide |
| 36 | 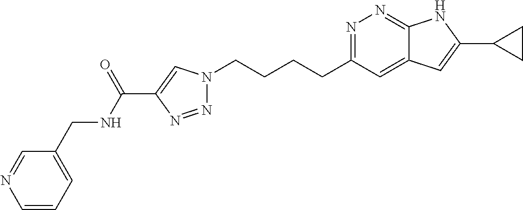 [SMILES: O=C(NCc1cccnc1)c1cn(CCCCc2cc3cc(C4CC4)[nH]c3nn2)nn1] | 1-(4-{6-cyclopropyl-7H- pyrrolo[2,3-c]pyridazin-3- yl}butyl)-N-(pyridin-3- ylmethyl)-1H-1,2,3-triazole- 4-carboxamide |
| 37 | 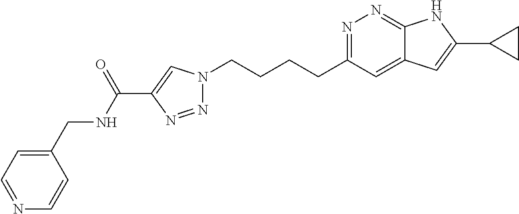 [SMILES: O=C(NCc1ccncc1)c1cn(CCCCc2cc3cc(C4CC4)[nH]c3nn2)nn1] | 1-(4-{6-cyclopropyl-7H- pyrrolo[2,3-c]pyridazin-3- yl}butyl)-N-(pyridin-4- ylmethyl)-1H-1,2,3-triazole- 4-carboxamide |
| 38 | 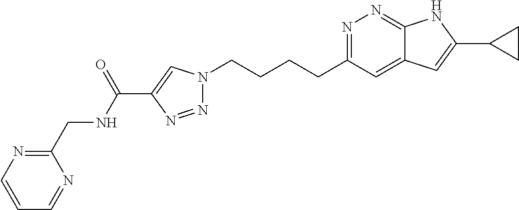 [SMILES: O=C(NCc1ncccn1)c1cn(CCCCc2cc3cc(C4CC4)[nH]c3nn2)nn1] | 1-(4-{6-cyclopropyl-7H- pyrrolo[2,3-c]pyridazin-3- yl}butyl)-N-(pyrimidin-2- ylmethyl)-1H-1,2,3-triazole- 4-carboxamide |
| 39 | 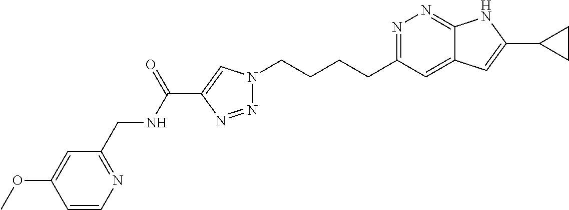 [SMILES: COc1ccnc(CNC(=O)c2cn(CCCCc3cc4cc(C5CC5)[nH]c4nn3)nn2)c1] | 1-(4-{6-cyclopropyl-7H- pyrrolo[2,3-c]pyridazin-3- yl}butyl)-N-[(4- methoxypyridin-2-yl)methyl]- 1H-1,2,3-triazole-4- carboxamide |
| 40 | 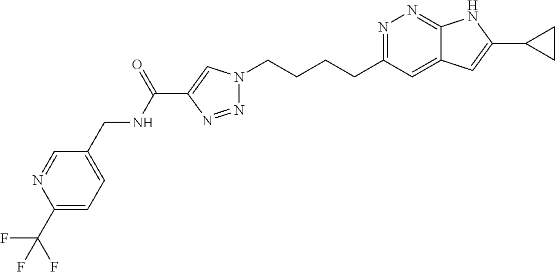 [SMILES: O=C(NCc1ccc(C(F)(F)F)nc1)c1cn(CCCCc2cc3cc(C4CC4)[nH]c3nn2)nn1] | 1-(4-{6-cyclopropyl-7H- pyrrolo[2,3-c]pyridazin-3- yl}butyl)-N-{[6- (trifluoromethyl)pyridin-3- yl]methyl}-1H-1,2,3-triazole- 4-carboxamide |
| 41 | 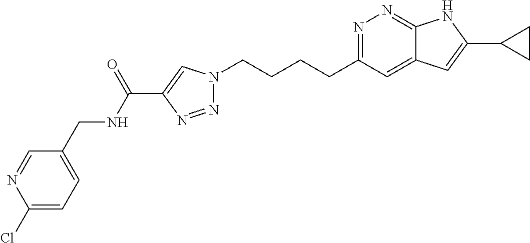 [SMILES: O=C(NCc1ccc(Cl)nc1)c1cn(CCCCc2cc3cc(C4CC4)[nH]c3nn2)nn1] | N-[(6-chloropyridin-3- yl)methyl]-1-(4-{6- cyclopropyl-7H-pyrrolo[2,3- c]pyridazin-3-yl}butyl)-1H- 1,2,3-triazole-4-carboxamide |
| 42 | 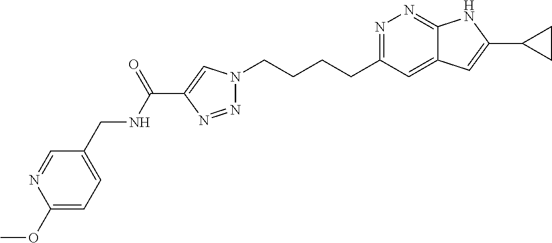 [SMILES: COc1ccc(CNC(=O)c2cn(CCCCc3cc4cc(C5CC5)[nH]c4nn3)nn2)cn1] | 1-(4-{6-cyclopropyl-7H- pyrrolo[2,3-c]pyridazin-3- yl}butyl)-N-[(6- methoxypyridin-3-yl)methyl]- 1H-1,2,3-triazole-4- carboxamide |
| 43 | 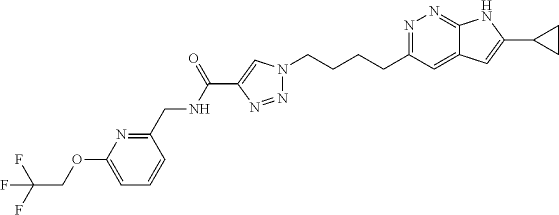 [SMILES: O=C(NCc1cccc(OCC(F)(F)F)n1)c1cn(CCCCc2cc3cc(C4CC4)[nH]c3nn2)nn1] | 1-(4-{6-cyclopropyl-7H- pyrrolo[2,3-c]pyridazin-3- yl}butyl)-N-{[6-(2,2,2- trifluoroethoxy)pyridin-2- yl]methyl}-1H-1,2,3-triazole- 4-carboxamide |
| 44 | 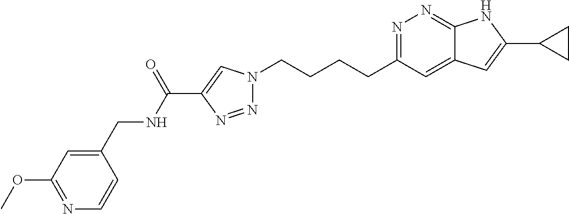 [SMILES: COc1cc(CNC(=O)c2cn(CCCCc3cc4cc(C5CC5)[nH]c4nn3)nn2)ccn1] | 1-(4-{6-cyclopropyl-7H- pyrrolo[2,3-c]pyridazin-3- yl}butyl)-N-[(2- methoxypyridin-4-yl)methyl]- 1H-1,2,3-triazole-4- carboxamide |
| 45 | 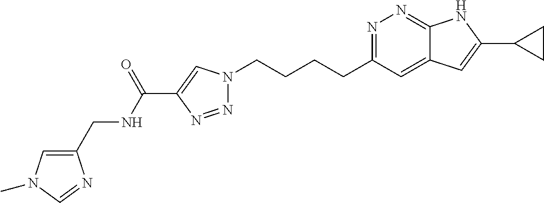 [SMILES: Cn1cnc(CNC(=O)c2cn(CCCCc3cc4cc(C5CC5)[nH]c4nn3)nn2)c1] | 1-(4-{6-cyclopropyl-7H- pyrrolo[2,3-c]pyridazin-3- yl}butyl)-N-[(1-methyl-1H- imidazol-4-yl)methyl]-1H- 1,2,3-triazole-4-carboxamide |
| 46 | 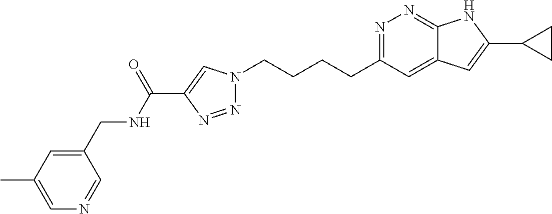 [SMILES: Cc1cncc(CNC(=O)c2cn(CCCCc3cc4cc(C5CC5)[nH]c4nn3)nn2)c1] | 1-(4-{6-cyclopropyl-7H- pyrrolo[2,3-c]pyridazin-3- yl}butyl)-N-[(5- methylpyridin-3-yl)methyl]- 1H-1,2,3-triazole-4- carboxamide |
| 47 | 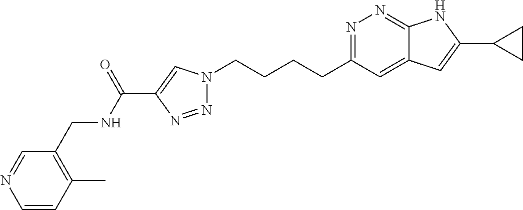 [SMILES: Cc1ccncc1CNC(=O)c1cn(CCCCc2cc3cc(C4CC4)[nH]c3nn2)nn1] | 1-(4-{6-cyclopropyl-7H- pyrrolo[2,3-c]pyridazin-3- yl}butyl)-N-[(4- methylpyridin-3-yl)methyl]- 1H-1,2,3-triazole-4- carboxamide |
| 48 | 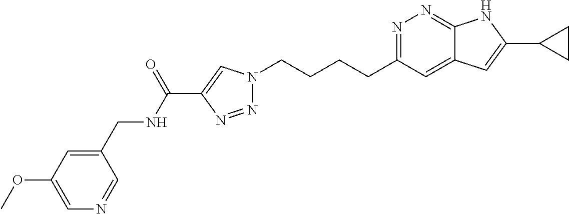 [SMILES: COc1cncc(CNC(=O)c2cn(CCCCc3cc4cc(C5CC5)[nH]c4nn3)nn2)c1] | 1-(4-{6-cyclopropyl-7H- pyrrolo[2,3-c]pyridazin-3- yl}butyl)-N-[(5- methoxypyridin-3-yl)methyl]- 1H-1,2,3-triazole-4- carboxamide |
| 49 | 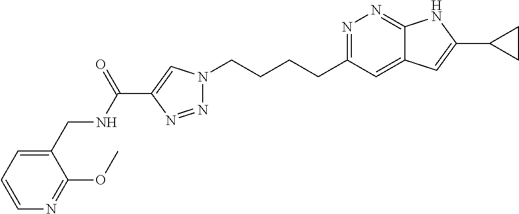 [SMILES: COc1ncccc1CNC(=O)c1cn(CCCCc2cc3cc(C4CC4)[nH]c3nn2)nn1] | 1-(4-{6-cyclopropyl-7H- pyrrolo[2,3-c]pyridazin-3- yl}butyl)-N-[(2- methoxypyridin-3-yl)methyl]- 1H-1,2,3-triazole-4- carboxamide |
| 50 | 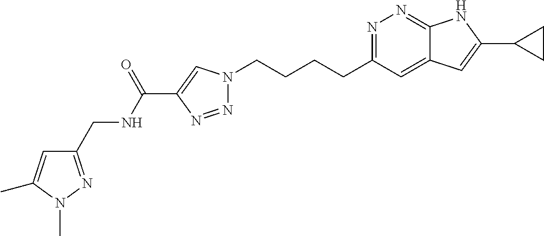 [SMILES: Cc1cc(CNC(=O)c2cn(CCCCc3cc4cc(C5CC5)[nH]c4nn3)nn2)nn1C] | 1-(4-{6-cyclopropyl-7H- pyrrolo[2,3-c]pyridazin-3- yl}butyl)-N-[(1,5-dimethyl- 1H-pyrazol-3-yl)methyl]-1H- 1,2,3-triazole-4-carboxamide |
| 51 | 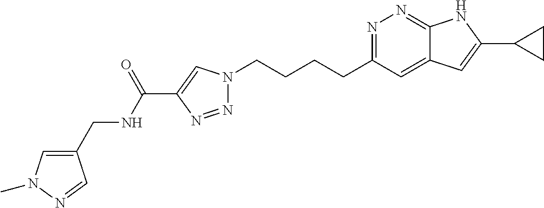 [SMILES: Cn1cc(CNC(=O)c2cn(CCCCc3cc4cc(C5CC5)[nH]c4nn3)nn2)cn1] | 1-(4-{6-cyclopropyl-7H- pyrrolo[2,3-c]pyridazin-3- yl}butyl)-N-[(1-methyl-1H- pyrazol-4-yl)methyl]-1H- 1,2,3-triazole-4-carboxamide |
| 52 | 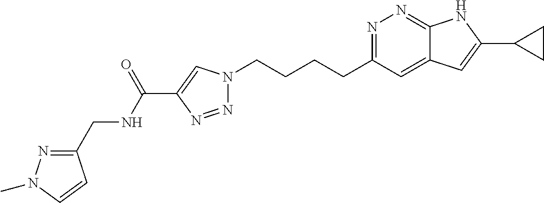 [SMILES: Cn1ccc(CNC(=O)c2cn(CCCCc3cc4cc(C5CC5)[nH]c4nn3)nn2)n1] | 1-(4-{6-cyclopropyl-7H- pyrrolo[2,3-c]pyridazin-3- yl}butyl)-N-[(1-methyl-1H- pyrazol-3-yl)methyl]-1H- 1,2,3-triazole-4-carboxamide |
| 53 | 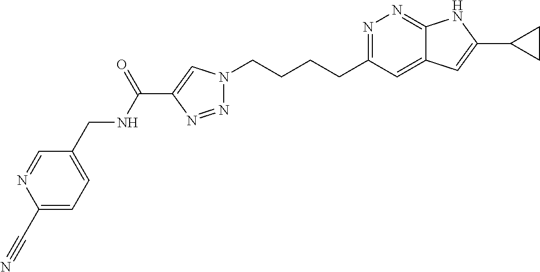 [SMILES: N#Cc1ccc(CNC(=O)c2cn(CCCCc3cc4cc(C5CC5)[nH]c4nn3)nn2)cn1] | N-[(6-cyanopyridin-3- yl)methyl]-1-(4-{6- cyclopropyl-7H-pyrrolo[2,3- c]pyridazin-3-yl}butyl)-1H- 1,2,3-triazole-4-carboxamide |
| 54 | 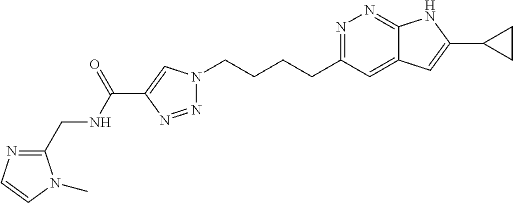 [SMILES: Cn1ccnc1CNC(=O)c1cn(CCCCc2cc3cc(C4CC4)[nH]c3nn2)nn1] | 1-(4-{6-cyclopropyl-7H- pyrrolo[2,3-c]pyridazin-3- yl}butyl)-N-[(1-methyl-1H- imidazol-2-yl)methyl]-1H- 1,2,3-triazole-4-carboxamide |
| 55 | 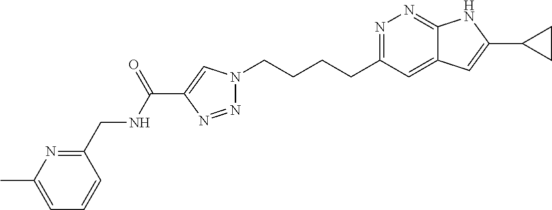 [SMILES: Cc1cccc(CNC(=O)c2cn(CCCCc3cc4cc(C5CC5)[nH]c4nn3)nn2)n1] | 1-(4-{6-cyclopropyl-7H- pyrrolo[2,3-c]pyridazin-3- yl}butyl)-N-[(6- methylpyridin-2-yl)methyl]- 1H-1,2,3-triazole-4- carboxamide |
| 56 | 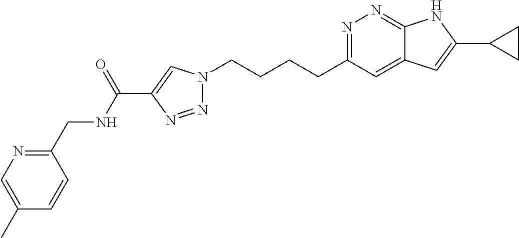 [SMILES: Cc1ccc(CNC(=O)c2cn(CCCCc3cc4cc(C5CC5)[nH]c4nn3)nn2)nc1] | 1-(4-{6-cyclopropyl-7H- pyrrolo[2,3-c]pyridazin-3- yl}butyl)-N-[(5- methylpyridin-2-yl)methyl]- 1H-1,2,3-triazole-4- carboxamide |
| 57 | 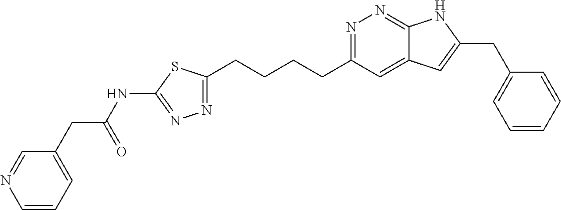 [SMILES: O=C(Cc1cccnc1)Nc1nnc(CCCCc2cc3cc(Cc4ccccc4)[nH]c3nn2)s1] | N-[5-(4-{6-benzyl-7H- pyrrolo[2,3-c]pyridazin-3- yl}butyl)-1,3,4-thiadiazol-2- yl]-2-(pyridin-3-yl)acetamide |
| 58 | 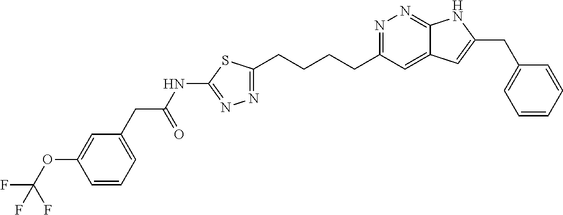 [SMILES: O=C(Cc1cccc(OC(F)(F)F)c1)Nc1nnc(CCCCc2cc3cc(Cc4ccccc4)[nH]c3nn2)s1] | N-[5-(4-{6-benzyl-7H- pyrrolo[2,3-c]pyridazin-3- yl}butyl)-1,3,4-thiadiazol-2- yl]-2-[3- (trifluoromethoxy)phenyl] acetamide |
| 59 | 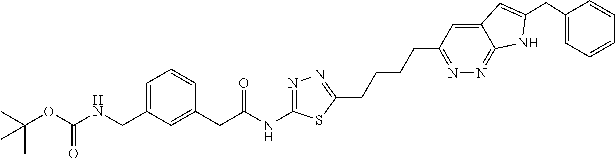 [SMILES: CC(C)(C)OC(=O)NCc1cccc(CC(=O)Nc2nnc(CCCCc3cc4cc(Cc5ccccc5)[nH]c4nn3)s2)c1] | tert-butyl N-{[3-({[5-(4-{6- benzyl-7H-pyrrolo[2,3- c]pyridazin-3-yl}butyl)-1,3,4- thiadiazol-2- yl]carbamoyl}methyl)phenyl] methyl}carbamate |
| 60 | 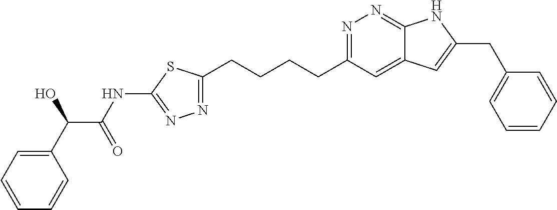 [SMILES: O=C(Nc1nnc(CCCCc2cc3cc(Cc4ccccc4)[nH]c3nn2)s1)[C@H](O)c1ccccc1] | (2R)-N-[5-(4-(6-benzyl-7H- pyrrolo[2,3-c]pyridazin-3- yl}butyl)-1,3,4-thiadiazol-2- yl]-2-hydroxy-2- phenylacetamide |
| 61 | 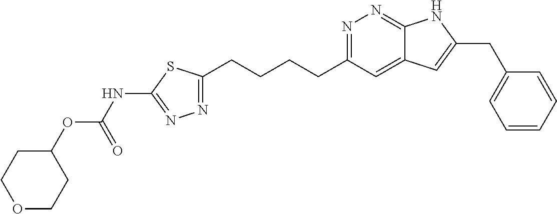 [SMILES: O=C(Nc1nnc(CCCCc2cc3cc(Cc4ccccc4)[nH]c3nn2)s1)OC1CCOCC1] | oxan-4-yl N-[5-(4-{6-benzyl- 7H-pyrrolo[2,3-c]pyridazin-3- yl}butyl)-1,3,4-thiadiazol-2- yl]carbamate |
| 62 | 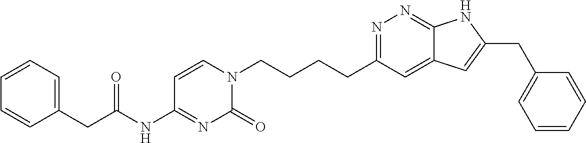 [SMILES: O=C(Cc1ccccc1)Nc1ccn(CCCCc2cc3cc(Cc4ccccc4)[nH]c3nn2)c(=O)n1] | N-[1-(4-{6-benzyl-7H- pyrrolo[2,3-c]pyridazin-3- yl}butyl)-2-oxo-1,2- dihydropyrimidin-4-yl]-2- phenylacetamide |
| 63 | 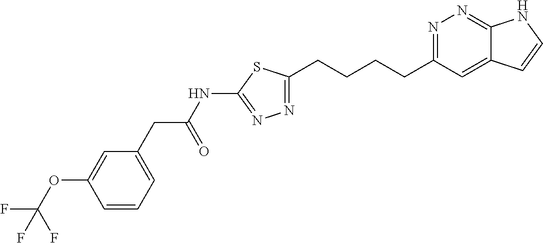 [SMILES: O=C(Cc1cccc(OC(F)(F)F)c1)Nc1nnc(CCCCc2cc3cc[nH]c3nn2)s1] | N-[5-(4-{7H-pyrrolo[2,3- c]pyridazin-3-yl}butyl)-1,3,4- thiadiazol-2-yl]-2-[3- (trifluoromethoxy)phenyl] acetamide |
| 64 | [symbol] | N-(5-(4-(7H-pyrrolo[2,3- |
| | | c]pyridazin-3-yl)butyl)-1,3,4- |
| | | thiadiazol-2-yl)-2- |
| | | phenylacetamide |
| 65 | 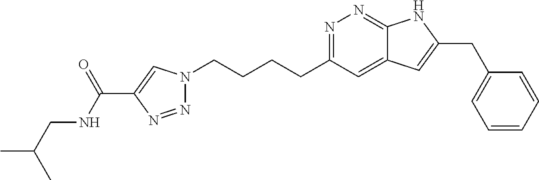 [SMILES: CC(C)CNC(=O)c1cn(CCCCc2cc3cc(Cc4ccccc4)[nH]c3nn2)nn1] | 1-(4-{6-benzyl-7H- pyrrolo[2,3-c]pyridazin-3- yl}butyl)-N-(2- methylpropyl)-1H-1,2,3- triazole-4-carboxamide |
| 66 | 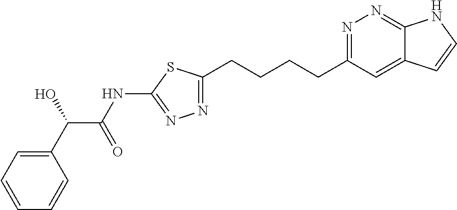 [SMILES: O=C(Nc1nnc(CCCCc2cc3cc[nH]c3nn2)s1)[C@@H](O)c1ccccc1] | (2S)-2-hydroxy-2-phenyl-N- [5-(4-{7H-pyrrolo[2,3- c]pyridazin-3-yl}butyl)-1,3,4- thiadiazol-2-yl]acetamide |
| 67 | 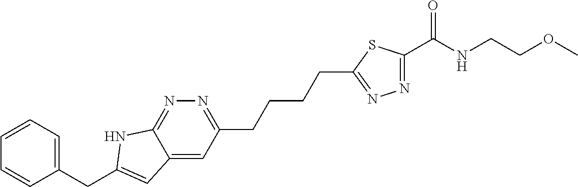 [SMILES: COCCNC(=O)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-(2- methoxyethyl)-1,3,4- thiadiazole-2-carboxamide |
| 68 | 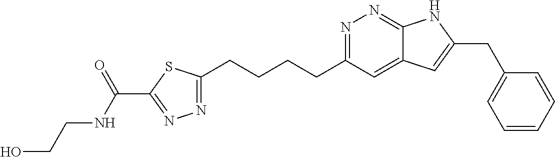 [SMILES: O=C(NCCO)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-(2- hydroxyethyl)-1,3,4- thiadiazole-2-carboxamide |
| 69 | 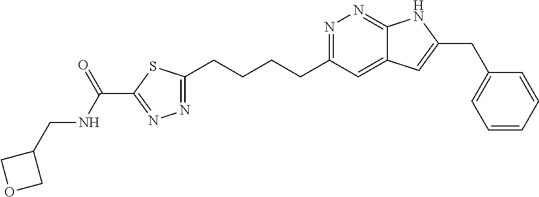 [SMILES: O=C(NCC1COC1)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-(oxetan-3- ylmethyl)-1,3,4-thiadiazole-2- carboxamide |
| 70 | 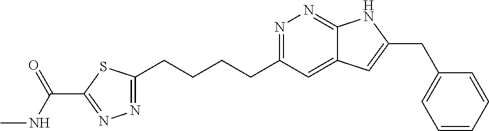 [SMILES: CNC(=O)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-methyl-1,3,4- thiadiazole-2-carboxamide |
| 71 | 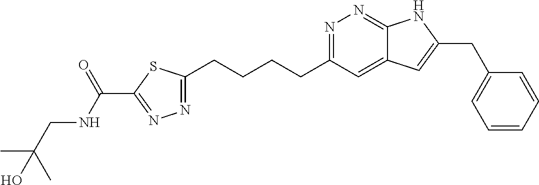 [SMILES: CC(C)(O)CNC(=O)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-(2-hydroxy-2- methylpropyl)-1,3,4- thiadiazole-2-carboxamide |
| 72 | 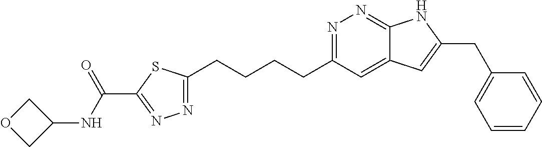 [SMILES: O=C(NC1COC1)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-(oxetan-3-yl)- 1,3,4-thiadiazole-2- carboxamide |
| 73 | 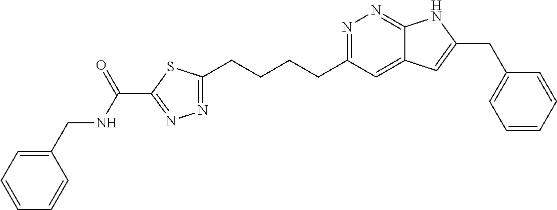 [SMILES: O=C(NCc1ccccc1)c1nnc(CCCCc2cc3cc(Cc4ccccc4)[nH]c3nn2)s1] | N-benzyl-5-(4-{6-benzyl-7H- pyrrolo[2,3-c]pyridazin-3- yl}butyl)-1,3,4-thiadiazole-2- carboxamide |
| 74 | 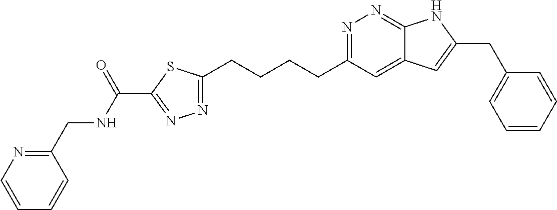 [SMILES: O=C(NCc1ccccn1)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-(pyridin-2- ylmethyl)-1,3,4-thiadiazole-2- carboxamide |
| 75 | 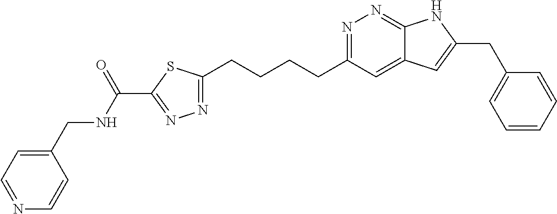 [SMILES: O=C(NCc1ccncc1)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-(pyridin-4- ylmethyl)-1,3,4-thiadiazole-2- carboxamide |
| 76 | 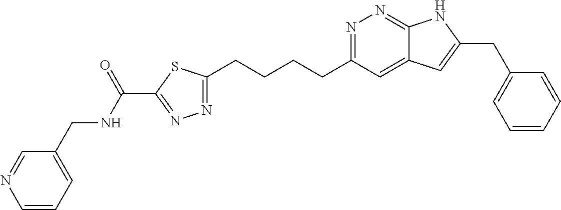 [SMILES: O=C(NCc1cccnc1)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-(pyridin-3- ylmethyl)-1,3,4-thiadiazole-2- carboxamide |
| 77 | 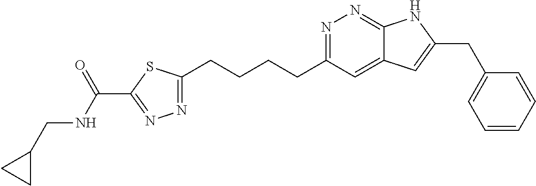 [SMILES: O=C(NCC1CC1)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N- (cyclopropylmethyl)-1,3,4- thiadiazole-2-carboxamide |
| 78 | 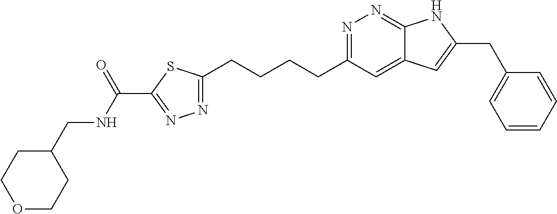 [SMILES: O=C(NCC1CCOCC1)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-(oxan-4- ylmethyl)-1,3,4-thiadiazole-2- carboxamide |
| 79 | 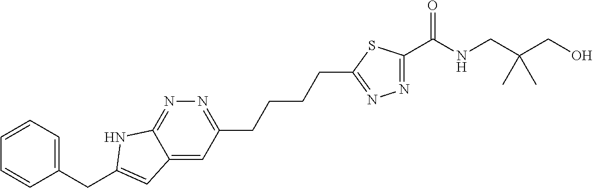 [SMILES: CC(C)(CO)CNC(=O)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-(3-hydroxy-2,2- dimethylpropyl)-1,3,4- thiadiazole-2-carboxamide |
| 80 | 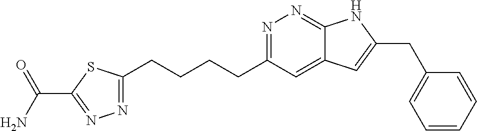 [SMILES: NC(=O)c1nnc(CCCCc2cc3cc(Cc4ccccc4)[nH]c3nn2)s1] | 5-(4-{6-benzyl-7H- pyrrolo[2,3-c]pyridazin-3- yl}butyl)-1,3,4-thiadiazole-2- carboxamide |
| 81 | 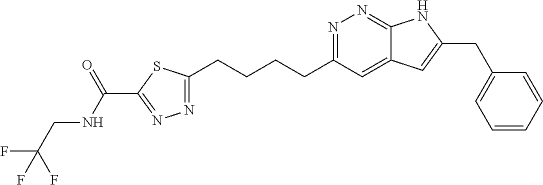 [SMILES: O=C(NCC(F)(F)F)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-(2,2,2- trifluoroethyl)-1,3,4- thiadiazole-2-carboxamide |
| 82 | 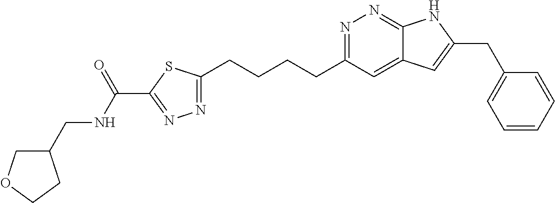 [SMILES: O=C(NCC1CCOC1)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-(oxolan-3- ylmethyl)-1,3,4-thiadiazole-2- carboxamide |
| 83 | 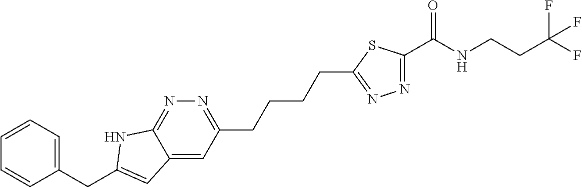 [SMILES: O=C(NCCC(F)(F)F)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-(3,3,3- trifluoropropyl)-1,3,4- thiadiazole-2-carboxamide |
| 84 | 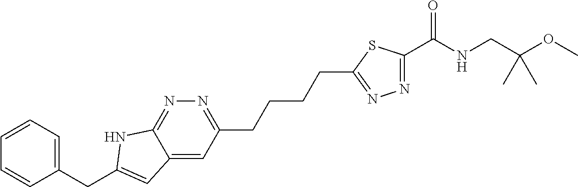 [SMILES: COC(C)(C)CNC(=O)c1nnc(CCCCc2cc3cc(Cc4ccccc4)[nH]c3nn2)s1] | 5-(4-{6-benzyl-7H- pyrrolo[2,3-c]pyridazin-3- yl}butyl)-N-(2-methoxy-2- methylpropyl)-1,3,4- thiadiazole-2-carboxamide |
| 85 | 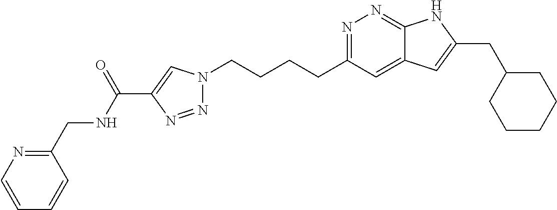 [SMILES: O=C(NCc1ccccn1)c1cn(CCCCc2cc3cc(CC4CCCCC4)[nH]c3nn2)nn1] | 1-(4-(6-(cyclohexylmethyl)- 7H-pyrrolo[2,3-c]pyridazin-3- yl)butyl)-N-(pyridin-2- ylmethyl)-1H-1,2,3-triazole- 4-carboxamide |
| 86 | 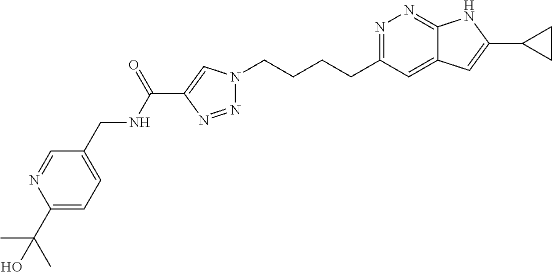 [SMILES: CC(C)(O)c1ccc(CNC(=O)c2cn(CCCCc3cc4cc(C5CC5)[nH]c4nn3)nn2)cn1] | 1-(4-{6-cyclopropyl-7H- pyrrolo[2,3-c]pyridazin-3- yl}butyl)-N-{[6-(2- hydroxypropan-2-yl)pyridin- 3-yl]methyl}-1H-1,2,3- triazole-4-carboxamide |
| 87 | 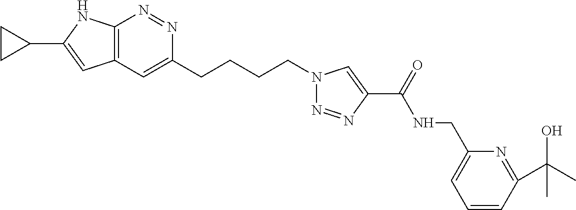 [SMILES: CC(C)(O)c1cccc(CNC(=O)c2cn(CCCCc3cc4cc(C5CC5)[nH]c4nn3)nn2)n1] | 1-(4-(6-cyclopropyl-7H- pyrrolo[2,3-c]pyridazin-3- yl)butyl)-N-((6-(2- hydroxypropan-2-yl)pyridin- 2-yl)methyl)-1H-1,2,3- triazole-4-carboxamide |
| 88 | 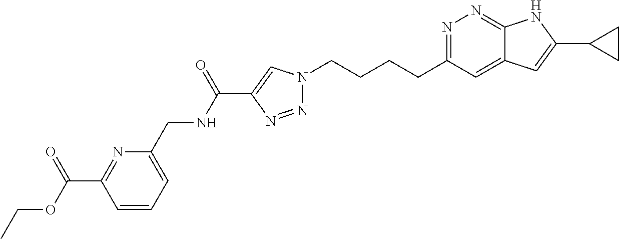 [SMILES: CCOC(=O)c1cccc(CNC(=O)c2cn(CCCCc3cc4cc(C5CC5)[nH]c4nn3)nn2)n1] | ethyl 6-({[1-(4-{6- cyclopropyl-7H-pyrrolo[2,3- c]pyridazin-3-yl}butyl)-1H- 1,2,3-triazol-4- yl]formamido}methyl) pyridine-2-carboxylate |
| 89 | 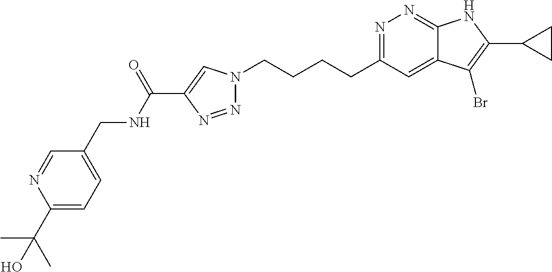 [SMILES: CC(C)(O)c1ccc(CNC(=O)c2cn(CCCCc3cc4c(Br)c(C5CC5)[nH]c4nn3)nn2)cn1] | 1-(4-{5-bromo-6- cyclopropyl-7H-pyrrolo[2,3- c]pyridazin-3-yl}butyl)-N- {[6-(2-hydroxypropan-2- yl)pyridin-3-yl]methyl}-1H- 1,2,3-triazole-4-carboxamide |
| 90 | 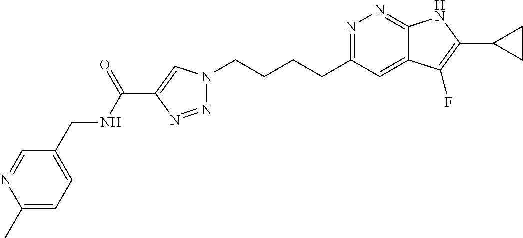 [SMILES: Cc1ccc(CNC(=O)c2cn(CCCCc3cc4c(F)c(C5CC5)[nH]c4nn3)nn2)cn1] | 1-(4-{6-cyclopropyl-5-fluoro- 7H-pyrrolo[2,3-c]pyridazin-3- yl}butyl)-N-[(6- methylpyridin-3-yl)methyl]- 1H-1,2,3-triazole-4- carboxamide |
| 91 | 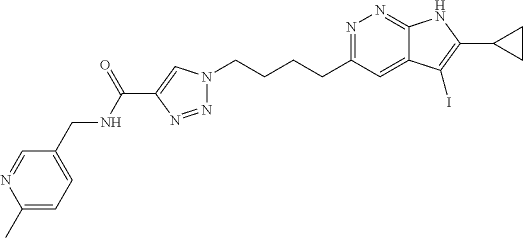 [SMILES: Cc1ccc(CNC(=O)c2cn(CCCCc3cc4c(I)c(C5CC5)[nH]c4nn3)nn2)cn1] | 1-(4-{6-cyclopropyl-5-iodo- 7H-pyrrolo[2,3-c]pyridazin-3- yl}butyl)-N-[(6- methylpyridin-3-yl)methyl]- 1H-1,2,3-triazole-4- carboxamide |
| 92 | 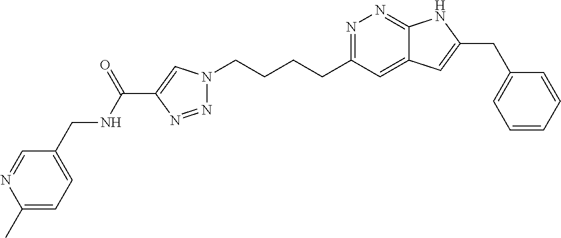 [SMILES: Cc1ccc(CNC(=O)c2cn(CCCCc3cc4cc(Cc5ccccc5)[nH]c4nn3)nn2)cn1] | 1-(4-{6-benzyl-7H- pyrrolo[2,3-c]pyridazin-3- yl}butyl)-N-[(6- methylpyridin-3-yl)methyl]- 1H-1,2,3-triazole-4- carboxamide |
| 93 | 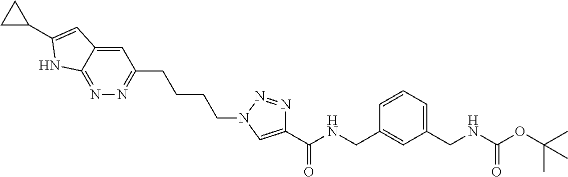 [SMILES: CC(C)(C)OC(=O)NCc1cccc(CNC(=O)c2cn(CCCCc3cc4cc(C5CC5)[nH]c4nn3)nn2)c1] | tert-butyl N-{[3-({[1-(4-{6- cyclopropyl-7H-pyrrolo[2,3- c]pyridazin-3-yl}butyl)-1H- 1,2,3-triazol-4- yl]formamido}methyl) phenyl]methyl}carbamate |
| 94 | 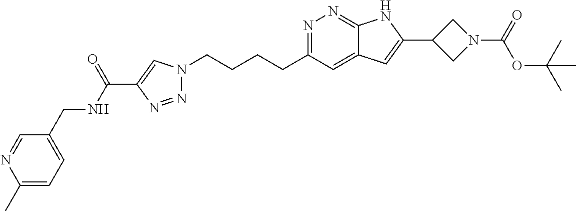 [SMILES: Cc1ccc(CNC(=O)c2cn(CCCCc3cc4cc(C5CN(C(=O)OC(C)(C)C)C5)[nH]c4nn3)nn2)cn1] | tert-butyl 3-{3-[4-(4-{[(6- methylpyridin-3- yl)methyl]carbamoyl}-1H- 1,2,3-triazol-1-yl)butyl]-7H- pyrrolo[2,3-c]pyridazin-6- yl}azetidine-1-carboxylate |
| 95 | 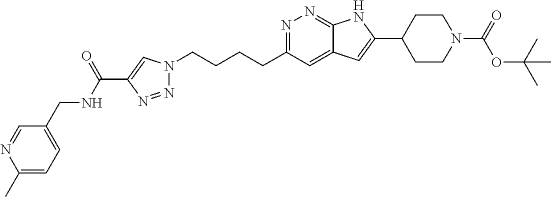 [SMILES: Cc1ccc(CNC(=O)c2cn(CCCCc3cc4cc(C5CCN(C(=O)OC(C)(C)C)CC5)[nH]c4nn3)nn2)cn1] | ten-butyl 4-{3-[4-(4-{[(6- methylpyridin-3- yl)methyl]carbamoyl}-1H- 1,2,3-triazol-1-yl)butyl]-7H- pyrrolo[2,3-c]pyridazin-6- yl}piperidine-1-carboxylate |
| 96 | 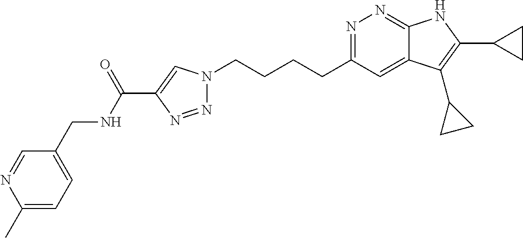 [SMILES: Cc1ccc(CNC(=O)c2cn(CCCCc3cc4c(C5CC5)c(C5CC5)[nH]c4nn3)nn2)cn1] | 1-(4-{5,6-dicyclopropyl-7H- pyrrolo[2,3-c]pyridazin-3- yl}butyl)-N-[(6- methylpyridin-3-yl)methyl]- 1H-1,2,3-triazole-4- carboxamide |
| 97 | 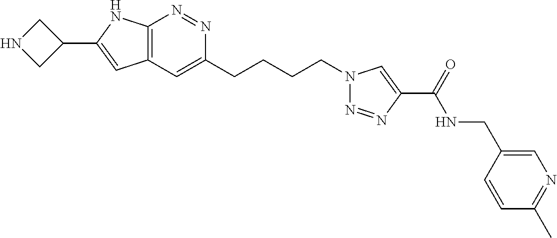 [SMILES: Cc1ccc(CNC(=O)c2cn(CCCCc3cc4cc(C5CNC5)[nH]c4nn3)nn2)cn1] | 1-{4-[6-(azetidin-3-yl)-7H- pyrrolo[2,3-c]pyridazin-3- yl]butyl}-N-[(6- methylpyridin-3-yl)methyl]- 1H-1,2,3-triazole-4- carboxamide |
| 98 | 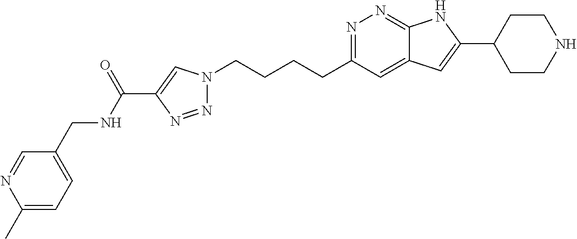 [SMILES: Cc1ccc(CNC(=O)c2cn(CCCCc3cc4cc(C5CCNCC5)[nH]c4nn3)nn2)cn1] | N-[(6-methylpyridin-3- yl)methyl]-1-{4-[6-(piperidin- 4-yl)-7H-pyrrolo[2,3- c]pyridazin-3-yl]butyl}-1H- 1,2,3-triazole-4-carboxamide |
| 99 | 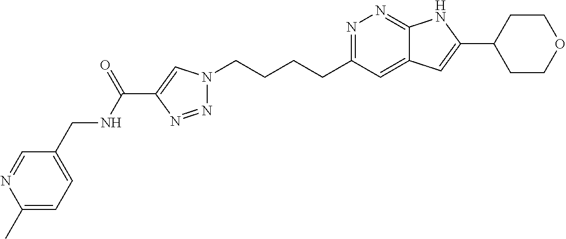 [SMILES: Cc1ccc(CNC(=O)c2cn(CCCCc3cc4cc(C5CCOCC5)[nH]c4nn3)nn2)cn1] | N-[(6-methylpyridin-3- yl)methyl]-1-{4-[6-(oxan-4- yl)-7H-pyrrolo[2,3- c]pyridazin-3-yl]butyl}-1H- 1,2,3-triazole-4-carboxamide |
| 100 | 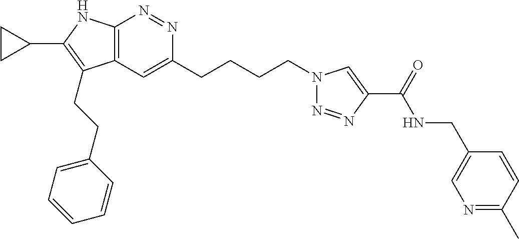 [SMILES: Cc1ccc(CNC(=O)c2cn(CCCCc3cc4c(CCc5ccccc5)c(C5CC5)[nH]c4nn3)nn2)cn1] | 1-{4-[6-cyclopropyl-5-(2- phenylethyl)-7H-pyrrolo[2,3- c]pyridazin-3-yl]butyl}-N- [(6-methylpyridin-3- yl)methyl]-1H-1,2,3-triazole- 4-carboxamide |
| 101 | 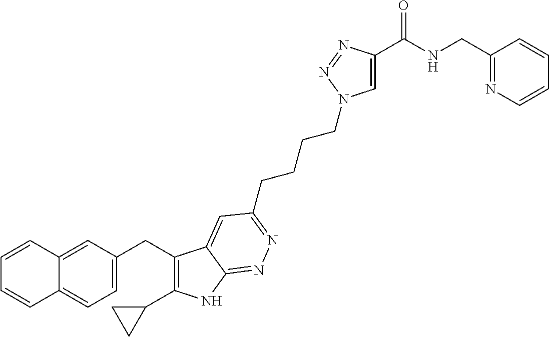 [SMILES: O=C(NCc1ccccn1)c1cn(CCCCc2cc3c(Cc4ccc5ccccc5c4)c(C4CC4)[nH]c3nn2)nn1] | 1-{4-[6-cyclopropyl-5- (naphthalen-2-ylmethyl)-7H- pyrrolo[2,3-c]pyridazin-3- yl]butyl}-N-(pyridin-2- ylmethyl)-1H-1,2,3-triazole- 4-carboxamide |
| 102 | 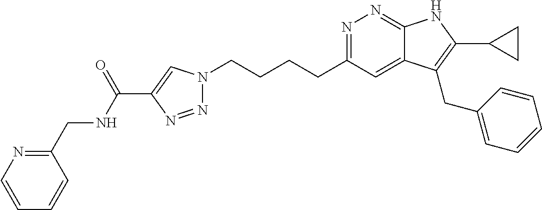 [SMILES: O=C(NCc1ccccn1)c1cn(CCCCc2cc3c(Cc4ccccc4)c(C4CC4)[nH]c3nn2)nn1] | 1-(4-{5-benzyl-6- cyclopropyl-7H-pyrrolo[2,3- c]pyridazin-3-yl}butyl)-N- (pyridin-2-ylmethyl)-1H- 1,2,3-triazole-4-carboxamide |
| 103 | 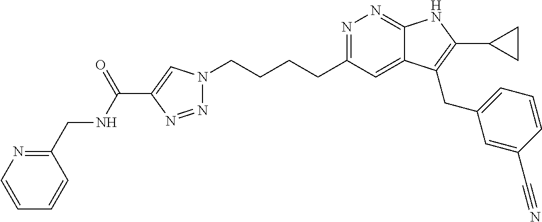 [SMILES: N#Cc1cccc(Cc2c(C3CC3)[nH]c3nnc(CCCCn4cc(C(=O)NCc5ccccn5)nn4)cc23)c1] | 1-(4-{5-[(3- cyanophenyl)methyl]-6- cyclopropyl-7H-pyrrolo[2,3- c]pyridazin-3-yl}butyl)-N- (pyridin-2-ylmethyl)-1H- 1,2,3-triazole-4-carboxamide |
| 104 | 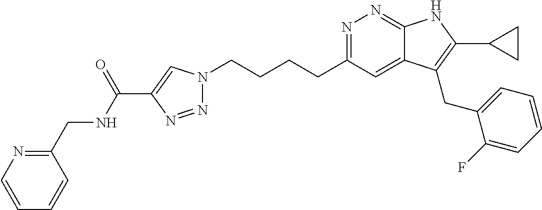 [SMILES: O=C(NCc1ccccn1)c1cn(CCCCc2cc3c(Cc4ccccc4F)c(C4CC4)[nH]c3nn2)nn1] | 1-(4-{6-cyclopropyl-5-[(2- fluorophenyl)methyl]-7H- pyrrolo[2,3-c]pyridazin-3- yl}butyl)-N-(pyridin-2- ylmethyl)-1H-1,2,3-triazole- 4-carboxamide |
| 105 | 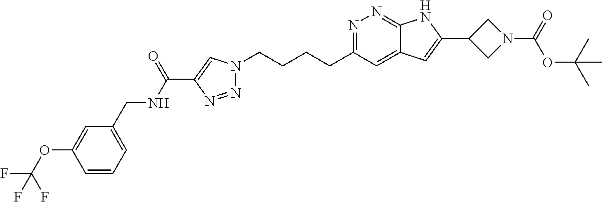 [SMILES: CC(C)(C)OC(=O)N1CC(c2cc3cc(CCCCn4cc(C(=O)NCc5cccc(OC(F)(F)F)c5)nn4)nnc3[nH]2)C1] | tert-butyl 3-(3-{4-[4-({[3- (trifluoromethoxy)phenyl] methyl}carbamoyl)-1H-1,2,3- triazol-1-yl]butyl}-7H- pyrrolo[2,3-c]pyridazin-6- yl)azetidine-1-carboxylate |
| 106 | 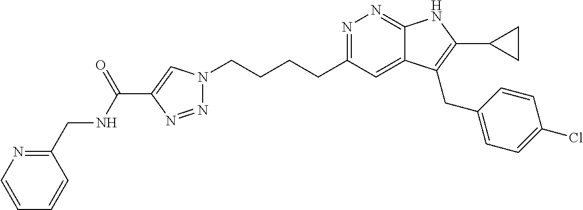 [SMILES: O=C(NCc1ccccn1)c1cn(CCCCc2cc3c(Cc4ccc(Cl)cc4)c(C4CC4)[nH]c3nn2)nn1] | 1-(4-{5-[(4- chlorophenyl)methyl]-6- cyclopropyl-7H-pyrrolo[2,3- c]pyridazin-3-yl}butyl)-N- (pyridin-2-ylmethyl)-1H- 1,2,3-triazole-4-carboxamide |
| 107 | 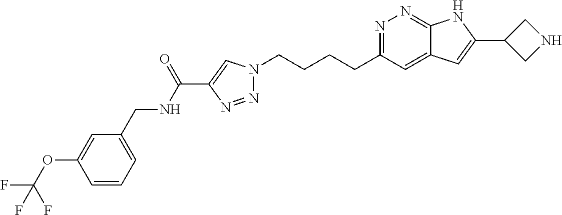 [SMILES: O=C(NCc1cccc(OC(F)(F)F)c1)c1cn(CCCCc2cc3cc(C4CNC4)[nH]c3nn2)nn1] | 1-{4-[6-(azetidin-3-yl)-7H- pyrrolo[2,3-c]pyridazin-3- yl]butyl}-N-{[3- (trifluoromethoxy)phenyl] methyl}-1H-1,2,3-triazole-4- carboxamide |
| 108 | 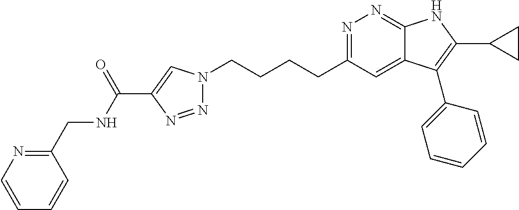 [SMILES: O=C(NCc1ccccn1)c1cn(CCCCc2cc3c(-c4ccccc4)c(C4CC4)[nH]c3nn2)nn1] | 1-(4-{6-cyclopropyl-5- phenyl-7H-pyrrolo[2,3- c]pyridazin-3-yl}butyl)-N- (pyridin-2-ylmethyl)-1H- 1,2,3-triazole-4-carboxamide |
| 109 | 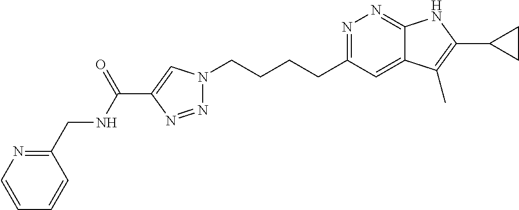 [SMILES: Cc1c(C2CC2)[nH]c2nnc(CCCCn3cc(C(=O)NCc4ccccn4)nn3)cc12] | 1-(4-{6-cyclopropyl-5- methyl-7H-pyrrolo[2,3- c]pyridazin-3-yl}butyl)-N- (pyridin-2-ylmethyl)-1H- 1,2,3-triazole-4-carboxamide |
| 110 | 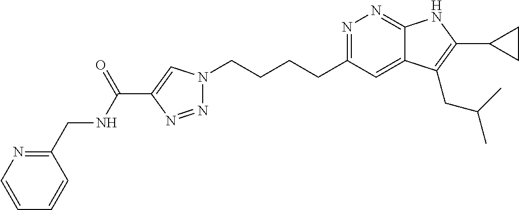 [SMILES: CC(C)Cc1c(C2CC2)[nH]c2nnc(CCCCn3cc(C(=O)NCc4ccccn4)nn3)cc12] | 1-{4-[6-cyclopropyl-5-(2- methylpropyl)-7H- pyrrolo[2,3-c]pyridazin-3- yl]butyl}-N-(pyridin-2- ylmethyl)-1H-1,2,3-triazole- 4-carboxamide |
| 111 | 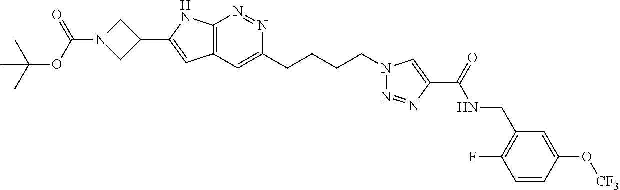 [SMILES: CC(C)(C)OC(=O)N1CC(c2cc3cc(CCCCn4cc(C(=O)NCc5cc(OC(F)(F)F)ccc5F)nn4)nnc3[nH]2)C1] | tert-butyl 3-(3-{4-[4-({[2- fluoro-5- (trifluoromethoxy)phenyl] methyl}carbamoyl)-1H-1,2,3- triazol-1-yl]butyl}-7H- pyrrolo[2,3-c]pyridazin-6- yl)azetidine-1-carboxylate |
| 112 | 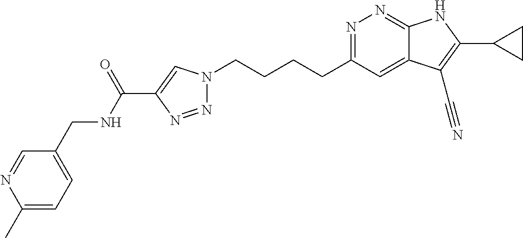 [SMILES: Cc1ccc(CNC(=O)c2cn(CCCCc3cc4c(C#N)c(C5CC5)[nH]c4nn3)nn2)cn1] | 1-(4-{5-cyano-6-cyclopropyl- 7H-pyrrolo[2,3-c]pyridazin-3- yl}butyl)-N-[(6- methylpyridin-3-yl)methyl]- 1H-1,2,3-triazole-4- carboxamide |
| 113 | 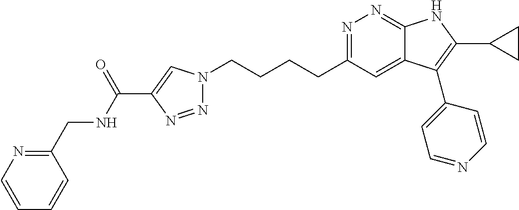 [SMILES: O=C(NCc1ccccn1)c1cn(CCCCc2cc3c(-c4ccncc4)c(C4CC4)[nH]c3nn2)nn1] | 1-{4-[6-cyclopropyl-5- (pyridin-4-yl)-7H- pyrrolo[2,3-c]pyridazin-3- yl]butyl}-N-(pyridin-2- ylmethyl)-1H-1,2,3-triazole- 4-carboxamide |
| 114 | 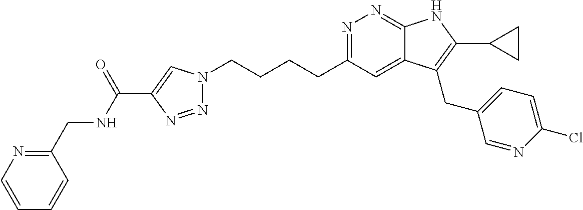 [SMILES: O=C(NCc1ccccn1)c1cn(CCCCc2cc3c(Cc4ccc(Cl)nc4)c(C4CC4)[nH]c3nn2)nn1] | 1-(4-{5-[(6-chloropyridin-3- yl)methyl]-6-cyclopropyl-7H- pyrrolo[2,3-c]pyridazin-3- yl}butyl)-N-(pyridin-2- ylmethyl)-1H-1,2,3-triazole- 4-carboxamide |
| 115 | 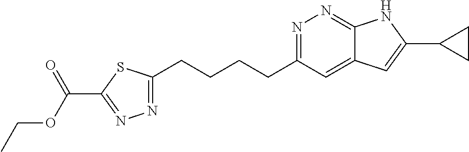 [SMILES: CCOC(=O)c1nnc(CCCCc2cc3cc(C4CC4)[nH]c3nn2)s1] | ethyl 5-(4-{6-cyclopropyl- 7H-pyrrolo[2,3-c]pyridazin-3- yl}butyl)-1,3,4-thiadiazole-2- carboxylate |
| 116 | 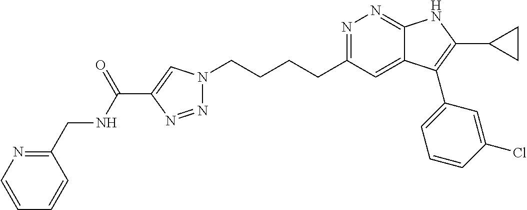 [SMILES: O=C(NCc1ccccn1)c1cn(CCCCc2cc3c(-c4cccc(Cl)c4)c(C4CC4)[nH]c3nn2)nn1] | 1-{4-[5-(3-chlorophenyl)-6- cyclopropyl-7H-pyrrolo[2,3- c]pyridazin-3-yl]butyl}-N- (pyridin-2-ylmethyl)-1H- 1,2,3-triazole-4-carboxamide |
| 117 | 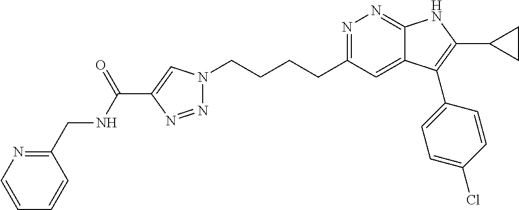 [SMILES: O=C(NCc1ccccn1)c1cn(CCCCc2cc3c(-c4ccc(Cl)cc4)c(C4CC4)[nH]c3nn2)nn1] | 1-{4-[5-(4-chlorophenyl)-6- cyclopropyl-7H-pyrrolo[2,3- c]pyridazin-3-yl]butyl}-N- (pyridin-2-ylmethyl)-1H- 1,2,3-triazole-4-carboxamide |
| 118 | 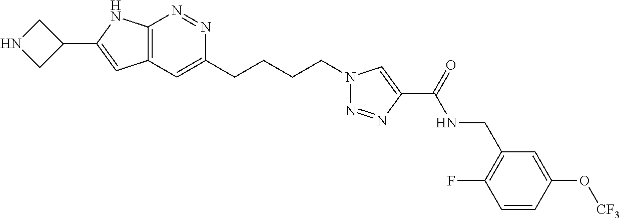 [SMILES: O=C(NCc1cc(OC(F)(F)F)ccc1F)c1cn(CCCCc2cc3cc(C4CNC4)[nH]c3nn2)nn1] | 1-{4-[6-(azetidin-3-yl)-7H- pyrrolo[2,3-c]pyridazin-3- yl]butyl}-N-{[2-fluoro-5- (trifluoromethoxy)phenyl] methyl}-1H-1,2,3-triazole-4- carboxamide |
| 119 | 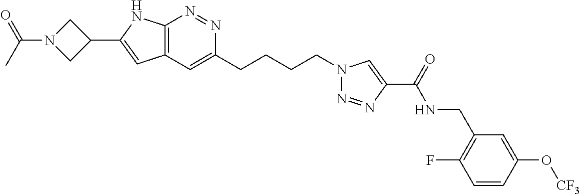 [SMILES: CC(=O)N1CC(c2cc3cc(CCCCn4cc(C(=O)NCc5cc(OC(F)(F)F)ccc5F)nn4)nnc3[nH]2)C1] | 1-{4-[6-(1-acetylazetidin-3- yl)-7H-pyrrolo[2,3- c]pyridazin-3-yl]butyl}-N- {[2-fluoro-5- (trifluoromethoxy)phenyl] methyl}-1H-1,2,3-triazole-4- carboxamide |
| 120 | 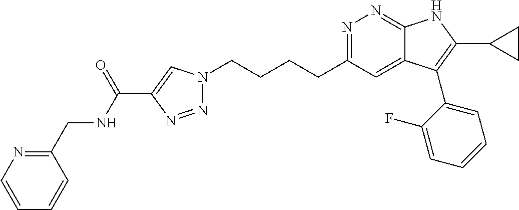 [SMILES: O=C(NCc1ccccn1)c1cn(CCCCc2cc3c(-c4ccccc4F)c(C4CC4)[nH]c3nn2)nn1] | 1-{4-[6-cyclopropyl-5-(2- fluorophenyl)-7H- pyrrolo[2,3-c]pyridazin-3- yl]butyl}-N-(pyridin-2- ylmethyl)-1H-1,2,3-triazole- 4-carboxamide |
| 121 | 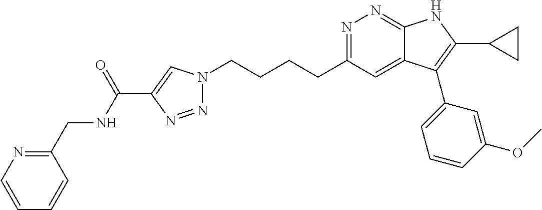 [SMILES: COc1cccc(-c2c(C3CC3)[nH]c3nnc(CCCCn4cc(C(=O)NCc5ccccn5)nn4)cc23)c1] | 1-{4-[6-cyclopropyl-5-(3- methoxyphenyl)-7H- pyrrolo[2,3-c]pyridazin-3- yl]butyl)-N-(pyridin-2- ylmethyl)-1H-1,2,3-triazole- 4-carboxamide |
| 122 | 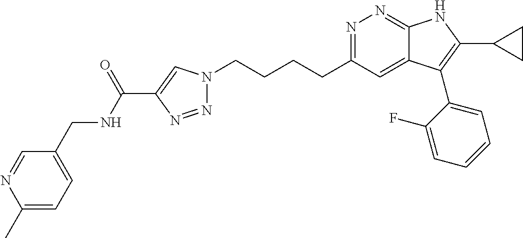 [SMILES: Cc1ccc(CNC(=O)c2cn(CCCCc3cc4c(-c5ccccc5F)c(C5CC5)[nH]c4nn3)nn2)cn1] | 1-{4-[6-cyclopropyl-5-(2- fluorophenyl)-7H- pyrrolo[2,3-c]pyridazin-3- yl]butyl}-N-[(6- methylpyridin-3-yl)methyl]- 1H-1,2,3-triazole-4- carboxamide |
| 123 | 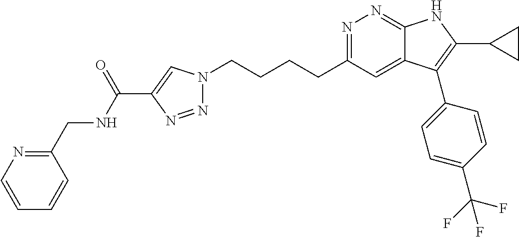 [SMILES: O=C(NCc1ccccn1)c1cn(CCCCc2cc3c(-c4ccc(C(F)(F)F)cc4)c(C4CC4)[nH]c3nn2)nn1] | 1-(4-{6-cyclopropyl-5-[4- (trifluoromethyl)phenyl]-7H- pyrrolo[2,3-c]pyridazin-3- yl}butyl)-N-(pyridin-2- ylmethyl)-1H-1,2,3-triazole- 4-carboxamide |
| 124 | 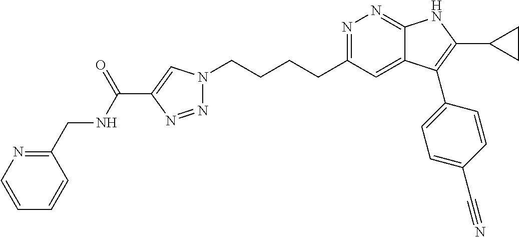 [SMILES: N#Cc1ccc(-c2c(C3CC3)[nH]c3nnc(CCCCn4cc(C(=O)NCc5ccccn5)nn4)cc23)cc1] | 1-{4-[5-(4-cyanophenyl)-6- cyclopropyl-7H-pyrrolo[2,3- c]pyridazin-3-yl]butyl}-N- (pyridin-2-ylmethyl)-1H- 1,2,3-triazole-4-carboxamide |
| 125 | 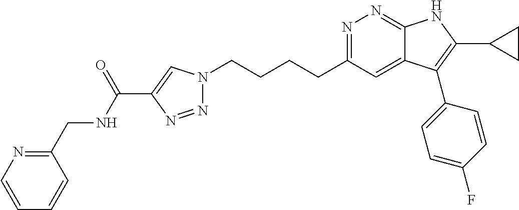 [SMILES: O=C(NCc1ccccn1)c1cn(CCCCc2cc3c(-c4ccc(F)cc4)c(C4CC4)[nH]c3nn2)nn1] | 1-{4-[6-cyclopropyl-5-(4- fluorophenyl)-7H- pyrrolo[2,3-c]pyridazin-3- yl]butyl}-N-(pyridin-2- ylmethyl)-1H-1,2,3-triazole- 4-carboxamide |
| 126 | 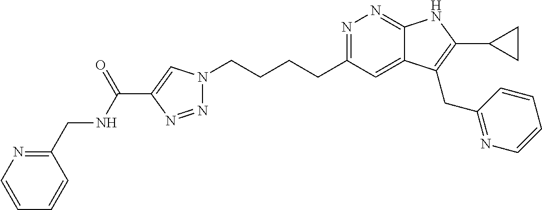 [SMILES: O=C(NCc1ccccn1)c1cn(CCCCc2cc3c(Cc4ccccn4)c(C4CC4)[nH]c3nn2)nn1] | 1-{4-[6-cyclopropyl-5- (pyridin-2-ylmethyl)-7H- pyrrolo[2,3-c]pyridazin-3- yl]butyl}-N-(pyridin-2- ylmethyl)-1H-1,2,3-triazole- 4-carboxamide |
| 127 | 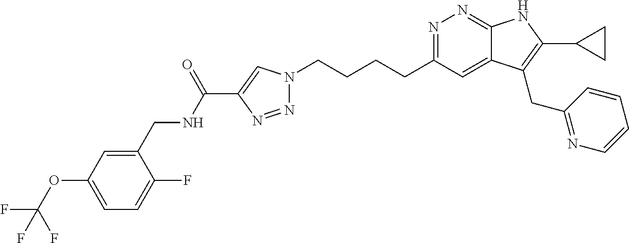 [SMILES: O=C(NCc1cc(OC(F)(F)F)ccc1F)c1cn(CCCCc2cc3c(Cc4ccccn4)c(C4CC4)[nH]c3nn2)nn1] | 1-{4-[6-cyclopropyl-5- (pyridin-2-ylmethyl)-7H- pyrrolo[2,3-c]pyridazin-3- yl]butyl}-N-{[2-fluoro-5- (trifluoromethoxy)phenyl] methyl}-1H-1,2,3-triazole-4- carboxamide |
| 128 | 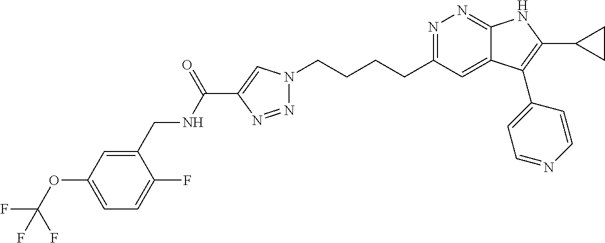 [SMILES: O=C(NCc1cc(OC(F)(F)F)ccc1F)c1cn(CCCCc2cc3c(-c4ccncc4)c(C4CC4)[nH]c3nn2)nn1] | 1-{4-[6-cyclopropyl-5- (pyridin-4-yl)-7H- pyrrolo[2,3-c]pyridazin-3- yl]butyl)-N-{[2-fluoro-5- (trifluoromethoxy)phenyl] methyl}-1H-1,2,3-triazole-4- carboxamide |
| 129 | 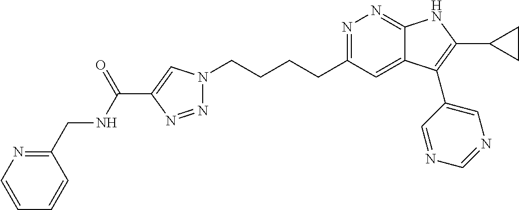 [SMILES: O=C(NCc1ccccn1)c1cn(CCCCc2cc3c(-c4cncnc4)c(C4CC4)[nH]c3nn2)nn1] | 1-{4-[6-cyclopropyl-5- (pyrimidin-5-yl)-7H- pyrrolo[2,3-c]pyridazin-3- yl]butyl)-N-(pyridin-2- ylmethyl)-1H-1,2,3-triazole- 4-carboxamide |
| 130 | 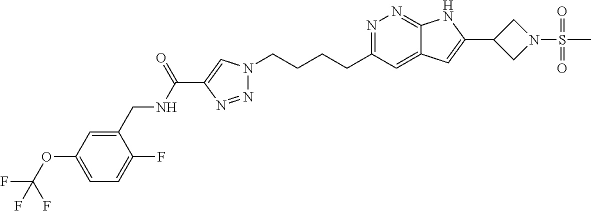 [SMILES: CS(=O)(=O)N1CC(c2cc3cc(CCCCn4cc(C(=O)NCc5cc(OC(F)(F)F)ccc5F)nn4)nnc3[nH]2)C1] | N-{[2-fluoro-5- (trifluoromethoxy)phenyl] methyl}-1-{4-[6-(1- methanesulfonylazetidin-3- yl)-7H-pyrrolo[2,3- c]pyridazin-3-yl]butyl}-1H- 1,2,3-triazole-4-carboxamide |
| 131 | 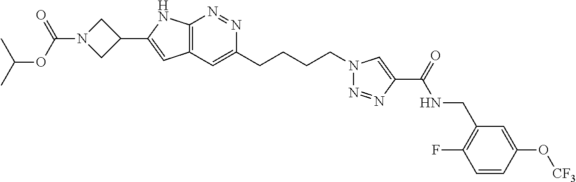 [SMILES: CC(C)OC(=O)N1CC(c2cc3cc(CCCCn4cc(C(=O)NCc5cc(OC(F)(F)F)ccc5F)nn4)nnc3[nH]2)C1] | propan-2-yl 3-(3-{4-[4-({[2- fluoro-5- (trifluoromethoxy)phenyl] methyl}carbamoyl)-1H-1,2,3- triazol-1-yl]butyl}-7H- pyrrolo[2,3-c]pyridazin-6- yl)azetidine-1-carboxylate |
| 132 | 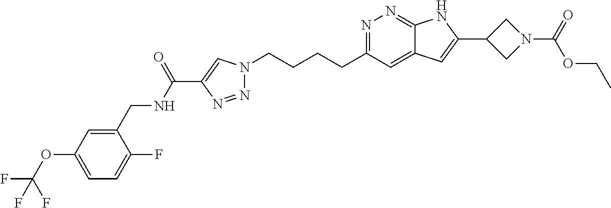 [SMILES: CCOC(=O)N1CC(c2cc3cc(CCCCn4cc(C(=O)NCc5cc(OC(F)(F)F)ccc5F)nn4)nnc3[nH]2)C1] | ethyl 3-(3-{4-[4-({[2-fluoro- 5- (trifluoromethoxy)phenyl] methyl}carbamoyl)-1H-1,2,3- triazol-1-yl]butyl)-7H- pyrrolo[2,3-c]pyridazin-6- yl)azetidine-1-carboxylate |
| 133 | 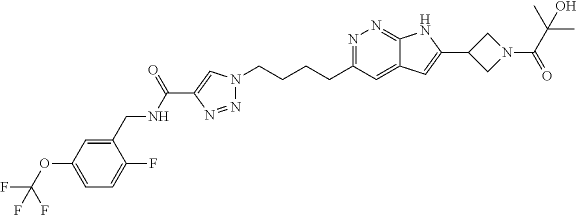 [SMILES: CC(C)(O)C(=O)N1CC(c2cc3cc(CCCCn4cc(C(=O)NCc5cc(OC(F)(F)F)ccc5F)nn4)nnc3[nH]2)C1] | N-{[2-fluoro-5- (trifluoromethoxy)phenyl] methyl}-1-(4-{6-[1-(2-hydroxy- 2-methylpropanoyl)azetidin- 3-yl]-7H-pyrrolo[2,3- c]pyridazin-3-yl}butyl)-1H- 1,2,3-triazole-4-carboxamide |
| 134 | 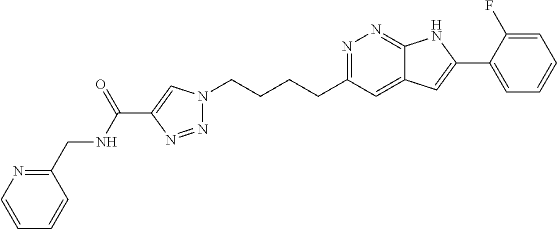 [SMILES: O=C(NCc1ccccn1)c1cn(CCCCc2cc3cc(-c4ccccc4F)[nH]c3nn2)nn1] | 1-{4-[6-(2-fluorophenyl)-7H- pyrrolo[2,3-c]pyridazin-3- yl]butyl}-N-(pyridin-2- ylmethyl)-1H-1,2,3-triazole- 4-carboxamide |
| 135 | 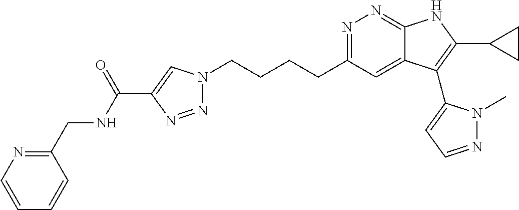 [SMILES: Cn1nccc1-c1c(C2CC2)[nH]c2nnc(CCCCn3cc(C(=O)NCc4ccccn4)nn3)cc12] | 1-{4-[6-cyclopropyl-5-(1- methyl-1H-pyrazol-5-yl)-7H- pyrrolo[2,3-c]pyridazin-3- yl]butyl}-N-(pyridin-2- ylmethyl)-1H-1,2,3-triazole- 4-carboxamide |
| 136 | 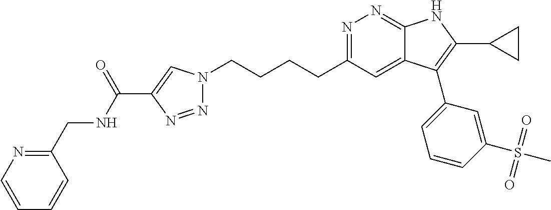 [SMILES: CS(=O)(=O)c1cccc(-c2c(C3CC3)[nH]c3nnc(CCCCn4cc(C(=O)NCc5ccccn5)nn4)cc23)c1] | 1-{4-[6-cyclopropyl-5-(3- methanesulfonylphenyl)-7H- pyrrolo[2,3-c]pyridazin-3- yl]butyl}-N-(pyridin-2- ylmethyl)-1H-1,2,3-triazole- 4-carboxamide |
| 137 | 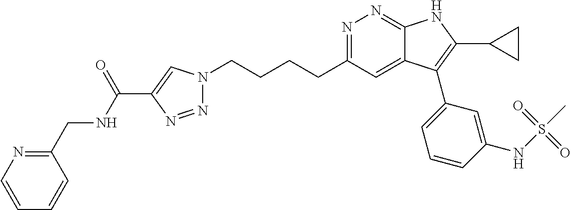 [SMILES: CS(=O)(=O)Nc1cccc(-c2c(C3CC3)[nH]c3nnc(CCCCn4cc(C(=O)NCc5ccccn5)nn4)cc23)c1] | 1-{4-[6-cyclopropyl-5-(3- methanesulfonamidophenyl)- 7H-pyrrolo[2,3-c]pyridazin-3- yl]butyl}-N-(pyridin-2- ylmethyl)-1H-1,2,3-triazole- 4-carboxamide |
| 138 | 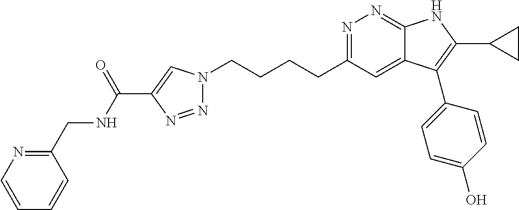 [SMILES: O=C(NCc1ccccn1)c1cn(CCCCc2cc3c(-c4ccc(O)cc4)c(C4CC4)[nH]c3nn2)nn1] | 1-{4-[6-cyclopropyl-5-(4- hydroxyphenyl)-7H- pyrrolo[2,3-c]pyridazin-3- yl]butyl}-N-(pyridin-2- ylmethyl)-1H-1,2,3-triazole- 4-carboxamide |
| 139 | 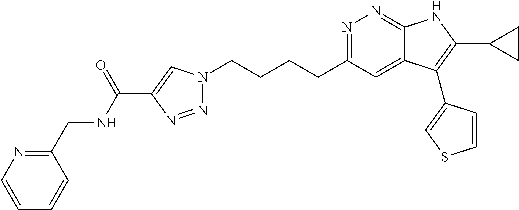 [SMILES: O=C(NCc1ccccn1)c1cn(CCCCc2cc3c(-c4ccsc4)c(C4CC4)[nH]c3nn2)nn1] | 1-{4-[6-cyclopropyl-5- (thiophen-3-yl)-7H- pyrrolo[2,3-c]pyridazin-3- yl]butyl}-N-(pyridin-2- ylmethyl)-1H-1,2,3-triazole- 4-carboxamide |
| 140 | 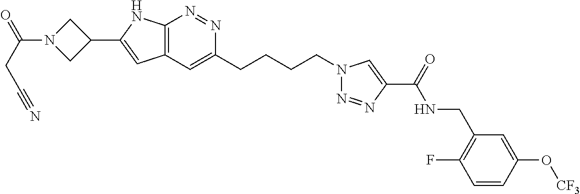 [SMILES: N#CCC(=O)N1CC(c2cc3cc(CCCCn4cc(C(=O)NCc5cc(OC(F)(F)F)ccc5F)nn4)nnc3[nH]2)C1] | 1-(4-{6-[1-(2- cyanoacetyl)azetidin-3-yl]- 7H-pyrrolo[2,3-c]pyridazin-3- yl}butyl)-N-{[2-fluoro-5- (trifluoromethoxy)phenyl] methyl}-1H-1,2,3-triazole-4- carboxamide |
| 141 | 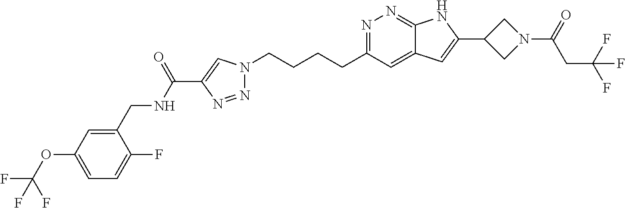 [SMILES: O=C(NCc1cc(OC(F)(F)F)ccc1F)c1cn(CCCCc2cc3cc(C4CN(C(=O)CC(F)(F)F)C4)[nH]c3nn2)nn1] | N-{[2-fluoro-5- (trifluoromethoxy)phenyl] methyl}-1-(4-{6-[1-(3,3,3- trifluoropropanoyl)azetidin-3- yl]-7H-pyrrolo[2,3- c]pyridazin-3-yl}butyl)-1H- 1,2,3-triazole-4-carboxamide |
| 142 | 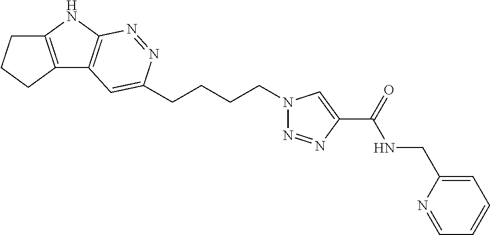 [SMILES: O=C(NCc1ccccn1)c1cn(CCCCc2cc3c4c([nH]c3nn2)CCC4)nn1] | N-(pyridin-2-ylmethyl)-1-(4- {7,9,10- triazatricyclo[6.4.0.0\hat{ }{2,6}] dodeca-1(12),2(6),8,10-tetraen- 11-yl}butyl)-1H-1,2,3- triazole-4-carboxamide |
| 143 | 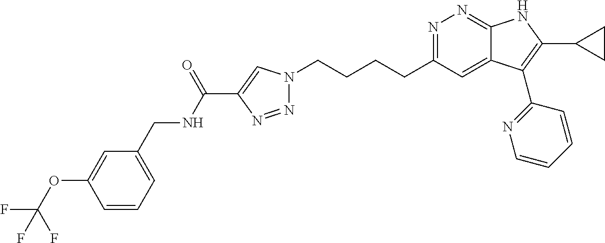 [SMILES: O=C(NCc1cccc(OC(F)(F)F)c1)c1cn(CCCCc2cc3c(-c4ccccn4)c(C4CC4)[nH]c3nn2)nn1] | 1-{4-[6-cyclopropyl-5- (pyridin-2-yl)-7H- pyrrolo[2,3-c]pyridazin-3- yl]butyl}-N-{[3- (trifluoromethoxy)phenyl] methyl}-1H-1,2,3-triazole-4- carboxamide |
| 144 | 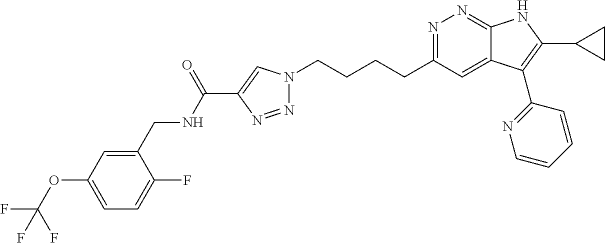 [SMILES: O=C(NCc1cc(OC(F)(F)F)ccc1F)c1cn(CCCCc2cc3c(-c4ccccn4)c(C4CC4)[nH]c3nn2)nn1] | 1-{4-[6-cyclopropyl-5- (pyridin-2-yl)-7H- pyrolo[2,3-c]pyridazin-3- yl]butyl}-N-{[2-fluoro-5- (trifluoromethoxy)phenyl] methyl}-1H-1,2,3-triazole-4- carboxamide |
| 145 | 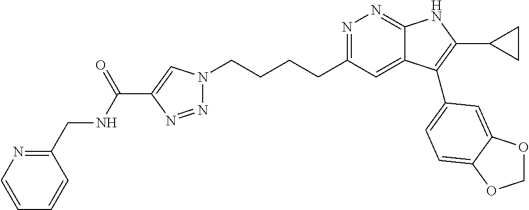 [SMILES: O=C(NCc1ccccn1)c1cn(CCCCc2cc3c(-c4ccc5c(c4)OCO5)c(C4CC4)[nH]c3nn2)nn1] | 1-{4-[5-(2H-1,3-benzodioxol- 5-yl)-6-cyclopropyl-7H- pyrrolo[2,3-c]pyridazin-3- yl]butyl}-N-(pyridin-2- ylmethyl)-1H-1,2,3-triazole- 4-carboxamide |
| 146 | 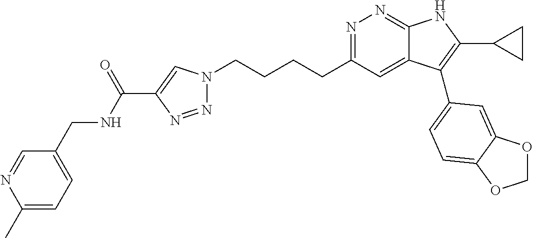 [SMILES: Cc1ccc(CNC(=O)c2cn(CCCCc3cc4c(-c5ccc6c(c5)OCO6)c(C5CC5)[nH]c4nn3)nn2)cn1] | 1-{4-[5-(2H-1,3-benzodioxol- 5-yl)-6-cyclopropyl-7H- pyrrolo[2,3-c]pyridazin-3- yl]butyl}-N-[(6- methylpyridin-3-yl)methyl]- 1H-1,2,3-triazole-4- carboxamide |
| 147 | 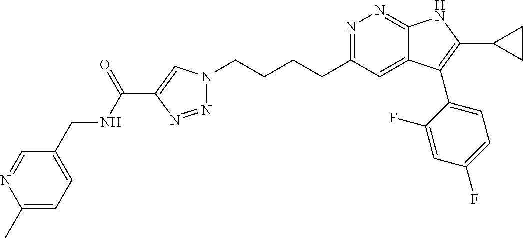 [SMILES: Cc1ccc(CNC(=O)c2cn(CCCCc3cc4c(-c5ccc(F)cc5F)c(C5CC5)[nH]c4nn3)nn2)cn1] | 1-{4-[6-cyclopropyl-5-(2,4- difluorophenyl)-7H- pyrrolo[2,3-c]pyridazin-3- yl]butyl}-N-[(6- methylpyridin-3-yl)methyl]- 1H-1,2,3-triazole-4- carboxamide |
| 148 | 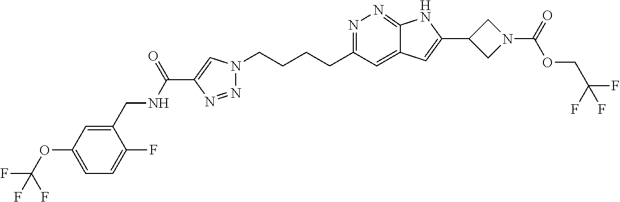 [SMILES: O=C(NCc1cc(OC(F)(F)F)ccc1F)c1cn(CCCCc2cc3cc(C4CN(C(=O)OCC(F)(F)F)C4)[nH]c3nn2)nn1] | 2,2,2-trifluoroethyl 3-(3-{4- [4-({[2-fluoro-5- (trifluoromethoxy)phenyl] methyl}carbamoyl)-1H-1,2,3- triazol-1-yl]butyl}-7H- pyrrolo[2,3-c]pyridazin-6- yl)azetidine-1-carboxylate |
| 149 | 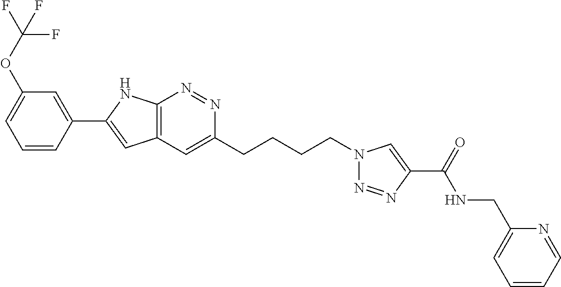 [SMILES: O=C(NCc1ccccn1)c1cn(CCCCc2cc3cc(-c4cccc(OC(F)(F)F)c4)[nH]c3nn2)nn1] | N-(pyridin-2-ylmethyl)-1-(4- {6-[3- (trifluoromethoxy)phenyl]- 7H-pyrrolo[2,3-c]pyridazin-3- yl}butyl)-1H-1,2,3-triazole-4- carboxamide |
| 150 | 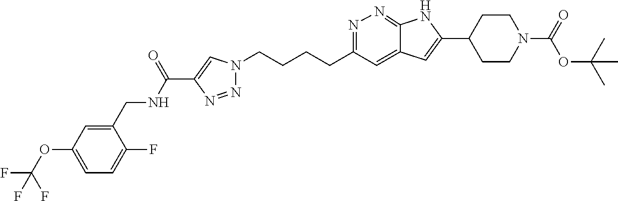 [SMILES: CC(C)(C)OC(=O)N1CCC(c2cc3cc(CCCCn4cc(C(=O)NCc5cc(OC(F)(F)F)ccc5F)nn4)nnc3[nH]2)CC1] | tert-butyl 4-(3-{4-[4-({[2- fluoro-5- (trifluoromethoxy)phenyl] methyl}carbamoyl)-1H-1,2,3- triazol-1-yl]butyl}-7H- pyrrolo[2,3-c]pyridazin-6- yl)piperidine-1-carboxylate |
| 151 | 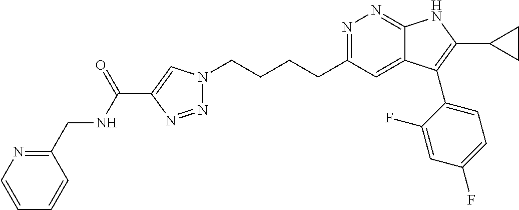 [SMILES: O=C(NCc1ccccn1)c1cn(CCCCc2cc3c(-c4ccc(F)cc4F)c(C4CC4)[nH]c3nn2)nn1] | 1-(4-[6-cyclopropyl-5-(2,4- difluorophenyl)-7H- pyrrolo[2,3-c]pyridazin-3- yl]butyl}-N-(pyridin-2- ylmethyl-1H-1,2,3-triazole- 4-carboxamide |
| 152 | 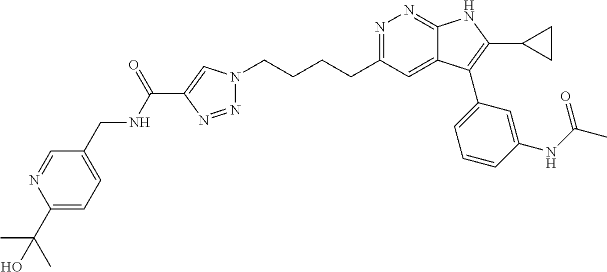 [SMILES: CC(=O)Nc1cccc(-c2c(C3CC3)[nH]c3nnc(CCCCn4cc(C(=O)NCc5ccc(C(C)(C)O)nc5)nn4)cc23)c1] | 1-{4-[6-cyclopropyl-5-(3- acetamidophenyl)-7H- pyrrolo[2,3-c]pyridazin-3- yl]butyl}-N-{[6-(2- hydroxypropan-2-yl)pyridin- 3-yl]methyl}-1H-1,2,3- triazole-4-carboxamide |
| 153 | 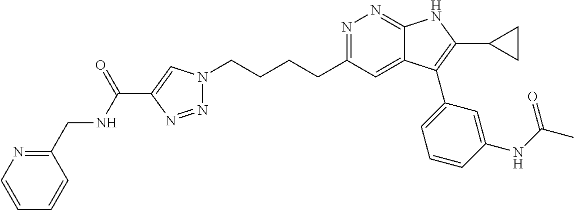 [SMILES: CC(=O)Nc1cccc(-c2c(C3CC3)[nH]c3nnc(CCCCn4cc(C(=O)NCc5ccccn5)nn4)cc23)c1] | 1-{4-[6-cyclopropyl-5-(3- acetamidophenyl)-7H- pyrrolo[2,3-clpyridazin-3- yl]butyl}-N-(pyridin-2- ylmethyl)-1H-1,2,3-triazole- 4-carboxamide |
| 154 | 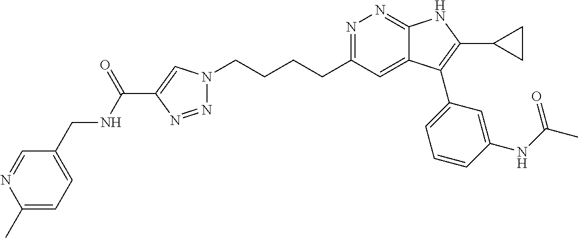 [SMILES: CC(=O)Nc1cccc(-c2c(C3CC3)[nH]c3nnc(CCCCn4cc(C(=O)NCc5ccc(C)nc5)nn4)cc23)c1] | 1-{4-[6-cyclopropyl-5-(3- acetamidophenyl)-7H- pyrrolo[2,3-c]pyridazin-3- yl]butyl}-N-[(6- methylpyridin-3-yl)methyl]- 1H-1,2,3-triazole-4- carboxamide |
| 155 | 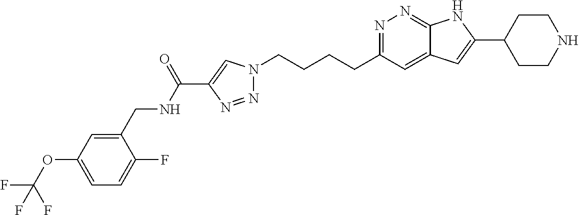 [SMILES: O=C(NCc1cc(OC(F)(F)F)ccc1F)c1cn(CCCCc2cc3cc(C4CCNCC4)[nH]c3nn2)nn1] | N-{[2-fluoro-5- (trifluoromethoxy)phenyl] methyl)-1-{4-[6-(piperidin-4-yl)- 7H-pyrrolo[2,3-c]pyridazin-3- yl]butyl)-1H-1,2,3-triazole-4- carboxamide |
| 156 | 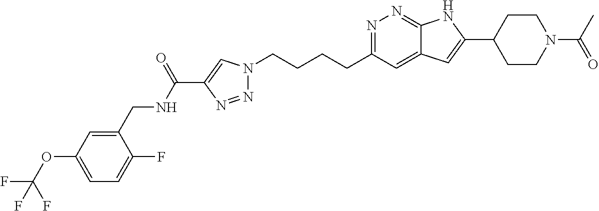 [SMILES: CC(=O)N1CCC(c2cc3cc(CCCCn4cc(C(=O)NCc5cc(OC(F)(F)F)ccc5F)nn4)nnc3[nH]2)CC1] | 1-{4-[6-(1-acetylpiperidin-4- yl)-7H-pyrrolo[2,3- c]pyridazin-3-yl]butyl}-N- {[2-fluoro-5- (trifluoromethoxy)phenyl] methyl}-1H-1,2,3-triazole-4- carboxamide |
| 157 | 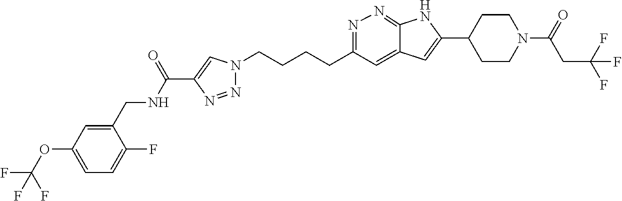 [SMILES: O=C(NCc1cc(OC(F)(F)F)ccc1F)c1cn(CCCCc2cc3cc(C4CCN(C(=O)CC(F)(F)F)CC4)[nH]c3nn2)nn1] | N-{[2-fluoro-5- (trifluoromethoxy)phenyl] methyl)-1-(4-{6-[1-(3,3,3- trifluoropropanoyl)piperidin- 4-yl]-7H-pyrrolo[2,3- c]pyridazin-3-yl}butyl)-1H- 1,2,3-triazole-4-carboxamide |
| 158 | 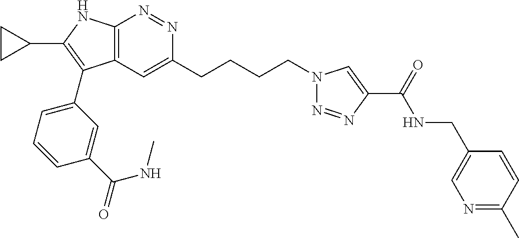 [SMILES: CNC(=O)c1cccc(-c2c(C3CC3)[nH]c3nnc(CCCCn4cc(C(=O)NCc5ccc(C)nc5)nn4)cc23)c1] | 1-(4-{6-cyclopropyl-5-[3- (methylcarbamoyl)phenyl]- 7H-pyrrolo[2,3-c]pyridazin-3- yl}butyl)-N-[(6- methylpyridin-3-yl)methyl]- 1H-1,2,3-triazole-4- carboxamide |
| 159 | 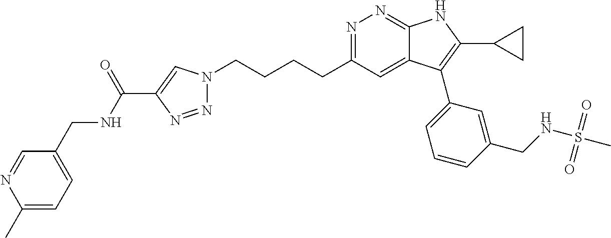 [SMILES: Cc1ccc(CNC(=O)c2cn(CCCCc3cc4c(-c5cccc(CNS(C)(=O)=O)c5)c(C5CC5)[nH]c4nn3)nn2)cn1] | 1-(4-{6-cyclopropyl-5-[3- (methanesulfonamidomethyl) phenyl]-7H-pyrrolo[2,3- c]pyridazin-3-yl}butyl)-N- [(6-methylpyridin-3- yl)methyl]-1H-1,2,3-triazole- 4-carboxamide |
| 160 | 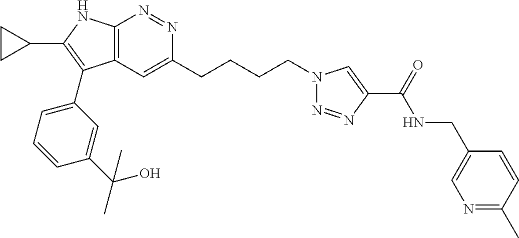 [SMILES: Cc1ccc(CNC(=O)c2cn(CCCCc3cc4c(-c5cccc(C(C)(C)O)c5)c(C5CC5)[nH]c4nn3)nn2)cn1] | 1-(4-{6-cyclopropyl-5-[3-(2- hydroxypropan-2-yl)phenyl]- 7H-pyrrolo[2,3-c]pyridazin-3- yl}butyl)-N-[(6- mcthylpyridin-3-yl)methyl]- 1H-1,2,3-triazole-4- carboxamide |
| 161 | 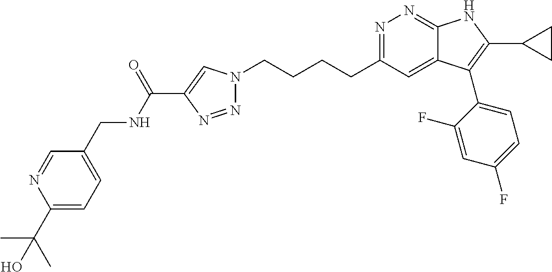 [SMILES: CC(C)(O)c1ccc(CNC(=O)c2cn(CCCCc3cc4c(-c5ccc(F)cc5F)c(C5CC5)[nH]c4nn3)nn2)cn1] | 1-{4-[6-cyclopropyl-5-(2,4- difluorophenyl)-7H- pyrrolo[2,3-c]pyridazin-3- yl]butyl}-N-{[6-(2- hydroxypropan-2-yl)pyridin- 3-yl]methyl}-1H-1,2,3- triazole-4-carboxamide |
| 162 | 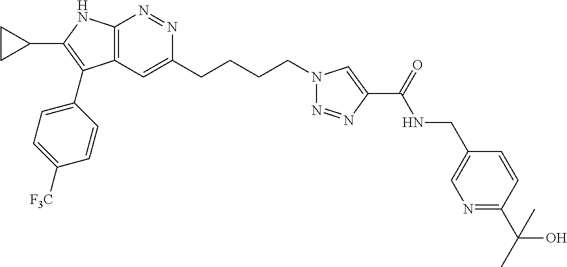 [SMILES: CC(C)(O)c1ccc(CNC(=O)c2cn(CCCCc3cc4c(-c5ccc(C(F)(F)F)cc5)c(C5CC5)[nH]c4nn3)nn2)cn1] | 1-(4-{6-cyclopropyl-5-[4- (trifluoromethyl)phenyl]-7H- pyrrolo[2,3-c]pyridazin-3- yl}butyl)-N-{[6-(2- hydroxypropan-2-yl)pyridin- 3-yl]methyl}-1H-1,2,3- triazole-4-carboxamide |
| 163 | 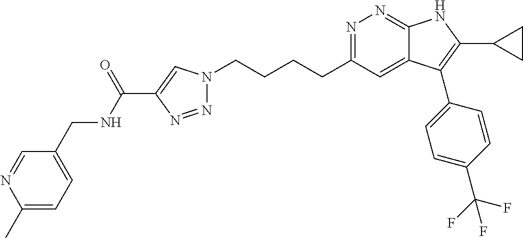 [SMILES: Cc1ccc(CNC(=O)c2cn(CCCCc3cc4c(-c5ccc(C(F)(F)F)cc5)c(C5CC5)[nH]c4nn3)nn2)cn1] | 1-(4-{6-cyclopropyl-5-[4- (trifluoromethyl)phenyl]-7H- pyrrolo[2,3-c]pyridazin-3- yl}butyl)-N-[(6- methylpyridin-3-yl)methyl]- 1H-1,2,3-triazole-4- carboxamide |
| 164 | 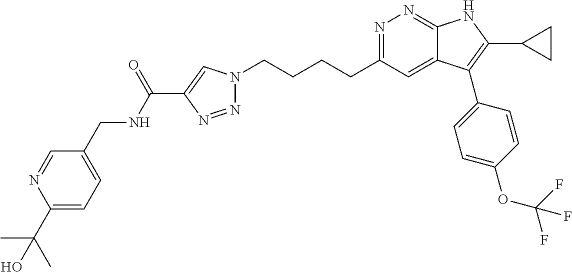 [SMILES: CC(C)(O)c1ccc(CNC(=O)c2cn(CCCCc3cc4c(-c5ccc(OC(F)(F)F)cc5)c(C5CC5)[nH]c4nn3)nn2)cn1] | 1-(4-{6-cyclopropyl-5-[4- (trifluoromethoxy)phenyl]- 7H-pyrrolo[2,3-c]pyridazin-3- yl}butyl)-N-{[6-(2- hydroxypropan-2-yl)pyridin- 3-yl]methyl}-1H-1,2,3- triazole-4-carboxamide |
| 165 | 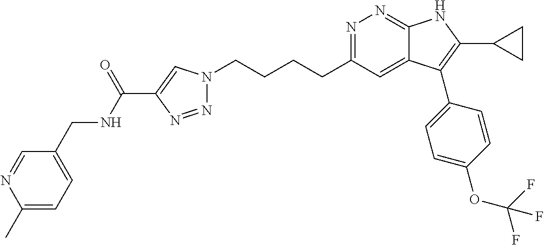 [SMILES: Cc1ccc(CNC(=O)c2cn(CCCCc3cc4c(-c5ccc(OC(F)(F)F)cc5)c(C5CC5)[nH]c4nn3)nn2)cn1] | 1-(4-{6-cyclopropyl-5-[4- (trifluoromethoxy)phenyl]- 7H-pyrrolo[2,3-c]pyridazin-3- yl}butyl)-N-[(6- methylpyridin-3-yl)methyl]- 1H-1,2,3-triazole-4- carboxamide |
| 166 | 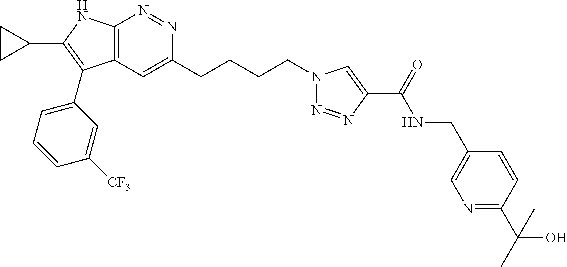 [SMILES: CC(C)(O)c1ccc(CNC(=O)c2cn(CCCCc3cc4c(-c5cccc(C(F)(F)F)c5)c(C5CC5)[nH]c4nn3)nn2)cn1] | 1-(4-{6-cyclopropyl-5-[3- (trifluoromethyl)phenyl]-7H- pyrrolo[2,3-c]pyridazin-3- yl}butyl)-N-{[6-(2- hydroxypropan-2-yl)pyridin- 3-yl]methyl}-1H-1,2,3- triazole-4-carboxamide |
| 167 | 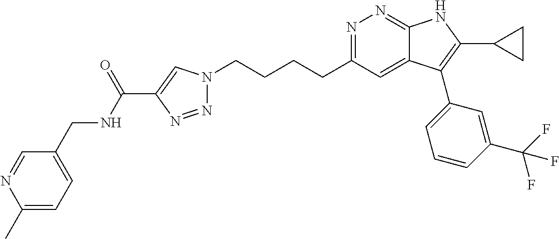 [SMILES: Cc1ccc(CNC(=O)c2cn(CCCCc3cc4c(-c5cccc(C(F)(F)F)c5)c(C5CC5)[nH]c4nn3)nn2)cn1] | 1-(4-{6-cyclopropyl-5-[3- (trifluoromethyl)phenyl]-7H- pyrrolo[2,3-c]pyridazin-3- yl}butyl)-N-[(6- methylpyridin-3-yl)methyl]- 1H-1,2,3-triazole-4- carboxamide |
| 168 | 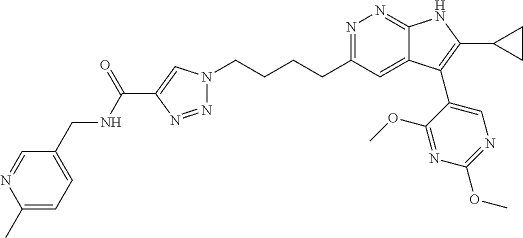 [SMILES: COc1ncc(-c2c(C3CC3)[nH]c3nnc(CCCCn4cc(C(=O)NCc5ccc(C)nc5)nn4)cc23)c(OC)n1] | 1-{4-[6-cyclopropyl-5-(2,4- dimethoxypyrimidin-5-yl)- 7H-pyrrolo[2,3-c]pyridazin-3- yl]butyl}-N-[(6- methylpyridin-3-yl)methyl]- 1H-1,2,3-triazole-4- carboxamide |
| 169 | 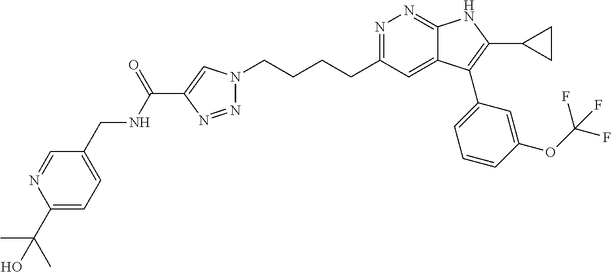 [SMILES: CC(C)(O)c1ccc(CNC(=O)c2cn(CCCCc3cc4c(-c5cccc(OC(F)(F)F)c5)c(C5CC5)[nH]c4nn3)nn2)cn1] | 1-(4-{6-cyclopropyl-5-[3- (trifluoromethoxy)phenyl]- 7H-pyrrolo[2,3-c]pyridazin-3- yl}butyl)-N-{[6-(2- hydroxypropan-2-yl)pyridin- 3-yl]methyl}-1H-1,2,3- triazole-4-carboxamide |
| 170 | 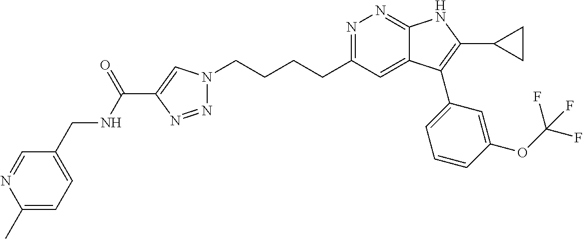 [SMILES: Cc1ccc(CNC(=O)c2cn(CCCCc3cc4c(-c5cccc(OC(F)(F)F)c5)c(C5CC5)[nH]c4nn3)nn2)cn1] | 1-(4-{6-cyclopropyl-5-[3- (trifluoromethoxy)phenyl]- 7H-pyrrolo[2,3-c]pyridazin-3- yl}butyl)-N-[(6- methylpyridin-3-yl)methyl]- 1H-1,2,3-triazole-4- carboxamide |
| 171 | 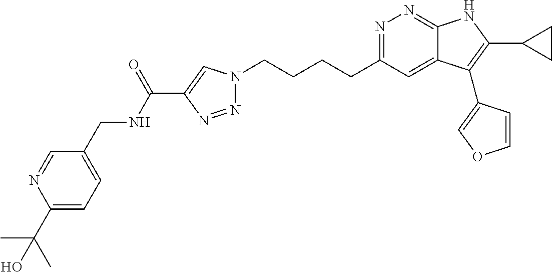 [SMILES: CC(C)(O)c1ccc(CNC(=O)c2cn(CCCCc3cc4c(-c5ccoc5)c(C5CC5)[nH]c4nn3)nn2)cn1] | 1-{4-[6-cyclopropyl-5-(furan- 3-yl)-7H-pyrrolo[2,3- c]pyridazin-3-yl]butyl}-N- {[6-(2-hydroxypropan-2- yl)pyridin-3-yl]methyl}-1H- 1,2,3-triazole-4-carboxamide |
| 172 | 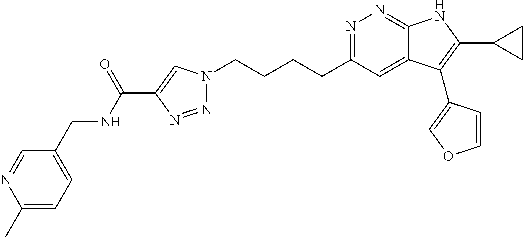 [SMILES: Cc1ccc(CNC(=O)c2cn(CCCCc3cc4c(-c5ccoc5)c(C5CC5)[nH]c4nn3)nn2)cn1] | 1-{4-[6-cyclopropyl-5-(furan- 3-yl)-7H-pyrrolo[2,3- c]pyridazin-3-yl]butyl}-N- [(6-methylpyridin-3- yl)methyl]-1H-1,2,3-triazole- 4-carboxamide |
| 173 | 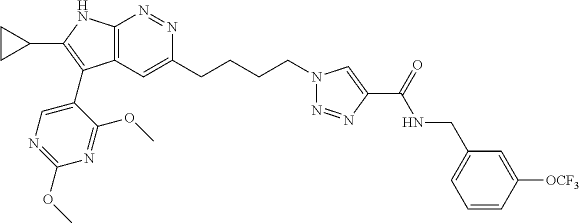 [SMILES: COc1ncc(-c2c(C3CC3)[nH]c3nnc(CCCCn4cc(C(=O)NCc5cccc(OC(F)(F)F)c5)nn4)cc23)c(OC)n1] | 1-{4-[6-cyclopropyl-5-(2,4- dimethoxypyrimidin-5-yl)- 7H-pyrrolo[2,3-c]pyridazin-3- yl]butyl}-N-{[3- (trifluoromethoxy)phenyl] methyl}-1H-1,2,3-triazole-4- carboxamide |
| 174 | 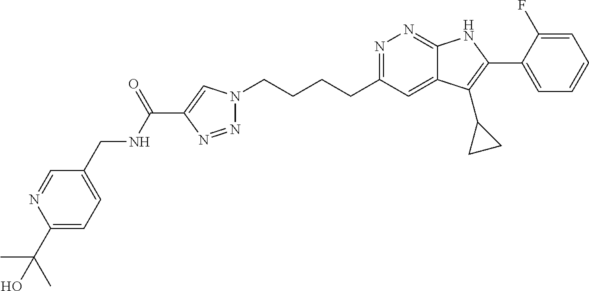 [SMILES: CC(C)(O)c1ccc(CNC(=O)c2cn(CCCCc3cc4c(C5CC5)c(-c5ccccc5F)[nH]c4nn3)nn2)cn1] | 1-{4-[5-cyclopropyl-6-(2- fluorophenyl)-7H- pyrrolo[2,3-c]pyridazin-3- yl]butyl}-N-{[6-(2- hydroxypropan-2-yl)pyridin- 3-yl]methyl}-1H-1,2,3- triazole-4-carboxamide |
| 175 | 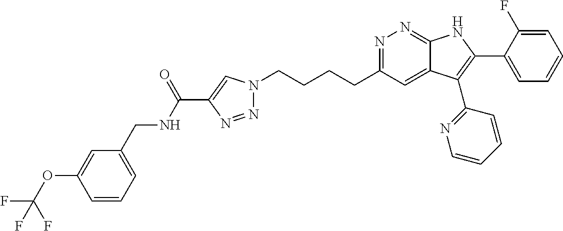 [SMILES: O=C(NCc1cccc(OC(F)(F)F)c1)c1cn(CCCCc2cc3c(-c4ccccn4)c(-c4ccccc4F)[nH]c3nn2)nn1] | 1-{4-[6-(2-fluorophenyl)-5- (pyridin-2-yl)-7H- pyrrolo[2,3-c]pyridazin-3- yl]butyl}-N-{[3- (trifluoromethoxy)phenyl] methyl}-1H-1,2,3-triazole-4- carboxamide |
| 176 | 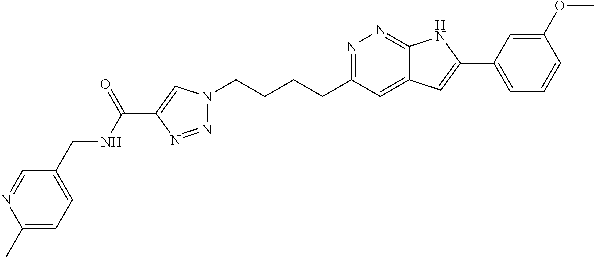 [SMILES: COc1cccc(-c2cc3cc(CCCCn4cc(C(=O)NCc5ccc(C)nc5)nn4)nnc3[nH]2)c1] | 1-{4-[6-(3-methoxyphenyl)- 7H-pyrrolo[2,3-c]pyridazin-3- yl]butyl}-N-[(6- methylpyridin-3-yl)methyl]- 1H-1,2,3-triazole-4- carboxamide |
| 177 | 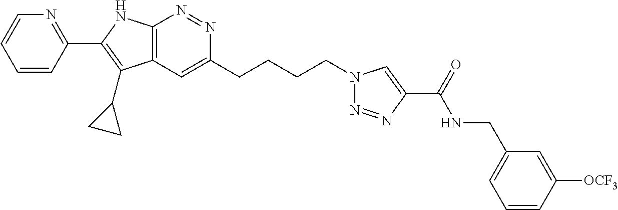 [SMILES: O=C(NCc1cccc(OC(F)(F)F)c1)c1cn(CCCCc2cc3c(C4CC4)c(-c4ccccn4)[nH]c3nn2)nn1] | 1-{4-[5-cyclopropyl-6- (pyridin-2-yl)-7H- pyrrolo[2,3-c]pyridazin-3- yl]butyl}-N-{[3- (trifluoromethoxy)phenyl] methyl}-1H-1,2,3-triazole-4- carboxamide |
| 178 | 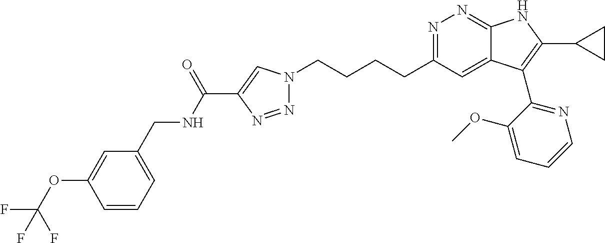 [SMILES: COc1cccnc1-c1c(C2CC2)[nH]c2nnc(CCCCn3cc(C(=O)NCc4cccc(OC(F)(F)F)c4)nn3)cc12] | 1-{4-[6-cyclopropyl-5-(3- methoxypyridin-2-yl)-7H- pyrrolo[2,3-c]pyridazin-3- yl]butyl}-N-{[3- (trifluoromethoxy)phenyl] methyl}-1H-1,2,3-triazole-4- carboxamide |
| 179 | 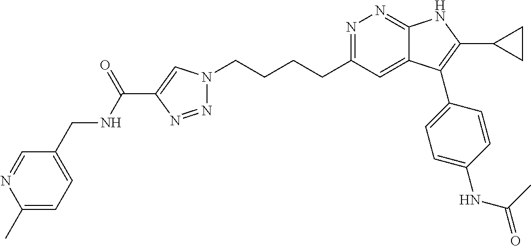 [SMILES: CC(=O)Nc1ccc(-c2c(C3CC3)[nH]c3nnc(CCCCn4cc(C(=O)NCc5ccc(C)nc5)nn4)cc23)cc1] | 1-{4-[6-cyclopropyl-5-(4- acetamidophenyl)-7H- pyrrolo[2,3-c]pyridazin-3- yl]butyl}-N-[(6- methylpyridin-3-yl)methyl]- 1H-1,2,3-triazole-4- carboxamide |
| 180 | 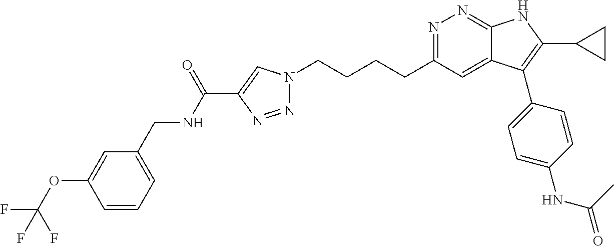 [SMILES: CC(=O)Nc1ccc(-c2c(C3CC3)[nH]c3nnc(CCCCn4cc(C(=O)NCc5cccc(OC(F)(F)F)c5)nn4)cc23)cc1] | 1-{4-[6-cyclopropyl-5-(4- acetamidophenyl)-7H- pyrrolo[2,3-c]pyridazin-3- yl]butyl}-N-{[3- (trifluoromethoxy)phenyl] methyl}-1H-1,2,3-triazole-4- carboxamide |
| 181 | 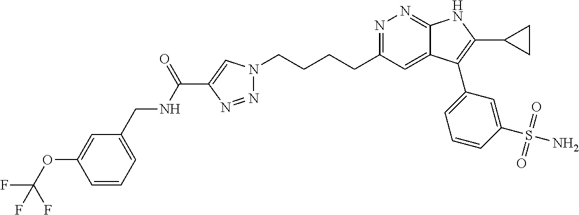 [SMILES: NS(=O)(=O)c1cccc(-c2c(C3CC3)[nH]c3nnc(CCCCn4cc(C(=O)NCc5cccc(OC(F)(F)F)c5)nn4)cc23)c1] | 1-{4-[6-cyclopropyl-5-(3- sulfamoylphenyl)-7H- pyrrolo[2,3-c]pyridazin-3- yl]butyl}-N-{[3- (trifluoromethoxy)phenyl] methyl}-1H-1,2,3-triazole-4- carboxamide |
| 182 | 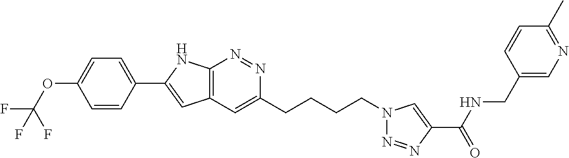 [SMILES: Cc1ccc(CNC(=O)c2cn(CCCCc3cc4cc(-c5ccc(OC(F)(F)F)cc5)[nH]c4nn3)nn2)cn1] | N-[(6-methylpyridin-3- yl)methyl]-1-(4-{6-[4- (trifluoromethoxy)phenyl]- 7H-pyrrolo[2,3-c]pyridazin-3- yl}butyl)-1H-1,2,3-triazole-4- carboxamide |
| 183 | 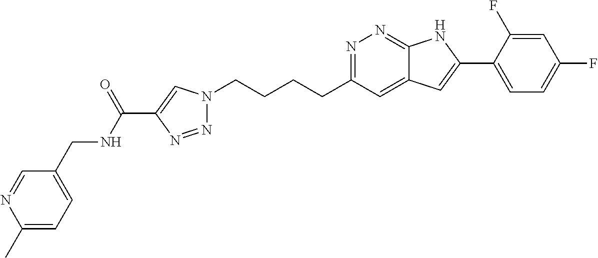 [SMILES: Cc1ccc(CNC(=O)c2cn(CCCCc3cc4cc(-c5ccc(F)cc5F)[nH]c4nn3)nn2)cn1] | 1-{4-[6-(2,4-difluorophenyl)- 7H-pyrrolo[2,3-c]pyridazin-3- yl]butyl}-N-[(6- methylpyridin-3-yl)methyl]- 1H-1,2,3-triazole-4- carboxamide |
| 184 | 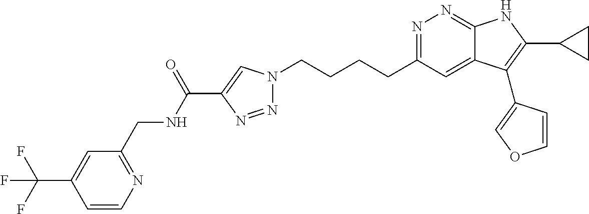 [SMILES: O=C(NCc1cc(C(F)(F)F)ccn1)c1cn(CCCCc2cc3c(-c4ccoc4)c(C4CC4)[nH]c3nn2)nn1] | 1-{4-[6-cyclopropyl-5-(furan- 3-yl)-7H-pyrrolo[2,3- c]pyridazin-3-yl]butyl}-N- {[4-(trifluoromethyl)pyridin- 2-yl]methyl}-1H-1,2,3- triazole-4-carboxamide |
| 185 | 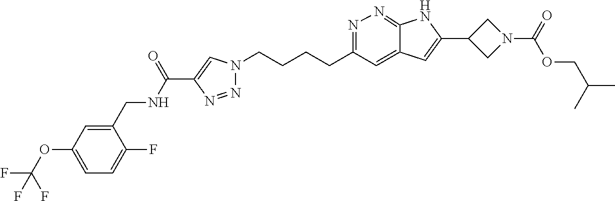 [SMILES: CC(C)COC(=O)N1CC(c2cc3cc(CCCCn4cc(C(=O)NCc5cc(OC(F)(F)F)ccc5F)nn4)nnc3[nH]2)C1] | 2-methylpropyl 3-(3-{4-[4- ({[2-fluoro-5- (trifluoromethoxy)phenyl] methyl}carbamoyl)-1H-1,2,3- triazol-1-yl]butyl)-7H- pyrrolo[2,3-c]pyridazin-6- yl)azetidine-1-carboxylate |
| 186 | 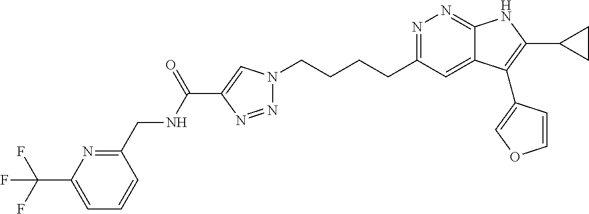 [SMILES: O=C(NCc1cccc(C(F)(F)F)n1)c1cn(CCCCc2cc3c(-c4ccoc4)c(C4CC4)[nH]c3nn2)nn1] | 1-{4-[6-cyclopropyl-5-(furan- 3-yl)-7H-pyrrolo[2,3- c]pyridazin-3-yl]butyl}-N- {[6-(trifluoromethyl)pyridin- 2-yl]methyl}-1H-1,2,3- triazole-4-carboxamide |
| 187 | 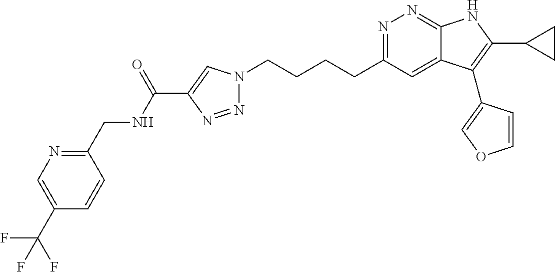 [SMILES: O=C(NCc1ccc(C(F)(F)F)cn1)c1cn(CCCCc2cc3c(-c4ccoc4)c(C4CC4)[nH]c3nn2)nn1] | 1-{4-[6-cyclopropyl-5-(furan- 3-yl)-7H-pyrrolo[2,3- c]pyridazin-3-yl]butyl}-N- {[5-(trifluoromethyl)pyridin- 2-yl]methyl}-1H-1,2,3- triazole-4-carboxamide |
| 188 | 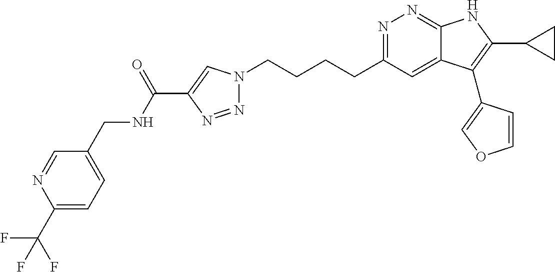 [SMILES: O=C(NCc1ccc(C(F)(F)F)nc1)c1cn(CCCCc2cc3c(-c4ccoc4)c(C4CC4)[nH]c3nn2)nn1] | 1-{4-[6-cyclopropyl-5-(furan- 3-yl)-7H-pyrrolo[2,3- c]pyridazin-3-yl]butyl}-N- {[6-(trifluoromethyl)pyridin- 3-yl]methyl}-1H-1,2,3- triazole-4-carboxamide |
| 189 | 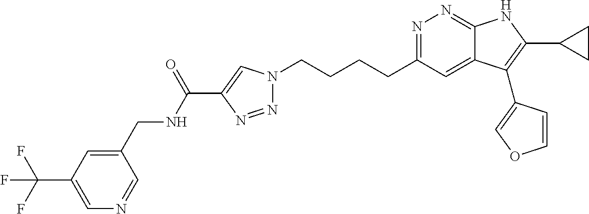 [SMILES: O=C(NCc1cncc(C(F)(F)F)c1)c1cn(CCCCc2cc3c(-c4ccoc4)c(C4CC4)[nH]c3nn2)nn1] | 1-{4-[6-cyclopropyl-5-(furan- 3-yl)-7H-pyrrolo[2,3- c]pyridazin-3-yl]butyl}-N- {[5-(trifluoromethyl)pyridin- 3-yl]methyl}-1H-1,2,3- triazole-4-carboxamide |
| 190 | 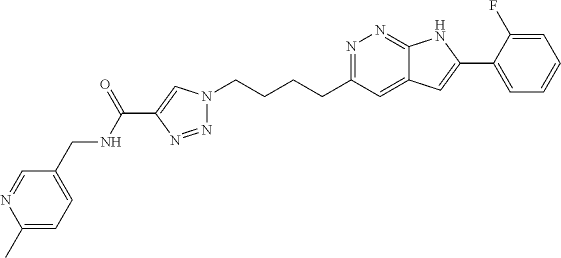 [SMILES: Cc1ccc(CNC(=O)c2cn(CCCCc3cc4cc(-c5ccccc5F)[nH]c4nn3)nn2)cn1] | 1-{4-[6-(2-fluorophenyl)-7H- pyrrolo[2,3-c]pyridazin-3- yl]butyl}-N-[(6- methylpyridin-3-yl)methyl]- 1H-1,2,3-triazole-4- carboxamide |
| 191 | 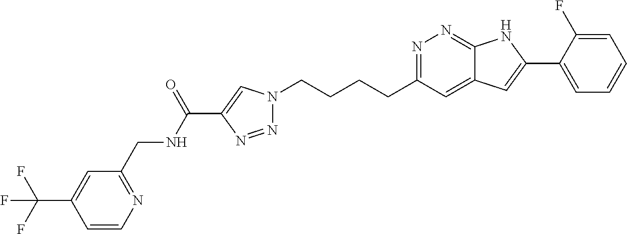 [SMILES: O=C(NCc1cc(C(F)(F)F)ccn1)c1cn(CCCCc2cc3cc(-c4ccccc4F)[nH]c3nn2)nn1] | 1-{4-[6-(2-trifluorophenyl)-7H- pyrrolo[2,3-c]pyridazin-3- yl]butyl}-N-{[4- (trifluoromethyl)pyridin-2- yl]methyl}-1H-1,2,3-triazole- 4-carboxamide |
| 192 | 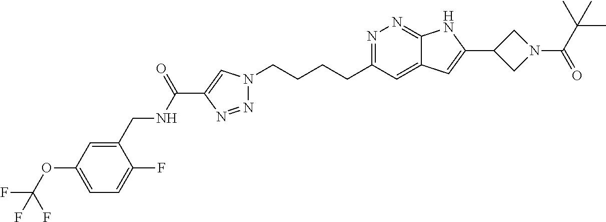 [SMILES: CC(C)(C)C(=O)N1CC(c2cc3cc(CCCCn4cc(C(=O)NCc5cc(OC(F)(F)F)ccc5F)nn4)nnc3[nH]2)C1] | 1-(4-(6-[1-(2,2- dimethylpropanoyl)azetidin- 3-yl]-7H-pyrrolo[2,3- c]pyridazin-3-yl}butyl)-N- {[2-fluoro-5- (trifluoromethoxy)phenyl] methyl}-1H-1,2,3-triazole-4- carboxamide |
| 193 | 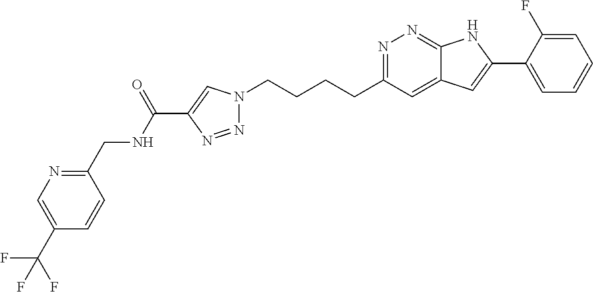 [SMILES: O=C(NCc1ccc(C(F)(F)F)cn1)c1cn(CCCCc2cc3cc(-c4ccccc4F)[nH]c3nn2)nn1] | 1-(4-[6-(2-fluorophenyl)-7H- pyrrolo[2,3-c]pyridazin-3- yl]butyl}-N-{[5- (trifluoromethyl)pyridin-2- yl]methyl}-1H-1,2,3-triazole- 4-carboxamide |
| 194 | 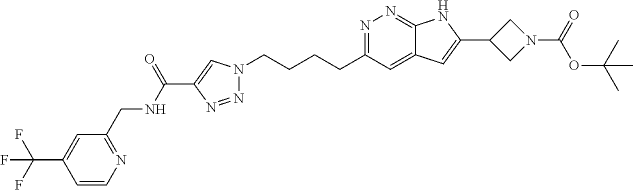 [SMILES: CC(C)(C)OC(=O)N1CC(c2cc3cc(CCCCn4cc(C(=O)NCc5cc(C(F)(F)F)ccn5)nn4)nnc3[nH]2)C1] | tert-butyl 3-(3-{4-[4-({[4- (trifluoromethyl)pyridin-2- yl]methyl}carbamoyl)-1H- 1,2,3-triazol-1-yl]butyl}-7H- pyrrolo[2,3-c]pyridazin-6- yl)azetidine-1-carboxylate |
| 195 | 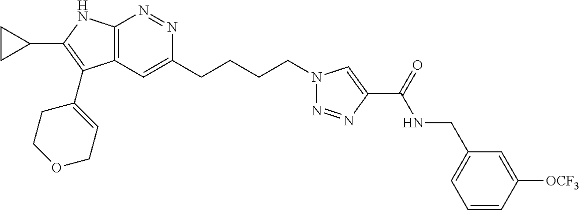 [SMILES: O=C(NCc1cccc(OC(F)(F)F)c1)c1cn(CCCCc2cc3c(C4=CCOCC4)c(C4CC4)[nH]c3nn2)nn1] | 1-{4-[6-cyclopropyl-5-(3,6- dihydro-2H-pyran-4-yl)-7H- pyrrolo[2,3-c]pyridazin-3- yl]butyl}-N-{[3- (trifluoromethoxy)phenyl] methyl}-1H-1,2,3-triazole-4- carboxamide |
| 196 | 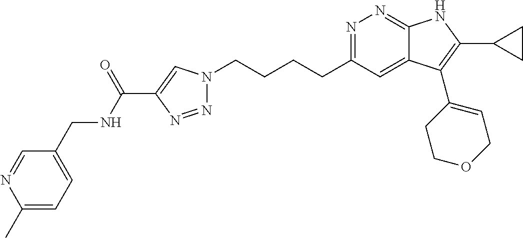 [SMILES: Cc1ccc(CNC(=O)c2cn(CCCCc3cc4c(C5=CCOCC5)c(C5CC5)[nH]c4nn3)nn2)cn1] | 1-{4-[6-cyclopropyl-5-(3,6- dihydro-2H-pyran-4-yl)-7H- pyrrolo[2,3-c]pyridazin-3- yl]butyl}-N-[(6- mcthylpyridin-3-yl)methyl]- 1H-1,2,3-triazole-4- carboxamide |
| 197 | 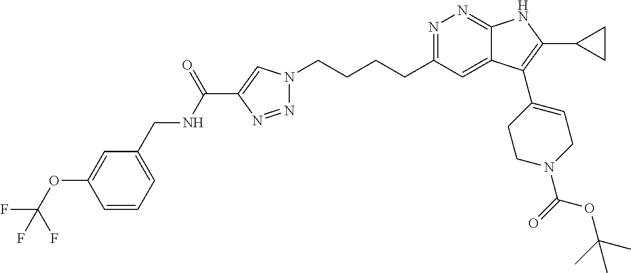 [SMILES: CC(C)(C)OC(=O)N1CC=C(c2c(C3CC3)[nH]c3nnc(CCCCn4cc(C(=O)NCc5cccc(OC(F)(F)F)c5)nn4)cc23)CC1] | tert-butyl 4-(6-cyclopropyl-3- {4-[4-({[3- (trifluoromethoxy)phenyl] methyl}carbamoyl)-1H-1,2,3- triazol-1-yl]butyl}-7H- pyrrolo[2,3-c]pyridazin-5-yl)- 1,2,3,6-tetrahydropyridine-1- carboxylate |
| 198 | 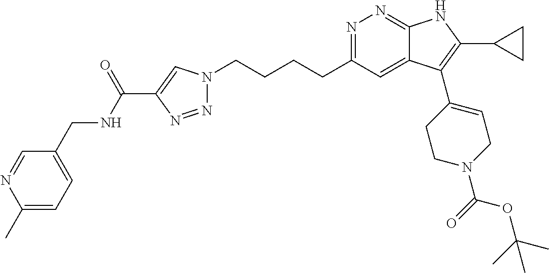 [SMILES: Cc1ccc(CNC(=O)c2cn(CCCCc3cc4c(C5=CCN(C(=O)OC(C)(C)C)CC5)c(C5CC5)[nH]c4nn3)nn2)cn1] | tert-butyl 4-(6-cyclopropyl-3- [4-(4-{[(6-methylpyridin-3- yl)methyl]carbamoyl}-1H- 1,2,3-triazol-1-yl)butyl]-7H- pyrrolo[2,3-c]pyridazin-5- yl}-1,2,3,6- tetrahydropyridine-1- carboxylate |
| 199 | 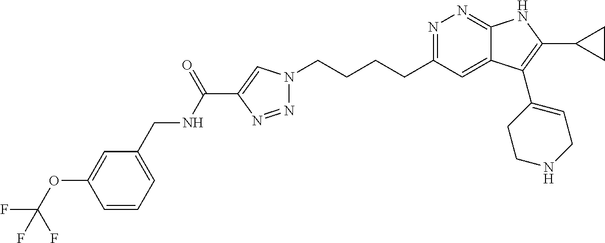 [SMILES: O=C(NCc1cccc(OC(F)(F)F)c1)c1cn(CCCCc2cc3c(C4=CCNCC4)c(C4CC4)[nH]c3nn2)nn1] | 1-{4-[6-cyclopropyl-5- (1,2,3,6-tetrahydropyridin-4- yl)-7H-pyrrolo[2,3- c]pyridazin-3-yl]butyl}-N- {[3- (trifluoromethoxy)phenyl] methyl}-1H-1,2,3-triazole-4- carboxamide |
| 200 | 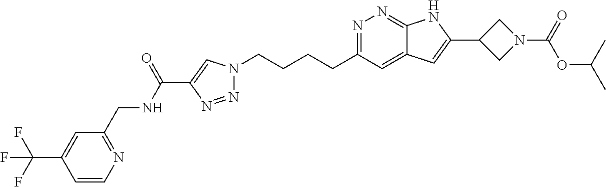 [SMILES: CC(C)OC(=O)N1CC(c2cc3cc(CCCCn4cc(C(=O)NCc5cc(C(F)(F)F)ccn5)nn4)nnc3[nH]2)C1] | propan-2-yl 3-(3-{4-[4-({[4- (trifluoromethyl)pyridin-2- yl]methyl}carbamoyl)-1H- 1,2,3-triazol-1-yl]butyl}-7H- pyrrolo[2,3-c]pyridazin-6- yl)azetidine-1-carboxylate |
| 201 | 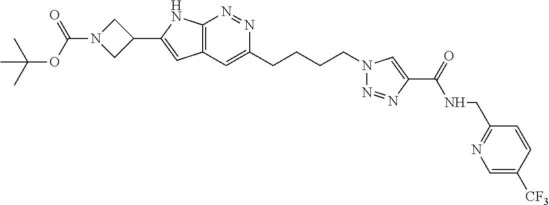 [SMILES: CC(C)(C)OC(=O)N1CC(c2cc3cc(CCCCn4cc(C(=O)NCc5ccc(C(F)(F)F)cn5)nn4)nnc3[nH]2)C1] | tert-butyl 3-(3-{4-[4-({[5- (trifluoromethyl)pyridin-2- yl]methyl)carbamoyl)-1H- 1,2,3-triazol-1-yl]butyl}-7H- pyrrolo[2,3-c]pyridazin-6- yl)azetidine-1-carboxylate |
| 202 | 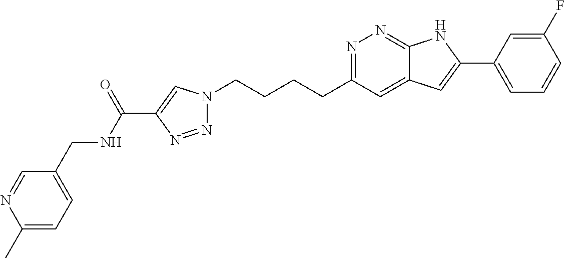 [SMILES: Cc1ccc(CNC(=O)c2cn(CCCCc3cc4cc(-c5cccc(F)c5)[nH]c4nn3)nn2)cn1] | 1-{4-[6-(3-fluorophenyl)-7H- pyrrolo[2,3-c]pyridazin-3- yl]butyl}-N-[(6- methylpyridin-3-yl)methyl]- 1H-1,2,3-triazole-4- carboxamide |
| 203 | 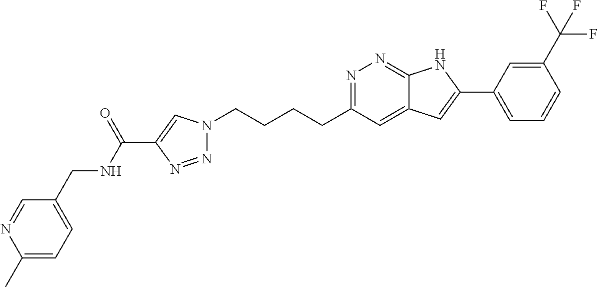 [SMILES: Cc1ccc(CNC(=O)c2cn(CCCCc3cc4cc(-c5cccc(C(F)(F)F)c5)[nH]c4nn3)nn2)cn1] | N-[(6-methylpyridin-3- yl)methyl]-1-(4-{6-[3- (trifluoromethyl)phenyl]-7H- pyrrolo[2,3-c]pyridazin-3- yl}butyl)-1H-1,2,3-triazole-4- carboxamide |
| 204 | 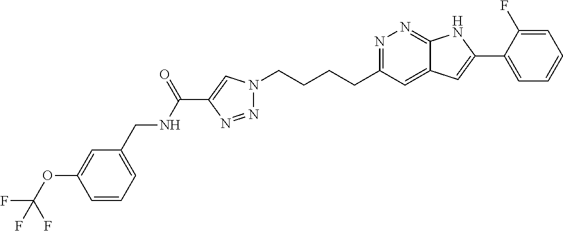 [SMILES: O=C(NCc1cccc(OC(F)(F)F)c1)c1cn(CCCCc2cc3cc(-c4ccccc4F)[nH]c3nn2)nn1] | 1-{4-[6-(2-fluorophenyl)-7H- pyrrolo[2,3-c]pyridazin-3- yl]butyl}-N-{[3- (trifluoromethoxy)phenyl] methyl}-1H-1,2,3-triazole-4- carboxamide |
| 205 | 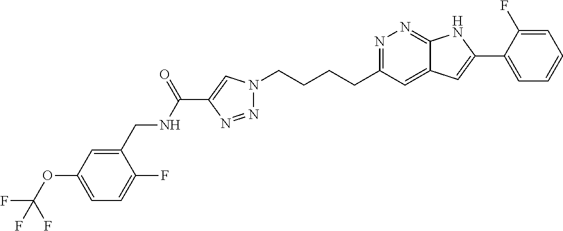 [SMILES: O=C(NCc1cc(OC(F)(F)F)ccc1F)c1cn(CCCCc2cc3cc(-c4ccccc4F)[nH]c3nn2)nn1] | N-{[2-fluoro-5- (trifluoromethoxy)phenyl] methyl}-1-(4-[6-(2- fluorophenyl)-7H- pyrrolo[2,3-c]pyridazin-3- yl]butyl)-1H-1,2,3-triazole-4- carboxamide |
| 206 | 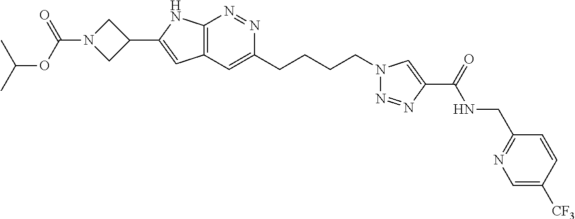 [SMILES: CC(C)OC(=O)N1CC(c2cc3cc(CCCCn4cc(C(=O)NCc5ccc(C(F)(F)F)cn5)nn4)nnc3[nH]2)C1] | propan-2-yl 3-(3-{4-[4-({[5- (trifluoromethyl)pyridin-2- yl]methyl}carbamoyl)-1H- 1,2,3-triazol-1-yl]butyl}-7H- pyrrolo[2,3-c]pyridazin-6- yl)azetidine-1-carboxylate |
| 207 | 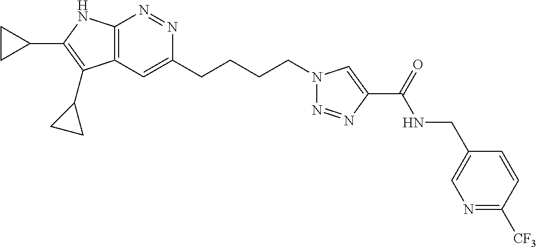 [SMILES: O=C(NCc1ccc(C(F)(F)F)nc1)c1cn(CCCCc2cc3c(C4CC4)c(C4CC4)[nH]c3nn2)nn1] | 1-(4-{5,6-dicyclopropyl-7H- pyrrolo[2,3-c]pyridazin-3- yl}butyl)-N-{[6- (trifluoromethyl)pyridin-3- yl]methyl}-1H-1,2,3-triazole- 4-carboxamide |
| 208 | 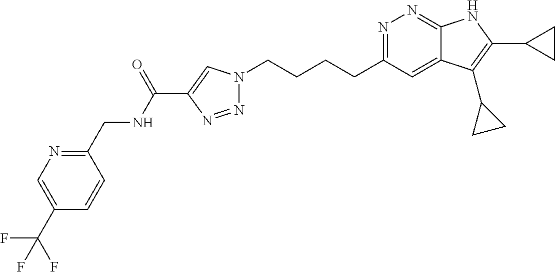 [SMILES: O=C(NCc1ccc(C(F)(F)F)cn1)c1cn(CCCCc2cc3c(C4CC4)c(C4CC4)[nH]c3nn2)nn1] | 1-(4-{5,6-dicyclopropyl-7H- pyrrolo[2,3-c]pyridazin-3- yl}butyl)-N-{[5- (trifluoromethyl)pyridin-2- yl]methyl}-1H-1,2,3-triazole- 4-carboxamide |
| 209 | 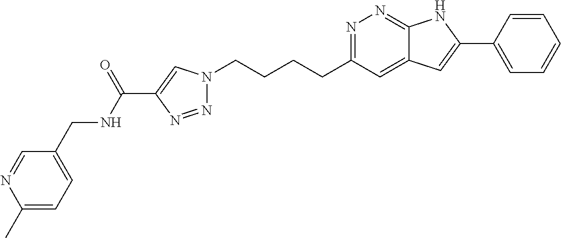 [SMILES: Cc1ccc(CNC(=O)c2cn(CCCCc3cc4cc(-c5ccccc5)[nH]c4nn3)nn2)cn1] | N-[(6-methylpyridin-3- yl)methyl]-1-(4-{6-phenyl- 7H-pyrrolo[2,3-c]pyridazin-3- yl}butyl)-1H-1,2,3-triazole-4- carboxamide |
| 210 | 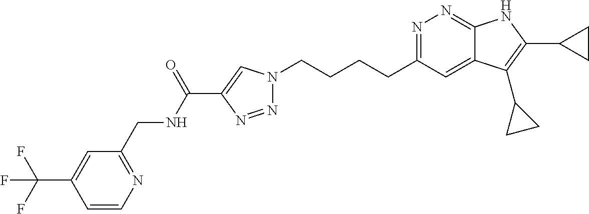 [SMILES: O=C(NCc1cc(C(F)(F)F)ccn1)c1cn(CCCCc2cc3c(C4CC4)c(C4CC4)[nH]c3nn2)nn1] | 1-(4-{5,6-dicyclopropyl-7H- pyrrolo[2,3-c]pyridazin-3- yl}butyl)-N-{[4- (trifluoromethyl)pyridin-2- yl]methyl}-1H-1,2,3-triazole- 4-carboxamide |
| 211 | 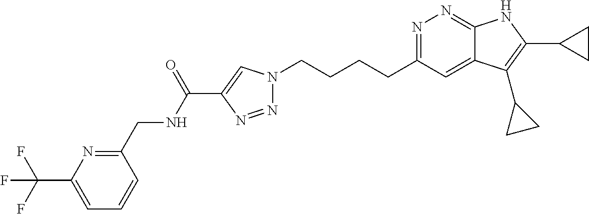 [SMILES: O=C(NCc1cccc(C(F)(F)F)n1)c1cn(CCCCc2cc3c(C4CC4)c(C4CC4)[nH]c3nn2)nn1] | 1-(4-{5,6-dicyclopropyl-7H- pyrrolo[2,3-c]pyridazin-3- yl}butyl)-N-{[6- (trifluoromethyl)pyridin-2- yl]methyl}-1H-1,2,3-triazole- 4-carboxamide |
| 212 | 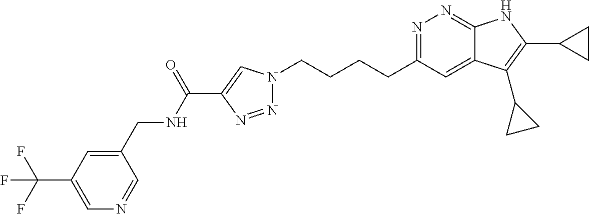 [SMILES: O=C(NCc1cncc(C(F)(F)F)c1)c1cn(CCCCc2cc3c(C4CC4)c(C4CC4)[nH]c3nn2)nn1] | 1-(4-{5,6-dicyclopropyl-7H- pyrrolo[2,3-c]pyridazin-3- yl}butyl)-N-{[5- (trifluoromethyl)pyridin-3- yl]methyl}-1H-1,2,3-triazole- 4-carboxamide |
| 213 | 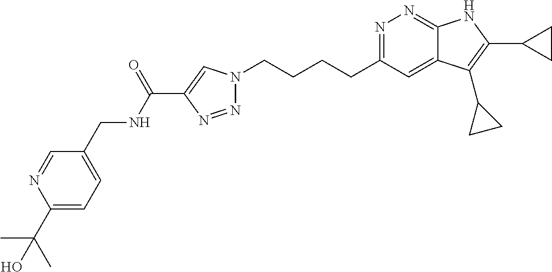 [SMILES: CC(C)(O)c1ccc(CNC(=O)c2cn(CCCCc3cc4c(C5CC5)c(C5CC5)[nH]c4nn3)nn2)cn1] | 1-(4-{5,6-dicyclopropyl-7H- pyrrolo[2,3-c]pyridazin-3- yl}butyl)-N-{[6-(2- hydroxypropan-2-yl)pyridin- 3-yl]methyl}-1H-1,2,3- triazole-4-carboxamide |
| 214 | 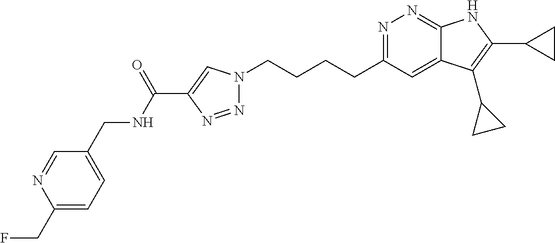 [SMILES: O=C(NCc1ccc(CF)nc1)c1cn(CCCCc2cc3c(C4CC4)c(C4CC4)[nH]c3nn2)nn1] | 1-(4-{5,6-dicyclopropyl-7H- pyrrolo[2,3-c]pyridazin-3- yl}butyl)-N-{[6- (fluoromethyl)pyridin-3- yl]methyl}-1H-1,2,3-triazole- 4-carboxamide |
| 215 | 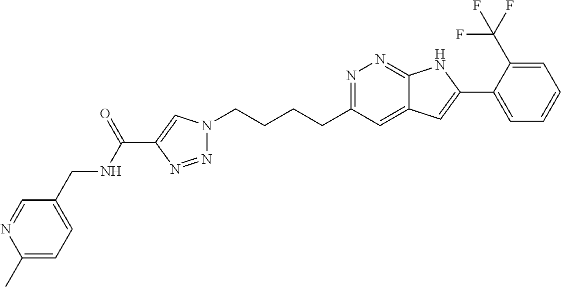 [SMILES: Cc1ccc(CNC(=O)c2cn(CCCCc3cc4cc(-c5ccccc5C(F)(F)F)[nH]c4nn3)nn2)cn1] | N-[(6-methylpyridin-3- yl)methyl]-1-(4-{6-[2- (trifluoromethyl)phenyl]-7H- pyrrolo[2,3-c]pyridazin-3- yl}butyl)-1H-1,2,3-triazole-4- carboxamide |
| 216 | 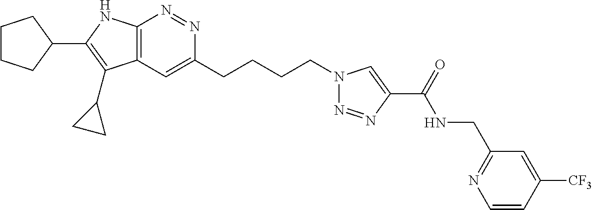 [SMILES: O=C(NCc1cc(C(F)(F)F)ccn1)c1cn(CCCCc2cc3c(C4CC4)c(C4CCCC4)[nH]c3nn2)nn1] | 1-(4-{6-cyclopentyl-5- cyclopropyl-7H-pyrrolo[2,3- c]pyridazin-3-yl}butyl)-N- {[4-(trifluoromethyl)pyridin- 2-yl]methyl}-1H-1,2,3- triazole-4-carboxamide |
| 217 | 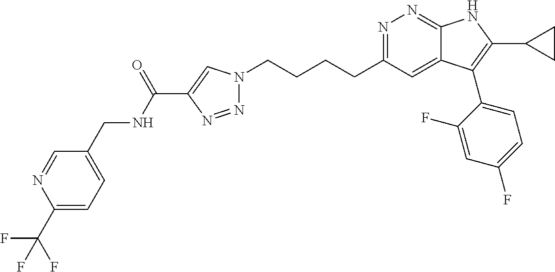 [SMILES: O=C(NCc1ccc(C(F)(F)F)nc1)c1cn(CCCCc2cc3c(-c4ccc(F)cc4F)c(C4CC4)[nH]c3nn2)nn1] | 1-{4-[6-cyclopropyl-5-(2,4- difluorophenyl)-7H- pyrrolo[2,3-c]pyridazin-3- yl]butyl}-N-{[6- (trifluoromethyl)pyridin-3- yl]methyl}-1H-1,2,3-triazole- 4-carboxamide |
| 218 | 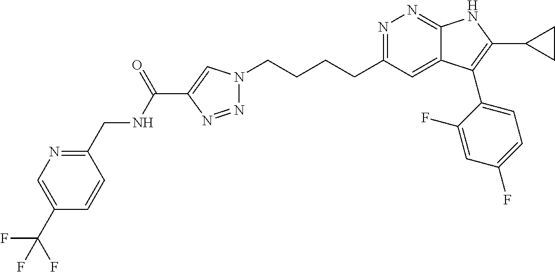 [SMILES: O=C(NCc1ccc(C(F)(F)F)cn1)c1cn(CCCCc2cc3c(-c4ccc(F)cc4F)c(C4CC4)[nH]c3nn2)nn1] | 1-{4-[6-cyclopropyl-5-(2,4- difluorophenyl)-7H- pyrrolo[2,3-c]pyridazin-3- yl]butyl}-N-{[5- (trifluoromethyl)pyridin-2- yl]methyl}-1H-1,2,3-triazole- 4-carboxamide |
| 219 | 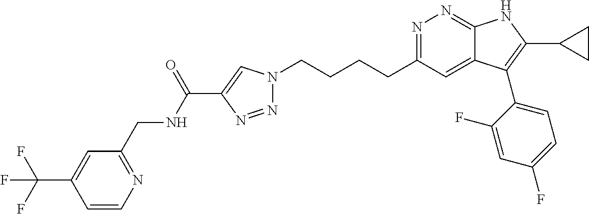 [SMILES: O=C(NCc1cc(C(F)(F)F)ccn1)c1cn(CCCCc2cc3c(-c4ccc(F)cc4F)c(C4CC4)[nH]c3nn2)nn1] | 1-{4-[6-cyclopropyl-5-(2,4- difluorophenyl)-7H- pyrrolo[2,3-c]pyridazin-3- yl]butyl}-N-{[4- (trifluoromethyl)pyridin-2- yl]methyl}-1H-1,2,3-triazole- 4-carboxamide |
| 220 | 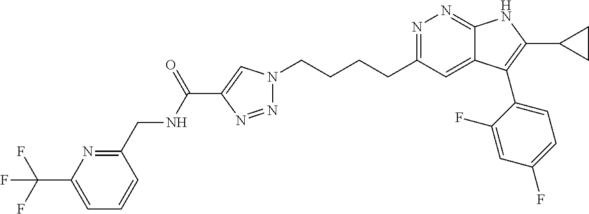 [SMILES: O=C(NCc1cccc(C(F)(F)F)n1)c1cn(CCCCc2cc3c(-c4ccc(F)cc4F)c(C4CC4)[nH]c3nn2)nn1] | 1-{4-[6-cyclopropyl-5-(2,4- difluorophenyl)-7H- pyrrolo[2,3-c]pyridazin-3- yl]butyl}-N-{[6- (trifluoromethyl)pyridin-2- yl]methyl}-1H-1,2,3-triazole- 4-carboxamide |
| 221 | 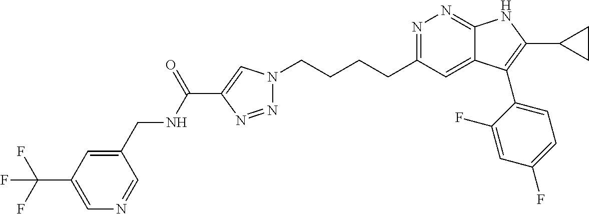 [SMILES: O=C(NCc1cncc(C(F)(F)F)c1)c1cn(CCCCc2cc3c(-c4ccc(F)cc4F)c(C4CC4)[nH]c3nn2)nn1] | 1-{4-[6-cyclopropyl-5-(2,4- difluorophenyl)-7H- pyrrolo[2,3-c]pyridazin-3- yl]butyl}-N-{[5- (trifluoromethyl)pyridin-3- yl]methyl}-1H-1,2,3-triazole- 4-carboxamide |
| 222 | 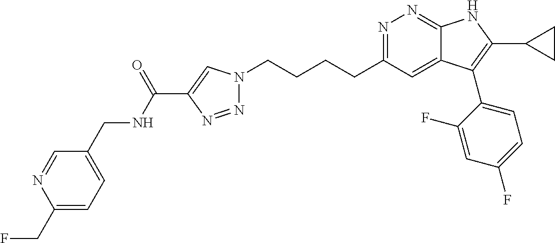 [SMILES: O=C(NCc1ccc(CF)nc1)c1cn(CCCCc2cc3c(-c4ccc(F)cc4F)c(C4CC4)[nH]c3nn2)nn1] | 1-{4-[6-cyclopropyl-5-(2,4- difluorophenyl)-7H- pyrrolo[2,3-c]pyridazin-3- yl]butyl}-N-{[6- (fluoromethyl)pyridin-3- yl]methyl}-1H-1,2,3-triazole- 4-carboxamide |
| 223 | 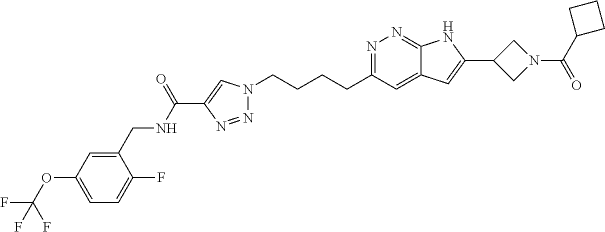 [SMILES: O=C(NCc1cc(OC(F)(F)F)ccc1F)c1cn(CCCCc2cc3cc(C4CN(C(=O)C5CCC5)C4)[nH]c3nn2)nn1] | 1-{4-[6-(1- cyclobutanecarbonylazetidin- 3-yl)-7H-pyrrolo[2,3- c]pyridazin-3-yl]butyl}-N- {[2-fluoro-5- (trifluoromethoxy)phenyl] methyl}-1H-1,2,3-triazole-4- carboxamide |
| 224 | 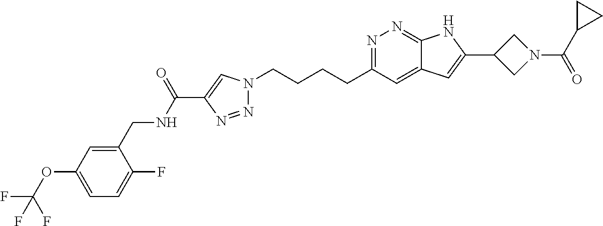 [SMILES: O=C(NCc1cc(OC(F)(F)F)ccc1F)c1cn(CCCCc2cc3cc(C4CN(C(=O)C5CC5)C4)[nH]c3nn2)nn1] | 1-{4-[6-(1- cyclopropanecarbonylazetidin- 3-yl)-7H-pyrrolo[2,3- c]pyridazin-3-yl]butyl}-N- {[2-fluoro-5- (trifluoromethoxy)phenyl] methyl}-1H-1,2,3-triazole-4- carboxamide |
| 225 | 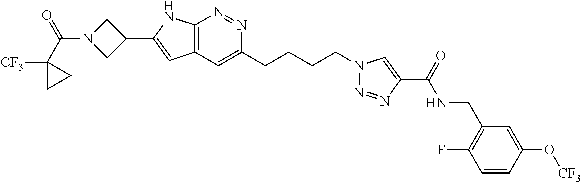 [SMILES: O=C(NCc1cc(OC(F)(F)F)ccc1F)c1cn(CCCCc2cc3cc(C4CN(C(=O)C5(C(F)(F)F)CC5)C4)[nH]c3nn2)nn1] | N-{[2-fluoro-5- (trifluoromethoxy)phenyl] methyl}-1-[4-(6-{1-[1- (trifluoromethyl)cyclopropane- carbonyl]azetidin-3-yl}-7H- pyrrolo[2,3-c]pyridazin-3- yl)butyl]-1H-1,2,3-triazole-4- carboxamide |
| 226 | 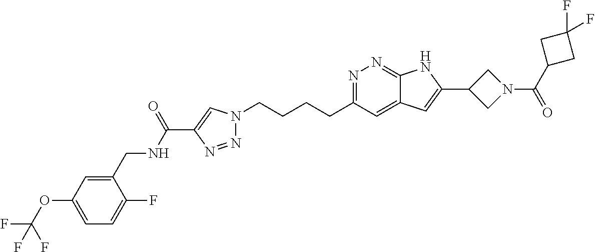 [SMILES: O=C(NCc1cc(OC(F)(F)F)ccc1F)c1cn(CCCCc2cc3cc(C4CN(C(=O)C5CC(F)(F)C5)C4)[nH]c3nn2)nn1] | 1-(4-{6-[1-(3,3- difluorocyclobutanecarbonyl) azetidin-3-yl]-7H- pyrrolo[2,3-c]pyridazin-3- yl}butyl)-N-{[2-fluoro-5- (trifluoromethoxy)phenyl] methyl}-1H-1,2,3-triazole-4- carboxamide |
| 227 | 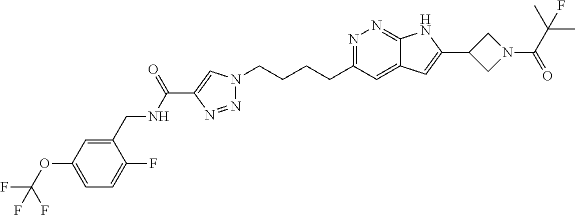 [SMILES: CC(C)(F)C(=O)N1CC(c2cc3cc(CCCCn4cc(C(=O)NCc5cc(OC(F)(F)F)ccc5F)nn4)nnc3[nH]2)C1] | 1-(4-{6-[1-(2-fluoro-2- methylpropanoyl)azetidin-3- yl]-7H-pyrrolo[2,3- c]pyridazin-3-yl}butyl)-N- {[2-fluoro-5- (trifluoromethoxy)phenyl] methyl}-1H-1,2,3-triazole-4- carboxamide |
| 228 | 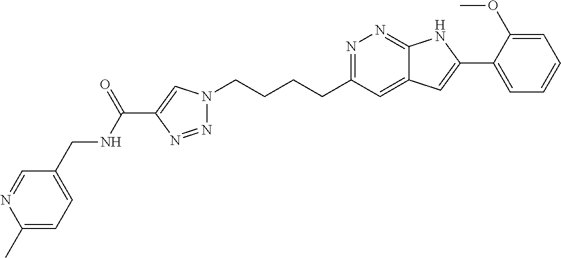 [SMILES: COc1ccccc1-c1cc2cc(CCCCn3cc(C(=O)NCc4ccc(C)nc4)nn3)nnc2[nH]1] | 1-{4-[6-(2-methoxyphenyl)- 7H-pyrrolo[2,3-c]pyridazin-3- yl]butyl}-N-[(6- methylpyridin-3-yl)methyl]- 1H-1,2,3-triazole-4- carboxamide |
| 229 | 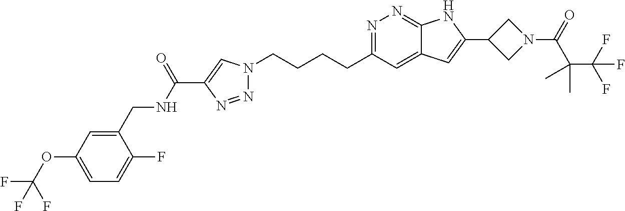 [SMILES: CC(C)(C(=O)N1CC(c2cc3cc(CCCCn4cc(C(=O)NCc5cc(OC(F)(F)F)ccc5F)nn4)nnc3[nH]2)C1)C(F)(F)F] | N-{[2-fluoro-5- (trifluoromethoxy)phenyl] methyl}-1-(4-{6-[1-(3,3,3- trifluoro-2,2- dimethylpropanoyl)azetidin- 3-yl}-7H-pyrrolo[2,3- c]pyridazin-3-yl}butyl)-1H- 1,2,3-triazole-4-carboxamide |
| 230 | 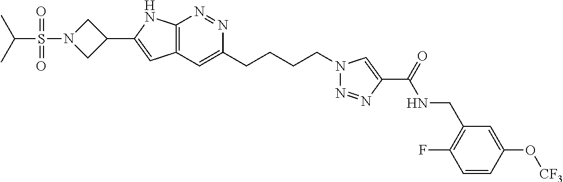 [SMILES: CC(C)S(=O)(=O)N1CC(c2cc3cc(CCCCn4cc(C(=O)NCc5cc(OC(F)(F)F)ccc5F)nn4)nnc3[nH]2)C1] | N-{[2-fluoro-5- (trifluoromethoxy)phenyl] methyl}-1-(4-{6-[1-(propane-2- sulfonyl)azetidin-3-yl]-7H- pyrrolo[2,3-c]pyridazin-3- yl}butyl)-1H-1,2,3-triazole-4- carboxamide |
| 231 | 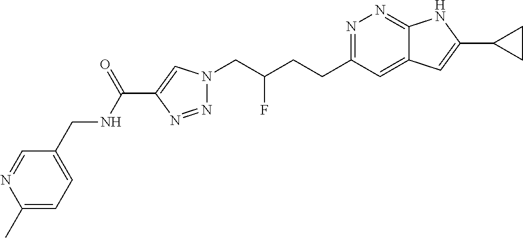 [SMILES: Cc1ccc(CNC(=O)c2cn(CC(F)CCc3cc4cc(C5CC5)[nH]c4nn3)nn2)cn1] | 1-(4-{6-cyclopropyl-7H- pyrrolo[2,3-c]pyridazin-3- yl)-2-fluorobutyl)-N-[(6- methylpyridin-3-yl)methyl]- 1H-1,2,3-triazole-4- carboxamide |
| 232 | 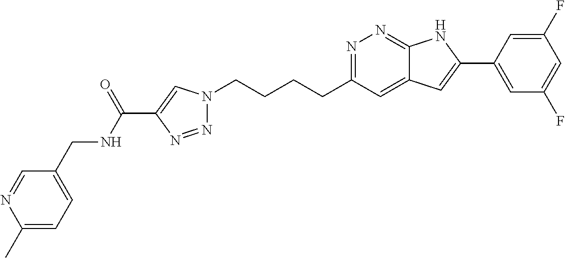 [SMILES: Cc1ccc(CNC(=O)c2cn(CCCCc3cc4cc(-c5cc(F)cc(F)c5)[nH]c4nn3)nn2)cn1] | 1-{4-[6-(3,5-difluorophenyl)- 7H-pyrrolo[2,3-c]pyridazin-3- yl]butyl}-N-[(6- methylpyridin-3-yl)methyl]- 1H-1,2,3-triazole-4- carboxamide |
| 233 | 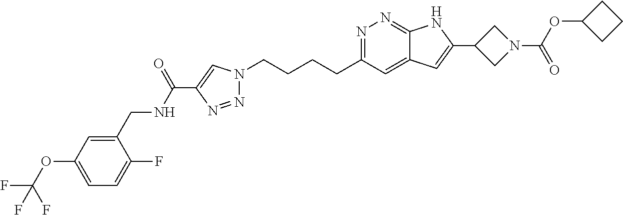 [SMILES: O=C(NCc1cc(OC(F)(F)F)ccc1F)c1cn(CCCCc2cc3cc(C4CN(C(=O)OC5CCC5)C4)[nH]c3nn2)nn1] | cyclobutyl 3-(3-{4-[4-({[2- fluoro-5- (trifluoromethoxy)phenyl] methyl}carbamoyl)-1H-1,2,3- triazol-1-yl]butyl}-7H- pyrrolo[2,3-c]pyridazin-6- yl)azetidine-1-carboxylate |
| 234 | 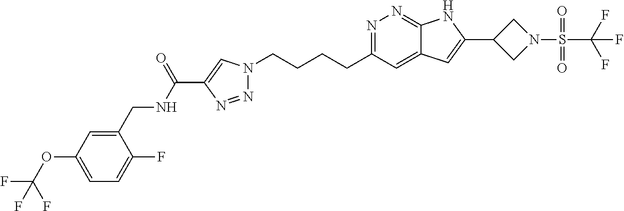 [SMILES: O=C(NCc1cc(OC(F)(F)F)ccc1F)c1cn(CCCCc2cc3cc(C4CN(S(=O)(=O)C(F)(F)F)C4)[nH]c3nn2)nn1] | N-{[2-fluoro-5- (trifluoromethoxy)phenyl] methyl}-1-{4-[6-(1- trifluoromethanesulfonyl- azetidin-3-yl)-7H-pyrrolo[2,3- c]pyridazin-3-yl]butyl}-1H- 1,2,3-triazole-4-carboxamide |
| 235 | 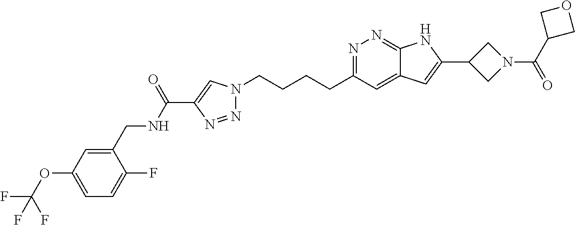 [SMILES: O=C(NCc1cc(OC(F)(F)F)ccc1F)c1cn(CCCCc2cc3cc(C4CN(C(=O)C5COC5)C4)[nH]c3nn2)nn1] | N-{[2-fluoro-5- (trifluoromethoxy)phenyl] methyl}-1-(4-{6-[1-(oxetane-3- carbonyl)azetidin-3-yl]-7H- pyrrolo[2,3-c]pyridazin-3- yl}butyl)-1H-1,2,3-triazole-4- carboxamide |
| 236 | 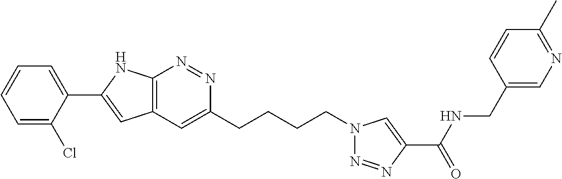 [SMILES: Cc1ccc(CNC(=O)c2cn(CCCCc3cc4cc(-c5ccccc5Cl)[nH]c4nn3)nn2)cn1] | 1-{4-[6-(2-chlorophenyl)-7H- pyrrolo[2,3-c]pyridazin-3- yl]butyl}-N-[(6- methylpyridin-3-yl)methyl]- 1H-1,2,3-triazole-4- carboxamide |
| 237 | 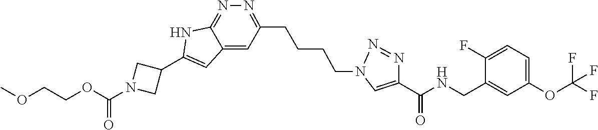 [SMILES: COCCOC(=O)N1CC(c2cc3cc(CCCCn4cc(C(=O)NCc5cc(OC(F)(F)F)ccc5F)nn4)nnc3[nH]2)C1] | 2-methoxyethyl 3-(3-{4-[4- ({[2-fluoro-5- (trifluoromethoxy)phenyl] methyl}carbamoyl)-1H-1,2,3- triazol-1-yl]butyl}-7H- pyrrolo[2,3-c]pyridazin-6- yl)azetidine-1-carboxylate |
| 238 | 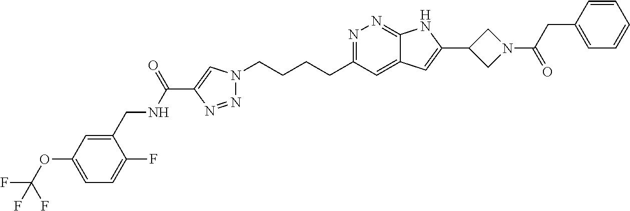 [SMILES: O=C(NCc1cc(OC(F)(F)F)ccc1F)c1cn(CCCCc2cc3cc(C4CN(C(=O)Cc5ccccc5)C4)[nH]c3nn2)nn1] | N-{[2-fluoro-5- (trifluoromethoxy)phenyl] methyl)-1-(4-{6-[1-(2- phenylacetyl)azetidin-3-yl]- 7H-pyrrolo[2,3-c]pyridazin-3- yl}butyl)-1H-1,2,3-triazole-4- carboxamide |
| 239 | 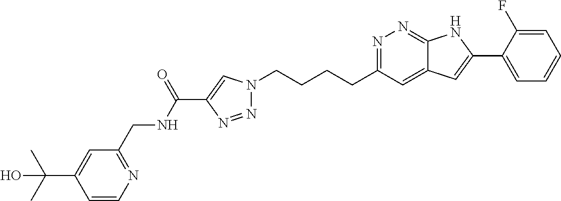 [SMILES: CC(C)(O)c1ccnc(CNC(=O)c2cn(CCCCc3cc4cc(-c5ccccc5F)[nH]c4nn3)nn2)c1] | 1-{4-[6-(2-fluorophenyl)-7H- pyrrolo[2,3-c]pyridazin-3- yl]butyl}-N-{[4-(2- hydroxypropan-2-yl)pyridin- 2-yl]methyl}-1H-1,2,3- triazole-4-carboxamide |
| 240 | 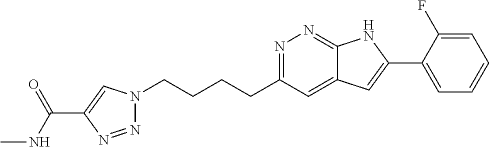 [SMILES: CNC(=O)c1cn(CCCCc2cc3cc(-c4ccccc4F)[nH]c3nn2)nn1] | 1-{4-[6-(2-fluorophenyl)-7H- pyrrolo[2,3-c]pyridazin-3- yl]butyl}-N-methyl-1H-1,2,3- triazole-4-carboxamide |
| 241 | 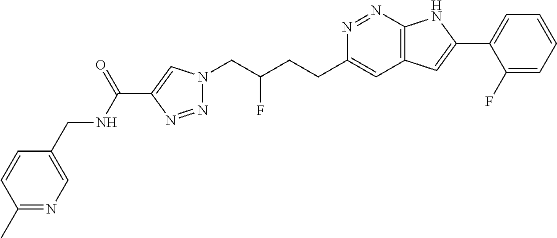 [SMILES: Cc1ccc(CNC(=O)c2cn(CC(F)CCc3cc4cc(-c5ccccc5F)[nH]c4nn3)nn2)cn1] | 1-{2-fluoro-4-[6-(2- fluorophenyl)-7H- pyrrolo[2,3-c]pyridazin-3- yl]butyl}-N-[(6- methylpyridin-3-yl)methyl]- 1H-1,2,3-triazole-4- carboxamide |
| 242 | 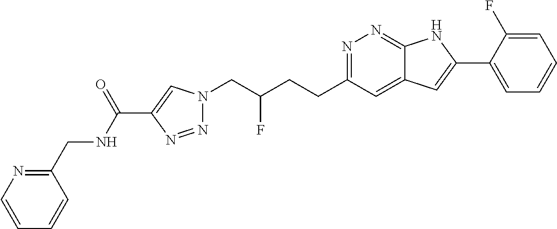 [SMILES: O=C(NCc1ccccn1)c1cn(CC(F)CCc2cc3cc(-c4ccccc4F)[nH]c3nn2)nn1] | 1-{2-fluoro-4-[6-(2- fluorophenyl)-7H- pyrrolo[2,3-c]pyridazin-3- yl]butyl}-N-(pyridin-2- ylmethyl)-1H-1,2,3-triazole- 4-carboxamide |
| 243 | 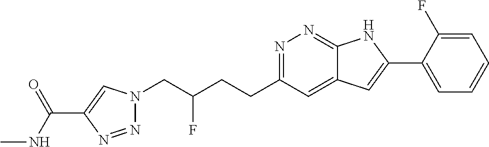 [SMILES: CNC(=O)c1cn(CC(F)CCc2cc3cc(-c4ccccc4F)[nH]c3nn2)nn1] | 1-{2-fluoro-4-[6-(2- fluorophenyl)-7H- pyrrolo[2,3-c]pyridazin-3- yl]butyl}-N-methyl-1H-1,2,3- triazole-4-carboxamide |
| 244 | 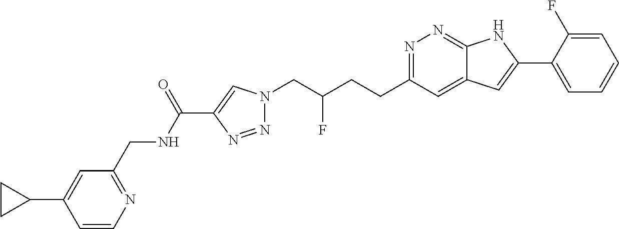 [SMILES: O=C(NCc1cc(C2CC2)ccn1)c1cn(CC(F)CCc2cc3cc(-c4ccccc4F)[nH]c3nn2)nn1] | N-[(4-cyclopropylpyridin-2- yl)methyl]-1-{2-fluoro-4-[6- (2-fluorophenyl)-7H- pyrrolo[2,3-c]pyridazin-3- yl]butyl}-1H-1,2,3-triazole-4- carboxamide |

[symbol]

indicates data missing or illegible when filed

Table 2 below reports the observed molecular ion (ES+) (Mass Spec) [M+H]+ of each Example, as well as the method by which each compound may be made by reference to each Example whose synthesis is substantially similar that one skilled in the art could produce the compound using, if necessary, variations know in the art.

TABLE 2
Observed Molecular Weight and Synthesis for Example

| | Calc. | Obs. | Synthesis, as in |
|---|---|---|---|
| Ex. No. | Mass | Mass | Ex. No. |
| 25 | 376 | 377 | 11 |
| 26 | 483 | 484 | 5 |
| 27 | 459 | 460 | 11 |
| 28 | 432 | 433 | 13 |
| 29 | 441 | 442 | 9 |
| 30 | 457 | 458 | 9 |
| 31 | 323 | 324 | 9 |
| 32 | 499 | 500 | 16 |
| 33 | 446 | 447 | 16 |
| 34 | 466 | 467 | 14 |
| 35 | 430 | 431 | 16 |
| 36 | 416 | 417 | 16 |
| 37 | 416 | 417 | 16 |
| 38 | 417 | 418 | 16 |
| 39 | 446 | 447 | 16 |
| 40 | 484 | 485 | 16 |
| 41 | 450 | 451 | 16 |
| 42 | 446 | 447 | 16 |
| 43 | 514 | 515 | 16 |
| 44 | 446 | 447 | 16 |
| 45 | 419 | 420 | 16 |
| 46 | 430 | 431 | 16 |
| 47 | 430 | 431 | 16 |
| 48 | 446 | 447 | 16 |
| 49 | 446 | 447 | 16 |
| 50 | 433 | 434 | 16 |
| 51 | 419 | 420 | 16 |
| 52 | 419 | 420 | 16 |
| 53 | 441 | 442 | 16 |
| 54 | 419 | 420 | 16 |
| 55 | 430 | 431 | 16 |
| 56 | 430 | 431 | 16 |
| 57 | 483 | 484 | 5 |
| 58 | 566 | 567 | 5 |
| 59 | 611 | 612 | 5 |
| 60 | 498 | 499 | 5 |
| 61 | 392 | 393 | 2 |
| 62 | 492 | 493 | 4 |
| 63 | 476 | 477 | 2 |
| 64 | 492 | 493 | 9 |
| 65 | 431 | 432 | 14 |
| 66 | 408 | 409 | 2 |
| 67 | 450 | 451 | 10 |
| 68 | 436 | 437 | 10 |
| 69 | 462 | 463 | 10 |
| 70 | 406 | 407 | 10 |
| 71 | 464 | 465 | 10 |
| 72 | 448 | 449 | 10 |
| 73 | 482 | 483 | 10 |
| 74 | 483 | 484 | 10 |
| 75 | 483 | 484 | 10 |
| 76 | 483 | 484 | 10 |
| 77 | 446 | 447 | 10 |
| 78 | 490 | 491 | 10 |
| 79 | 495 | 496 | 10 |
| 80 | 392 | 393 | 10 |
| 81 | 474 | 475 | 10 |
| 82 | 476 | 477 | 10 |
| 83 | 488 | 489 | 10 |
| 84 | 478 | 479 | 10 |
| 85 | 472 | 473 | 21 |
| 86 | 474 | 475 | 16 |
| 87 | 474 | 475 | 16 |
| 88 | 488 | 489 | 16 |
| 89 | 552 | 553 | 18 |
| 90 | 448 | 449 | 90 |
| 91 | 556 | 557 | 96 |
| 92 | 520 | 521 | 23 |
| 93 | 544 | 545 | 16 |
| 94 | 545 | 546 | 111 |
| 95 | 573 | 574 | 111 |
| 96 | 470 | 471 | 96 |
| 97 | 445 | 446 | 229 |
| 98 | 473 | 474 | 229 |
| 99 | 474 | 475 | 21 |
| 100 | 534 | 535 | 96 |
| 101 | 556 | 557 | 23 |
| 102 | 506 | 507 | 96 |
| 103 | 531 | 532 | 96 |
| 104 | 524 | 525 | 96 |
| 105 | 614 | 615 | 111 |
| 106 | 540 | 541 | 96 |
| 107 | 514 | 515 | 229 |
| 108 | 492 | 493 | 217 |
| 109 | 430 | 431 | 109 |
| 110 | 472 | 473 | 96 |
| 111 | 632 | 633 | 111 |
| 112 | 493 | 494 | 217 |
| 113 | 493 | 494 | 217 |
| 114 | 541 | 542 | 114 |
| 115 | 531 | 532 | 111 |
| 116 | 526 | 527 | 217 |
| 117 | 526 | 527 | 217 |
| 118 | 532 | 533 | 229 |
| 119 | 574 | 575 | 229 |
| 120 | 510 | 511 | 217 |
| 121 | 522 | 523 | 217 |
| 122 | 524 | 525 | 217 |
| 123 | 560 | 561 | 217 |
| 124 | 517 | 518 | 217 |
| 125 | 510 | 511 | 217 |
| 126 | 507 | 508 | 114 |
| 127 | 608 | 609 | 96 |
| 128 | 594 | 595 | 217 |
| 129 | 494 | 495 | 217 |
| 130 | 610 | 611 | 229 |
| 131 | 618 | 619 | 229 |
| 132 | 604 | 605 | 229 |
| 133 | 618 | 619 | 229 |
| 134 | 470 | 471 | 134 |
| 135 | 496 | 497 | 217 |
| 136 | 570 | 571 | 217 |
| 137 | 585 | 586 | 217 |
| 138 | 508 | 509 | 217 |
| 139 | 498 | 499 | 217 |
| 140 | 599 | 600 | 229 |
| 141 | 642 | 643 | 229 |
| 142 | 416 | 417 | 142 |
| 143 | 576 | 577 | 114 |
| 144 | 594 | 595 | 114 |
| 145 | 536 | 537 | 217 |
| 146 | 550 | 551 | 217 |
| 147 | 542 | 543 | 217 |
| 148 | 658 | 659 | 229 |
| 149 | 536 | 537 | 134 |
| 150 | 660 | 661 | 111 |
| 151 | 528 | 529 | 217 |
| 152 | 607 | 608 | 217 |
| 153 | 549 | 550 | 217 |
| 154 | 563 | 564 | 217 |
| 155 | 560 | 561 | 229 |
| 156 | 602 | 603 | 229 |
| 157 | 670 | 671 | 229 |
| 158 | 563 | 564 | 217 |
| 159 | 613 | 614 | 217 |
| 160 | 564 | 565 | 217 |
| 161 | 586 | 587 | 217 |
| 162 | 618 | 619 | 217 |
| 163 | 574 | 575 | 217 |
| 164 | 634 | 635 | 217 |
| 165 | 590 | 591 | 217 |
| 166 | 618 | 619 | 217 |
| 167 | 574 | 575 | 217 |
| 168 | 568 | 569 | 217 |
| 169 | 634 | 635 | 217 |
| 170 | 590 | 591 | 217 |
| 171 | 540 | 541 | 217 |
| 172 | 496 | 497 | 217 |
| 173 | 637 | 638 | 217 |
| 174 | 568 | 569 | 96 |
| 175 | 630 | 631 | 114 |
| 176 | 496 | 497 | 134 |
| 177 | 576 | 577 | 177 |
| 178 | 606 | 607 | 114 |
| 179 | 563 | 564 | 217 |
| 180 | 632 | 633 | 217 |
| 181 | 654 | 655 | 217 |
| 182 | 550 | 551 | 134 |
| 183 | 502 | 503 | 134 |
| 184 | 550 | 551 | 217 |
| 185 | 632 | 633 | 229 |
| 186 | 550 | 551 | 217 |
| 187 | 550 | 551 | 217 |
| 188 | 550 | 551 | 217 |
| 189 | 550 | 551 | 217 |
| 190 | 484 | 485 | 134 |
| 191 | 538 | 539 | 134 |
| 192 | 616 | 617 | 229 |
| 193 | 538 | 539 | 134 |
| 194 | 599 | 600 | 111 |
| 195 | 581 | 582 | 217 |
| 196 | 512 | 513 | 217 |
| 197 | 680 | 681 | 217 |
| 198 | 611 | 612 | 217 |
| 199 | 580 | 581 | 217 |
| 200 | 585 | 586 | 229 |
| 201 | 599 | 600 | 111 |
| 202 | 484 | 485 | 134 |
| 203 | 534 | 535 | 134 |
| 204 | 553 | 554 | 134 |
| 205 | 571 | 572 | 134 |
| 206 | 585 | 586 | 229 |
| 207 | 524 | 525 | 96 |
| 208 | 524 | 525 | 96 |
| 209 | 466 | 467 | 134 |
| 210 | 524 | 525 | 96 |
| 211 | 524 | 525 | 96 |
| 212 | 524 | 525 | 96 |
| 213 | 514 | 515 | 96 |
| 214 | 488 | 489 | 96 |
| 215 | 534 | 535 | 134 |
| 216 | 552 | 553 | 177 |
| 217 | 596 | 597 | 217 |
| 218 | 596 | 597 | 217 |
| 219 | 596 | 597 | 217 |
| 220 | 596 | 597 | 217 |
| 221 | 596 | 597 | 217 |
| 222 | 560 | 561 | 217 |
| 223 | 614 | 615 | 229 |
| 224 | 600 | 601 | 229 |
| 225 | 668 | 669 | 229 |
| 226 | 650 | 651 | 229 |
| 227 | 620 | 621 | 229 |
| 228 | 496 | 497 | 134 |
| 229 | 670 | 671 | 229 |
| 230 | 638 | 639 | 229 |
| 231 | 448 | 449 | 241 |
| 232 | 502 | 503 | 134 |
| 233 | 630 | 631 | 229 |
| 234 | 664 | 665 | 229 |
| 235 | 616 | 617 | 229 |
| 236 | 500 | 501 | 134 |
| 237 | 634 | 635 | 229 |
| 238 | 650 | 651 | 229 |
| 239 | 528 | 529 | 134 |
| 240 | 393 | 394 | 134 |
| 241 | 502 | 503 | 241 |
| 242 | 488 | 489 | 241 |
| 243 | 411 | 412 | 241 |
| 244 | 528 | 529 | 241 |

### Biological Activity Assays

The following are assays that may be used to evaluate the biological efficacy of compounds of Formula (I).

### GLS1 Enzymatic Activity Assay

The inhibition of purified recombinant human GAC by varying concentrations of inhibitors is assessed via a dual-coupled enzymatic assay. The glutamate produced by the glutaminase reaction is used by glutamate oxidase to produce α-ketoglutarate, ammonia, and hydrogen peroxide, with this hydrogen peroxide subsequently being used by horseradish peroxidase to produce resorufin in the presence of Amplex UltraRed. The assay buffer consisted of 50 mM Hepes (pH 7.4), 0.25 mM EDTA and 0.1 mM Triton X-100. GAC was incubated with potassium phosphate (10 minutes at room temperature) prior to incubation with inhibitor (10 minutes at room temperature). The final reaction conditions were as follows: 2 nM GAC, 50 mM potassium phosphate, 100 mU/mL glutamate oxidase (Sigma), 1 mM glutamine (Sigma), 100 mU/mL horseradish peroxidase (Sigma), 75 μM Amplex UltraRed (Life Technologies), and 1% (v/v) DMSO. The production of resorufin was monitored on a Perkin Elmer Envision plate reader (excitation 530 nm, emission 590 nm) either in a kinetics or endpoint mode (at 20 minutes). IC₅₀ values were calculated using a four-parameter logistic curve fit.

### Proliferation Assay

A549 cells were routinely maintained in RPMI 1640 media (Gibco catalog number 11875-093) supplemented with 10% dialyzed fetal bovine serum using a humidified incubator (37° C., 5% CO₂ and ambient O₂). In preparation for the viability assay, cells were inoculated into 384-well black CulturPlates (Perkin Elmer) at a density of 1000 cells/well in a volume of 40 uL. Following a 24-hour incubation at 37° C., 5% CO₂ and ambient O₂, cells were treated with compound (10 uL) in a final DMSO concentration of 0.5% (v/v). The microplates were then incubated for 72 hours (37° C., 5% CO₂ and ambient O₂). Cell Titer Fluor (Promega) was subsequently added (10 uL of 6× reagent) and mixed for 15 minutes at room temperature. The plates were then incubated for 30 minutes (37° C., 5% CO₂ and ambient O₂) and fluorescence was subsequently read on the Perkin Elmer Envision plate reader. EC₅₀ values were calculated using a four-parameter logistic curve fit.

Table 3 below reports the IC50 against GLS1 and the EC50 against A549 cell proliferation, both in nanomolar, and both wherein A=<100 nM, B=100-500 nM, and C<500-5000 nM, and D>5000 nM. Non-limiting examples include the following compounds and pharmaceutically acceptable salts thereof. “ND” indicates no data.

TABLE 3
Biological Data

| | GLS1 | A549 |
|---|---|---|
| Ex. | IC₅₀ | EC₅₀ |
| 1 | C | ND |
| 2 | C | C |
| 3 | ND | ND |
| 4 | A | B |
| 5 | A | B |
| 6 | A | C |
| 7 | C | ND |
| 8 | A | C |
| 9 | A | C |
| 10 | B | C |
| 11 | C | ND |
| 12 | B | C |
| 13 | B | C |
| 14 | A | C |
| 15 | B | C |
| 16 | B | B |
| 17 | B | C |
| 18 | A | B |
| 19 | A | C |
| 20 | C | D |
| 21 | B | D |
| 22 | C | ND |
| 23 | C | ND |
| 24 | B | D |
| 25 | C | ND |
| 26 | A | B |
| 27 | B | C |
| 28 | C | B |
| 29 | B | C |
| 30 | B | C |
| 31 | C | ND |
| 32 | A | B |
| 33 | B | C |
| 34 | A | C |
| 35 | A | B |
| 36 | B | B |
| 37 | B | C |
| 38 | C | C |
| 39 | B | B |
| 40 | A | C |
| 41 | B | C |
| 42 | A | C |
| 43 | A | C |
| 44 | B | C |
| 45 | B | C |
| 46 | A | C |
| 47 | B | C |
| 48 | A | C |
| 49 | C | C |
| 50 | B | C |
| 51 | B | C |
| 52 | B | C |
| 53 | B | C |
| 54 | C | D |
| 55 | B | C |
| 56 | B | C |
| 57 | A | ND |
| 58 | A | B |
| 59 | A | A |
| 60 | A | C |
| 61 | A | C |
| 62 | A | C |
| 63 | B | C |
| 64 | A | C |
| 65 | C | B |
| 66 | B | C |
| 67 | B | B |
| 68 | C | C |
| 69 | B | C |
| 70 | B | B |
| 71 | C | ND |
| 72 | B | D |
| 73 | A | C |
| 74 | A | B |
| 75 | B | C |
| 76 | A | B |
| 77 | B | C |
| 78 | B | C |
| 79 | B | C |
| 80 | B | C |
| 81 | B | D |
| 82 | B | C |
| 83 | B | D |
| 84 | B | C |
| 85 | A | A |
| 86 | A | B |
| 87 | B | C |
| 88 | A | C |
| 89 | A | B |
| 90 | A | C |
| 91 | A | B |
| 92 | A | B |
| 93 | A | A |
| 94 | A | B |
| 95 | A | B |
| 96 | A | B |
| 97 | B | C |
| 98 | C | D |
| 99 | B | C |
| 100 | A | B |
| 101 | A | B |
| 102 | A | C |
| 103 | A | C |
| 104 | A | C |
| 105 | A | A |
| 106 | A | B |
| 107 | B | C |
| 108 | A | B |
| 109 | B | C |
| 110 | A | B |
| 111 | A | A |
| 112 | A | C |
| 113 | A | C |
| 114 | A | C |
| 115 | B | C |
| 116 | A | C |
| 117 | A | B |
| 118 | A | C |
| 119 | A | C |
| 120 | A | B |
| 121 | A | B |
| 122 | A | A |
| 123 | A | B |
| 124 | A | C |
| 125 | A | B |
| 126 | B | C |
| 127 | A | B |
| 128 | A | A |
| 129 | B | C |
| 130 | A | B |
| 131 | A | A |
| 132 | A | A |
| 133 | A | B |
| 134 | A | A |
| 135 | A | C |
| 136 | A | C |
| 137 | A | C |
| 138 | A | C |
| 139 | A | B |
| 140 | A | C |
| 141 | A | B |
| 142 | C | ND |
| 143 | A | A |
| 144 | A | A |
| 145 | A | B |
| 146 | A | A |
| 147 | A | A |
| 148 | A | A |
| 149 | A | C |
| 150 | A | A |
| 151 | A | B |
| 152 | A | C |
| 153 | A | C |
| 154 | A | C |
| 155 | B | C |
| 156 | A | B |
| 157 | A | B |
| 158 | A | C |
| 159 | A | C |
| 160 | A | B |
| 161 | A | B |
| 162 | A | B |
| 163 | A | B |
| 164 | A | B |
| 165 | A | B |
| 166 | A | B |
| 167 | A | B |
| 168 | A | C |
| 169 | A | B |
| 170 | A | B |
| 171 | A | A |
| 172 | A | A |
| 173 | A | B |
| 174 | A | B |
| 175 | A | C |
| 176 | A | B |
| 177 | A | C |
| 178 | A | B |
| 179 | A | C |
| 180 | A | C |
| 181 | A | C |
| 182 | A | B |
| 183 | A | B |
| 184 | A | A |
| 185 | A | A |
| 186 | A | A |
| 187 | A | B |
| 188 | A | B |
| 189 | A | A |
| 190 | A | A |
| 191 | A | B |
| 192 | A | A |
| 193 | A | B |
| 194 | A | A |
| 195 | A | A |
| 196 | A | B |
| 197 | A | A |
| 198 | A | C |
| 199 | B | ND |
| 200 | A | B |
| 201 | A | B |
| 202 | A | B |
| 203 | A | B |
| 204 | A | C |
| 205 | A | B |
| 206 | A | B |
| 207 | A | B |
| 208 | A | B |
| 209 | A | B |
| 210 | A | B |
| 211 | A | B |
| 212 | A | B |
| 213 | A | B |
| 214 | A | B |
| 215 | A | B |
| 216 | A | B |
| 217 | A | B |
| 218 | A | B |
| 219 | A | A |
| 220 | A | B |
| 221 | A | A |
| 222 | A | B |
| 223 | A | A |
| 224 | A | B |
| 225 | A | A |
| 226 | A | B |
| 227 | A | A |
| 228 | A | A |
| 229 | A | A |
| 230 | A | B |
| 231 | A | B |
| 232 | A | B |
| 233 | A | A |
| 234 | A | A |
| 235 | A | C |
| 236 | A | B |
| 237 | A | B |
| 238 | A | A |
| 239 | A | B |
| 240 | B | B |
| 241 | A | A |
| 242 | A | B |
| 243 | B | C |
| 244 | A | A |

### OTHER EMBODIMENTS

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

What is claimed is:

1. A compound of structural Formula I: 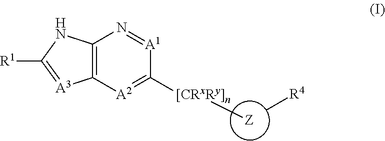
or a salt thereof, wherein:
n is chosen from 3, 4, and 5;
each R^{x} and R^{y} is independently chosen from alkyl, cyano, H, and halo, wherein two R^{x} groups together with the atoms to which they are attached optionally form a cycloalkyl ring;
A¹ and A² are independently chosen from N and CH;
A³ is chosen from N and CR²;
R¹ is chosen from alkenyl, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, C(O)N(R³)₂, and C(O)C(R³)₃, wherein R¹ may be optionally substituted with between 0 and 3 R^{z} groups;
R² is chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, C(O)N(R³)₂, C(O)C(R³)₃, C(O)OH, C(O)OC(R³)₃, wherein R¹ and R² together with the atoms to which they are attached optionally form an form an aryl, cycloalkyl, heteroaryl, or heterocycloalkyl ring, which may be optionally substituted with between 0 and 3 R^{z} groups;
each R³ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein each R³ may be optionally substituted with between 0 and 3 R^{z} groups, wherein two R³ groups together with the atoms to which they are attached optionally form an aryl, cycloalkyl, heteroaryl, or heterocycloalkyl ring, which may be optionally substituted with between 0 and 3 R^{z} groups;
R⁴ is chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, N(R³)₂, NR³C(O)C(R³)₃, NR³C(O)OC(R³)₃, NR³C(O)N(R³)₂, NR³S(O)C(R³)₃, NR³S(O)₂C(R³)₃, C(O)N(R³)₂, S(O)N(R³)₂, S(O)₂N(R³)₂, C(O)C(R³)₃, SC(R³)₃, S(O)C(R³)₃, and S(O)₂C(R³)₃, wherein R⁴ may be optionally substituted with between 0 and 3 R^{z} groups;
each R^{z} group is independently chosen from alkenyl, alkoxy, alkyl, aryl, arylalkyl, cyano, cycloalkyl, cycloalkylalkyl, halo, haloalkyl, heteroaryl, heteroarylalkyl, heterocycloalkyl, heterocycloalkylalkyl, hydroxyl, oxo, N(R⁶)₂, NR⁶C(O)C(R⁶)₃, NR⁶C(O)OC(R⁶)₃, NR⁶C(O)N(R⁶)₂, NR⁶S(O)C(R⁶)₃, NR⁶S(O)₂C(R⁶)₃, C(O)N(R⁶)₂, S(O)N(R⁶)₂, S(O)₂N(R⁶)₂, C(O)C(R⁶)₃, SC(R⁶)₃, S(O)C(R⁶)₃, and S(O)₂C(R⁶)₃;
each R⁶ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein two R⁶ groups together with the atoms to which they are attached optionally form an aryl, cycloalkyl, heteroaryl, or heterocycloalkyl ring, which may be optionally substituted with between 0 and 3 R^{x} groups; and Z is heteroaryl, which may be optionally substituted.

2. The compound as recited in claim 1, wherein the compound has structural Formula II 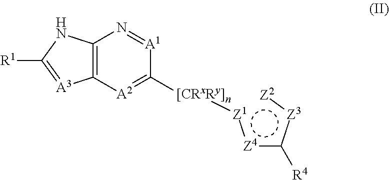
or a salt thereof, wherein:
n is chosen from 3, 4, and 5;
each R^{x} and R^{y} is independently chosen from alkyl, cyano, H, and halo, wherein two R^{x} groups together with the atoms to which they are attached optionally form a cycloalkyl ring;
A¹ and A² are independently chosen from N and CH;
A³ is chosen from N and CR²;
Z¹ is chosen from C and N;
Z², Z³, and Z⁴ are independently chosen from N, O, S, and CH, wherein at least one of Z¹, Z², Z³, and Z⁴ is chosen from N, O, and S;
R¹ is chosen from alkenyl, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, C(O)N(R³)₂, and C(O)C(R³)₃, wherein R¹ may be optionally substituted with between 0 and 3 R^{z} groups;
R² is chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, C(O)N(R³)₂, C(O)C(R³)₃, C(O)OH, C(O)OC(R³)₃, wherein R¹ and R² together with the atoms to which they are attached optionally form an form an aryl, cycloalkyl, heteroaryl, or heterocycloalkyl ring, which may be optionally substituted with between 0 and 3 R^{z} groups;
each R³ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein each R³ may be optionally substituted with between 0 and 3 R^{z} groups, wherein two R³ groups together with the atoms to which they are attached optionally form an aryl, cycloalkyl, heteroaryl, or heterocycloalkyl ring, which may be optionally substituted with between 0 and 3 R^{z} groups;
R⁴ is chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, N(R³)₂, NR³C(O)C(R³)₃, NR³C(O)OC(R³)₃, NR³C(O)N(R³)₂, NR³S(O)C(R³)₃, NR³S(O)₂C(R³)₃, C(O)N(R³)₂, S(O)N(R³)₂, S(O)₂N(R³)₂, C(O)C(R³)₃, SC(R³)₃, S(O)C(R³)₃, and S(O)₂C(R³)₃, wherein R⁴ may be optionally substituted with between 0 and 3 R^{z} groups;
each R^{z} group is independently chosen from alkenyl, alkoxy, alkyl, aryl, arylalkyl, cyano, cycloalkyl, cycloalkylalkyl, halo, haloalkyl, heteroaryl, heteroarylalkyl, heterocycloalkyl, heterocycloalkylalkyl, hydroxyl, oxo, N(R⁶)₂, NR⁶C(O)C(R⁶)₃, NR⁶C(O)OC(R⁶)₃, NR⁶C(O)N(R⁶)₂, NR⁶S(O)C(R⁶)₃, NR⁶S(O)₂C(R⁶)₃, C(O)N(R⁶)₂, S(O)N(R⁶)₂, S(O)₂N(R⁶)₂, C(O)C(R⁶)₃, SC(R⁶)₃, S(O)C(R⁶)₃, and S(O)₂C(R⁶)₃; and
each R⁶ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein two R⁶ groups together with the atoms to which they are attached optionally form an aryl, cycloalkyl, heteroaryl, or heterocycloalkyl ring, which may be optionally substituted with between 0 and 3 R^{x} groups.

3. The compound as recited in claim 2, wherein:
n is 4; and
A¹, A², and A³ are CH.

4. The compound as recited in claim 2, wherein:
n is 4;
A¹ and A³ are N; and
A² is CH.

5. The compound as recited in claim 2, wherein:
n is 4;
A¹ and A² are CH; and
A³ is N.

6. The compound as recited in claim 2, wherein:
n is 4;
A¹ is N;
A² is CH; and
A³ is CR².

7. The compound as recited in claim 2, wherein:
Z¹ is C;
Z² and Z³ are N;
Z⁴ is S; and
R⁴ is chosen from N(R³)₂, NR³C(O)C(R³)₃, NR³C(O)OC(R³)₃, and NR³C(O)N(R³)₂.

8. The compound as recited in claim 2, wherein:
n is 4;
A¹ is N;
A² is CH;
A³ is CR²;
Z¹ is C;
Z² and Z³ are N;
Z⁴ is S; and
R⁴ is chosen from N(R³)₂, NR³C(O)C(R³)₃, NR³C(O)OC(R³)₃, and NR³C(O)N(R³)₂.

9. The compound as recited in claim 2, wherein:
Z¹ is C;
Z² and Z³ are N;
Z⁴ is S; and
R⁴ is C(O)N(R³)₂.

10. The compound as recited in claim 2, wherein:
n is 4;
A¹ is N;
A² is CH;
A³ is CR²;
Z¹ is C;
Z² and Z³ are N;
Z⁴ is S; and
R⁴ is C(O)N(R³)₂.

11. The compound as recited in claim 2, wherein:
Z¹, Z², and Z³ are N;
Z⁴ is CH; and
R⁴ is chosen from N(R³)₂, NR³C(O)C(R³)₃, NR³C(O)OC(R³)₃, and NR³C(O)N(R³)₂.

12. The compound as recited in claim 2, wherein:
n is 4;
A¹ is N;
A² is CH;
A³ is CR²;
Z¹, Z², and Z³ are N;
Z⁴ is CH; and
R⁴ is chosen from N(R³)₂, NR³C(O)C(R³)₃, NR³C(O)OC(R³)₃, and NR³C(O)N(R³)₂.

13. The compound as recited in claim 2, wherein:
Z¹, Z², and Z³ are N;
Z⁴ is CH; and
R⁴ is C(O)N(R³)₂.

14. The compound as recited in claim 2, wherein:
n is 4;
A¹ is N;
A² is CH;
A³ is CR²;
Z¹, Z², and Z³ are N;
Z⁴ is CH; and
R⁴ is C(O)N(R³)₂.

15. The compound as recited in claim 1, wherein the compound has structural Formula III: 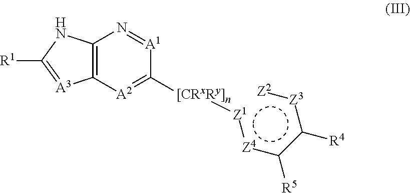
or a salt thereof, wherein:
n is chosen from 3, 4, and 5;
each R^{x} and R^{y} is independently chosen from alkyl, cyano, H, and halo, wherein two R^{x} groups together with the atoms to which they are attached optionally form a cycloalkyl ring;
A¹ and A² are independently chosen from N and CH;
A³ is chosen from N and CR²;
Z¹ is chosen from C and N;
Z² is chosen from N, CH, and C(O);
Z³, and Z⁴ are independently chosen from N and CH, wherein at least one of Z¹, Z², Z³, and Z⁴ is N;
R¹ is chosen from alkenyl, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, C(O)N(R³)₂, and C(O)C(R³)₃, wherein R¹ may be optionally substituted with between 0 and 3 R^{z} groups;
R² is chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, C(O)N(R³)₂, C(O)C(R³)₃, C(O)OH, C(O)OC(R³)₃, wherein R¹ and R² together with the atoms to which they are attached optionally form an form an aryl, cycloalkyl, heteroaryl, or heterocycloalkyl ring, which may be optionally substituted with between 0 and 3 R^{z} groups;
each R³ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein each R³ may be optionally substituted with between 0 and 3 R^{z} groups, wherein two R³ groups together with the atoms to which they are attached optionally form an aryl, cycloalkyl, heteroaryl, or heterocycloalkyl ring, which may be optionally substituted with between 0 and 3 R^{z} groups;
R⁴ is chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, hydroxyl, N(R³)₂, NR³C(O)C(R³)₃, NR³C(O)OC(R³)₃, NR³C(O)N(R³)₂, NR³S(O)C(R³)₃, NR³S(O)₂C(R³)₃, C(O)N(R³)₂, S(O)N(R³)₂, S(O)₂N(R³)₂, C(O)C(R³)₃, SC(R³)₃, S(O)C(R³)₃, and S(O)₂C(R³)₃, wherein R⁴ may be optionally substituted with between 0 and 3 R^{z} groups;
each R^{z} group is independently chosen from alkenyl, alkoxy, alkyl, aryl, arylalkyl, cyano, cycloalkyl, cycloalkylalkyl, halo, haloalkyl, heteroaryl, heteroarylalkyl, heterocycloalkyl, heterocycloalkylalkyl, hydroxyl, oxo, N(R⁶)₂, NR⁶C(O)C(R⁶)₃, NR⁶C(O)OC(R⁶)₃, NR⁶C(O)N(R⁶)₂, NR⁶S(O)C(R⁶)₃, NR⁶S(O)₂C(R⁶)₃, C(O)N(R⁶)₂, S(O)N(R⁶)₂, S(O)₂N(R⁶)₂, C(O)C(R⁶)₃, SC(R⁶)₃, S(O)C(R⁶)₃, and S(O)₂C(R⁶)₃; and
each R⁶ is independently chosen from alkenyl, alkoxy, alkyl, aryl, arylalkyl, cyano, cycloalkyl, cycloalkylalkyl, H, halo, haloalkyl, heteroaryl, heteroarylalkyl, heterocycloalkyl, heterocycloalkylalkyl, and hydroxyl, wherein two R⁶ groups together with the atoms to which they are attached optionally form an aryl, cycloalkyl, heteroaryl, or heterocycloalkyl ring, which may be optionally substituted with between 0 and 3 R^{x} groups.

16. The compound as recited in claim 15, wherein:
n is 4; and
A¹, A2 and A³ are CH.

17. The compound as recited in claim 15, wherein:
n is 4;
A¹ and A³ are N; and
A² is CH.

18. The compound as recited in claim 15, wherein:
n is 4;
A¹ and A² are CH; and
A³ is N.

19. The compound as recited in claim 15, wherein:
n is 4;
A¹ is N;
A² is CH; and
A³ is CR².

20. The compound as recited in claim 15, wherein:
Z¹ is C;
Z² and Z³ are N;
Z⁴ is CH;
R⁴ is chosen from N(R³)₂, NR³C(O)C(R³)₃, NR³C(O)OC(R³)₃, and NR³C(O)N(R³)₂; and
R⁵ is H.

21. The compound as recited in claim 15, wherein:
n is 4;
A¹ is N;
A² is CH;
A³ is CR²;
Z¹ is C;
Z² and Z³ are N;
Z⁴ is CH;
R⁴ is chosen from N(R³)₂, NR³C(O)C(R³)₃, NR³C(O)OC(R³)₃, and NR³C(O)N(R³)₂; and
R⁵ is H.

22. The compound as recited in claim 15, wherein:
Z¹ is C;
Z² and Z³ are N;
Z⁴ is CH;
R⁴ is C(O)N(R³)₂; and
R⁵ is H.

23. The compound as recited in claim 15, wherein:
n is 4;
A¹ is N;
A² is CH;
A³ is CR²;
Z¹ is C;
Z² and Z³ are N;
Z⁴ is CH;
R⁴ is C(O)N(R³)₂; and
R⁵ is H.

24. The compound as recited in claim 15, wherein:
Z¹ is N;
Z² is C(O);
Z⁴ is CH;
R⁴ is chosen from N(R³)₂, NR³C(O)C(R³)₃, NR³C(O)OC(R³)₃, and NR³C(O)N(R³)₂; and
R⁵ is H.

25. The compound as recited in claim 15, wherein:
n is 4;
A¹ is N;
A² is CH;
A³ is CR²;
Z¹ is N;
Z² is C(O);
Z⁴ is CH;
R⁴ is chosen from N(R³)₂, NR³C(O)C(R³)₃, NR³C(O)OC(R³)₃, and NR³C(O)N(R³)₂; and
R⁵ is H.

26. The compound as recited in claim 15, wherein:
Z¹ is N;
Z² is C(O);
Z⁴ is CH;
R⁴ is C(O)N(R³)₂; and
R⁵ is H.

27. The compound as recited in claim 15, wherein:
n is 4;
A¹ is N;
A² is CH;
A³ is CR²;
Z¹ is N;
Z² is C(O);
Z⁴ is CH;
R⁴ is C(O)N(R³)₂; and
R⁵ is H.

28. A pharmaceutical composition comprising a compound as recited in claim 1 and a pharmaceutically acceptable carrier, adjuvant, or vehicle.

29. The compound as recited in claim 1, or a salt thereof, wherein the compound is chosen from Examples 1-244.

30. A method of inhibiting GLS1 activity in a biological sample comprising contacting the biological sample with a compound as recited in claim 1.

31. A method of treating a GLS1-mediated disorder in a subject in need thereof, comprising the step of administering to the subject a compound as recited in claim 1.

32. The method as recited in claim 31, wherein the subject is a human.

33. The method as recited in claim 31, wherein the GLS1-mediated disorder is chosen from cancer, immunological disorders, and neurological disorders.

34. The method as recited in claim 33, wherein the GLS1-mediated disorder is cancer.

35. The method as recited in claim 34, wherein the cancer is chosen from Acute Lymphoblastic Leukemia (ALL), Acute Myeloid Leukemia (AML), Adrenocortical Carcinoma, AIDS-Related Cancers (Kaposi Sarcoma and Lymphoma), Anal Cancer, Appendix Cancer, Atypical Teratoid/Rhabdoid Tumor, Basal Cell Carcinoma, Bile Duct Cancer (including Extrahepatic), Bladder Cancer, Bone Cancer (including Osteosarcoma and Malignant Fibrous Histiocytoma), Brain Tumor (such as Astrocytomas, Brain and Spinal Cord Tumors, Brain Stem Glioma, Central Nervous System Atypical Teratoid/Rhabdoid Tumor, Central Nervous System Embryonal Tumors, Craniopharyngioma, Ependymoblastoma, Ependymoma, Medulloblastoma, Medulloepithelioma, Pineal Parenchymal Tumors of Intermediate Differentiation, Supratentorial Primitive Neuroectodermal Tumors and Pineoblastoma), Breast Cancer, Bronchial Tumors, Burkitt Lymphoma, Basal Cell Carcinoma, Bile Duct Cancer (including Extrahepatic), Bladder Cancer, Bone Cancer (including Osteosarcoma and Malignant Fibrous Histiocytoma), Carcinoid Tumor, Carcinoma of Unknown Primary, Central Nervous System (such as Atypical Teratoid/Rhabdoid Tumor, Embryonal Tumors and Lymphoma), Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma (Mycosis Fungoides and Sézary Syndrome), Duct, Bile (Extrahepatic), Ductal Carcinoma In Situ (DCIS), Embryonal Tumors (Central Nervous System), Endometrial Cancer, Ependymoblastoma, Ependymoma, Esophageal Cancer, Esthesioneuroblastoma, Ewing Sarcoma Family of Tumors, Extracranial Germ Cell Tumor, Extragonadal Germ Cell Tumor, Extrahepatic Bile Duct Cancer, Eye Cancer (like Intraocular Melanoma, Retinoblastoma), Fibrous Histiocytoma of Bone (including Malignant and Osteosarcoma) Gallbladder Cancer, Gastric (Stomach) Cancer, Gastrointestinal Carcinoid Tumor, Gastrointestinal Stromal Tumors (GIST), Germ Cell Tumor (Extracranial, Extragonadal, Ovarian), Gestational Trophoblastic Tumor, Glioma, Hairy Cell Leukemia, Head and Neck Cancer, Heart Cancer, Hepatocellular (Liver) Cancer, Histiocytosis, Langerhans Cell, Hodgkin Lymphoma, Hypopharyngeal Cancer, Intraocular Melanoma, Islet Cell Tumors (Endocrine, Pancreas), Kaposi Sarcoma, Kidney (including Renal Cell), Langerhans Cell Histiocytosis, Laryngeal Cancer, Leukemia (including Acute Lymphoblastic (ALL), Acute Myeloid (AML), Chronic Lymphocytic (CLL), Chronic Myelogenous (CML), Hairy Cell), Lip and Oral Cavity Cancer, Liver Cancer (Primary), Lobular Carcinoma In Situ (LCIS), Lung Cancer (Non-Small Cell and Small Cell), Lymphoma (AIDS-Related, Burkitt, Cutaneous T-Cell (Mycosis Fungoides and Sézary Syndrome), Hodgkin, Non-Hodgkin, Primary Central Nervous System (CNS), Macroglobulinemia, Waldenström, Male Breast Cancer, Malignant Fibrous Histiocytoma of Bone and Osteosarcoma, Medulloblastoma, Medulloepithelioma, Melanoma (including Intraocular (Eye)), Merkel Cell Carcinoma, Mesothelioma (Malignant), Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic (CML), Myeloid Leukemia, Acute (AML), Myeloma and Multiple Myeloma, Myeloproliferative Disorders (Chronic), Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and, Oropharyngeal Cancer, Osteosarcoma and Malignant Fibrous Histiocytoma of Bone, Ovarian Cancer (such as Epithelial, Germ Cell Tumor, and Low Malignant Potential Tumor), Pancreatic Cancer (including Islet Cell Tumors), Papillomatosis, Paraganglioma, Paranasal Sinus and Nasal Cavity Cancer, Parathyroid Cancer, Penile Cancer, Pharyngeal Cancer, Pheochromocytoma, Pineal Parenchymal Tumors of Intermediate Differentiation, Pineoblastoma and Supratentorial Primitive Neuroectodermal Tumors, Pituitary Tumor, Plasma Cell Neoplasm/Multiple Myeloma, Pleuropulmonary Blastoma, Pregnancy and Breast Cancer, Primary Central Nervous System (CNS) Lymphoma, Prostate Cancer, Rectal Cancer, Renal Cell (Kidney) Cancer, Renal Pelvis and Ureter, Transitional Cell Cancer, Retinoblastoma, Rhabdomyosarcoma, Salivary Gland Cancer, Sarcoma (like Ewing Sarcoma Family of Tumors, Kaposi, Soft Tissue, Uterine), Sézary Syndrome, Skin Cancer (such as Melanoma, Merkel Cell Carcinoma, Nonmelanoma), Small Cell Lung Cancer, Small Intestine Cancer, Soft Tissue Sarcoma, Squamous Cell Carcinoma, Squamous Neck Cancer with Occult Primary, Metastatic, Stomach (Gastric) Cancer, Supratentorial Primitive Neuroectodermal Tumors, T-Cell Lymphoma (Cutaneous, Mycosis Fungoides and Sézary Syndrome), Testicular Cancer, Throat Cancer, Thymoma and Thymic Carcinoma, Thyroid Cancer, Transitional Cell Cancer of the Renal Pelvis and Ureter, Trophoblastic Tumor (Gestational), Unknown Primary, Unusual Cancers of Childhood, Ureter and Renal Pelvis, Transitional Cell Cancer, Urethral Cancer, Uterine Cancer, Endometrial, Uterine Sarcoma, Waldenström Macroglobulinemia and Wilms Tumor, or a variant thereof.

36. A method of treating a GLS1-mediated disorder in a subject in need thereof, comprising the sequential or co-administration of a compound as recited in claim 1 or a pharmaceutically acceptable salt thereof, and another therapeutic agent.

37. The method as recited in claim 36, wherein the therapeutic agent is chosen from a taxane, inhibitor of bcr-abl, inhibitor of EGFR, DNA damaging agent, and antimetabolite.

38. The method as recited in claim 36, wherein the therapeutic agent is chosen from aminoglutethimide, amsacrine, anastrozole, asparaginase, bcg, bicalutamide, bleomycin, buserelin, busulfan, campothecin, capecitabine, carboplatin, carmustine, chlorambucil, chloroquine, cisplatin, cladribine, clodronate, colchicine, cyclophosphamide, cyproterone, cytarabine, dacarbazine, dactinomycin, daunorubicin, demethoxyviridin, dichloroacetate, dienestrol, diethylstilbestrol, docetaxel, doxorubicin, epirubicin, estradiol, estramustine, etoposide, everolimus, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, lonidamine, mechlorethamine, medroxyprogesterone, megestrol, melphalan, mercaptopurine, mesna, metformin, methotrexate, mitomycin, mitotane, mitoxantrone, nilutamide, nocodazole, octreotide, oxaliplatin, paclitaxel, pamidronate, pentostatin, perifosine, plicamycin, porfimer, procarbazine, raltitrexed, rituximab, sorafenib, streptozocin, sunitinib, suramin, tamoxifen, temozolomide, temsirolimus, teniposide, testosterone, thioguanine, thiotepa, titanocene dichloride, topotecan, trastuzumab, tretinoin, vinblastine, vincristine, vindesine, and vinorelbine.

39. The method as recited in claim 34, wherein the method further comprises administering non-chemical methods of cancer treatment.

40. The method as recited in claim 39, wherein the method further comprises administering radiation therapy.

41. The method as recited in claim 40, wherein the method further comprises administering surgery, thermoablation, focused ultrasound therapy, cryotherapy, or any combination thereof.

42. A compound of any as recited in claim 1 for use in human therapy.

43. A compound of any as recited in claim 1 for use in treating a GLS1-mediated disease.

44. Use of a compound as recited in claim 1 for the manufacture of a medicament to treat a GLS1-mediated disease.
